(19) United States
(12) Patent Application Publication
(10) Pub. No.: US 2015/0152187 A1
(43) Pub. Date: Jun. 4, 2015

(54) Anti-PSMA Antibodies Conjugated to Nuclear Receptor Ligand Polypeptides

(71) Applicant: Ambrx, Inc., La Jolla, CA (US)

(72) Inventors: Ying Sun, San Diego, CA (US); Ning Zou, San Diego, CA (US); Amha Hewet, Chula Vista, CA (US); Jason K. Pinkstaff, San Rafael, CA (US); Shailaja Srinagesh, San Mateo, CA (US); Richard S. Barnett, San Marcos, CA (US); Feng Tian, San Diego, CA (US); Anna-Maria A. Hays Putnam, San Diego, CA (US); Marco Gymnopoulos, San Diego, CA (US); Nick Knudsen, Escondido, CA (US); Andrew Beck, San Diego, CA (US)

(73) Assignee: Ambrx, Inc., La Jolla, CA (US)

(21) Appl. No.: 14/407,792

(22) PCT Filed: Jun. 14, 2013
(86) PCT No.: PCT/US2013/045834

§ 371 Date: Dec. 12, 2014

Related U.S. Application Data
(60) Provisional application No. 61/659,937, filed on Jun. 14, 2012.
(60) Provisional application No. 61/766,564, filed on Feb. 19, 2013.
(60) Provisional application No. 61/806,338, filed on Mar. 28, 2013.

(51) Int. Cl. C07K 16/30; A61K 47/48

(52) U.S. Cl. CPC C07K 16/3076; A61K 47/48715; A61K 47/48638; C07K 2317/92; A61K 2039/505

(57) ABSTRACT

This invention relates to anti-prostate-specific membrane antigen antibodies (αPSMA) and αPSMA antibody—nuclear receptor ligand (NRL) conjugates comprising at least one non-naturally-encoded amino acid.

### FIELD OF THE INVENTION

This invention relates to anti-prostate-specific membrane antigen antibodies (αPSMA) and αPSMA antibody—nuclear receptor ligand (NRL) conjugates comprising at least one non-naturally-encoded amino acid.

### BACKGROUND OF THE INVENTION

Prostate cancer is the most commonly diagnosed non-skin related malignancy in males in developed countries. It is estimated that one in six males will be diagnosed with prostate cancer. The diagnosis of prostate cancer has greatly improved following the use of serum-based markers such as the prostate-specific antigen (PSA). In addition, prostate tumor-associated antigens offer targets for tumor imaging, diagnosis, and targeted therapies. The prostate specific membrane antigen (PSMA), a prostate tumor associated marker, is such a target.

PSMA is a glycoprotein highly restricted to prostate secretory epithelial cell membranes. Its expression level has been correlated with tumor aggressiveness. Various immunohistological studies have demonstrated increased PSMA levels in virtually all cases of prostatic carcinoma compared to those levels in benign prostate epithelial cells. Intense PSMA staining is found in all stages of the disease, including prostatic intraepithelial neoplasia, late stage androgen-independent prostate cancer and secondary prostate tumors localized to lymph nodes, bone, soft tissue, and lungs.

PSMA forms a noncovalent homodimer that possesses glutamate carboxypeptidase activity based on its ability to process the neuropeptide N-acetylaspartylglutamate and glutamate-conjugated folate derivatives. Although the precise biological role played by PSMA in disease pathogenesis remains unknown, its overexpression in prostate tumors is well known. It has been suggested that PSMA performs multiple physiological functions related to cell survival and migration.

Antibody-based therapeutics have emerged as important components of therapies for an increasing number of human malignancies in such fields as oncology, inflammatory and infectious diseases. In most cases, the basis of the therapeutic function is the high degree of specificity and affinity the antibody-based drug has for its target antigen. Arming monoclonal antibodies with drugs, toxins, or radionuclides is yet another strategy by which mAbs may induce therapeutic effect. By combining the exquisite targeting specificity of antibody with the tumor killing power of toxic effector molecules, immunoconjugates permit sensitive discrimination between target and normal tissue thereby resulting in fewer side effects than most conventional chemotherapeutic drugs.

Given the physical properties of PSMA and its expression pattern in relation to prostate cancer progression PSMA is an excellent target in the development of antibody-drug conjugates for imaging, diagnostic and therapeutic uses. The first PSMA-specific MAb reported, 7E11, was subsequently developed and commercialized as a diagnostic agent for tumor imaging (ProstaScint, Cytogen, Princeton, N.J.). However, this antibody recognizes an intracellular epitope of PSMA which limits its usefulness as an imaging agent for the detection of PSMA. More recently, MAbs such as J591 that recognize the extracellular portion of PSMA have been identified. Anti-PSMA antibody conjugates that can be utilized for imaging, diagnostic and/or therapeutic uses are therefore needed. The present invention provides such antibody conjugates for use in prostate cancer.

### SUMMARY OF THE INVENTION

Provided herein are targeting moiety peptides conjugated to glucocorticoids and glucocorticoid analogs via a linker. In some embodiments, the targeting moiety is an anti-prostate-specific membrane antigen antibody. In some embodiments, the glucocorticoids and glucocorticoid analogs (also referred to as nuclear receptor ligands or NRLs) may include, but are not limited to, FK506, rapamycin, cyclosporine A, dasatinib, dexamethasone, and analogs. By way of non-limiting example, the present invention includes:

AFg-L₁-L₂-D)ₘ
wherein A is an αPSMA antibody;
Fg is functional group connecting antibody and linker, which is selected from:

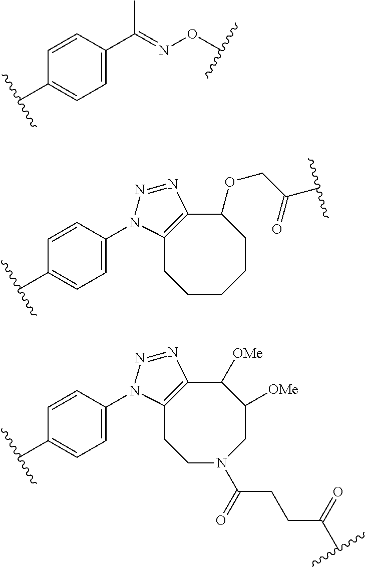 [SMILES: *O/N=C(\C)c1ccc(*)cc1 ; *C(=O)COC1CCCCCc2c1nnn2-c1ccc(*)cc1 ; *C(=O)CCC(=O)N1CCc2c(nnn2-c2ccc(*)cc2)C(OC)C(OC)C1]

L1 and L2 are linkers;
D is selected from: glucocorticoids; fluorinated 4-azasteroids; fluorinated 4-azasteroids derivatives; antiandrogens; alpha-substituted steroids; carbonylamino-benzimidazole; 17-hydroxy 4-aza androstan-3-ones; antiandrogenic biphenyls; goserelin; nilutamid; decursin; flutamide; p,p′-DDE; vinclozolin; cyproterone acetate; linuron; kinase inhibitors; staurosporine, saracatinib, fingolimod, and dexamethasone:

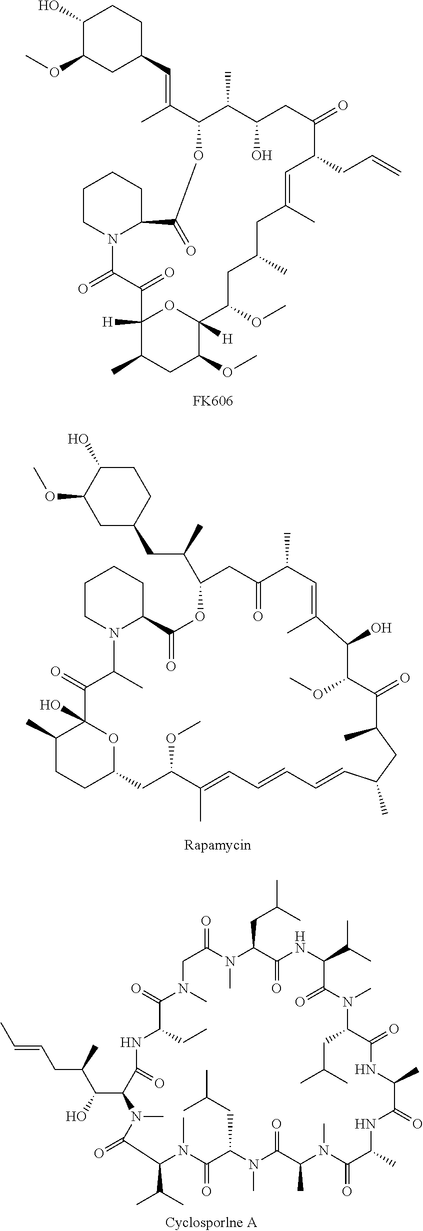

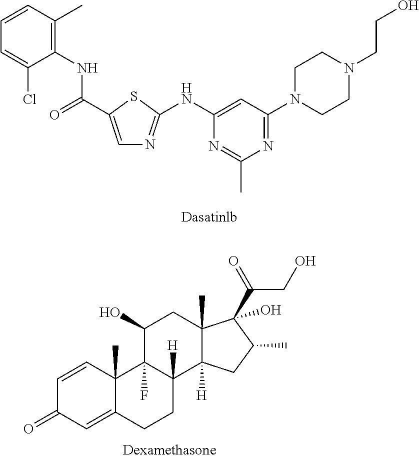

m=1-4

In some of the embodiments of the present invention,

G-L₁-L₂-D
wherein G is functional group for conjugation to connect antibody and linker, which is selected from:

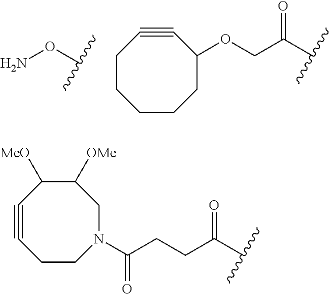

L1 is selected from
J is selected from: —C₁-C₃₀ alkylene-, —C₂-C₃₀ alkenylene-containing 0 to 20 heteroatoms selected from O, S or N; substituted —C₁-C₃₀ alkylene, substituted —C₂-C₃₀ alkenylene containing 0 to 20 heteroatoms selected from O, S or N;
W is selected from none, —CO—, —NHCO—, —OCO—
L2 is selected from -(E-Q)ₖ-,
E is an enzyme cleavage substrate: a dipeptide up to hexapeptide with or without para aminobenzyl alcohol, selected from:
-ValCit-(p-amino-benzylalcohol-CO)k-, -ValLys-(p-amino-benzylalcohol-CO)k-,
-ValArg-(p-amino-benzylalcohol-CO)k-, -PheLys-(p-amino-benzylalcohol-CO)k-,
-PheArg-(p-amino-benzylalcohol-CO)k-,
k=0.1;
Q is a spacer, selected from:

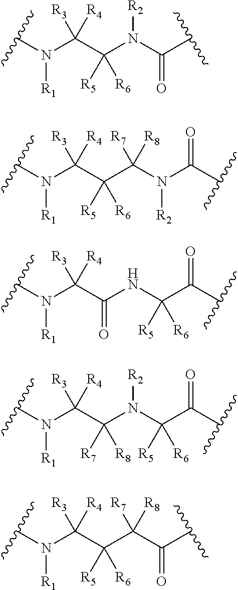

and R1, R2, R3, R4, R5, R6, R7, R8 is independently selected from H, CH3, (C1-C6) alkyl

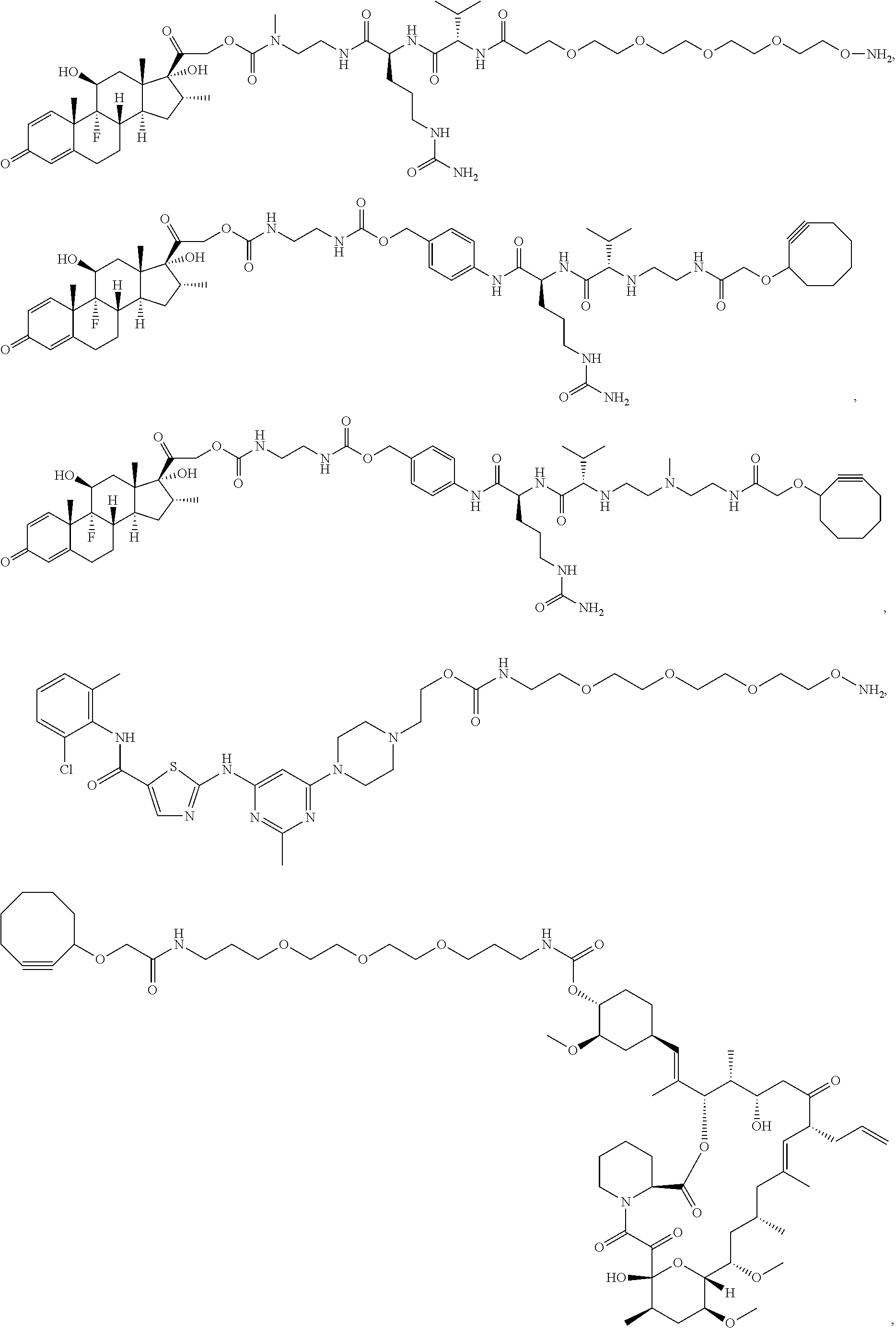

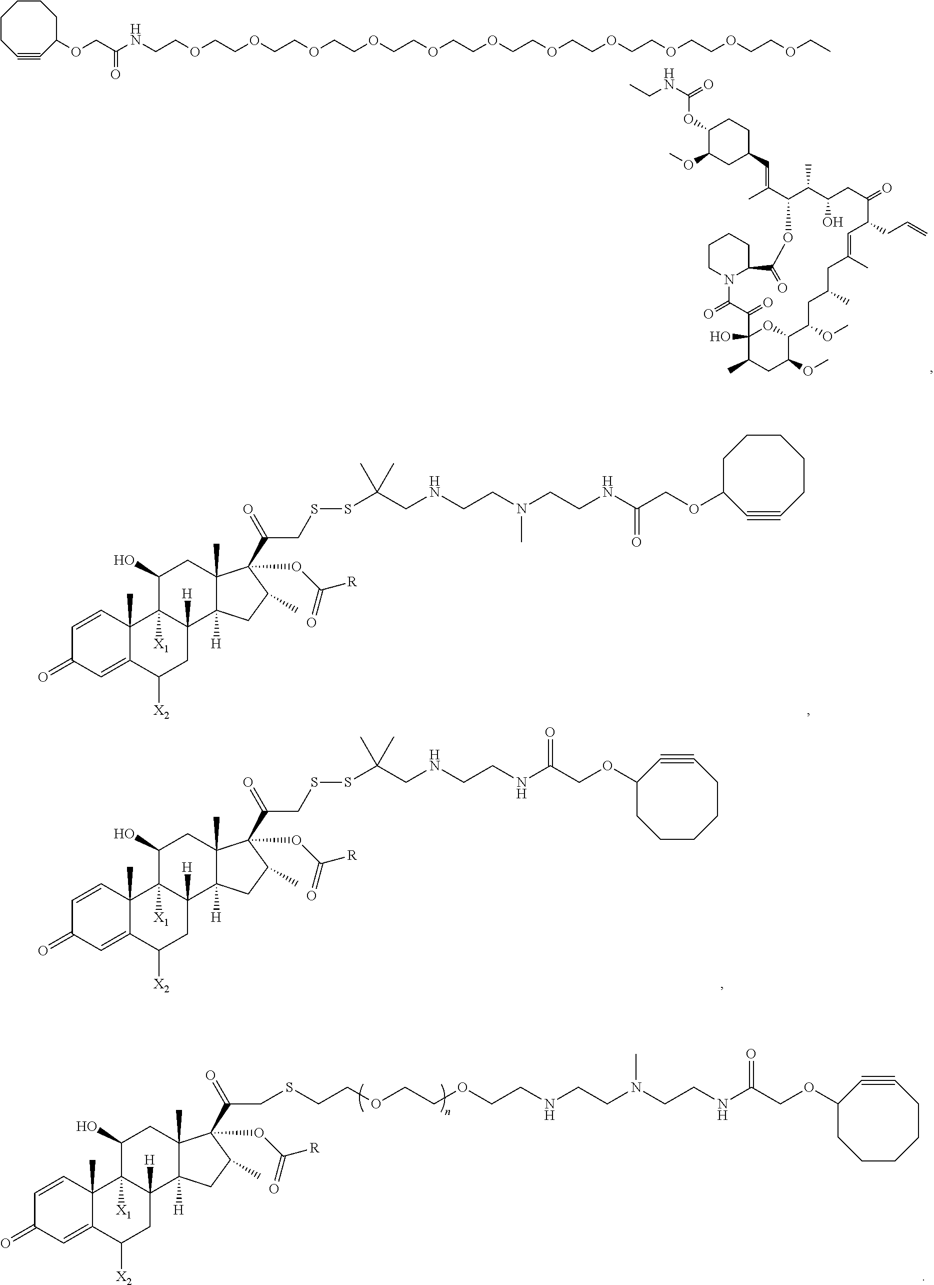

These conjugates with plural activities are useful for the treatment of a variety of diseases,

The nuclear receptor ligand conjugates of the invention can also be represented by the following formula:

Ab-L-Y

wherein Ab is a targeting moiety peptide, in comes embodiments an αPSMA antibody; Y is a nuclear receptor ligand (NRL); and L is a linking group or a bond.

In some embodiments, Ab is a polypeptide. In specific embodiments, the polypeptide is an antibody. In certain specific embodiments, the antibody is αPSMA. The activity of the antibody at the receptor can be in accordance with any of the teachings set forth herein.

The nuclear receptor ligand (Y) is wholly or partly non-peptidic and acts at a nuclear receptor or nuclear hormone receptor with an activity in accordance with any of the teachings set forth herein. In some embodiments the NRL has an EC50 or IC50 of about 1 mM or less, or 100 μM or less, or 10 μM or less, or 1 μM or less. In some embodiments, the NRL has a molecular weight of up to about 5000 daltons, or up to about 2000 daltons, or up to about 1000 daltons, or up to about 500 daltons. The NRL may act at any of the nuclear hormone receptors described herein or have any of the structures described herein.

In some embodiments, the antibody has an EC50 (or IC50) at the receptor within about 100-fold, or within about 75-fold, or within about 50-fold, or within about 40-, 30-, 25-, 20-, 15-, 10- or 5-fold of the EC50 or IC50 of the NRL at its nuclear receptor. In some embodiments, the antibody has an EC50 (or 1050) at its receptor within about 100-fold, or within about 75-fold, or within about 50-fold, or within about 40-, 30-, 25-, 20-, 15-, 10- or 5-fold of the EC50 or 1050 of the NRL at its nuclear receptor. In some embodiments, the antibody has an EC50 (or IC50) at the receptor within about 100-fold, or within about 75-fold, or within about 50-fold, or within about 40-, 30-, 25-, 20-, 15-, 10- or 5-fold of the EC50 or 1050 of the NRL at its nuclear receptor,

In some aspects of the invention, prodrugs of Ab-L-Y are provided wherein the prodrug comprises a dipeptide prodrug element (A-B) covalently linked to an active site of Ab via an amide linkage. Subsequent removal of the dipeptide under physiological conditions and in the absence of enzymatic activity restores full activity to the Ab-L-Y conjugate.

In some aspects of the invention, pharmaceutical compositions comprising the Ab-L-Y conjugate and a pharmaceutically acceptable carrier are also provided.

In other aspects of the invention, methods are provided for administering a therapeutically effective amount of a Ab-L-Y conjugate described herein for treating a disease or medical condition in a patient. In some embodiments, the disease or medical condition is selected from the group consisting of metabolic syndrome, diabetes, obesity, liver steatosis, and a neurodegenerative disease.

Disclosed herein are embodiments of the present invention for use in the treatment of conditions related to immunology. In some embodiments of the present invention, glucocorticoids with one or more linker(s) are linked to non-natural amino acids, and methods for making such non-natural amino acids and polypeptides.

In some embodiments, a compound is described comprising Formula (XXXI-A):

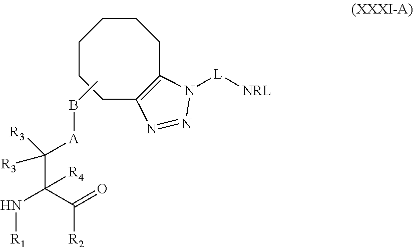

wherein:
NRL is any nuclear receptor ligand;
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R₁ is 1-1, an amino protecting group, resin, at least one amino acid, polypeptide, or polynucleotide;
R₂ is OH, an ester protecting group, resin, at least one amino acid, polypeptide, or polynucleotide;
R₃ and R₄ are each independently H, halogen, lower alkyl, or substituted lower alkyl, or R₃ and R₄ or two R₃ groups optionally form a cycloalkyl or a heterocycloalkyl;
Z has the structure of:

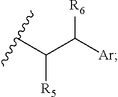

R₅ is H, CO₂H, C₁-C₆alkyl, or thiazole;
R₆ is OH or H;
Ar is phenyl or pyridine;
R₇ is C₁-C₆alkyl or hydrogen;
L is a linker selected from the group consisting of -alkylene-, -alkylene-C(O)—, -(alkylene-O)ₙ-alkylene-, -(alkylene-O)ₙ-alkylene-C(O)—, -(alkylene-O)ₙ—(CH₂)_{n′}—NHC(O)—(CH₂)_{n″}—C(Me)₂-S—S—(CH₂)_{n′″}—NHC(O)-(alkylene-O)_{n″″}-alkylene, -(alkylene-O)ₙ-alkylene-W—, -alkylene-C(O)—W—, -(alkylene-O)ₙ-alkylene-U-alkylene-C(O)—, and -(alkylene-O)ₙ-alkylene-U-alkylene-;
   W has the structure of:

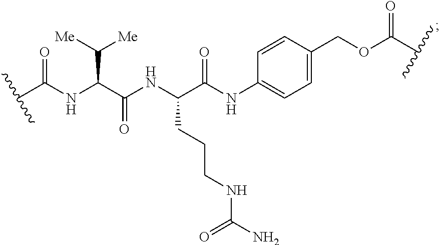 [SMILES: *C(=O)N[C@H](C(=O)N[C@@H](CCCNC(N)=O)C(=O)Nc1ccc(COC(*)=O)cc1)C(C)C]

U has the structure of:

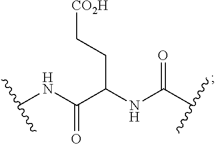 [SMILES: *NC(=O)C(CCC(=O)O)NC(*)=O]

and
each n, n′, n″, n′″ and n″″ are independently integers greater than or equal to one;

or an active metabolite, or a pharmaceutically acceptable prodrug or solvate thereof.

In certain embodiments, a pharmaceutical composition is provided comprising any of the compounds described and a pharmaceutically acceptable carrier, excipient, or binder.

In further or alternative embodiments are methods for detecting the presence of a polypeptide in a patient, the method comprising administering a polypeptide comprising at least one heterocycle-containing non-natural amino acid and the resulting heterocycle-containing non-natural amino acid polypeptide modulates the immunogenicity of the polypeptide relative to the homologous naturally-occurring amino acid polypeptide.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

As used herein and in the appended claims, the singular forms “a,” “an,” and “the” include plural reference unless the context clearly indicates otherwise.

Unless defined otherwise, all technical and scientific terms used herein have the same meaning as commonly understood to one of ordinary skill in the art to which the inventions described herein belong. Although any methods, devices, and materials similar or equivalent to those described herein can be used in the practice or testing of the inventions described herein, the preferred methods, devices and materials are now described,

All publications and patents mentioned herein are incorporated herein by reference in their entirety for the purpose of describing and disclosing, for example, the constructs and methodologies that are described in the publications, which might be used in connection with the presently described inventions. The publications discussed herein are provided solely for their disclosure prior to the filing date of the present application. Nothing herein is to be construed as an admission that the inventors described herein are not entitled to antedate such disclosure by virtue of prior invention or for any other reason.

The term “targeting moiety” as used herein, refers to any molecule or agent that specifically recognizes and binds to a cell-surface receptor, such that the targeting moiety directs the delivery of the conjugate of the present disclosures to a population of cells on which surface the receptor (e.g. PSMA, CD45, CD70, CD74, CD22) is expressed. Targeting moieties include, but are not limited to, antibodies, αPSMA antibodies, or fragments thereof, peptides, hormones, growth factors, cytokines, and any other natural or non-natural ligands, which bind to cell surface receptors (e.g., Epithelial Growth Factor Receptor (EGFR), T-cell receptor (TCR), B-cell receptor (BCR), CD28, Platelet-derived Growth Factor Receptor (PDGF), nicotinic acetylcholine receptor (nAChR), etc.).

As used herein a “linker” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties, hydrolyzable groups, and enzyme-cleavable groups. The term “linker” in some embodiments refers to any agent or molecule that bridges the conjugate of the present disclosures to the targeting moiety. One of ordinary skill in the art recognizes that sites on the conjugate of the present disclosures, which are not necessary for the function of the conjugate of the present disclosures, are ideal sites for attaching a linker and/or a targeting moiety, provided that the linker and/or targeting moiety, once attached to the conjugate of the present disclosures, do(es) not interfere with the function of the conjugate of the present disclosures, i.e., the ability to stimulate cAMP secretion from cells, to treat diabetes or obesity.

As used herein, “nuclear receptors” (NRs) refers to ligand-activated proteins that regulate gene expression within the cell nucleus, sometimes in concert with other co-activators and co-repressors. Nuclear receptors are a class of proteins found within cells that are responsible for sensing, as a non-limiting example, steroid and thyroid hormones and certain other molecules. In response, these receptors work with other proteins to regulate the expression of specific genes, thereby controlling the development, homeostasis, and metabolism of the organism, Nuclear receptors have the ability to directly bind to DNA and regulate the expression of adjacent genes, hence these receptors are classified as transcription factors. The regulation of gene expression by nuclear receptors generally only happens when a ligand—a molecule that affects the receptor's behavior—is present. More specifically, ligand binding to a nuclear receptor results in a conformational change in the receptor, which, in turn, activates the receptor, resulting in modulation, up-regulation or down-regulation, of gene expression. A unique property of nuclear receptors that differentiates them from other classes of receptors is their ability to directly interact with and control the expression of genomic DNA. As a consequence, nuclear receptors play key roles in both embryonic development and adult homeostasis. Some nuclear receptors may be classified according to either mechanism or homology.

As used herein, “NR ligand”, “nuclear receptor ligand”, and “NRL” refers to a molecule that interacts with a nuclear receptor, and may comprise a hydrophobic or lipophilic moiety and that has biological activity (either agonist or antagonist) at one or more nuclear receptor (NR). The NRL may be wholly or partly non-peptidic. In some embodiments, the NRL is an agonist that binds to and activates the NR. In other embodiments, the NRL is an antagonist. In some embodiments, the NRL is an antagonist that acts by competing with or blocking binding of native or non-native ligand to the active site. In some embodiments, the NRL is an antiandrogenic compound. In certain embodiments, the antiandrogenic NRL is selected from the group consisting of antiandrogens; alpha-substituted steroids; carbonylamino-benzimidazole; 17-hydroxy 4-aza androstan-3-ones; antiandrogenic biphenyls; goserelin; nilutamid; decursin; flutamide; p,p′-DDE; vinclozolin; cyproterone acetate; linuron. In certain embodiments, the antiandrogenic NRL is selected from the group consisting of fluorinated 4-azasteroids; fluorinated 4-azasteroids derivatives; antiandrogens; alpha-substituted steroids; carbonylamino-benzimidazole; 17-hydroxy 4-aza androstan-3-ones; antiandrogenic biphenyls; goserelin; nilutamid; decursin; flutamide; p,p′-DDE; vinclozolin; cyproterone acetate; and linuron. In other embodiments, the NRL is an antagonist that acts by binding to the active site or an allosteric site and preventing activation of, or de-activating, the NR.

As used herein, “steroids and derivatives thereof refers to compounds, either naturally occurring or synthesized, having a structure of Formula A:

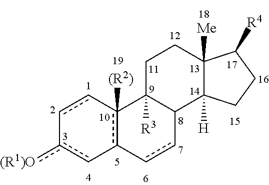

wherein R¹ and R², when present, are independently moieties that permit or promote agonist or antagonist activity upon binding of the compound of Formula A to a nuclear hormone receptor; R³ and R⁴ are independently moieties that permit or promote agonist or antagonist activity upon binding of the compound of Formula A to a nuclear hormone receptor; and each dashed line represents an optional double bond. Formula A may further comprise one or more substituents at one or more of positions 1, 2, 3, 4, 5, 6, 7, 8, 9, 11, 12, 14, 15, 16, and 17. Contemplated optional substituents include, but are not limited to, OH, NH₂, ketone, and C₁-C₁₈ alkyl groups. Specific, nonlimiting examples of steroids and derivatives thereof include cholesterol, cholic acid estradiol, testosterone, and hydrocortisone,

As used herein, “anti-androgen” refers to a group of hormone receptor antagonist compounds that are capable of preventing or inhibiting the biologic effects of androgens, male sex hormones, on normally responsive tissues in the body. An “anti-androgen” can be any pharmaceutically acceptable active agent that inhibits competitively the effect of androgens at their target site of action. Examples of antiandrogenic hormones that can be used in the present invention include, but are not limited to, coumarins, hydroxyflutamide, nilutamide, cyproterone acetate, ketoconazole, finasteride, bicalutamide, novaldex, nilandron, flutamide, progesterone, spironolactone, fluconazole, dutasteride, harman, norharman, harmine, harmaline, tetrahydroharmine, harmol, harmalol, ethyl harmol, n-butyl harmol and other beta-carboline derivatives or combinations thereof.

As used herein, “bile acids and derivatives thereof refers to compounds, either naturally occurring or synthesized, of Formula M:

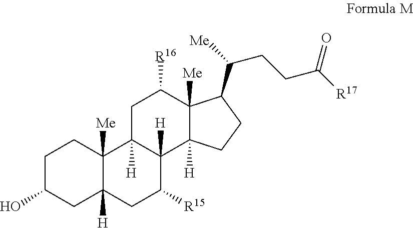

wherein each of R¹⁵, R¹⁶, and R¹⁷ are independently moieties that permit or promote agonist or antagonist activity upon binding of the compound of Formula M to a nuclear hormone receptor. In some embodiments, each of R¹⁵ and R¹⁶ are independently hydrogen, (C₀-C₈ alkyl)halo, C₁-C₁₈ alkyl, C₂-C₁₈ alkenyl, C₂-C₁₈ alkynyl, hetero alkyl, or (C₀-C₈ alkyl)OH; and R¹⁷ is OH, (C₀-C₈ alkyl)NH(C₁-C₄ alkyl)SO₃H, or (C₀-C₈ alkyl)NH(C₁-C₄ alkyl)COOH. Formula M may further comprise one or more substituents at one or more of positions 1, 2, 3, 4, 5, 6, 7, 8, 9, 11, 12, 14, 15, 16, and 17. Nonlimiting examples of bile acids include cholic acid, deoxycholic acid, lithocholic acid, chenodeoxycholic acid, taurocolic acid, and glycocholic acid.

As used herein, “cholesterol and derivatives thereof refers to compounds, either naturally occurring or synthesized, comprising a structure similar to that of cholesterol, as shown below:

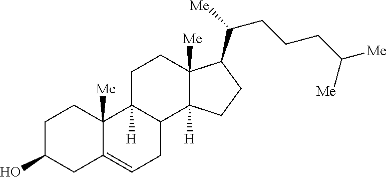

Derivatives of cholesterol can include oxysterols, such as hydroxycholesterol, 24(S)-hydroxycholesterol, 27-hydroxycholesterol, and cholestenoic acid.

As used herein, “fatty acids and derivatives thereof” refers to carboxylic acids comprising a long unbranched C₁ to C₂₈ alkyl or C₂ to C₂₈ alkenyl moiety and can optionally comprise one or more halo substituents and/or optionally comprise one or more substituents other than halo. In some embodiments, the long unbranched alkyl or alkenyl moiety can be wholly halo substituted (e.g., all hydrogens replaced with halo atoms). A short chain fatty acid comprises 1-5 carbon atoms. A medium chain fatty acid comprises 6-12 carbon. A long chain fatty acid comprises 13-22 carbon atoms. A very long chain fatty acid comprises 23-28 carbon atoms. Specific, nonlimiting examples of fatty acids include formic acid, acetic acid, n-caproic acid, heptanoic acid, caprylic acid, nonanoic acid, capric acid, undecanoic acid, laurie acid, tridecanoic acid, myristic acid, pentadeconoic acid, palmitic acid, heptadecanoic acid, stearic acid, nonadecanoic acid, arachidic acid, heneicosanoic acid, behenic acid, tricosanoic acid, mead acid, myristoleic acid, palmitoleic acid, sapienic acid, oleic acid, linoleic acid, a-linolenic acid, elaidic acid, petroselinic acid, arachidonic acid, dihydroxyeicosatetraenoic acid (DiHETE), octadecynoic acid, eicosatriynoic acid, eicosadienoic acid, eicosatrienoic acid, eicosapentaenoic acid, erucic acid, dihomolinolenic acid, docosatrienoic acid, docosapentaenoic acid, docosahexaenoic acid, and adrenic acid.

As used herein, “Cortisol and derivatives thereof refers to compounds, either naturally occurring or synthesized, of Formula C:

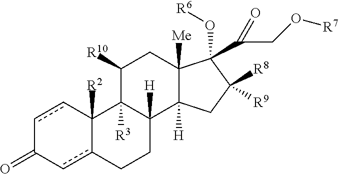

wherein R², R³, R⁶, R⁷, R⁸, R⁹, and R¹⁰ are each independently moieties that permit or promote agonist or antagonist activity upon the binding of the compound of Formula C to a nuclear hormone receptor; and each dash represents an optional double bond. In some embodiments, the structure of Formula C is substituted with one or more substituents at one or more positions of the tetracyclic ring, such as, for example, positions 1, 2, 4, 5, 6, 7, 8, 11, 12, 14, and 15. Specific, nonlimiting examples of derivatives of Cortisol and derivatives thereof include Cortisol, cortisone acetate, beclometasone, prednisone, prednisolone, methylprednisolone, betamethasone, trimcinolone, and dexamethasone,

As used herein, “linking group” is a molecule or group of molecules that binds two separate entities to one another. Linking groups may provide for optimal spacing of the two entities or may further supply a labile linkage that allows the two entities to be separated from each other. Labile linkages include hydrolyzable groups, photocleavable groups, acid-labile moieties, base-labile moieties and enzyme cleavable groups.

As used herein, a “dipeptide” is the result of the linkage of an a-amino acid or a-hydroxyl acid to another amino acid, through a peptide bond.

As used herein the term “chemical cleavage” absent any further designation encompasses a non-enzymatic reaction that results in the breakage of a covalent chemical bond.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

The terms “aldol-based linkage” or “mixed aldol-based linkage” refers to the acid- or base-catalyzed condensation of one carbonyl compound with the enolate/enol of another carbonyl compound, which may or may not be the same, to generate a β-hydroxy carbonyl compound—an aldol.

The term “affinity label,” as used herein, refers to a label which reversibly or irreversibly binds another molecule, either to modify it, destroy it, or form a compound with it. By way of example, affinity labels include enzymes and their substrates, or antibodies and their antigens.

The terms “alkoxy,” “alkylamino” and “alkylthio” (or thioalkoxy) are used in their conventional sense, and refer to those alkyl groups linked to molecules via an oxygen atom, an amino group, or a sulfur atom, respectively.

The term “alkyl,” by itself or as part of another molecule means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, pentadienyl), ethynyl, 1- and 3-propynyl, 3-butynyl, and the higher homologs and isomers. The term “alkyl,” unless otherwise noted, is also meant to include those derivatives of alkyl defined in more detail herein, such as “heteroalkyl”, “haloalkyl” and “homoalkyl”.

The term “alkylene” by itself or as part of another molecule means a divalent radical derived from an alkane, as exemplified, by (—CH₂—)ₙ, wherein n may be 1 to about 24. By way of example only, such groups include, but are not limited to, groups having 10 or fewer carbon atoms such as the structures —CH₂CH₂— and —CH₂CH₂CH₂CH₂—. A “lower alkyl” or “lower alkylene” is a shorter chain alkyl or alkylene group, generally having eight or fewer carbon atoms. The term “alkylene,” unless otherwise noted, is also meant to include those groups described herein as “heteroalkylene.”

The term “amino acid” refers to naturally occurring and non-natural amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenoeysteine. Amino acid analogs refers to compounds that have the same basic chemical structure as a naturally occurring amino acid, by way of example only, an α-carbon that is bound to a hydrogen, a carboxyl group, an amino group, and an R group. Such analogs may have modified R groups (by way of example, norleucine) or may have modified peptide backbones while still retaining the same basic chemical structure as a naturally occurring amino acid. Non-limiting examples of amino acid analogs include homoserine, norleucine, methionine sulfoxide, methionine methyl sulfonium.

Amino acids may be referred to herein by either their name, their commonly known three letter symbols or by the one-letter symbols recommended by the IUPAC-IUB Biochemical Nomenclature Commission. Additionally, nucleotides, may be referred to by their commonly accepted single-letter codes.

An “amino terminus modification group” refers to any molecule that can be attached to a terminal amine group. By way of example, such terminal amine groups may be at the end of polymeric molecules, wherein such polymeric molecules include, but are not limited to, polypeptides, polynucleotides, and polysaccharides. Terminus modification groups include but are not limited to various water soluble polymers, peptides or proteins. By way of example only, terminus modification groups include polyethylene glycol or serum albumin. Terminus modification groups may be used to modify therapeutic characteristics of the polymeric molecule, including but not limited to increasing the serum half-life of peptides.

The term “antigen-binding fragment”, as used herein, refers to one or more fragments of an antibody that retain the ability to bind to an antigen. It has been shown that the antigen-binding function of an antibody can be performed by fragments of an intact antibody. Examples of binding fragments encompassed within the term “antigen-binding fragment” of an antibody include (i) a Fab fragment, a monovalent fragment consisting of the V.sub.L, V.sub.H, C.sub.L and C.sub.H1 domains; (ii) a F(ab′).sub.2 fragment, a bivalent fragment comprising two Fab fragments linked by a disulfide bridge at the hinge region; (iii) a Fd fragment consisting of the V.sub.H and C.sub.H1 domains; (iv) a Fv fragment consisting of the V.sub.L and V.sub.H domains of a single arm of an antibody, (v) a dAb fragment (Ward et al., (1989) Nature 341:544-546), which consists of a V.sub.H domain; (vi) an isolated complementarity determining region (CDR), e.g., V.sub.H CDR3 comprising or not additional sequence (linker, framework region(s) etc.) and (v) a combination of two to six isolated CDRs comprising or not additional sequence (linker, framework region(s) etc.). Furthermore, although the two domains of the Fv fragment, V.sub.L and V.sub.H, are coded for by separate genes, they can be joined, using recombinant methods, by a synthetic linker that enables them to be made as a single polypeptide chain in which the V.sub.L and V.sub.H regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. Furthermore, the antigen-binding fragments include binding-domain immunoglobulin fusion proteins comprising (i) a binding domain polypeptide (such as a heavy chain variable region, a light chain variable region, or a heavy chain variable region fused to a light chain variable region via a linker peptide) that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The hinge region may be modified by replacing one or more cysteine residues with serine residues so as to prevent dimerization. Such binding-domain immunoglobulin fusion proteins are further disclosed in US 20030118592 and US 20030133939. These antibody fragments are obtained using conventional techniques known to those with skill in the art, and the fragments are screened for utility in the same manner as are intact antibodies.

A typical antigen binding site is comprised of the variable regions formed by the pairing of a light chain immunoglobulin and a heavy chain immunoglobulin. The structure of the antibody variable regions is very consistent and exhibits very similar structures. These variable regions are typically comprised of relatively homologous framework regions (FR) interspaced with three hypervariable regions termed Complementarity Determining Regions (CDRs). The overall binding activity of the antigen binding fragment is often dictated by the sequence of the CDRs. The FRs often play a role in the proper positioning and alignment in three dimensions of the CDRs for optimal antigen binding.

In fact, because CDR sequences are responsible for most antibody-antigen interactions, it is possible to express recombinant antibodies that shows the properties of specific naturally occurring antibodies by constructing expression vectors that include CDR sequences from the specific naturally occurring antibody grafted onto framework sequences from a different antibody with different properties (see, e.g., Riechmann, L. et al., 1998, Nature 332:323-327; Jones, P. et al., 1986, Nature 321:522-525; and Queen, C. et al., 1989, Proc. Natl. Acad. See. U.S.A. 86:10029-10033). Such framework sequences can be obtained from public DNA databases that include germline antibody gene sequences. These germline sequences will differ from mature antibody gene sequences because they will not include completely assembled variable genes, which are formed by V(D)J joining during B cell maturation. Germline gene sequences will also differ from the sequences of a high affinity secondary repertoire antibody which contains mutations throughout the variable gene but typically clustered in the CDRs. For example, somatic mutations are relatively infrequent in the amino terminal portion of framework region 1 and in the carboxy-terminal portion of framework region 4. Furthermore, many somatic mutations do not significantly alter the binding properties of the antibody. For this reason, it is not necessary to obtain the entire DNA sequence of a particular antibody in order to recreate an intact recombinant antibody having binding properties similar to those of the original antibody. Partial heavy and light chain sequence spanning the CDR regions is typically sufficient for this purpose. The partial sequence is used to determine which germline variable and joining gene segments contributed to the recombined antibody variable genes. The germline sequence is then used to fill in missing portions of the variable regions. Heavy and light chain leader sequences are cleaved during protein maturation and do not contribute to the properties of the final antibody. To add missing sequences, cloned cDNA sequences can be combined with synthetic oligonucleotides by ligation or PCR amplification. Alternatively, the entire variable region can be synthesized to create an entirely synthetic variable region clone. This process has certain advantages such as elimination or inclusion of particular restriction sites, or optimization of particular codons,

By “antibody” herein is meant a protein consisting of one or more polypeptides substantially encoded by all or part of the antibody genes. The immunoglobulin genes include, but are not limited to, the kappa, lambda, alpha, gamma (IgG1, IgG2, IgG3, and IgG4), delta, epsilon and mu constant region genes, as well as the myriad immunoglobulin variable region genes. Antibody herein is meant to include full-length antibodies and antibody fragments, and include antibodies that exist naturally in any organism or are engineered (e.g. are variants).

The term “antibody” refers to a substantially intact antibody molecule. As used herein, the phrase “antibody fragment” refers to a functional fragment of an antibody that is capable of binding to a surface marker of the present invention. Suitable antibody fragments for practicing the present invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv, an Fab, an Fab′, and an F(ab′)₂. Functional antibody fragments comprising whole or essentially whole variable regions of both light and heavy chains are defined as follows:
(i) Fv, defined as a genetically engineered fragment consisting of the variable region of the light chain and the variable region of the heavy chain expressed as two chains;
(ii) single chain Fv (“scFv”), a genetically engineered single chain molecule including the variable region of the light chain and the variable region of the heavy chain, linked by a suitable polypeptide linker.
(iii) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and C.sub.H1 domains thereof;
(iv) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule); and
(v) F(ab′)₂, a fragment of an antibody molecule containing a monovalent antigen-binding portion of an antibody molecule which can be obtained by treating whole antibody with the enzyme pepsin (i.e., a dimer of Fab′ fragments held together by two disulfide bonds).

Methods of generating antibodies (i.e., monoclonal and polyclonal) are well known in the art. Antibodies may be generated via any one of several methods known in the art, which methods can employ induction of in-vivo production of antibody molecules, screening of immunoglobulin libraries (Orlandi D. R. et al., 1989. Proc. Natl. Acad. Sci. U.S.A. 86:3833-3837; Winter G, et al., 1991. Nature 349:293-299) or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler G. et al., 1975. Nature 256:495-497; Kozbor D, et al., 1985. J. Immunol. Methods 81:31-42; Cote R J. et al., 1983. Proc. Natl. Acad. Sci. U.S.A. 80:2026-2030; Cole S P. et al., 1984. Mol. Cell. Biol. 62:109-120).

In cases where target antigens are too small to elicit an adequate immunogenic response when generating antibodies in-vivo, such antigens (haptens) can be coupled to antigenically neutral carriers such as keyhole limpet hemocyanin (KLH) or serum albumin [e.g., bovine serum albumin (BSA)] carriers (see, for example, U.S. Pat. Nos. 5,189,178 and 5,239,078]. Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art. The antisera obtained can be used directly or monoclonal antibodies may be obtained as described hereinabove. Antibody fragments can be obtained using methods well known in the art [(see, for example, Harlow and Lane, “Antibodies: A Laboratory Manual”, Cold Spring Harbor Laboratory, New York, (1988)]. For example, antibody fragments according to the present invention can be prepared by proteolytic hydrolysis of the antibody or by expression in E. coli or mammalian cells (e.g., Chinese hamster ovary cell culture or other protein expression systems) of DNA encoding the fragment.

Alternatively, antibody fragments can be obtained by pepsin or papain digestion of whole antibodies by conventional methods. As described hereinabove, an (Fab′)₂ antibody fragments can be produced by enzymatic cleavage of antibodies with pepsin to provide a 5S fragment. This fragment can be further cleaved using a thiol reducing agent, and optionally a blocking group for the sulfhydryl groups resulting from cleavage of disulfide linkages to produce 3.5S Fab′ monovalent fragments. Alternatively, enzymatic cleavage using pepsin produces two monovalent Fab′ fragments and an Fe fragment directly. Ample guidance for practicing such methods is provided in the literature of the art (for example, refer to: Goldenberg, U.S. Pat. Nos. 4,036,945 and 4,331,647; Porter, R R., 1959. Biochem. J. 73:119-126). Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

As described hereinabove, an Fv is composed of paired heavy chain variable and light chain variable domains. This association may be noncovalent (see, for example, Inbar et al., 1972. Proc. Natl. Acad. Sci. USA. 69:2659-62). Alternatively, as described hereinabove the variable domains can be linked to generate a single chain Fv by an intermolecular disulfide bond, or alternately, such chains may be cross-linked by chemicals such as glutaraldehyde. Preferably, the Fv is a single chain Fv. Single chain Fv's are prepared by constructing a structural gene comprising DNA sequences encoding the heavy chain variable and light chain variable domains connected by an oligonucleotide encoding a peptide linker. The structural gene is inserted into an expression vector, which is subsequently introduced into a host cell such as E. coli. The recombinant host cells synthesize a single polypeptide chain with a linker peptide bridging the two variable domains. Ample guidance for producing single chain Fv's is provided in the literature of the art (for example, refer to: Whitlow and Filpula, 1991. Methods 2:97-105; Bird et al., 1988. Science 242:423-426; Pack et al., 1993. BioTechnology 11:1271-77; and Ladner et al., U.S. Pat. No. 4,946,778). Isolated complementarity determining region peptides can be obtained by constructing genes encoding the complementarity determining region of an antibody of interest. Such genes may be prepared, for example, by RT-PCR of mRNA of an antibody-producing cell. Ample guidance for practicing such methods is provided in the literature of the art (for example, refer to Larrick and Fry, 1991. Methods 2:106-10).

It will be appreciated that for human therapy or diagnostics, humanized antibodies are preferably used. Humanized forms of non human (e.g., murine) antibodies are genetically engineered chimeric antibodies or antibody fragments having-preferably minimal-portions derived from non human antibodies. Humanized antibodies include antibodies in which complementary determining regions of a human antibody (recipient antibody) are replaced by residues from a complementarity determining region of a non human species (donor antibody) such as mouse, rat or rabbit having the desired functionality. In some instances, Fv framework residues of the human antibody are replaced by corresponding non human residues. Humanized antibodies may also comprise residues which are found neither in the recipient antibody nor in the imported complementarity determining region or framework sequences. In general, the humanized antibody will comprise substantially all of at least one, and typically two, variable domains, in which all or substantially all of the complementarity determining regions correspond to those of a non human antibody and all, or substantially all, of the framework regions correspond to those of a relevant human consensus sequence. Humanized antibodies optimally also include at least a portion of an antibody constant region, such as an Fe region, typically derived from a human antibody (see, for example, Jones et al., 1986. Nature 321:522-525; Riechmann et al., 1988. Nature 332:323-329; and Presta, 1992. Curr. Op. Struct. Biol. 2:593-596).

Methods for humanizing non human antibodies are well known in the art. Generally, a humanized antibody has one or more amino acid residues introduced into it from a source which is non human. These non human amino acid residues are often referred to as imported residues which are typically taken from an imported variable domain. Humanization can be essentially performed as described (see, for example: Jones et al., 1986. Nature 321; 522-525; Riechmann et al., 1988. Nature 332:323-327; Verhoeyen et al., 1988. Science 239:1534-1536; U.S. Pat. No. 4,816,567) by substituting human complementarity determining regions with corresponding rodent complementarity determining regions. Accordingly, such humanized antibodies are chimeric antibodies, wherein substantially less than an intact human variable domain has been substituted by the corresponding sequence from a non human species. In practice, humanized antibodies may be typically human antibodies in which some complementarity determining region residues and possibly some framework residues are substituted by residues from analogous sites in rodent antibodies.

Human antibodies can also be produced using various techniques known in the art, including phage display libraries [see, for example, Hoogenboom and Winter, 1991. J. Mol. Biol. 227:381; Marks et al., 1991. J. Mol. Biol. 222:581; Cole et al., “Monoclonal Antibodies and Cancer Therapy”, Alan R. Liss, pp. 77 (1985); Boerner et al., 1991, J. Immunol. 147:86-95). Humanized antibodies can also be made by introducing sequences encoding human immunoglobulin loci into transgenic animals, e.g., Into mice in which the endogenous immunoglobulin genes have been partially or completely inactivated. Upon antigenic challenge, human antibody production is observed in such animals which closely resembles that seen in humans in all respects, including gene rearrangement, chain assembly, and antibody repertoire. Ample guidance for practicing such an approach is provided in the literature of the art (for example, refer to; U.S. Pat. Nos. 5,545,807, 5,545,806, 5,569,825, 5,625,126, 5,633,425, and 5,661,016; Marks et al., 1992. BioTechnology 10:779-783; Lonberg et al., 1994. Nature 368:856-859; Morrison, 1994. Nature 368:812-13; Fishwild et al., 1996. Nature Biotechnology 14:845-51; Neuberger, 1996. Nature Biotechnology 14:826; Lonberg and Huszar, 1995. Intern. Rev. Immunol. 13:65-93). Once antibodies are obtained, they may be tested for activity, for example via ELISA. As described hereinabove, since a targeting moiety capable of targeting to essentially any desired surface marker can be obtained by the ordinarily skilled artisan, the method of the present invention may be employed to kill a target cell/tissue specifically displaying essentially any such surface marker, and, as such, can be used for treating essentially any disease associated with a cell/tissue displaying such a surface marker.

Ample guidance regarding surface markers specifically overexpressed in diseases such as cancer, and antibodies specific for such surface markers is provided in the literature of the art (for example, refer to: A M Scott, C Renner. “Tumour Antigens Recognised by Antibodies.” In: Encyclopedia of Life Sciences, Nature Publishing Group, Macmillan, London, UK, wwwdotelsdotnet, 2001). Preferably, the method is used to treat a disease associated with a target cell/tissue specifically displaying a surface marker which is a growth factor receptor and/or a tumor associated antigen (TAA).

Diseases associated with a target cell/tissue specifically displaying a growth factor receptor/TAA surface marker which are amenable to treatment by the method of the present invention include, for example, some of the numerous diseases which specifically display growth factor receptors/TAAs, such as EGF receptor, platelet derived growth factor (PDGF) receptor, insulin like growth factor receptor, vascular endothelial growth factor (VEGF) receptor, fibroblast growth factor (FGF) receptor, transferrin receptor, and folic acid receptor. Specific examples of such diseases and the growth factor receptors/TAAs which these specifically display are listed in Table 1, below.

By “antibody fragment” is meant any form of an antibody other than the full-length form. Antibody fragments herein include antibodies that are smaller components that exist within full-length antibodies, and antibodies that have been engineered. Antibody fragments include but are not limited to Fv, Fc, Fab, and (Fab′)2, single chain Fv (scFv), diabodies, triabodies, tetrabodies, bifunctional hybrid antibodies, CDR1, CDR2, CDR3, combinations of CDR's, variable regions, framework regions, constant regions, heavy chains, light chains, and variable regions, and alternative scaffold non-antibody molecules, bispecific antibodies, and the like (Maynard & Georgiou, 2000, Annu. Rev. Biomed. Eng. 2:339-76; Hudson, 1998, Curr. Opin, Biotechnol. 9; 395-402). Another functional substructure is a single chain Fv (scFv), comprised of the variable regions of the immunoglobulin heavy and light chain, covalently connected by a peptide linker (S-z Hu et al., 1996, Cancer Research, 56, 3055-3061). These small (Mr 25,000) proteins generally retain specificity and affinity for antigen in a single polypeptide and can provide a convenient building block for larger, antigen-specific molecules, Unless specifically noted otherwise, statements and claims that use the term “antibody” or “antibodies” specifically includes “antibody fragment” and “antibody fragments.”

In certain embodiments, the antibody or antigen-binding fragment thereof is selected for its ability to bind live cells, such as a tumor cell or a prostate cell, for example LNCaP cells. In other embodiments, the antibody or antigen-binding fragment thereof mediates cytolysis of cells expressing PSMA. In some embodiments cytolysis of cells expressing PSMA is mediated by effector cells or is complement mediated in the presence of effector cells.

In other embodiments, the antibody or antigen-binding fragment thereof inhibits the growth of cells expressing PSMA. In some embodiments, the antibody or antigen-binding fragment thereof does not require cell lysis to bind to the extracellular domain of PSMA.

In further embodiments, the antibody or antigen-binding fragment thereof is selected from the group consisting of IgG1, IgG2, IgG3, IgG4, IgM, IgA1, IgA2, IgAsec, IgD, IgE or has immunoglobulin constant and/or variable domain of IgG1, IgG2, IgG3, IgG4, IgM, IgA1, IgA2, IgAsec, IgD or IgE. In other embodiments, the antibody is a bispecific or multispecific antibody.

In still other embodiments, the antibody is a recombinant antibody, a polyclonal antibody, a monoclonal antibody, a humanized antibody or a chimeric antibody, or a mixture of these. In particularly preferred embodiments, the antibody is a human antibody, e.g., a monoclonal antibody, polyclonal antibody or a mixture of monoclonal and polyclonal antibodies. In still other embodiments, the antibody is a bispecific or multispecific antibody.

In one embodiment of the present invention, antigen-binding fragments include a Fab fragment, a F(ab′).sub.2 fragment, and a Fv fragment CDR3.

In certain other embodiments, the antibody or antigen-binding fragment thereof binds to a conformational epitope and/or is internalized into a cell along with the prostate specific membrane antigen. In other embodiments, the isolated antibody or antigen-binding fragment thereof is bound to a label, in some embodiments the label is selected from the group consisting of a fluorescent label, an enzyme label, a radioactive label, a nuclear magnetic resonance active label, a luminescent label, and a chromophore label.

In still other embodiments, the isolated antibody or antigen-binding fragment thereof is bound to at least one therapeutic moiety, such as a drug, preferably a cytotoxic drug, a replication-selective virus, a toxin or a fragment thereof, or an enzyme or a fragment thereof. Preferred cytotoxic drug include: calicheamicin, esperamicin, methotrexate, doxorubicin, melphalan, chlorambucil, ARA-C, vindesine, mitomycin C, cis-platinum, etoposide, bleomycin, 5-fluorouracil, estramustine, vincristine, etoposide, doxorubicin, paclitaxel, docetaxel, dolastatin 10, auristatin E and auristatin PHE. In other embodiments, the therapeutic moiety is an immunostimulatory or immunomodulating agent, preferably one selected from the group consisting of: a cytokine, chemokine and adjuvant.

In some embodiments, the antibodies or antigen-binding fragments of the invention specifically bind cell-surface PSMA and/or rsPSMA with a binding affinity of about 1×10⁻⁹M or less. In some embodiments, the binding affinity is about 1×10⁻¹⁰M or less. In some embodiments the binding affinity is about 1×10⁻¹¹M or less. In other embodiments the binding affinity is less than about 5×10⁻¹⁰M. In additional embodiments, the antibodies or antigen-binding fragments of the invention mediate specific cell killing of PSMA-expressing cells with an IC₅₀s of less than about 1×10⁻¹⁰M. In some embodiments the IC₅₀ is less than about 1×10⁻¹¹M. In some embodiments the IC₅₀ is less than about 1×10⁻¹²M. In other embodiments the IC₅₀ is less than about 1.5×10⁻¹¹M.

In one embodiment, the modified antibody or functional antibody fragment is an anti-PSMA minibody. In one embodiment, the anti-PSMA antibody is a J591 minibody. The anti-PSMA minibody has an anti-PSMA antibody fragment with optimized pharmacodynamic properties for in vivo imaging and biodistribution as described below. A “minibody” is a homodimer, wherein each monomer is a single-chain variable fragment (scFv) linked to a human IgG1 CH3 domain by a linker, such as ana hinge sequence. In another embodiment, the anti-PSMA antibody fragment comprises one non-naturally encoded amino acid. In other embodiments, the anti-PSMA minibody comprises more than one non-naturally encoded amino acid.

In another embodiment, the modified antibody or functional antibody fragment is an anti-PSMA cys-diabody (CysDB) is provided. A “diabody” comprises a first polypeptide chain which comprises a heavy (VH) chain variable domain connected to a light chain variable domain (VL) on the first polypeptide chain (VH-VL) connected by a peptide linker that is too short to allow pairing between the two domains on the first polypeptide chain and a second polypeptide chain comprising a light chain variable domain (VL) linked to a heavy chain variable domain VH on the second polypeptide chain (VL-VH) connected by a peptide linker that is too short to allow pairing between the two domains on the second polypeptide chain. In another embodiment, the diabody comprises a non-naturally encoded amino acid. In another embodiment, the diabody contains more than one non-naturally encoded amino acid. The short linkages force chain pairing between the complementary domains of the first and the second polypeptide chains and promotes the assembly of a dimeric molecule with two functional antigen binding sites. Therefore, a peptide linker may be any suitable length that promotes such assembly, for example, between 5 and 10 amino acids in length. As described further below, some cys-diabodies may include a peptide linker that is 5 or 8 amino acids in length. In another embodiment, the linker contains a non-naturally encoded amino acid. In other embodiments, the linker contains more than one non-naturally occurring amino acid. The anti-PSMA CysDB is a homodimer antibody format formed with two identical monomers that include single chain Fv (scFv) fragments with an approximate molecular weight of 55 kDa. In one embodiment, the anti-PSMA is a J591 CysDB. Like the anti-PSMA minibodies described above, the anti-PSMA CysDBs described herein have an anti-PSMA antibody fragment with optimized pharmacodynamic properties that may be used for in vivo imaging and biodistribution.

By “antibody-drug conjugate, or “ADC”, as used herein, refers to an antibody molecule, or fragment thereof, that is covalently bonded to one or more biologically active molecule(s). The biologically active molecule may be conjugated to the antibody through a linker, polymer, or other covalent bond.

As used herein an “acylated” amino acid is an amino acid comprising an acyl group which is non-native to a naturally-occurring amino acid, regardless by the means by which it is produced. Exemplary methods of producing acylated amino acids and acylated peptides are known in the art and include acylating an amino acid before inclusion in the peptide or peptide synthesis followed by chemical acylation of the peptide. In some embodiments, the acyl group causes the peptide to have one or more of (i) a prolonged half-life in circulation, (ii) a delayed onset of action, (iii) an extended duration of action, (iv) an improved resistance to proteases, such as DPP-IV, and (v) increased potency at the glucagon superfamily peptide receptor.

As used herein, an “alkylated” amino acid is an amino acid comprising an alkyl group which is non-native to a naturally-occurring amino acid, regardless of the means by which it is produced. Exemplary methods of producing alkylated amino acids and alkylated peptides are known in the art and including alkylating an amino acid before inclusion in the peptide or peptide synthesis followed by chemical alkylation of the peptide. Without being held to any particular theory, it is believed that alkylation of peptides will achieve similar, if not the same, effects as acylation of the peptides, e.g., a prolonged half-life in circulation, a delayed onset of action, an extended duration of action, an improved resistance to proteases, such as DPP-IV, and increased potency at the glucagon superfamily peptide receptor.

The term “C₁-Cₙ alkyl” wherein n can be from 1 through 18, as used herein, represents a branched or linear alkyl group having from one to the specified number of carbon atoms. For example, C₁-C₆ alkyl represents a branched or linear alkyl group having from 1 to 6 carbon atoms, Typical C₁-C₁₈ alkyl groups include, but are not limited to, methyl, ethyl, n-propyl, iso-propyl, butyl, iso-butyl, sec-butyl, tert-butyl, pentyl, hexyl and the like. Alkyl groups optionally can be substituted, for example, with hydroxy (OH), halo, aryl, carboxyl, thio, C₃-C₈ cycloalkyl, and amino.

The term “C₀-Cₙ alkyl” wherein n can be from 1-18, as used herein, represents a branched or linear alkyl group having up to 18 carbon atoms. For example, the term “(C₀-C₆ alkyl)OH” represents a hydroxyl parent moiety attached to an alkyl substituent having up to 6 carbon atoms (e.g. —OH, —CH₂OH, —C₂H₄OH, —C₃H₆OH, —C₄H₈OH, —C₈H₁₀OH, —C₆H₁₂OH).

The term “C₂-Cₙ alkenyl” wherein n can be from 2 through 18, as used herein, represents an unsaturated branched or linear group having from 2 to the specified number of carbon atoms and at least one double bond, Examples of such groups include, but are not limited to, 1-propenyl, 2-propenyl (—CH₂—CH═CH₂), 1,3-butadienyl, (—CH═CHCH═CH₂), 1-butenyl (—CH═CHCH₂CH₃), hexenyl, pentenyl, and the like. Alkenyl groups optionally can be substituted, for example, with hydroxy (OH), halo, aryl, carboxyl, thio, C₃-C₈ cycloalkyl, and amino.

The term “C₂-Cₙ alkynyl” wherein n can be from 2 to 18, refers to an unsaturated branched or linear group having from 2 to n carbon atoms and at least one triple bond. Examples of such groups include, but are not limited to, 1-propynyl, 2-propynyl, 1-butynyl, 2-butynyl, 1-pentynyl, and the like. Alkynyl groups optionally can be substituted, for example, with hydroxy (OH), halo, aryl, carboxyl, thio, C₃-C₈ cycloalkyl, and amino.

The term “aromatic” or “aryl”, as used herein, refers to a closed ring structure which has at least one ring having a conjugated pi electron system and includes both carbocyclic aryl and heterocyclic aryl (or “heteroaryl” or “heteroaromatic”) groups. The carbocyclic or heterocyclic aromatic group may contain from 5 to 20 ring atoms. The term includes monocyclic rings linked covalently or fused-ring polycyclic (i.e., rings which share adjacent pairs of carbon atoms) groups. An aromatic group can be unsubstituted or substituted. Non-limiting examples of “aromatic” or “aryl”, groups include phenyl, 1-naphthyl, 2-naphthyl, 4-biphenyl, anthracenyl, and phenanthracenyl. Substituents for each of the above noted aryl and heteroaryl ring systems are selected from the group of acceptable substituents described herein.

For brevity, the term “aromatic” or “aryl” when used in combination with other terms (including but not limited to, aryloxy, arylthioxy, aralkyl) includes both aryl and heteroaryl rings as defined above. Thus, the term “aralkyl” or “alkaryl” is meant to include those radicals in which an aryl group is attached to an alkyl group (including but not limited to, benzyl, phenethyl, pyridylmethyl and the like) including those alkyl groups in which a carbon atom (including but not limited to, a methylene group) has been replaced by a heteroatom, by way of example only, by an oxygen atom. Examples of such aryl groups include, but are not limited to, phenoxymethyl, 2-pyridyloxymethyl, 3-(1-naphthyloxyl)propyl, and the like.

The term “arylene”, as used herein, refers to a divalent aryl radical. Non-limiting examples of “arylene” include phenylene, pyridinylene, pyrimidinylene and thiophenylene, Substituents for arylene groups are selected from the group of acceptable substituents described herein.

A “bifunctional polymer”, also referred to as a “bifunctional linker”, refers to a polymer comprising two functional groups that are capable of reacting specifically with other moieties to form covalent or non-covalent linkages. Such moieties may include, but are not limited to, the side groups on natural or non-natural amino acids or peptides which contain such natural or non-natural amino acids. The other moieties that may be linked to the bifunctional linker or bifunctional polymer may be the same or different moieties. By way of example only, a bifunctional linker may have a functional group reactive with a group on a first peptide, and another functional group which is reactive with a group on a second peptide, whereby forming a conjugate that includes the first peptide, the bifunctional linker and the second peptide. Many procedures and linker molecules for attachment of various compounds to peptides are known. See, e.g., European Patent Application No, 188,256; U.S. Pat. Nos. 4,671,958, 4,659,839, 4,414,148, 4,699,784; 4,680,338; and 4,569,789 which are incorporated by reference herein in their entirety. A “multi-functional polymer” also referred to as a “multi-functional linker”, refers to a polymer comprising two or more functional groups that are capable of reacting with other moieties. Such moieties may include, but are not limited to, the side groups on natural or non-natural amino acids or peptides which contain such natural or non-natural amino acids. (including but not limited to, amino acid side groups) to form covalent or non-covalent linkages. A bi-functional polymer or multi-functional polymer may be any desired length or molecular weight, and may be selected to provide a particular desired spacing or conformation between one or more molecules linked to a compound and molecules it binds to or the compound.

The term “bioavailability,” as used herein, refers to the rate and extent to which a substance or its active moiety is delivered from a pharmaceutical dosage form and becomes available at the site of action or in the general circulation. Increases in bioavailability refers to increasing the rate and extent a substance or its active moiety is delivered from a pharmaceutical dosage form and becomes available at the site of action or in the general circulation. By way of example, an increase in bioavailability may be indicated as an increase in concentration of the substance or its active moiety in the blood when compared to other substances or active moieties. A non-limiting example of a method to evaluate increases in bioavailability is given in examples 21-25. This method may be used for evaluating the bioavailability of any polypeptide.

The term “biologically active molecule”, “biologically active moiety” or “biologically active agent” when used herein means any substance which can affect any physical or biochemical properties of a biological system, pathway, molecule, or interaction relating to an organism, including but not limited to, viruses, bacteria, bacteriophage, transposon, prion, insects, fungi, plants, animals, and humans. In particular, as used herein, biologically active molecules include but are not limited to any substance intended for diagnosis, cure, mitigation, treatment, or prevention of disease in humans or other animals, or to otherwise enhance physical or mental well-being of humans or animals, Examples of biologically active molecules include, but are not limited to, peptides, proteins, enzymes, small molecule drugs, hard drugs, soft drugs, prodrugs, carbohydrates, inorganic atoms or molecules, dyes, lipids, nucleosides, radionuclides, oligonucleotides, cells, viruses, liposomes, microparticles and micelles. Classes of biologically active agents that are suitable for use with the methods and compositions described herein include, but are not limited to, drugs, prodrugs, radionuclides, imaging agents, polymers, antibiotics, fungicides, anti-viral agents, anti-inflammatory agents, anti-tumor agents, cardiovascular agents, anti-anxiety agents, hormones, growth factors, steroidal agents, and the like.

By “modulating biological activity” is meant increasing or decreasing the reactivity of a polypeptide, altering the selectivity of the polypeptide, enhancing or decreasing the substrate selectivity of the polypeptide. Analysis of modified biological activity can be performed by comparing the biological activity of the non-natural polypeptide to that of the natural polypeptide.

The term “biomaterial,” as used herein, refers to a biologically-derived material, including but not limited to material obtained from bioreactors and/or from recombinant methods and techniques.

The term “biophysical probe,” as used herein, refers to probes which can detect or monitor structural changes in molecules. Such molecules include, but are not limited to, proteins and the “biophysical probe” may be used to detect or monitor interaction of proteins with other macromolecules. Examples of biophysical probes include, but are not limited to, spin-labels, a fluorophores, and photoactivatible groups,

The term “biosynthetically,” as used herein, refers to any method utilizing a translation system (cellular or non-cellular), including use of at least one of the following components: a polynucleotide, a codon, a tRNA, and a ribosome, By way of example, non-natural amino acids may be “biosynthetically incorporated” into non-natural amino acid polypeptides using the methods and techniques described herein, “In vivo generation of polypeptides comprising non-natural amino acids”, and in the non-limiting example 20. Additionally, the methods for the selection of useful non-natural amino acids which may be “biosynthetically incorporated” into non-natural amino acid polypeptides are described in the non-limiting examples 20.

The term “biotin analogue,” or also referred to as “biotin mimic”, as used herein, is any molecule, other than biotin, which bind with high affinity to avidin and/or streptavidin.

The term “carbonyl” as used herein refers to a group containing at a moiety selecting from the group consisting of —C(O)—, —S(O)—, —S(O)2-, and —C(S)—, including, but not limited to, groups containing a least one ketone group, and/or at least one aldehyde groups, and/or at least one ester group, and/or at least one carboxylic acid group, and/or at least one thioester group. Such carbonyl groups include ketones, aldehydes, carboxylic acids, esters, and thioesters. In addition, such groups may be part of linear, branched, or cyclic molecules,

The term “carboxy terminus modification group” refers to any molecule that can be attached to a terminal carboxy group. By way of example, such terminal carboxy groups may be at the end of polymeric molecules, wherein such polymeric molecules include, but are not limited to, polypeptides, polynucleotides, and polysaccharides. Terminus modification groups include but are not limited to, various water soluble polymers, peptides or proteins. By way of example only, terminus modification groups include polyethylene glycol or serum albumin. Terminus modification groups may be used to modify therapeutic characteristics of the polymeric molecule, including but not limited to increasing the serum half-life of peptides.

The term “chemically cleavable group,” also referred to as “chemically labile”, as used herein, refers to a group which breaks or cleaves upon exposure to acid, base, oxidizing agents, reducing agents, chemical initiators, or radical initiators.

The term “chemiluminescent group,” as used herein, refers to a group which emits light as a result of a chemical reaction without the addition of heat, By way of example only, luminol (5-amino-2,3-dihydro-1,4-phthalazinedione) reacts with oxidants like hydrogen peroxide (H₂O₂) in the presence of a base and a metal catalyst to produce an excited state product (3-aminophthalate, 3-APA).

The term “chromophore,” as used herein, refers to a molecule which absorbs light of visible wavelengths, UV wavelengths or IR wavelengths,

The term “cofactor,” as used herein, refers to an atom or molecule essential for the action of a large molecule. Cofactors include, but are not limited to, inorganic ions, coenzymes, proteins, or some other factor necessary for the activity of enzymes. Examples include, heme in hemoglobin, magnesium in chlorophyll, and metal ions for proteins.

“Cofolding,” as used herein, refers to refolding processes, reactions, or methods which employ at least two molecules which interact with each other and result in the transformation of unfolded or improperly folded molecules to properly folded molecules. By way of example only, “cofolding,” employ at least two polypeptides which interact with each other and result in the transformation of unfolded or improperly folded polypeptides to native, properly folded polypeptides. Such polypeptides may contain natural amino acids and/or at least one non-natural amino acid.

A “comparison window,” as used herein, refers a segment of any one of contiguous positions used to compare a sequence to a reference sequence of the same number of contiguous positions after the two sequences are optimally aligned. Such contiguous positions include, but are not limited to a group consisting of from about 20 to about 600 sequential units, including about 50 to about 200 sequential units, and about 100 to about 150 sequential units. By way of example only, such sequences include polypeptides and polypeptides containing non-natural amino acids, with the sequential units include, but are not limited to natural and non-natural amino acids. In addition, by way of example only, such sequences include polynucleotides with nucleotides being the corresponding sequential units. Methods of alignment of sequences for comparison are well-known in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

By way of example, an algorithm which may be used to determine percent sequence identity and sequence similarity are the BLAST and BLAST 2.0 algorithms, which are described in Altschul et al. (1997) Nuc, Acids Res. 25:3389-3402, and Altschul et al. (1990) J. Mol. Biol. 215:403-410, respectively. Software for performing BLAST analyses is publicly available through the National Center for Biotechnology Information. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1992) Proc. Natl. Acad. Sci. USA 89:10915) alignments (B) of 50, expectation (E) of 10, M=5, N=−4, and a comparison of both strands. The BLAST algorithm is typically performed with the “low complexity” filter turned off.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

The term “conservatively modified variants” applies to both natural and non-natural amino acid and natural and non-natural nucleic acid sequences, and combinations thereof. With respect to particular nucleic acid sequences, “conservatively modified variants” refers to those natural and non-natural nucleic acids which encode identical or essentially identical natural and non-natural amino acid sequences, or where the natural and non-natural nucleic acid does not encode a natural and non-natural amino acid sequence, to essentially identical sequences. By way of example, because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Thus by way of example every natural or non-natural nucleic acid sequence herein which encodes a natural or non-natural polypeptide also describes every possible silent variation of the natural or non-natural nucleic acid. One of ordinary skill in the art will recognize that each codon in a natural or non-natural nucleic acid (except AUG, which is ordinarily the only codon for methionine, and TGG, which is ordinarily the only codon for tryptophan) can be modified to yield a functionally identical molecule. Accordingly, each silent variation of a natural and non-natural nucleic acid which encodes a natural and non-natural polypeptide is implicit in each described sequence.

As to amino acid sequences, individual substitutions, deletions or additions to a nucleic acid, peptide, polypeptide, or protein sequence which alters, adds or deletes a single natural and non-natural amino acid or a small percentage of natural and non-natural amino acids in the encoded sequence is a “conservatively modified variant” where the alteration results in the deletion of an amino acid, addition of an amino acid, or substitution of a natural and non-natural amino acid with a chemically similar amino acid. Conservative substitution tables providing functionally similar natural amino acids are well known in the art. Such conservatively modified variants are in addition to and do not exclude polymorphic variants, interspecies homologs, and alleles of the methods and compositions described herein.

Conservative substitution tables providing functionally similar amino acids are known to those of ordinary skill in the art. The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins:Structures and Molecular Properties (W H Freeman & Co.; 2nd edition (December 1993)

The terms “cycloalkyl” and “heterocycloalkyl”, by themselves or in combination with other terms, represent, unless otherwise stated, cyclic versions of “alkyl” and “heteroalkyl”, respectively. Thus, a cycloalkyl or heterocycloalkyl include saturated, partially unsaturated and fully unsaturated ring linkages. Additionally, for heterocycloalkyl, a heteroatom can occupy the position at which the heterocycle is attached to the remainder of the molecule. The heteroatom may include, but is not limited to, oxygen, nitrogen or sulfur. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. Additionally, the term encompasses multicyclic structures, including but not limited to, bicyclic and tricyclic ring structures. Similarly, the term “heterocycloalkylene” by itself or as part of another molecule means a divalent radical derived from heterocycloalkyl, and the term “cycloalkylene” by itself or as part of another molecule means a divalent radical derived from cycloalkyl.

The term “cyclodextrin,” as used herein, refers to cyclic carbohydrates consisting of at least six to eight glucose molecules in a ring formation. The outer part of the ring contains water soluble groups; at the center of the ring is a relatively nonpolar cavity able to accommodate small molecules.

The term “cytotoxic,” as used herein, refers to a compound which harms cells.

“Denaturing agent” or “denaturant,” as used herein, refers to any compound or material which will cause a reversible unfolding of a polymer. By way of example only, “denaturing agent” or “denaturants,” may cause a reversible unfolding of a protein. The strength of a denaturing agent or denaturant will be determined both by the properties and the concentration of the particular denaturing agent or denaturant. By way of example, denaturing agents or denaturants include, but are not limited to, chaotropes, detergents, organic, water miscible solvents, phospholipids, or a combination thereof. Non-limiting examples of chaotropes include, but are not limited to, urea, guanidine, and sodium thiocyanate. Non-limiting examples of detergents may include, but are not limited to, strong detergents such as sodium dodecyl sulfate, or polyoxyethylene ethers (e.g. Tween or Triton detergents), Sarkosyl, mild non-ionic detergents (e.g., digitonin), mild cationic detergents such as N->2,3-(Dioleyoxy)-propyl-N,N,N-trimethylammonium, mild ionic detergents (e.g. sodium cholate or sodium deoxycholate) or zwitterionic detergents including, but not limited to, sulfobetaines (Zwittergent), 3-(3-chlolamidopropyl)dimethylammonio-1-propane sulfate (CHAPS), and 3-(3-chlolamidopropyl)dimethyl ammonio-2-hydroxy-1-propane sulfonate (CHAPSO). Non-limiting examples of organic, water miscible solvents include, but are not limited to, acetonitrile, lower alkanols (especially C2-C4 alkanols such as ethanol or isopropanol), or lower alkandiols (C2-C4 alkandiols such as ethylene-glycol) may be used as denaturants. Non-limiting examples of phospholipids include, but are not limited to, naturally occurring phospholipids such as phosphatidylethanolamine, phosphatidylcholine, phosphatidylserine, and phosphatidylinositol or synthetic phospholipid derivatives or variants such as dihexanoylphosphatidylcholine or diheptanoylphosphatidylcholine.

The term “desired functionality” as used herein refers to any group selected from a label; a dye; a polymer; a water-soluble polymer; a derivative of polyethylene glycol; a photocrosslinker; an affinity label; a photoaffinity label; a reactive compound; a resin; a second protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide, a water-soluble dendrimer, a cyclodextrin, a biomaterial; a nanoparticle; a spin label; a fluorophore; a metal-containing moiety; a radioactive moiety; a novel functional group; a group that covalently or noncovalently interacts with other molecules; a photocaged moiety; an actinic radiation excitable moiety; a ligand; a photoisomerizable moiety; biotin; a biotin analogue; a moiety incorporating a heavy atom; a chemically cleavable group; a photocleavable group; an elongated side chain; a carbon-linked sugar; a redox-active agent; an amino thioacid; an isotopically labeled moiety; a biophysical probe; a phosphorescent group; a chemiluminescent group; an electron dense group; a magnetic group; an intercalating group; a chromophore; an energy transfer agent; a biologically active agent (in which case, the biologically active agent can include an agent with therapeutic activity and the non-natural amino acid polypeptide or modified non-natural amino acid can serve either as a co-therapeutic agent with the attached therapeutic agent or as a means for delivery the therapeutic agent to a desired site within an organism); a detectable label; a small molecule; an inhibitory ribonucleic acid; a radionucleotide; a neutron-capture agent; a derivative of biotin; quantum dot(s); a nanotransmitter; a radiotransmitter; an abzyme, an activated complex activator, a virus, an adjuvant, an aglycan, an allergan, an angiostatin, an antihormone, an antioxidant, an aptamer, a guide RNA, a saponin, a shuttle vector, a macromolecule, a mimotope, a receptor, a reverse micelle, and any combination thereof.

The term “diamine,” as used herein, refers to groups/molecules comprising at least two amine functional groups, including, but not limited to, a hydrazine group, an amidine group, an imine group, a 1,1-diamine group, a 1,2-diamine group, a 1,3-diamine group, and a 1,4-diamine group. In addition, such groups may be part of linear, branched, or cyclic molecules.

The term “detectable label,” as used herein, refers to a label which may be observable using analytical techniques including, but not limited to, fluorescence, chemiluminescence, electron-spin resonance, ultraviolet/visible absorbance spectroscopy, mass spectrometry, nuclear magnetic resonance, magnetic resonance, and electrochemical methods.

The term “dicarbonyl” as used herein refers to a group containing at least two moieties selected from the group consisting of —C(O)—, —S(O)—, —S(O)₂—, and —C(S)—, including, but not limited to, 1,2-dicarbonyl groups, a 1,3-dicarbonyl groups, and 1,4-dicarbonyl groups, and groups containing a least one ketone group, and/or at least one aldehyde groups, and/or at least one ester group, and/or at least one carboxylic acid group, and/or at least one thioester group. Such dicarbonyl groups include diketones, ketoaldehydes, ketoacids, ketoesters, and ketothioesters. In addition, such groups may be part of linear, branched, or cyclic molecules. The two moieties in the dicarbonyl group may be the same or different, and may include substituents that would produce, by way of example only, an ester, a ketone, an aldehyde, a thioester, or an amide, at either of the two moieties.

The term “drug,” as used herein, refers to any substance used in the prevention, diagnosis, alleviation, treatment, or cure of a disease or condition.

The term “dye,” as used herein, refers to a soluble, coloring substance which contains a chromophore.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. By way of example, an agent or a compound being administered includes, but is not limited to, a natural amino acid polypeptide, non-natural amino acid polypeptide, modified natural amino acid polypeptide, or modified non-amino acid polypeptide. Compositions containing such natural amino acid polypeptides, non-natural amino acid polypeptides, modified natural amino acid polypeptides, or modified non-natural amino acid polypeptides can be administered for prophylactic, enhancing, and/or therapeutic treatments. An appropriate “effective” amount in any individual case may be determined using techniques, such as a dose escalation study.

The term “electron dense group,” as used herein, refers to a group which scatters electrons when irradiated with an electron beam. Such groups include, but are not limited to, ammonium molybdate, bismuth subnitrate cadmium iodide, 99%, carbohydrazide, ferric chloride hexahydrate, hexamethylene tetramine, 98.5%, indium trichloride anhydrous, lanthanum nitrate, lead acetate trihydrate, lead citrate trihydrate, lead nitrate, periodic acid, phosphomolybdic acid, phosphotungstic acid, potassium ferricyanide, potassium ferrocyanide, ruthenium red, silver nitrate, silver proteinate (Ag Assay: 8.0-8.5%) “Strong”, silver tetraphenylporphin (S-TPPS), sodium chloroaurate, sodium tungstate, thallium nitrate, thiosemicarbazide (TSC), uranyl acetate, uranyl nitrate, and vanadyl sulfate.

The term “energy transfer agent,” as used herein, refers to a molecule which can either donate or accept energy from another molecule. By way of example only, fluorescence resonance energy transfer (FRET) is a dipole-dipole coupling process by which the excited-state energy of a fluorescence donor molecule is non-radiatively transferred to an unexcited acceptor molecule which then fluorescently emits the donated energy at a longer wavelength.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

As used herein, the term “eukaryote” refers to organisms belonging to the phylogenetic domain Eucarya, including but not limited to animals (including but not limited to, mammals, insects, reptiles, birds, etc.), ciliates, plants (including but not limited to, monocots, dicots, and algae), fungi, yeasts, flagellates, microsporidia, and protists.

The term “fatty acid,” as used herein, refers to carboxylic acids with about C6 or longer hydrocarbon side chain.

The term “fluorophore,” as used herein, refers to a molecule which upon excitation emits photons and is thereby fluorescent.

The terms “functional group”, “active moiety”, “activating group”, “leaving group”, “reactive site”, “chemically reactive group” and “chemically reactive moiety,” as used herein, refer to portions or units of a molecule at which chemical reactions occur. The terms are somewhat synonymous in the chemical arts and are used herein to indicate the portions of molecules that perform some function or activity and are reactive with other molecules.

The term “halogen” includes fluorine, chlorine, iodine, and bromine.

The term “haloacyl,” as used herein, refers to acyl groups which contain halogen moieties, including, but not limited to, —C(O)CH₃, —C(O)CF₃, —C(O)CH₂OCH₃, and the like.

The term “haloalkyl,” as used herein, refers to alkyl groups which contain halogen moieties, including, but not limited to, —CF₃ and —CH₂CF₃ and the like.

The term “heteroalkyl,” as used herein, refers to straight or branched chain, or cyclic hydrocarbon radicals, or combinations thereof, consisting of an alkyl group and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule, Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and CH═CH—N(CH₃)—CH₃, In addition, up to two heteroatoms may be consecutive, such as, by way of example, —CH₂—NH—OCH₃ and CH₂—O—Si(CH₃)₃.

The terms “heterocyclic-based linkage” or “heterocycle linkage” refers to a moiety formed from the reaction of a dicarbonyl group with a diamine group. The resulting reaction product is a heterocycle, including a heteroaryl group or a heterocycloalkyl group. The resulting heterocycle group serves as a chemical link between a non-natural amino acid or non-natural amino acid polypeptide and another functional group. In one embodiment, the heterocycle linkage includes a nitrogen-containing heterocycle linkage, including by way of example only a pyrazole linkage, a pyrrole linkage, an indole linkage, a benzodiazepine linkage, and a pyrazalone linkage.

Similarly, the term “heteroalkylene” refers to a divalent radical derived from heteroalkyl, as exemplified, but not limited by, —CH₂—CH₂—S—CH₂—CH₂— and CH₂—S—CH₂—CH₂—NH—CH₂—. For heteroalkylene groups, the same or different heteroatoms can also occupy either or both of the chain termini (including but not limited to, alkyleneoxy, alkylenedioxy, alkyleneamino, alkylenediamino, aminooxyalkylene, and the like). Still further, for alkylene and heteroalkylene linking groups, no orientation of the linking group is implied by the direction in which the formula of the linking group is written. By way of example, the formula —C(O)₂R′— represents both C(O)₂R′— and —R′C(O)₂—.

The term “heteroaryl” or “heteroaromatic,” as used herein, refers to aryl groups which contain at least one heteroatom selected from N, O, and S; wherein the nitrogen and sulfur atoms may be optionally oxidized, and the nitrogen atom(s) may be optionally quaternized, Heteroaryl groups may be substituted or unsubstituted. A heteroaryl group may be attached to the remainder of the molecule through a heteroatom. Non-limiting examples of heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

The term “homoalkyl,” as used herein refers to alkyl groups which are hydrocarbon groups.

The term “identical,” as used herein, refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using comparison algorithms or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence, By way of example only, two or more polypeptide sequences are identical when the amino acid residues are the same, while two or more polypeptide sequences are “substantially identical” if the amino acid residues are about 60% identical, about 65% identical, about 70% identical, about 75% identical, about 80% identical, about 85% identical, about 90% identical, or about 95% identical over a specified region. The identity can exist over a region that is at least about 75 to about 100 amino acids in length, over a region that is about 50 amino acids in length, or, where not specified, across the entire sequence of a polypeptide sequence. In addition, by way of example only, two or more polynucleotide sequences are identical when the nucleic acid residues are the same, while two or more polynucleotide sequences are “substantially identical” if the nucleic acid residues are about 60% identical, about 65% identical, about 70% identical, about 75% identical, about 80% identical, about 85% identical, about 90% identical, or about 95% identical over a specified region. The identity can exist over a region that is at least about 75 to about 100 nucleic acids in length, over a region that is about 50 nucleic acids in length, or, where not specified, across the entire sequence of a polynucleotide sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The term “immunogenicity,” as used herein, refers to an antibody response to administration of a therapeutic drug. The immunogenicity toward therapeutic non-natural amino acid polypeptides can be obtained using quantitative and qualitative assays for detection of anti-non-natural amino acid polypeptides antibodies in biological fluids. Such assays include, but are not limited to, Radioimmunoassay (RIA), Enzyme-linked immunosorbent assay (ELISA), luminescent immunoassay (LIA), and fluorescent immunoassay (FIA). Analysis of immunogenicity toward therapeutic non-natural amino acid polypeptides involves comparing the antibody response upon administration of therapeutic non-natural amino acid polypeptides to the antibody response upon administration of therapeutic natural amino acid polypeptides.

The term “intercalating agent,” also referred to as “intercalating group,” as used herein, refers to a chemical that can insert into the intramolecular space of a molecule or the intermolecular space between molecules. By way of example only an intercalating agent or group may be a molecule which inserts into the stacked bases of the DNA double helix.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. Purity and homogeneity may be determined using analytical chemistry techniques including, but not limited to, polyacrylamide gel electrophoresis or high performance liquid chromatography. In addition, when a component of interest is isolated and is the predominant species present in a preparation, the component is described herein as substantially purified. The term “purified,” as used herein, may refer to a component of interest which is at least 85% pure, at least 90% pure, at least 95% pure, at least 99% or greater pure. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

The term “label,” as used herein, refers to a substance which is incorporated into a compound and is readily detected, whereby its physical distribution may be detected and/or monitored.

The term “linkage,” as used herein to refer to bonds or chemical moiety formed from a chemical reaction between the functional group of a linker and another molecule. Such bonds may include, but are not limited to, covalent linkages and non-covalent bonds, while such chemical moieties may include, but are not limited to, esters, carbonates, imines phosphate esters, hydrazones, acetals, orthoesters, peptide linkages, and oligonucleotide linkages. Hydrolytically stable linkages means that the linkages are substantially stable in water and do not react with water at useful pH values, including but not limited to, under physiological conditions for an extended period of time, perhaps even indefinitely. Hydrolytically unstable or degradable linkages means that the linkages are degradable in water or in aqueous solutions, including for example, blood. Enzymatically unstable or degradable linkages means that the linkage can be degraded by one or more enzymes. By way of example only, PEG and related polymers may include degradable linkages in the polymer backbone or in the linker group between the polymer backbone and one or more of the terminal functional groups of the polymer molecule. Such degradable linkages include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

The terms “medium” or “media,” as used herein, refer to any culture medium used to grow and harvest cells and/or products expressed and/or secreted by such cells. Such “medium” or “media” include, but are not limited to, solution, solid, semi-solid, or rigid supports that may support or contain any host cell, including, by way of example, bacterial host cells, yeast host cells, insect host cells, plant host cells, eukaryotic host cells, mammalian host cells, CHO cells, prokaryotic host cells, E. coli, or Pseudomonas host cells, and cell contents. Such “medium” or “media” includes, but is not limited to, medium or media in which the host cell has been grown into which a polypeptide has been secreted, including medium either before or after a proliferation step. Such “medium” or “media” also includes, but is not limited to, buffers or reagents that contain host cell lysates, by way of example a polypeptide produced intracellularly and the host cells are lysed or disrupted to release the polypeptide.

The term “metabolite,” as used herein, refers to a derivative of a compound, by way of example natural amino acid polypeptide, a non-natural amino acid polypeptide, a modified natural amino acid polypeptide, or a modified non-natural amino acid polypeptide, that is formed when the compound, by way of example natural amino acid polypeptide, non-natural amino acid polypeptide, modified natural amino acid polypeptide, or modified non-natural amino acid polypeptide, is metabolized. The term “pharmaceutically active metabolite” or “active metabolite” refers to a biologically active derivative of a compound, by way of example natural amino acid polypeptide, a non-natural amino acid polypeptide, a modified natural amino acid polypeptide, or a modified non-natural amino acid polypeptide, that is formed when such a compound, by way of example a natural amino acid polypeptide, non-natural amino acid polypeptide, modified natural amino acid polypeptide, or modified non-natural amino acid polypeptide, is metabolized.

The term “metabolized,” as used herein, refers to the sum of the processes by which a particular substance is changed by an organism, Such processes include, but are not limited to, hydrolysis reactions and reactions catalyzed by enzymes. Further information on metabolism may be obtained from The Pharmacological Basis of Therapeutics, 9th Edition, McGraw-Hill (1996). By way of example only, metabolites of natural amino acid polypeptides, non-natural amino acid polypeptides, modified natural amino acid polypeptides, or modified non-natural amino acid polypeptides may be identified either by administration of the natural amino acid polypeptides, non-natural amino acid polypeptides, modified natural amino acid polypeptides, or modified non-natural amino acid polypeptides to a host and analysis of tissue samples from the host, or by incubation of natural amino acid polypeptides, non-natural amino acid polypeptides, modified natural amino acid polypeptides, or modified non-natural amino acid polypeptides with hepatic cells in vitro and analysis of the resulting compounds.

The term “metal chelator,” as used herein, refers to a molecule which forms a metal complex with metal ions. By way of example, such molecules may form two or more coordination bonds with a central metal ion and may form ring structures.

The term “metal-containing moiety,” as used herein, refers to a group which contains a metal ion, atom or particle. Such moieties include, but are not limited to, cisplatin, chelated metals ions (such as nickel, iron, and platinum), and metal nanoparticles (such as nickel, iron, and platinum).

The term “moiety incorporating a heavy atom,” as used herein, refers to a group which incorporates an ion of atom which is usually heavier than carbon. Such ions or atoms include, but are not limited to, silicon, tungsten, gold, lead, and uranium.

The term “modified,” as used herein refers to the presence of a change to a natural amino acid, a non-natural amino acid, a natural amino acid polypeptide or a non-natural amino acid polypeptide. Such changes, or modifications, may be obtained by post synthesis modifications of natural amino acids, non-natural amino acids, natural amino acid polypeptides or non-natural amino acid polypeptides, or by co-translational, or by post-translational modification of natural amino acids, non-natural amino acids, natural amino acid polypeptides or non-natural amino acid polypeptides. The form “modified or unmodified” means that the natural amino acid, non-natural amino acid, natural amino acid polypeptide or non-natural amino acid polypeptide being discussed are optionally modified, that is, he natural amino acid, non-natural amino acid, natural amino acid polypeptide or non-natural amino acid polypeptide under discussion can be modified or unmodified.

As used herein, the term “modulated serum half-life” refers to positive or negative changes in the circulating half-life of a modified biologically active molecule relative to its non-modified form. By way of example, the modified biologically active molecules include, but are not limited to, natural amino acid, non-natural amino acid, natural amino acid polypeptide or non-natural amino acid polypeptide. By way of example, serum half-life is measured by taking blood samples at various time points after administration of the biologically active molecule or modified biologically active molecule, and determining the concentration of that molecule in each sample. Correlation of the serum concentration with time allows calculation of the serum half-life. By way of example, modulated serum half-life may be an increased in serum half-life, which may enable an improved dosing regimens or avoid toxic effects. Such increases in serum may be at least about two fold, at least about three-fold, at least about five-fold, or at least about ten-fold. A non-limiting example of a method to evaluate increases in serum half-life is given in example 33. This method may be used for evaluating the serum half-life of any polypeptide.

The term “modulated therapeutic half-life,” as used herein, refers to positive or negative change in the half-life of the therapeutically effective amount of a modified biologically active molecule, relative to its non-modified form. By way of example, the modified biologically active molecules include, but are not limited to, natural amino acid, non-natural amino acid, natural amino acid polypeptide or non-natural amino acid polypeptide. By way of example, therapeutic half-life is measured by measuring pharmacokinetic and/or pharmacodynamic properties of the molecule at various time points after administration. Increased therapeutic half-life may enable a particular beneficial dosing regimen, a particular beneficial total dose, or avoids an undesired effect. By way of example, the increased therapeutic half-life may result from increased potency, increased or decreased binding of the modified molecule to its target, an increase or decrease in another parameter or mechanism of action of the non-modified molecule, or an increased or decreased breakdown of the molecules by enzymes such as, by way of example only, proteases. A non-limiting example of a method to evaluate increases in therapeutic half-life is given in example 33, This method may be used for evaluating the therapeutic half-life of any polypeptide.

The term “nanoparticle,” as used herein, refers to a particle which has a particle size between about 500 nm to about 1 nm.

The term “near-stoichiometric,” as used herein, refers to the ratio of the moles of compounds participating in a chemical reaction being about 0.75 to about 1.5.

As used herein, the term “non-eukaryote” refers to non-eukaryotic organisms. By way of example, a non-eukaryotic organism may belong to the Eubacteria, (which includes but is not limited to, Escherichia coli. Thermus thermophilus, or Bacillus stearothermophilus, Pseudomonas fluorescens, Pseudomonas aeruginosa, Pseudomonas putida), phylogenetic domain, or the Archaea, which includes, but is not limited to, Methanococcus jannaschii, Methanobacterium thermoautotrophicum, Archaeoglobus fulgidus, Pyrococcus furiosus, Pyrococcus horikoshii, Aeuropyrum pernix, or Halobacterium such as Haloferax volcanii and Halobacterium species NRC-1, or phylogenetic domain.

A “non-natural amino acid” refers to an amino acid that is not one of the 20 common amino acids or pyrolysine or selenocysteine. Other terms that may be used synonymously with the term “non-natural amino acid” is “non-naturally encoded amino acid,” “unnatural amino acid,” “non-naturally-occurring amino acid,” and variously hyphenated and non-hyphenated versions thereof. The term “non-natural amino acid” includes, but is not limited to, amino acids which occur naturally by modification of a naturally encoded amino acid (including but not limited to, the 20 common amino acids or pyrrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine. Additionally, the term “non-natural amino acid” includes, but is not limited to, amino acids which do not occur naturally and may be obtained synthetically or may be obtained by modification of non-natural amino acids.

The term “nucleic acid,” as used herein, refers to deoxyribonucleotides, deoxyribonucleosides, ribonucleosides or ribonucleotides and polymers thereof in either single- or double-stranded form. By way of example only, such nucleic acids and nucleic acid polymers include, but are not limited to, (i) analogues of natural nucleotides which have similar binding properties as a reference nucleic acid and are metabolized in a manner similar to naturally occurring nucleotides; (ii) oligonucleotide analogs including, but are not limited to, PNA (peptidonucleic acid), analogs of DNA used in antisense technology (phosphorothioates, phosphoroamidates, and the like); (iii) conservatively modified variants thereof (including but not limited to, degenerate codon substitutions) and complementary sequences and sequence explicitly indicated. By way of example, degenerate codon substitutions may be achieved by generating sequences in which the third position of one or more selected (or all) codons is substituted with mixed-base and/or deoxyinosine residues (Batter et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “oxidizing agent,” as used herein, refers to a compound or material which is capable of removing an electron from a compound being oxidized. By way of example oxidizing agents include, but are not limited to, oxidized glutathione, cystine, cystamine, oxidized dithiothreitol, oxidized erythreitol, and oxygen. A wide variety of oxidizing agents are suitable for use in the methods and compositions described herein.

The term “pharmaceutically acceptable”, as used herein, refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “photoaffinity label,” as used herein, refers to a label with a group, which, upon exposure to light, forms a linkage with a molecule for which the label has an affinity. By way of example only, such a linkage may be covalent or non-covalent.

The term “photocaged moiety,” as used herein, refers to a group which, upon illumination at certain wavelengths, covalently or non-covalently binds other ions or molecules.

The term “photocleavable group,” as used herein, refers to a group which breaks upon exposure to light.

The term “photocrosslinker,” as used herein, refers to a compound comprising two or more functional groups which, upon exposure to light, are reactive and form a covalent or non-covalent linkage with two or more monomeric or polymeric molecules.

The term “photoisomerizable moiety,” as used herein, refers to a group wherein upon illumination with light changes from one isomeric form to another.

The term “polyalkylene glycol,” as used herein, refers to linear or branched polymeric polyether polyols. Such polyalkylene glycols, including, but are not limited to, polyethylene glycol, polypropylene glycol, polybutylene glycol, and derivatives thereof. Other exemplary embodiments are listed, for example, in commercial supplier catalogs, such as Shearwater Corporation's catalog “Polyethylene Glycol and Derivatives for Biomedical Applications” (2001). By way of example only, such polymeric polyether polyols have average molecular weights between about 0.05 kDa to about 100 kDa. By way of example, such polymeric polyether polyols include, but are not limited to, between about 50 Da and about 100,000 Da or more. The molecular weight of the polymer may be between about 50 Da and about 100,000 Da, including but not limited to, about 100,000 Da, about 95,000 Da, about 90,000 Da, about 85,000 Da, about 80,000 Da, about 75,000 Da, about 70,000 Da, about 65,000 Da, about 60,000 Da, about 55,000 Da, about 50,000 Da, about 45,000 Da, about 40,000 Da, about 35,000 Da, about 30,000 Da, about 25,000 Da, about 20,000 Da, about 15,000 Da, about 10,000 Da, about 9,000 Da, about 8,000 Da, about 7,000 Da, about 6,000 Da, about 5,000 Da, about 4,000 Da, about 3,000 Da, about 2,000 Da, about 1,000 Da, about 900 Da, about 800 Da, about 700 Da, about 600 Da, about 500 Da, 400 Da, about 300 Da, about 200 Da, about 100 Da, and about 50 Da. In some embodiments molecular weight of the polymer is between about 50 Da and about 50,000 Da. In some embodiments, the molecular weight of the polymer is between about 50 Da and about 40,000 Da. In some embodiments, the molecular weight of the polymer is between about 50 Da and about 1,000 Da. In some embodiments, the molecular weight of the polymer is between about 100 Da and about 500 Da. In some embodiments, the molecular weight of the polymer is between about 1,000 Da and about 40,000 Da. In some embodiments, the molecular weight of the polymer is between about 2,000 to about 50,000 Da. In some embodiments, the molecular weight of the polymer is between about 5,000 Da and about 40,000 Da. In some embodiments, the molecular weight of the polymer is between about 10,000 Da and about 40,000 Da. In some embodiments, the poly(ethylene glycol) molecule is a branched polymer. The molecular weight of the branched chain PEG may be between about 50 Da and about 100,000 Da, including but not limited to, about 100,000 Da, about 95,000 Da, about 90,000 Da, about 85,000 Da, about 80,000 Da, about 75,000 Da, about 70,000 Da, about 65,000 Da, about 60,000 Da, about 55,000 Da, about 50,000 Da, about 45,000 Da, about 40,000 Da, about 35,000 Da, about 30,000 Da, about 25,000 Da, about 20,000 Da, about 15,000 Da, about 10,000 Da, about 9,000 Da, about 8,000 Da, about 7,000 Da, about 6,000 Da, about 5,000 Da, about 4,000 Da, about 3,000 Da, about 2,000 Da, about 1,000 Da, about 900 Da, about 800 Da, about 700 Da, about 600 Da, about 500 Da, about 400 Da, about 300 Da, about 250 Da, about 200 Da, about 150 Da, about 100 Da, about 75 Da, and about 50 Da. In some embodiments, the molecular weight of the branched chain PEG is between about 50 Da and about 50,000 Da. In some embodiments, the molecular weight of the branched chain PEG is between about 100 Da and about 1,000 Da. In some embodiments, the molecular weight of the branched chain PEG is between about 5,000 Da and about 40,000 Da. In some embodiments, the molecular weight of the branched chain PEG is between about 5,000 Da and about 20,000 Da. In other embodiments, the molecular weight of the branched chain PEG is between about 2,000 to about 50,000 Da.

The term “polymer,” as used herein, refers to a molecule composed of repeated subunits. Such molecules include, but are not limited to, polypeptides, polynucleotides, or polysaccharides or polyalkylene glycols.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-natural amino acid. Additionally, such “polypeptides,” “peptides” and “proteins” include amino acid chains of any length, including full length proteins, wherein the amino acid residues are linked by covalent peptide bonds,

As used herein, “partly non-peptidic” refers to a molecule wherein a portion of the molecule is a chemical compound or substituent that has biological activity and that does not comprises a sequence of amino acids.

As used herein, “non-peptidic” refers to a molecule has biological activity and that does not comprise a sequence of amino acids.

The term “post-translationally modified” refers to any modification of a natural or non-natural amino acid which occurs after such an amino acid has been translationally incorporated into a polypeptide chain. Such modifications include, but are not limited to, co-translational in vivo modifications, co-translational in vitro modifications (such as in a cell-free translation system), post-translational in vivo modifications, and post-translational in vitro modifications.

The terms “prodrug” or “pharmaceutically acceptable prodrug,” as used herein, refers to an agent that is converted into the parent drug in vivo or in vitro, wherein which does not abrogate the biological activity or properties of the drug, and is relatively nontoxic, i.e., the material may be administered to an individual without causing undesirable biological effects or interacting in a deleterious manner with any of the components of the composition in which it is contained. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are converted into active drug within the body through enzymatic or non-enzymatic reactions. Prodrugs may provide improved physiochemical properties such as better solubility, enhanced delivery characteristics, such as specifically targeting a particular cell, tissue, organ or ligand, and improved therapeutic value of the drug. The benefits of such prodrugs include, but are not limited to, (i) ease of administration compared with the parent drug; (ii) the prodrug may be bioavailable by oral administration whereas the parent is not; and (iii) the prodrug may also have improved solubility in pharmaceutical compositions compared with the parent drug. A pro-drug includes a pharmacologically inactive, or reduced-activity, derivative of an active drug. Prodrugs may be designed to modulate the amount of a drug or biologically active molecule that reaches a desired site of action through the manipulation of the properties of a drug, such as physiochemical, biopharmaceutical, or pharmacokinetic properties. An example, without limitation, of a prodrug would be a non-natural amino acid polypeptide which is administered as an ester (the “prodrug”) to facilitate transmittal across a cell membrane where water solubility is detrimental to mobility but which then is metabolically hydrolyzed to the carboxylic acid, the active entity, once inside the cell where water solubility is beneficial. Prodrugs may be designed as reversible drug derivatives, for use as modifiers to enhance drug transport to site-specific tissues.

The term “prophylactically effective amount,” as used herein, refers that amount of a composition containing at least one non-natural amino acid polypeptide or at least one modified non-natural amino acid polypeptide prophylactically applied to a patient which will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

The term “protected,” as used herein, refers to the presence of a “protecting group” or moiety that prevents reaction of the chemically reactive functional group under certain reaction conditions. The protecting group will vary depending on the type of chemically reactive group being protected. By way of example only, (i) if the chemically reactive group is an amine or a hydrazide, the protecting group may be selected from tert-butyloxycarbonyl (t-Boc) and 9-fluorenylmethoxycarbonyl (Fmoc); (ii) if the chemically reactive group is a thiol, the protecting group may be orthopyridyldisulfide; and (iii) if the chemically reactive group is a carboxylic acid, such as butanoic or propionic acid, or a hydroxyl group, the protecting group may be benzyl or an alkyl group such as methyl, ethyl, or tert-butyl.

By way of example only, blocking/protecting groups may be selected from:

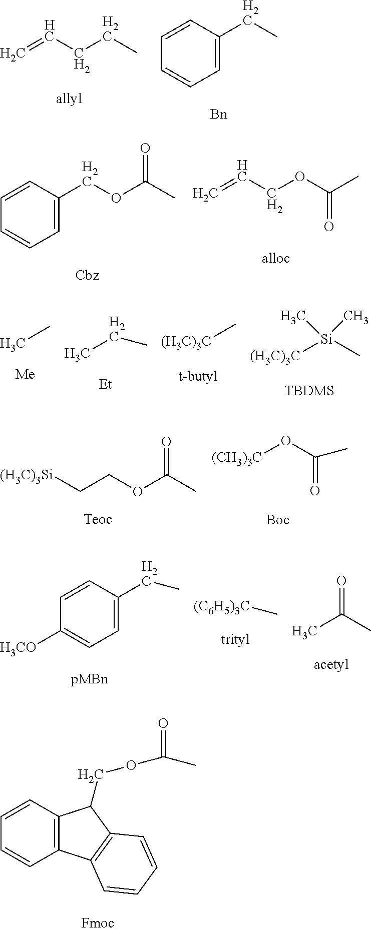

Additionally, protecting groups include, but are not limited to, including photolabile groups such as Nvoc and MeNvoc and other protecting groups known in the art. Other protecting groups are described in Greene and Wuts, Protective Groups in Organic Synthesis, 3rd Ed., John Wiley & Sons, New York, N.Y., 1999, which is incorporated herein by reference in its entirety.

The term “radioactive moiety,” as used herein, refers to a group whose nuclei spontaneously give off nuclear radiation, such as alpha, beta, or gamma particles; wherein, alpha particles are helium nuclei, beta particles are electrons, and gamma particles are high energy photons.

The term “reactive compound,” as used herein, refers to a compound which under appropriate conditions is reactive toward another atom, molecule or compound.

The term “recombinant host cell,” also referred to as “host cell,” refers to a cell which includes an exogenous polynucleotide, wherein the methods used to insert the exogenous polynucleotide into a cell include, but are not limited to, direct uptake, transduction, f-mating, or other methods known in the art to create recombinant host cells. By way of example only, such exogenous polynucleotide may be a nonintegrated vector, including but not limited to a plasmid, or may be integrated into the host genome.

The term “redox-active agent,” as used herein, refers to a molecule which oxidizes or reduces another molecule, whereby the redox active agent becomes reduced or oxidized.

Examples of redox active agent include, but are not limited to, ferrocene, quinones, Ru^{2+/3+} complexes, Co^{2+/3+} complexes, and Os^{2+/3+} complexes.

The term “reducing agent,” as used herein, refers to a compound or material which is capable of adding an electron to a compound being reduced. By way of example reducing agents include, but are not limited to, dithiothreitol (DTT), 2-mercaptoethanol, dithioerythritol, cysteine, cysteamine (2-aminoethanethiol), and reduced glutathione. Such reducing agents may be used, by way of example only, to maintain sulfhydryl groups in the reduced state and to reduce intra- or intermolecular disulfide bonds.

“Refolding,” as used herein describes any process, reaction or method which transforms an improperly folded or unfolded state to a native or properly folded conformation. By way of example only, refolding transforms disulfide bond containing polypeptides from an improperly folded or unfolded state to a native or properly folded conformation with respect to disulfide bonds. Such disulfide bond containing polypeptides may be natural amino acid polypeptides or non-natural amino acid polypeptides.

The term “resin,” as used herein, refers to high molecular weight, insoluble polymer beads. By way of example only, such beads may be used as supports for solid phase peptide synthesis, or sites for attachment of molecules prior to purification.

The term “saccharide,” as used herein, refers to a series of carbohydrates including but not limited to sugars, monosaccharides, oligosaccharides, and polysaccharides.

The term “safety” or “safety profile,” as used herein, refers to side effects that might be related to administration of a drug relative to the number of times the drug has been administered. By way of example, a drug which has been administered many times and produced only mild or no side effects is said to have an excellent safety profile. A non-limiting example of a method to evaluate the safety profile is given in example 26. This method may be used for evaluating the safety profile of any polypeptide.

The phrase “selectively hybridizes to” or “specifically hybridizes to,” as used herein, refers to the binding, duplexing, or hybridizing of a molecule to a particular nucleotide sequence under stringent hybridization conditions when that sequence is present in a complex mixture including but not limited to, total cellular or library DNA or RNA.

The term “spin label,” as used herein, refers to molecules which contain an atom or a group of atoms exhibiting an unpaired electron spin (i.e. a stable paramagnetic group) that can be detected by electron spin resonance spectroscopy and can be attached to another molecule. Such spin-label molecules include, but are not limited to, nitryl radicals and nitroxides, and may be single spin-labels or double spin-labels.

The term “stoichiometric,” as used herein, refers to the ratio of the moles of compounds participating in a chemical reaction being about 0.9 to about 1,1.

The term “stoichiometric-like,” as used herein, refers to a chemical reaction which becomes stoichiometric or near-stoichiometric upon changes in reaction conditions or in the presence of additives. Such changes in reaction conditions include, but are not limited to, an increase in temperature or change in pH. Such additives include, but are not limited to, accelerants.

The phrase “stringent hybridization conditions” refers to hybridization of sequences of DNA, RNA, PNA or other nucleic acid mimics, or combinations thereof, under conditions of low ionic strength and high temperature. By way of example, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. By way of example, longer sequences hybridize specifically at higher temperatures. Stringent hybridization conditions include, but are not limited to, (i) about 5-10° C. lower than the thermal melting point (Tm) for the specific sequence at a defined ionic strength and pH; (ii) the salt concentration is about 0.01 M to about 1.0 M at about pH 7.0 to about pH 8.3 and the temperature is at least about 30° C. for short probes (including but not limited to, about 10 to about 50 nucleotides) and at least about 60° C. for long probes (including but not limited to, greater than 50 nucleotides); (iii) the addition of destabilizing agents including, but not limited to, formamide, (iv) 50% formamide, 5×SSC, and 1% SDS, incubating at 42° C., or 5×SSC, about 1% SDS, incubating at 65° C., with wash in 0,2×SSC, and about 0.1% SDS at 65° C. for between about 5 minutes to about 120 minutes. By way of example only, detection of selective or specific hybridization, includes, but is not limited to, a positive signal at least two times background. An extensive guide to the hybridization of nucleic acids is found in Tijssen, Laboratory Techniques in Biochemistry and Molecular Biology—Hybridization with Nucleic Probes, “Overview of principles of hybridization and the strategy of nucleic acid assays” (1993).

The term “subject” as used herein, refers to an animal which is the object of treatment, observation or experiment. By way of example only, a subject may be, but is not limited to, a mammal including, but not limited to, a human.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater. By way of example only, a natural amino acid polypeptide or a non-natural amino acid polypeptide may be purified from a native cell, or host cell in the case of recombinantly produced natural amino acid polypeptides or non-natural amino acid polypeptides. By way of example a preparation of a natural amino acid polypeptide or a non-natural amino acid polypeptide may be “substantially purified” when the preparation contains less than about 30%, less than about 25%, less than about 20%, less than about 15%, less than about 10%, less than about 5%, less than about 4%, less than about 3%, less than about 2%, or less than about 1% (by dry weight) of contaminating material. By way of example when a natural amino acid polypeptide or a non-natural amino acid polypeptide is recombinantly produced by host cells, the natural amino acid polypeptide or non-natural amino acid polypeptide may be present at about 30%, about 25%, about 20%, about 15%, about 10%, about 5%, about 4%, about 3%, about 2%, or about 1% or less of the dry weight of the cells. By way of example when a natural amino acid polypeptide or a non-natural amino acid polypeptide is recombinantly produced by host cells, the natural amino acid polypeptide or non-natural amino acid polypeptide may be present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. By way of example, “substantially purified” natural amino acid polypeptides or non-natural amino acid polypeptides may have a purity level of about 30%, about 35%, about 40%, about 45%, about 50%, about 55%, about 60%, about 65%, about 70%, about 75%, about 80%, about 85%, about 90%, about 95%, about 99% or greater as determined by appropriate methods, including, but not limited to, SDSPAGE analysis, RP-HPLC, SEC, and capillary electrophoresis.

The term “substituents” also referred to as “non-interfering substituents” “refers to groups which may be used to replace another group on a molecule. Such groups include, but are not limited to, halo, C₁-C₁₀ alkyl, C₂-C₁₀ alkenyl, C₂-C₁₀ alkynyl, C₁-C₁₀ alkoxy, C₈-C₁₂ aralkyl, C₃-C₁₂ cycloalkyl, C₄-C₁₂ cycloalkenyl, phenyl, substituted phenyl, toluolyl, xylenyl, biphenyl, C₂-C₁₂ alkoxyalkyl, C₈-C₁₂ alkoxyaryl, C₈-C₁₂ aryloxyalkyl, C₇-C₁₂ oxyaryl, C₁-C₆ alkylsulfinyl, C₁-C₁₀ alkylsulfonyl, —(CH₂)ₘ—O—(C₁-C₁₀ alkyl) wherein m is from 1 to 8, aryl, substituted aryl, substituted alkoxy, fluoroalkyl, heterocyclic radical, substituted heterocyclic radical, nitroalkyl, —NO₂, —CN, —NRC(O)—(C₁-C₁₀ alkyl), —C(O)—(C₁-C₁₀ alkyl), C₂-C₁₀ alkthioalkyl, —C(O)O—(C₁-C₁₀alkyl), —OH, —SO₂, ═S, —COOH, —NR₂, carbonyl, —C(O)—(C₁-C₁₀ alkyl)-CF₃, —C(O)—CF₃, —C(O)NR₂, —(C₁-C₁₀ aryl)-S—(C₆-C₁₀ aryl), —C(O)—(C₆-C₁₀ aryl), —(CH₂)ₘ—O—(CH₂)ₘ—O—(C₁-C₁₀ alkyl) wherein each m is from 1 to 8, —C(O)NR₂, —C(S)NR₂, —SO₂NR₂, —NRC(O)NR₂, —NRC(S)NR₂, salts thereof, and the like. Each R group in the preceding list includes, but is not limited to, H, alkyl or substituted alkyl, aryl or substituted aryl, or alkaryl. Where substituent groups are specified by their conventional chemical formulas, written from left to right, they equally encompass the chemically identical substituents that would result from writing the structure from right to left; for example, —CH₂O— is equivalent to —OCH₂—.

By way of example only, substituents for alkyl and heteroalkyl radicals (including those groups referred to as alkylene, alkenyl, heteroalkylene, heteroalkenyl, alkynyl, cycloalkyl, heterocycloalkyl, cycloalkenyl, and heterocycloalkenyl) includes, but is not limited to: —OR, ═O, ═NR, ═N—OR, —NR₂, —SR, -halogen, —SiR₃, —OC(O)R, —C(O)R, —CO₂R, —CONR₂, —OC(O)NR₂, —NRC(O)R, —NRC(O)NR₂, —NR(O)₂R, —NR—C(NR₂)═NR, —S(O)R, —S(O)₂R, —S(O)₂NR₂, —NRSO₂R, —CN and —NO₂. Each R group in the preceding list includes, but is not limited to, hydrogen, substituted or unsubstituted heteroalkyl, substituted or unsubstituted aryl, including but not limited to, aryl substituted with 1-3 halogens, substituted or unsubstituted alkyl, alkoxy or thioalkoxy groups, or aralkyl groups. When two R groups are attached to the same nitrogen atom, they can be combined with the nitrogen atom to form a 5-, 6-, or 7-membered ring. For example, —NR₂ is meant to include, but not be limited to, 1-pyrrolidinyl and 4-morpholinyl.

By way of example, substituents for aryl and heteroaryl groups include, but are not limited to, —OR, ═O, ═NR, —NR₂, —SR, -halogen, —SiR₃, —OC(O)R, —C(O)R, —CO₂R, —CONR₂, —OC(O)NR₂, —NRC(O)R, —NRC(O)NR₂, —NR(O)₂R, —NR—C(NR₂)═NR, —S(O)R, —S(O)₂R, —S(O)₂NR₂, —NRSO₂R, —CN, —NO₂, —R, —N₃, —CH(Ph)₂, fluoro(C₁-C₄)alkoxy, and fluoro(C₁-C₄)alkyl, in a number ranging from zero to the total number of open valences on the aromatic ring system; and where each R group in the preceding list includes, but is not limited to, hydrogen, alkyl, heteroalkyl, aryl and heteroaryl.

The term “therapeutically effective amount,” as used herein, refers to the amount of a composition containing at least one non-natural amino acid polypeptide and/or at least one modified non-natural amino acid polypeptide administered to a patient already suffering from a disease, condition or disorder, sufficient to cure or at least partially arrest, or relieve to some extent one or more of the symptoms of the disease, disorder or condition being treated. The effectiveness of such compositions depend conditions including, but not limited to, the severity and course of the disease, disorder or condition, previous therapy, the patient's health status and response to the drugs, and the judgment of the treating physician. By way of example only, therapeutically effective amounts may be determined by routine experimentation, including but not limited to a dose escalation clinical trial.

The term “thioalkoxy,” as used herein, refers to sulfur containing alkyl groups linked to molecules via an oxygen atom.

The term “thermal melting point” or Tm is the temperature (under defined ionic strength, pH, and nucleic concentration) at which 50% of probes complementary to a target hybridize to the target sequence at equilibrium.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the term “water soluble polymer” refers to any polymer that is soluble in aqueous solvents. Such water soluble polymers include, but are not limited to, polyethylene glycol, polyethylene glycol propionaldehyde, mono C₁-C₁₀ alkoxy or aryloxy derivatives thereof (described in U.S. Pat. No. 5,252,714 which is incorporated by reference herein), monomethoxy-polyethylene glycol, polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polypropylene oxide/ethylene oxide copolymer, polyoxyethylated polyol, heparin, heparin fragments, polysaccharides, oligosaccharides, glycans, cellulose and cellulose derivatives, including but not limited to methylcellulose and carboxymethyl cellulose, serum albumin, starch and starch derivatives, polypeptides, polyalkylene glycol and derivatives thereof, copolymers of polyalkylene glycols and derivatives thereof, polyvinyl ethyl ethers, and alpha-beta-poly[(2-hydroxyethyl)-DL-aspartamide, and the like, or mixtures thereof. By way of example only, coupling of such water soluble polymers to natural amino acid polypeptides or non-natural polypeptides may result in changes including, but not limited to, increased water solubility, increased or modulated serum half-life, increased or modulated therapeutic half-life relative to the unmodified form, increased bioavailability, modulated biological activity, extended circulation time, modulated immunogenicity, modulated physical association characteristics including, but not limited to, aggregation and multimer formation, altered receptor binding, altered binding to one or more binding partners, and altered receptor dimerization or multimerization. In addition, such water soluble polymers may or may not have their own biological activity.

Unless otherwise indicated, conventional methods of mass spectroscopy, NMR, HPLC, protein chemistry, biochemistry, recombinant DNA techniques and pharmacology, within the skill of the art are employed.

Compounds, (including, but not limited to non-natural amino acids, non-natural amino acid polypeptides, modified non-natural amino acid polypeptides, and reagents for producing the aforementioned compounds) presented herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

Some of the compounds herein (including, but not limited to non-natural amino acids, non-natural amino acid polypeptides and modified non-natural amino acid polypeptides, and reagents for producing the aforementioned compounds) have asymmetric carbon atoms and can therefore exist as enantiomers or diastereomers. Diasteromeric mixtures can be separated into their individual diastereomers on the basis of their physical chemical differences by methods known, for example, by chromatography and/or fractional crystallization. Enantiomers can be separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

In additional or further embodiments, the compounds described herein (including, but not limited to non-natural amino acids, non-natural amino acid polypeptides and modified non-natural amino acid polypeptides, and reagents for producing the aforementioned compounds) are used in the form of pro-drugs. In additional or further embodiments, the compounds described herein ((including, but not limited to non-natural amino acids, non-natural amino acid polypeptides and modified non-natural amino acid polypeptides, and reagents for producing the aforementioned compounds) are metabolized upon administration to an organism in need to produce a metabolite that is then used to produce a desired effect, including a desired therapeutic effect. In further or additional embodiments are active metabolites of non-natural amino acids and “modified or unmodified” non-natural amino acid polypeptides.

The methods and formulations described herein include the use of N-oxides, crystalline forms (also known as polymorphs), or pharmaceutically acceptable salts of non-natural amino acids, non-natural amino acid polypeptides and modified non-natural amino acid polypeptides. In certain embodiments, non-natural amino acids, non-natural amino acid polypeptides and modified non-natural amino acid polypeptides may exist as tautomers. All tautomers are included within the scope of the non-natural amino acids, non-natural amino acid polypeptides and modified non-natural amino acid polypeptides presented herein. In addition, the non-natural amino acids, non-natural amino acid polypeptides and modified non-natural amino acid polypeptides described herein can exist in unsolvated as well as solvated forms with pharmaceutically acceptable solvents such as water, ethanol, and the like. The solvated forms of the non-natural amino acids, non-natural amino acid polypeptides and modified non-natural amino acid polypeptides presented herein are also considered to be disclosed herein.

Some of the compounds herein (including, but not limited to non-natural amino acids, non-natural amino acid polypeptides and modified non-natural amino acid polypeptides and reagents for producing the aforementioned compounds) may exist in several tautomeric forms. All such tautomeric forms are considered as part of the compositions described herein. Also, for example all enol-keto forms of any compounds (including, but not limited to non-natural amino acids, non-natural amino acid polypeptides and modified non-natural amino acid polypeptides and reagents for producing the aforementioned compounds) herein are considered as part of the compositions described herein.

Some of the compounds herein (including, but not limited to non-natural amino acids, non-natural amino acid polypeptides and modified non-natural amino acid polypeptides and reagents for producing either of the aforementioned compounds) are acidic and may form a salt with a pharmaceutically acceptable cation. Some of the compounds herein (including, but not limited to non-natural amino acids, non-natural amino acid polypeptides and modified non-natural amino acid polypeptides and reagents for producing the aforementioned compounds) can be basic and accordingly, may form a salt with a pharmaceutically acceptable anion. All such salts, including di-salts are within the scope of the compositions described herein and they can be prepared by conventional methods. For example, salts can be prepared by contacting the acidic and basic entities, in either an aqueous, non-aqueous or partially aqueous medium. The salts are recovered by using at least one of the following techniques: filtration, precipitation with a non-solvent followed by filtration, evaporation of the solvent, or, in the case of aqueous solutions, lyophilization.

Pharmaceutically acceptable salts of the non-natural amino acid polypeptides disclosed herein may be formed when an acidic proton present in the parent non-natural amino acid polypeptides either is replaced by a metal ion, by way of example an alkali metal ion, an alkaline earth ion, or an aluminum ion; or coordinates with an organic base. In addition, the salt forms of the disclosed non-natural amino acid polypeptides can be prepared using salts of the starting materials or intermediates. The non-natural amino acid polypeptides described herein may be prepared as a pharmaceutically acceptable acid addition salt (which is a type of a pharmaceutically acceptable salt) by reacting the free base form of non-natural amino acid polypeptides described herein with a pharmaceutically acceptable inorganic or organic acid. Alternatively, the non-natural amino acid polypeptides described herein may be prepared as pharmaceutically acceptable base addition salts (which are a type of a pharmaceutically acceptable salt) by reacting the free acid form of non-natural amino acid polypeptides described herein with a pharmaceutically acceptable inorganic or organic base.

The type of pharmaceutical acceptable salts, include, but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like.

The corresponding counterions of the non-natural amino acid polypeptide pharmaceutical acceptable salts may be analyzed and identified using various methods including, but not limited to, ion exchange chromatography, ion chromatography, capillary electrophoresis, inductively coupled plasma, atomic absorption spectroscopy, mass spectrometry, or any combination thereof. In addition, the therapeutic activity of such non-natural amino acid polypeptide pharmaceutical acceptable salts may be tested using the techniques and methods described in examples 87-91.

It should be understood that a reference to a salt includes the solvent addition forms or crystal forms thereof, particularly solvates or polymorphs. Solvates contain either stoichiometric or non-stoichiometric amounts of a solvent, and are often formed during the process of crystallization with pharmaceutically acceptable solvents such as water, ethanol, and the like. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The screening and characterization of non-natural amino acid polypeptide pharmaceutical acceptable salts polymorphs and/or solvates may be accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UVIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

### INCORPORATION BY REFERENCE

All publications and patent applications mentioned in this specification are herein incorporated by reference to the same extent as if each individual publication or patent application was specifically and individually indicated to be incorporated by reference.ate of crystallization, and storage temperature may cause a single crystal form to dominate.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 presents dexamethasone-hydroxylamine linker conjugation with para-acetyl phenylalanine (pAF).

FIG. 2 (A) is an SDS-PAGE analysis of the FIG. 1 conjugation. The left-most arrow shows pAF; the middle arrow shows dexamethasone-hydroxylamine; the peak indicated by the right-most arrow shows the dexamethasone-hydroxylamine linker conjugation with pAF.

FIG. 2 (B) is an SDS-PAGE analysis of the FIG. 1 conjugation. The left-most arrow shows pAF; the middle arrow shows dexamethasone-hydroxylamine; the peak indicated by the right-most arrow shows the dexamethasone-hydroxylamine linker conjugation with pAF.

FIG. 2 (C) is an SDS-PAGE analysis of the FIG. 1 conjugation. The peak indicated by the right-most arrow shows the dexamethasone-hydroxylamine linker conjugation with pAF.

FIG. 3 (A) is a mass spectra analysis of the intact mass of the heavy chain of the monoclonal antibody plus dexamethasone conjugation reaction (reduced) and the peaks represent different conjugations including, in the far right peak, dexamethasone-linker oligomers.

FIG. 3 (B) is a mass spectra analysis of the intact mass of the light chain of the monoclonal antibody plus dexamethasone conjugation reaction (reduced).

FIG. 4 is a schematic of dexamethasone and cleavable linkers with [2+3] chemistry.

FIG. 5 is a schematic showing new analogs and linkers based on Mometasone furoate.

FIG. 6 is a schematic of a non-limiting example of a linker designed for dexamethasone.

FIG. 7 is a schematic of the chemical structures of SAR and Dexamethasone analogs, including: Dexamethasone (receptor affinity of 100); Budesonide (receptor affinity 855); Mometasone furoate (receptor affinity 2245); and Fluticasone furoate (receptor affinity 2989).

FIG. 8 is a schematic of the synthesis detailed in Example 1 (below).

FIG. 9 is a schematic of the synthesis detailed in Example 2 (below).

FIG. 10 is a schematic of the synthesis detailed in Example 3 (below).

FIG. 11 is a schematic of the synthesis detailed in Example 4 (below).

FIG. 12 is a schematic of the synthesis detailed in Example 5 (below).

FIG. 13 is a schematic of the synthesis detailed in Example 6 (below).

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

I. Introduction

Recently, an entirely new technology in the protein sciences has been reported, which promises to overcome many of the limitations associated with site-specific modifications of proteins. Specifically, new components have been added to the protein biosynthetic machinery of the prokaryote Escherichia coli (E. coli) (e.g., L. Wang, et al., (2001), Science 292:498-500) and the eukaryote Sacchromyces cerevisiae (S. cerevisiae) (e.g., J. Chin et al., Science 301:964-7 (2003)), which has enabled the incorporation of non-natural amino acids to proteins in vivo. A number of new amino acids with novel chemical, physical or biological properties, including photoaffinity labels and photoisomerizable amino acids, keto amino acids, and glycosylated amino acids have been incorporated efficiently and with high fidelity into proteins in E. coli and in yeast in response to the amber codon, TAG, using this methodology. See, e.g., J. W. Chin et al., (2002), Journal of the American Chemical Society 124:9026-9027 (incorporated by reference in its entirety); J. W. Chin, & P. G. Schultz, (2002), ChemBioChem 3(11):1135-1137 (incorporated by reference in its entirety); J. W. Chin, et al., (2002), PNAS United States of America 99(17):11020-11024 (incorporated by reference in its entirety); and, L. Wang, & P. G. Schultz, (2002), Chem. Comm., 1-11 (incorporated by reference in its entirety). These studies have demonstrated that it is possible to selectively and routinely introduce chemical functional groups that are not found in proteins, that are chemically inert to all of the functional groups found in the 20 common, genetically-encoded amino acids and that may be used to react efficiently and selectively to form stable covalent linkages.

II. Overview

At one level, described herein are the tools (methods, compositions, techniques) for creating and using NRL conjugates including nuclear receptor ligand (NRL) linker derivatives or analogs, comprising at least one carbonyl, dicarbonyl, oxime, hydroxylamine, aldehyde, protected aldehyde, ketone, protected ketone, thioester, ester, dicarbonyl, hydrazine, azide, amidine, imine, diamine, keto-amine, keto-alkyne, alkyne, cycloalkyne, or ene-dione. At another level, described herein are the tools (methods, compositions, techniques) for creating and using NRL conjugates including NRL linker derivatives or analogs, comprising at least one non-natural amino acid or modified non-natural amino acid with an oxime, aromatic amine, heterocycle (e.g., indole, quinoxaline, phenazine, pyrazole, triazole, etc.).

Such NRL conjugates comprising non-natural amino acids may contain further functionality, including but not limited to, a polymer; a water-soluble polymer; a derivative of polyethylene glycol; a second protein or polypeptide or polypeptide analog; an antibody or antibody fragment; and any combination thereof. Note that the various aforementioned functionalities are not meant to imply that the members of one functionality cannot be classified as members of another functionality. Indeed, there will be overlap depending upon the particular circumstances. By way of example only, a water-soluble polymer overlaps in scope with a derivative of polyethylene glycol, however the overlap is not complete and thus both functionalities are cited above.

III. Nuclear Receptor Ligand Conjugates and Derivatives

At one level, described herein are the tools (methods, compositions, techniques) for creating and using NRL conjugates, including NRL linker derivatives or analogs, comprising at least one non-natural amino acid or modified non-natural amino acid with a carbonyl, dicarbonyl, oxime or hydroxylamine group. Such NRL conjugates comprising non-natural amino acids may contain further functionality, including but not limited to, a polymer; a water-soluble polymer; a derivative of polyethylene glycol; a second protein or polypeptide or polypeptide analog; an antibody or antibody fragment; and any combination thereof. Note that the various aforementioned functionalities are not meant to imply that the members of one functionality cannot be classified as members of another functionality. Indeed, there will be overlap depending upon the particular circumstances. By way of example only, a water-soluble polymer overlaps in scope with a derivative of polyethylene glycol, however the overlap is not complete and thus both functionalities are cited above.

In one aspect are methods for selecting and designing NRL conjugates including NRL linker derivatives to be modified using the methods, compositions and techniques described herein. The new NRL conjugate or NRL linker derivative may be designed de novo, including by way of example only, as part of high-throughput screening process (in which case numerous polypeptides may be designed, synthesized, characterized and/or tested) or based on the interests of the researcher. The new NRL conjugate may also be designed based on the structure of a known or partially characterized polypeptide. The principles for selecting which amino acid(s) to substitute and/or modify are described separately herein. The choice of which modification to employ is also described herein, and can be used to meet the need of the experimenter or end user. Such needs may include, but are not limited to, manipulating the therapeutic effectiveness of the polypeptide, improving the safety profile of the polypeptide, adjusting the pharmacokinetics, pharmacologics and/or pharmacodynamics of the polypeptide, such as, by way of example only, increasing water solubility, bioavailability, increasing serum half-life, increasing therapeutic half-life, modulating immunogenicity, modulating biological activity, or extending the circulation time. In addition, such modifications include, by way of example only, providing additional functionality to the polypeptide, incorporating an antibody, and any combination of the aforementioned modifications.

Also described herein are NRL conjugates that have or can be modified to contain an oxime, carbonyl, dicarbonyl, or hydroxylamine group. Included with this aspect are methods for producing, purifying, characterizing and using such NRL conjugates.

The NRL conjugate may contain at least one, at least two, at least three, at least four, at least five, at least six, at least seven, at least eight, at least nine, or ten or more of a carbonyl or dicarbonyl group, oxime group, hydroxylamine group, or protected forms thereof. The NRL conjugate can be the same or different, for example, there can be 1, 2, 3, 4, 5, 6, 7, 8, 9, 10, 11, 12, 13, 14, 15, 16, 17, 18, 19, 20, or more different sites in the derivative that comprise 1, 2, 3, 4, 5, 6, 7, 8, 9, 10, 11, 12, 13, 14, 15, 16, 17, 18, 19, 20, or more different reactive groups.

A. Structure and Synthesis of Nuclear Receptor Ligand Conjugates: Electrophilic and Nucleophilic Groups

Nuclear receptor ligand conjugates with linkers containing a hydroxylamine (also called an aminooxy) group allow for reaction with a variety of electrophilic groups to form conjugates (including but not limited to, with PEG or other water soluble polymers). Like hydrazines, hydrazides and semicarbazides, the enhanced nucleophilicity of the aminooxy group permits it to react efficiently and selectively with a variety of molecules that contain carbonyl- or dicarbonyl-groups, including but not limited to, ketones, aldehydes or other functional groups with similar chemical reactivity. See, e.g., Shao, J. and Tam, J., J. Am. Chem. Soc. 117:3893-3899 (1995); H. Hang and C. Bertozzi, Ace. Chem. Res. 34(9): 727-736 (2001). Whereas the result of reaction with a hydrazine group is the corresponding hydrazone, however, an oxime results generally from the reaction of an aminooxy group with a carbonyl- or dicarbonyl-containing group such as, by way of example, a ketones, aldehydes or other functional groups with similar chemical reactivity. In some embodiments of NRL conjugates with linkers, the conjugate comprises an azide, alkyne or cycloalkyne allow for linking of molecules via cycloaddition reactions (e.g., 1,3-dipolar cycloadditions, azide-alkyne Huisgen cycloaddition, etc.). (Described in U.S. Pat. No. 7,807,619 which is incorporated by reference herein to the extent relative to the reaction).

Thus, in certain embodiments described herein are NRL conjugates with linkers comprising a hydroxylamine, aldehyde, protected aldehyde, ketone, protected ketone, thioester, ester, dicarbonyl, hydrazine, amidine, imine, diamine, keto-amine, keto-alkyne, and ene-dione hydroxylamine group, a hydroxylamine-like group (which has reactivity similar to a hydroxylamine group and is structurally similar to a hydroxylamine group), a masked hydroxylamine group (which can be readily converted into a hydroxylamine group), or a protected hydroxylamine group (which has reactivity similar to a hydroxylamine group upon deprotection). In some embodiments, the NRL conjugates comprise azides, alkynes or cycloalkynes. Such NRL conjugates include compounds having the structure of Formula (I), (III), (IV), (V), and (VI) wherein NRL is any nuclear receptor ligand:

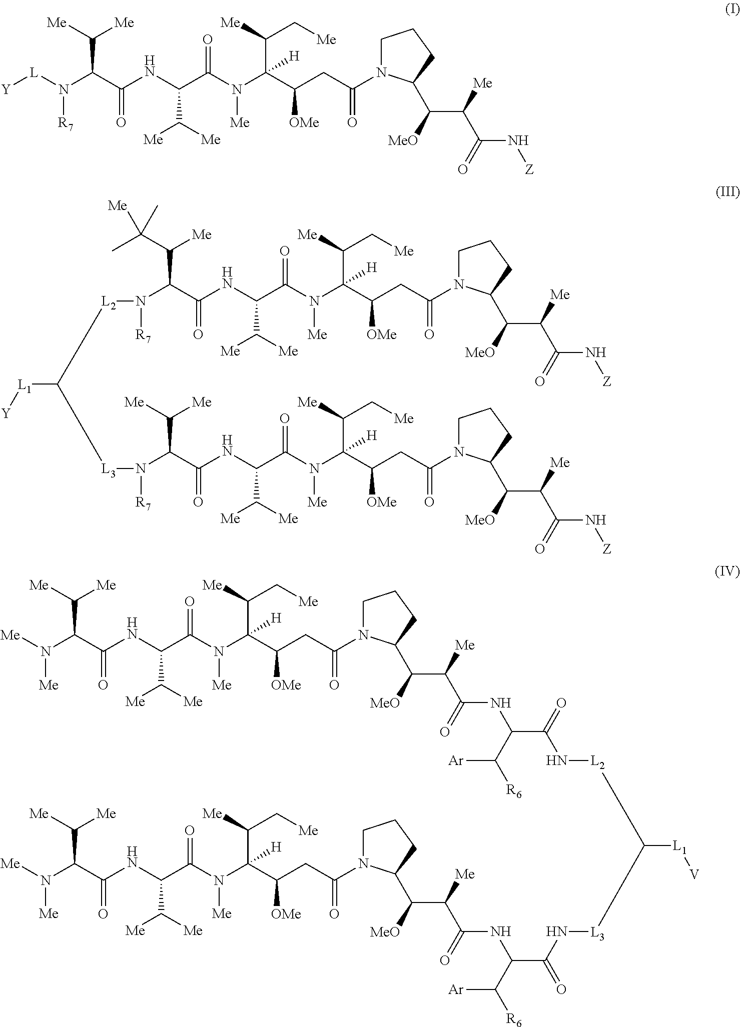

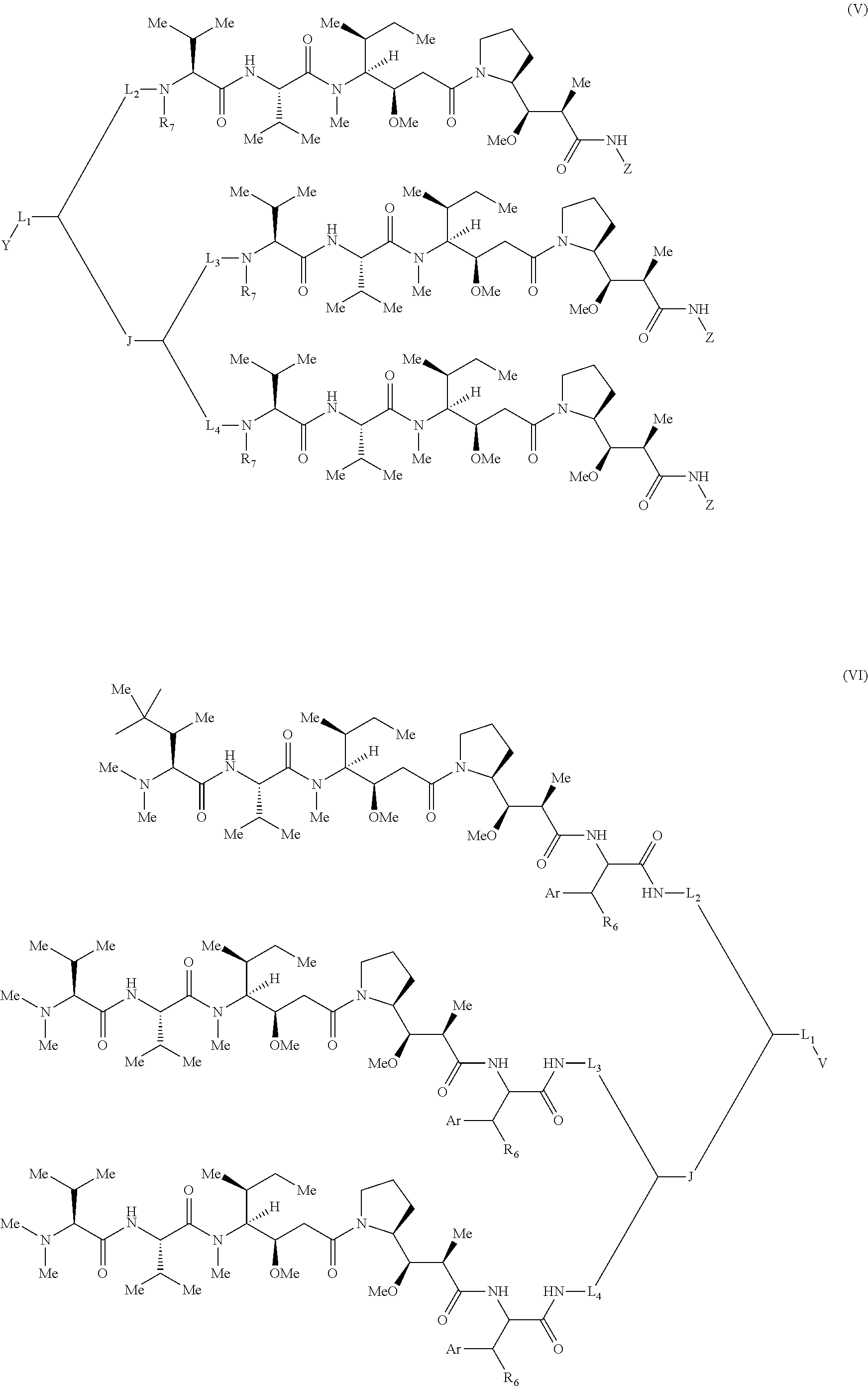

wherein:
Y and V are each selected from the group consisting of an hydroxylamine, methyl, aldehyde, protected aldehyde, ketone, protected ketone, thioester, ester, dicarbonyl, hydrazine, azide, amidine, imine, diamine, keto-amine, keto-alkyne, alkyne, cycloalkyne, and ene-dione;
L, L₁, L₂, L₃, and L₄ are each linkers selected from the group consisting of a bond, -alkylene-, -alkylene-C(O)—, -alkylene-J-, -(alkylene-O)ₙ-alkylene-, -(alkylene-O)ₙ-alkylene-C(O)—, -(alkylene-O)ₙ-J-, -(alkylene-O)ₙ-J-alkylene-, -(alkylene-O)ₙ—(CH₂)_{2′}—NHC(O)—(CH₂)_{n″}—C(Me)₂-S—S—(CH₂)_{n′″}—NHC(O)-(alkylene-O)_{n′″}-alkylene-, -(alkylene-O)ₙ-alkylene-W—, -alkylene-C(O)—W—, -(alkylene-O)ₙ-alkylene-J-, -alkylene′-J-(alkylene-O)_{n′}-alkylene-, -(alkylene-O)ₙ-alkylene-J-alkylene′, -J-(alkylene-O)ₙ-alkylene-, -(alkylene-O)ₙ-alkylene-J-(alkylene-O)ₙ′-alkylene-J′-, —W—, -alkylene-W—, alkylene′-J-(alkylene-NMe)ₙ-alkylene-W—, -J-(alkylene-NMe)ₙ-alkylene-W—, -(alkylene-O)ₙ-alkylene-U-alkylene-C(O)—, -(alkylene-O)ₙ-alkylene-U-alkylene-; -J-alkylene-NMe-alkylene″-NMe-alkylene″W—, and -alkylene-J-alkylene′-NMe-alkylene″-NMe-alkylene′″-W-;
   W has the structure of:

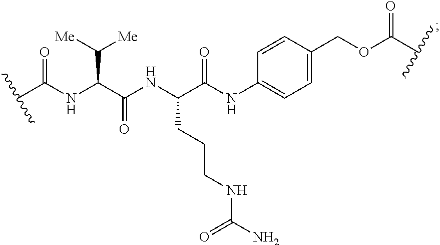 [SMILES: *C(=O)N[C@H](C(=O)N[C@@H](CCCNC(N)=O)C(=O)Nc1ccc(COC(*)=O)cc1)C(C)C]

U has the structure of:

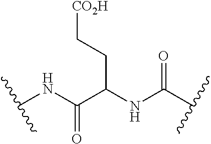 [SMILES: *NC(=O)C(CCC(=O)O)NC(*)=O]

each J and J′ independently have the structure of:

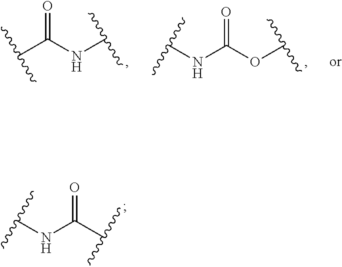 [SMILES: *NC(*)=O ; *NC(=O)O* ; *NC(*)=O]

each n, n′ n″, n′″ and n″″ are independently integers greater than or equal to one. Such NRL conjugates may be in the form of a salt, or may be incorporated into a non-natural amino acid polypeptide, polymer, polysaccharide, or a polynucleotide and optionally post translationally modified,

In some embodiments, Y is azide. In other embodiments, Y is cycloalkyne. In specific embodiments, the cyclooctyne has a structure of:

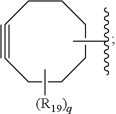

each R₁₉ is independently selected from the group consisting of C₁-C₆ alkyl, C₁-C₆ alkoxy, ester, ether, thioether, aminoalkyl, halogen, alkyl ester, aryl ester, amide, aryl amide, alkyl halide, alkyl amine, alkyl sulfonic acid, alkyl nitro, thioester, sulfonyl ester, halosulfonyl, nitrile, alkyl nitrile, and nitro; and
q is 0, 1, 2, 3, 4, 5, 6, 7, 8, 9, 10 or 11.

In certain embodiments of compounds of Formula (I), (III), and (V), Y is hydroxylamine, aldehyde, protected aldehyde, ketone, protected ketone, thioester, ester, dicarbonyl, hydrazine, amidine, imine, diamine, keto-amine, keto-alkyne, or ene-dione.

In certain embodiments of compounds of Formula (IV) and (VI), V is a hydroxylamine, methyl, aldehyde, protected aldehyde, ketone, protected ketone, thioester, ester, dicarbonyl, hydrazine, amidine, imine, diamine, keto-amine, keto-alkyne, and ene-dione.

In certain embodiments of compounds of Formula (I), (III), (IV), (V), and (VI), each L, L₁, L₂, L₃, and L₄ is independently a cleavable linker or non-cleavable linker. In certain embodiments of compounds of Formula (I), (III), (IV), (V), and (VI), each L, L₁, L₂, L₃, and L₄ is independently a oligo(ethylene glycol) derivatized linker,

In certain embodiments of compounds of Formula (I), (III), (IV), (V), and (VI), each alkylene, alkylene′, alkylene″, and alkylene′″ independently is —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —CH₂CH₂CH₂CH₂CH₂CH₂CH₂CH₂CH₂—, —CH₂CH₂CH₂CH₂CH₂CH₂CH₂CH₂CH₂CH₂—, CH₂CH₂CH₂CH₂CH₂CH₂CH₂CH₂CH₂CH₂CH₂—, or —CH₂CH₂CH₂CH₂CH₂CH₂CH₂CH₂CH₂CH₂CH₂CH₂—. In certain embodiments of compounds of Formula (XIV), (XV), (XVI), (XVII), and (XVIII), each n, n′, n″, n′″, and n″″ is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

B. Structure and Synthesis of Nuclear Receptor Ligand Conjugates: Hydroxylamine Groups

Thus, in certain embodiments described herein are NRL conjugates comprising a hydroxylamine group, a hydroxylamine-like group (which has reactivity similar to a hydroxylamine group and is structurally similar to a hydroxylamine group), a masked hydroxylamine group (which can be readily converted into a hydroxylamine group), or a protected hydroxylamine group (which has reactivity similar to a hydroxylamine group upon deprotection). Such NRL conjugates include compounds having the structure of Formula (I):

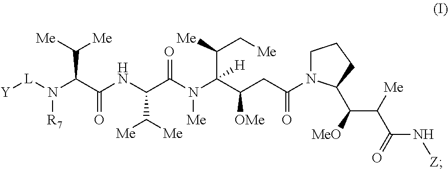

wherein:
Y is NH₂—O— or methyl;
L is a linker selected from the group consisting of -alkylene-, -alkylene-C(O)—, -(alkylene-O)ₙ-alkylene-, -(alkylene-O)ₙ-alkylene-C(O)—, -(alkylene-O)ₙ(CH₂)_{n′}—NHC(O)—(CH₂)_{n″}—C(Me)₂-S—S—(CH₂)_{n′″}—NHC(O)-(alkylene-O)_{n′″}alkylene-, -(alkylene-O)ₙ-alkylene-W—, alkylene-C(O)—W—, -(alkylene-O)ₙ-alkylene-U-alkylene-C(O)—, and -(alkylene-O)ₙ-alkylene-U-alkylene-;
   W has the structure of:

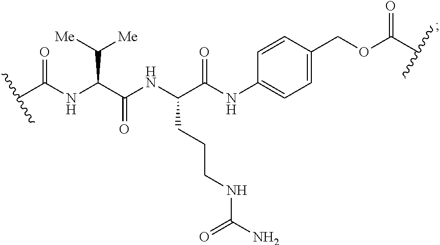 [SMILES: *C(=O)N[C@H](C(=O)N[C@@H](CCCNC(N)=O)C(=O)Nc1ccc(COC(*)=O)cc1)C(C)C]

U has the structure of:

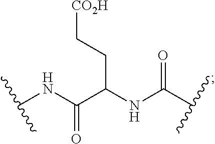 [SMILES: *NC(=O)C(CCC(=O)O)NC(*)=O]

or L is absent, Y is methyl, R₅ is COR₈, and R₈ is —NH-(alkylene-O)ₙ—NH₂; and
each n, n′, n″, n′″ and n″″ are independently integers greater than or equal to one.

In certain embodiments of compounds of Formula (I), Y is hydroxylamine, aldehyde, protected aldehyde, ketone, protected ketone, thioester, ester, dicarbonyl, hydrazine, amidine, imine, diamine, keto-amine, keto-alkyne, or ene-dione. In certain embodiments of compounds of Formula (I), V is a hydroxylamine, methyl, aldehyde, protected aldehyde, ketone, protected ketone, thioester, ester, dicarbonyl, hydrazine, amidine, imine, diamine, keto-amine, keto-alkyne, and ene-dione.

In certain embodiments of compounds of Formula (I), each L is independently a cleavable linker or non-cleavable linker. In certain embodiments of compounds of Formula (I), each L is independently a oligo(ethylene glycol) derivatized linker,

In certain embodiments of compounds of Formula (I), alkylene is —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —CH₂CH₂CH₂CH₂CH₂CH₂CH₂CH₂CH₂—, —CH₂CH₂CH₂CH₂CH₂CH₂CH₂CH₂CH₂CH₂—, —CH₂CH₂CH₂CH₂CH₂CH₂CH₂CH₂CH₂CH₂CH₂—, or CH₂CH₂CH₂CH₂CH₂CH₂CH₂CH₂CH₂CH₂CH₂CH₂—. In certain embodiments of compounds of Formula (I), each n, n′, n″, n′″, and n″″ is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In certain embodiments, NRL conjugates include compounds having the structure of Formula (II):

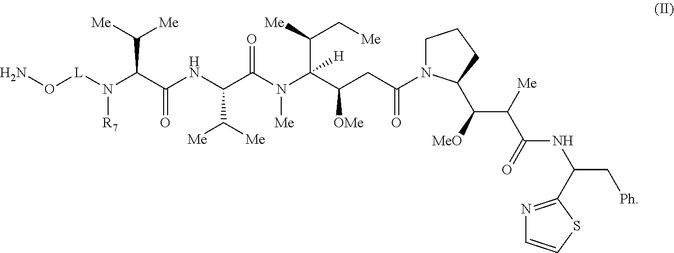

In some embodiments of compounds of Formula (II), L is -(alkylene-O)ₙ-alkylene-. In some embodiments, each alkylene is —CH₂CH₂—, n is equal to 3, and R₇ is methyl. In some embodiments, L is -alkylene-. In some embodiments of compounds of Formula (II), each alkylene is —CH₂CH₂— and R₇ is methyl or hydrogen. In some embodiments of compounds of Formula (II), L is -(alkylene-O)ₙ-alkylene-C(O)—. In some embodiments of compounds of Formula (II), each alkylene is —CH₂CH₂—, n is equal to 4, and R₇ is methyl. In some embodiments of compounds of Formula (II), L is -(alkylene-O)ₙ—(CH₂)ₙ—NHC(O)—(CH₂)_{n″}—C(Me)₂-S—S—(CH₂)_{n′″}—NHC(O)-(alkylene-O)_{n″″}-alkylene-. In some embodiments of compounds of Formula (II), each alkylene is —CH₂CH₂—, n is equal to 1, n′ is equal to 2, n″ is equal to 1, n′″ is equal to 2, n″″ is equal to 4, and R₇ is methyl. Such NRL conjugates may be in the form of a salt, or may be incorporated into a non-natural amino acid polypeptide, polymer, polysaccharide, or a polynucleotide and optionally post translationally modified.

In certain embodiments of compounds of Formula (II), each L is independently a cleavable linker or non-cleavable linker. In certain embodiments of compounds of Formula (II), each L is independently a oligo(ethylene glycol) derivatized linker.

Such NRL conjugates include compounds having the structure of Formula (III), (IV), (V) or (VI):

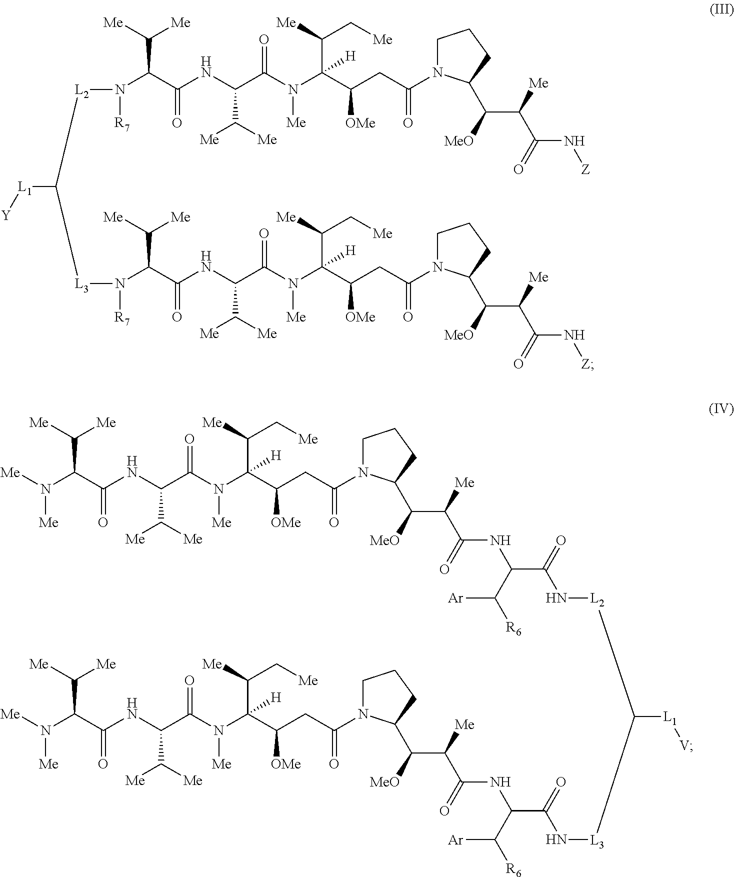

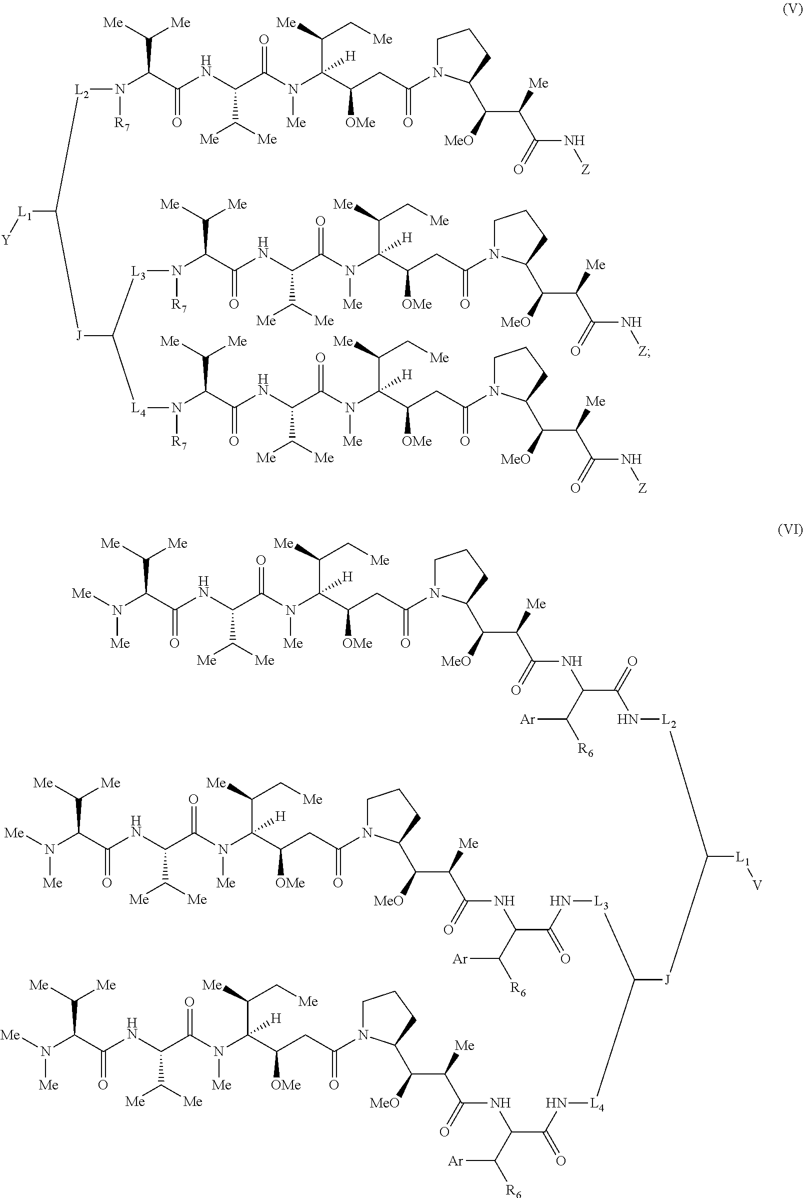

wherein:
Y is NH₂—O—;
V is —O—NH₂
L₁, L₂, L₃, and L₄ are each linkers independently selected from the group consisting of a bond, -alkylene-, -(alkylene-O)ₙ-alkylene-J-, -alkylene′-J-(alkylene-O)ₙ-alkylene-, -J-(alkylene-O)ₙ-alkylene-, -(alkylene-O)ₙ-alkylene-J-(alkylene-O)ₙ′alkylene-J′-, -(alkylene-O)ₙ-alkylene-J-alkylene′-, —W—, -alkylene-W—, alkylene′J-(alkylene-NMe)ₙ-alkylene-W—, -J-(alkylene-NMe)ₙ-alkylene-W—, -J-alkylene-NMe-alkylene′-NMe-alkylene″-W-, and -alkylene-J-alkylene′-NMe-alkylene″-NMe-alkylene′″-W-;
   W has the structure of:

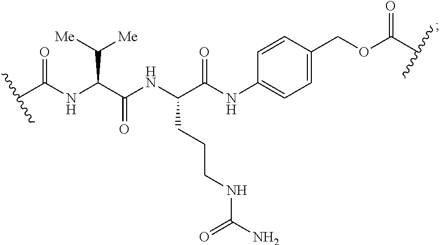 [SMILES: *C(=O)N[C@H](C(=O)N[C@@H](CCCNC(N)=O)C(=O)Nc1ccc(COC(*)=O)cc1)C(C)C]

each J and J′ independently have the structure of:

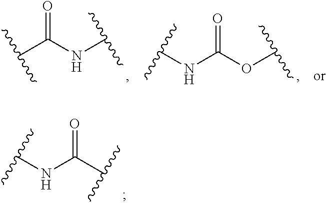 [SMILES: *NC(*)=O ; *NC(=O)O* ; *NC(*)=O]

and
   each n and n′ are independently integers greater than or equal to one.
      Such NRL conjugates may be in the form of a salt, or may be incorporated into a non-natural amino acid polypeptide, polymer, polysaccharide, or a polynucleotide and optionally post translationally modified.

In certain embodiments of compounds of Formula (III) and (V), Y is hydroxylamine, aldehyde, protected aldehyde, ketone, protected ketone, thioester, ester, dicarbonyl, hydrazine, amidine, imine, diamine, keto-amine, keto-alkyne, or ene-dione. In certain embodiments of compounds of Formula (IV) and (VI), V is a hydroxylamine, methyl, aldehyde, protected aldehyde, ketone, protected ketone, thioester, ester, dicarbonyl, hydrazine, amidine, imine, diamine, keto-amine, keto-alkyne, and ene-dione.

In certain embodiments of compounds of Formula (XIV), (XV), (XVI), (XVII), and (XVIII), each L, L₁, L₂, L₃, and L₄ is independently a cleavable linker or non-cleavable linker. In certain embodiments of compounds of Formula (XIV), (XV), (XVI), (XVII), and (XVIII), each L, L₁, L₂, L₃, and L₄ is independently a oligo(ethylene glycol) derivatized linker,

In certain embodiments of compounds of Formula (III), (IV), (V) and (VI), each alkylene, alkylene′, alkylene″, and alkylene′″ independently is —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —CH₂CH₂CH₂CH₂CH₂CH₂CH₂CH₂CH₂—, —CH₂CH₂CH₂CH₂CH₂CH₂CH₂CH₂CH₂CH₂—, —CH₂CH₂CH₂CH₂CH₂CH₂CH₂CH₂CH₂CH₂CH₂—, or —CH₂CH₂CH₂CH₂CH₂CH₂CH₂CH₂CH₂CH₂CH₂CH₂—. In certain embodiments of compounds of Formula (III), (IV), (V) and (VI), alkylene is methylene, ethylene, propylene, butylenes, pentylene, hexylene, or heptylene.

In certain embodiments of compounds of Formula (III), (IV), (V) and (VI), each n and n′ independently is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In certain embodiments, NRL conjugates include compounds having the structure of Formula (VII):

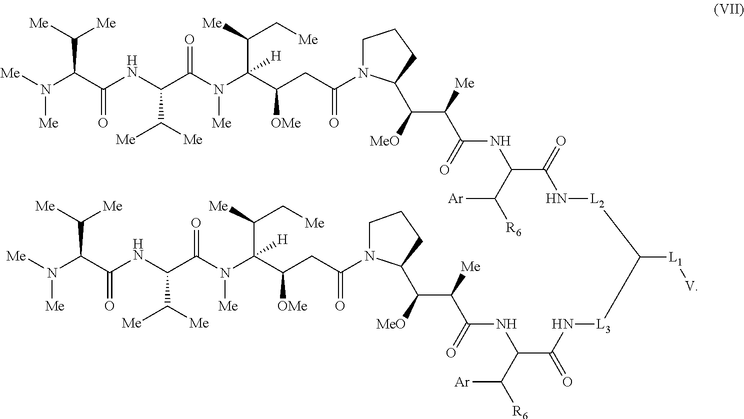

In certain embodiments of compounds of Formula (VII), L₁ is -(alkylene-O)ₙ-alkylene-J-, L₂ is -alkylene′-J′-(alkylene-O)ₙ′-alkylene-, L₃ is -J″-(alkylene-O)ₙ″-alkylene-, alkylene is —CH₂CH₂—, alkylene′ is —(CH₂)₄—, n is 1, n′ and n″ are 3, J has the structure of

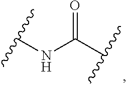 [SMILES: *NC(*)=O]

J′ and J″ have the structure of

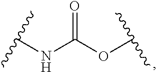 [SMILES: *NC(=O)O*]

and R₇ is methyl. In certain embodiments of compounds of Formula (VII), L₁ is -J-(alkylene-O)ₙ-alkylene-, L₂ is -(alkylene-O)_{n′}-alkylene-J′-alkylene′-, L₃ is -(alkylene-O)_{n″}-alkylene-J″-, alkylene is —CH₂CH₂—, alkylene′ is —(CH₂)₄—, n is 1, n′ and n″ are 4, and J, J′ and J″ have the structure of

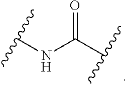 [SMILES: *NC(*)=O]

Such NRL conjugates may be in the form of a salt, or may be incorporated into a non-natural amino acid polypeptide, polymer, polysaccharide, or a polynucleotide and optionally post translationally modified.

In certain embodiments, compounds of Formula (I)-(VII) are stable in aqueous solution for at least 1 month under mildly acidic conditions. In certain embodiments, compounds of Formula (I)-(VII) are stable for at least 2 weeks under mildly acidic conditions. In certain embodiments, compound of Formula (I)-(VII) are stable for at least 5 days under mildly acidic conditions. In certain embodiments, such acidic conditions are pH 2 to 8.

The methods and compositions provided and described herein include polypeptides comprising an NRL conjugate containing at least one carbonyl or dicarbonyl group, oxime group, hydroxylamine group, or protected or masked forms thereof. Introduction of at least one reactive group into a NRL conjugate, or to any one or two components of the Ab-L-Y conjugate, can allow for the application of conjugation chemistries that involve specific chemical reactions, including, but not limited to, with one or more NRL conjguate(s) while not reacting with the commonly occurring amino acids. Once incorporated, the NRL conjugate side chains can also be modified by utilizing chemistry methodologies described herein or suitable for the particular functional groups or substituents present in the NRL conjugate.

The NRL conjugate methods and compositions described herein provide conjugates of substances having a wide variety of functional groups, substituents or moieties, with other substances including but not limited to a polymer; a water-soluble polymer; a derivative of polyethylene glycol; a second protein or polypeptide or polypeptide analog; an antibody or antibody fragment; and any combination thereof.

In certain embodiments, the NRL conjugates, linkers and reagents described herein, including compounds of Formulas (I)-(VII) are stable in aqueous solution under mildly acidic conditions (including but not limited to pH 2 to 8). In other embodiments, such compounds are stable for at least one month under mildly acidic conditions. In other embodiments, such compounds are stable for at least 2 weeks under mildly acidic conditions. In other embodiments, such compounds are stable for at least 5 days under mildly acidic conditions.

In another aspect of the compositions, methods, techniques and strategies described herein are methods for studying or using any of the aforementioned “modified or unmodified” non-natural amino acid NRL conjugates, Included within this aspect, by way of example only, are therapeutic, diagnostic, assay-based, industrial, cosmetic, plant biology, environmental, energy-production, consumer-products, and/or military uses which would benefit from a NRL conjugate comprising a “modified or unmodified” non-natural amino acid polypeptide or protein.

Non-limiting examples of NRL conjugates are given below. For example, if:

AFg-L₁-L₂-D)ₘ

and A is a antibody;
Fg is functional group connecting antibody and linker, which is selected from:

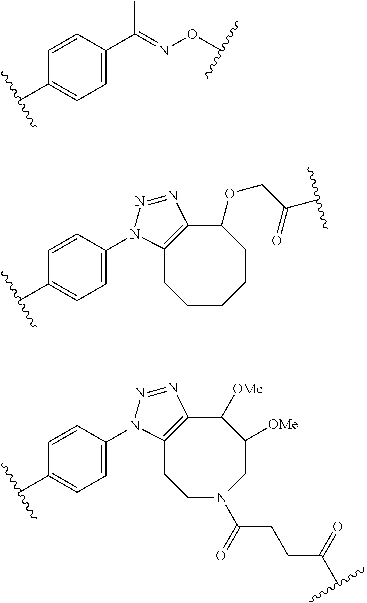 [SMILES: *O/N=C(\C)c1ccc(*)cc1 ; *C(=O)COC1CCCCCc2c1nnn2-c1ccc(*)cc1 ; *C(=O)CCC(=O)N1CCc2c(nnn2-c2ccc(*)cc2)C(OC)C(OC)C1]

and L1 and L2 are linkers;

then non-limiting examples of D include: antiandrogens; alpha-substituted steroids; carbonylamino -benzimidazole; 17-hydroxy 4-aza androstan-3-ones; antiandrogenic biphenyls; goserelin; nilutamid; decursin; flutamide; p,p′-DDE; vinclozolin; cyproterone acetate; linuron; fluorinated 4-azasteroids; fluorinated 4-azasteroids derivatives; antiandrogens; alpha-substituted steroids; carbonylamino-benzimidazole; 17-hydroxy 4-aza androstan-3-ones; antiandrogenic biphenyls; goserelin; nilutamid; decursin; flutamide; p,p′-DDE; vinclozolin; cyproterone acetate; linuron; other kinase inhibitors, staurosporine, saracatinib, fingolimod, and other glucocorticoids

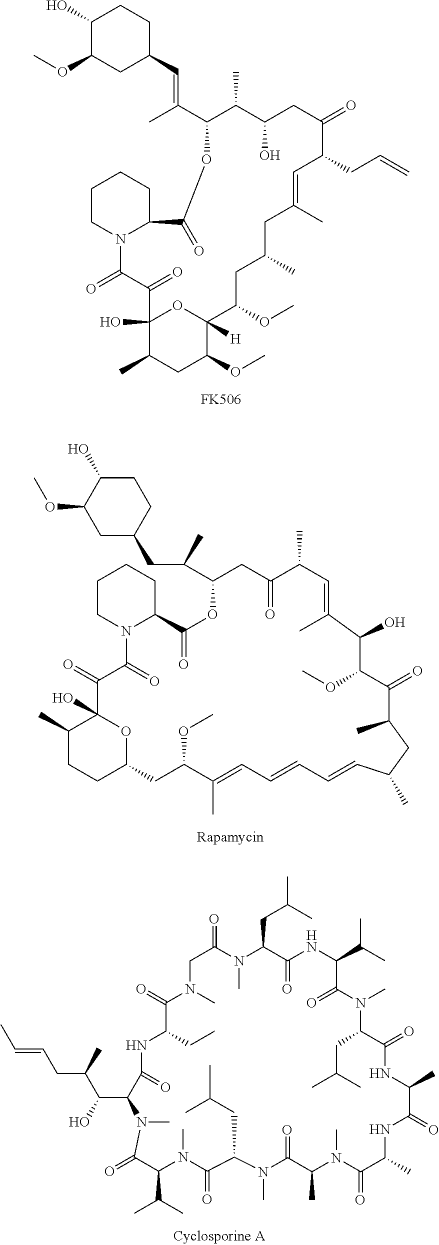

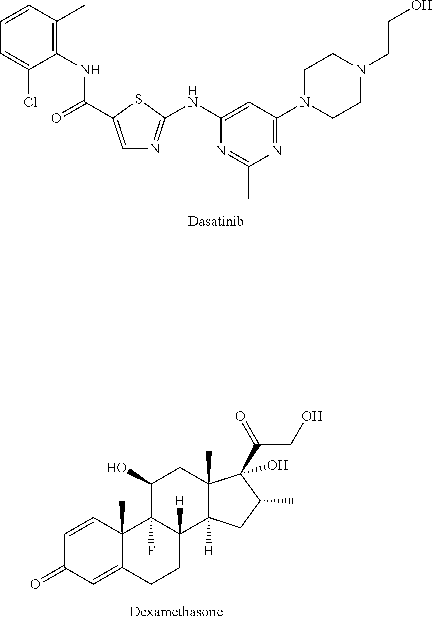

m=1-4

Other non-limiting examples of NRL conjugates are given below. For example, if:

G-L₁-L₂-D
wherein G is functional group for conjugation to connect antibody and linker, which is selected from:

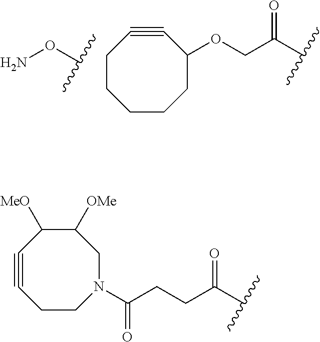

L1 is selected from -J-W—, —NH-J-W—,
J is selected from: —C₁-C₃₀ alkylene-, —C₂-C₃₀ alkenylene-containing 0 to 20 heteroatoms selected from O, S or N; substituted C₁-C₃₀ alkylene, substituted C₂-C₃₀ alkenylene containing 0 to 20 heteroatoms selected from O, S or N;
W is selected from none, —CO—, —NHCO—, —OCO—
L2 is selected from -(E-Q)ₖ-,
E is an enzyme cleavage substrate: a dipeptide up to hexapeptide with or without para aminobenzyl alcohol, selected from:
-ValCit-(p-amino-benzylalcohol-CO)k-, -ValLys-(p-amino-benzylalcohol-CO)k-, -ValArg-(p-amino-benzylalcohol-CO)k-, -PheLys-(p-amino-benzylalcohol-CO)k-, -PheArg-(p-amino-benzylalcohol-CO)k-,
k=0,1
Q is a spacer, selected from:

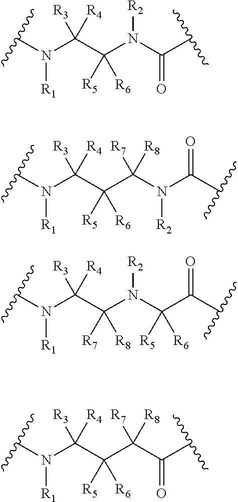

R1, R2, R3, R4, R5, R6, R7, R8 is independently selected from H, CH3, (C1-C6) alkyl

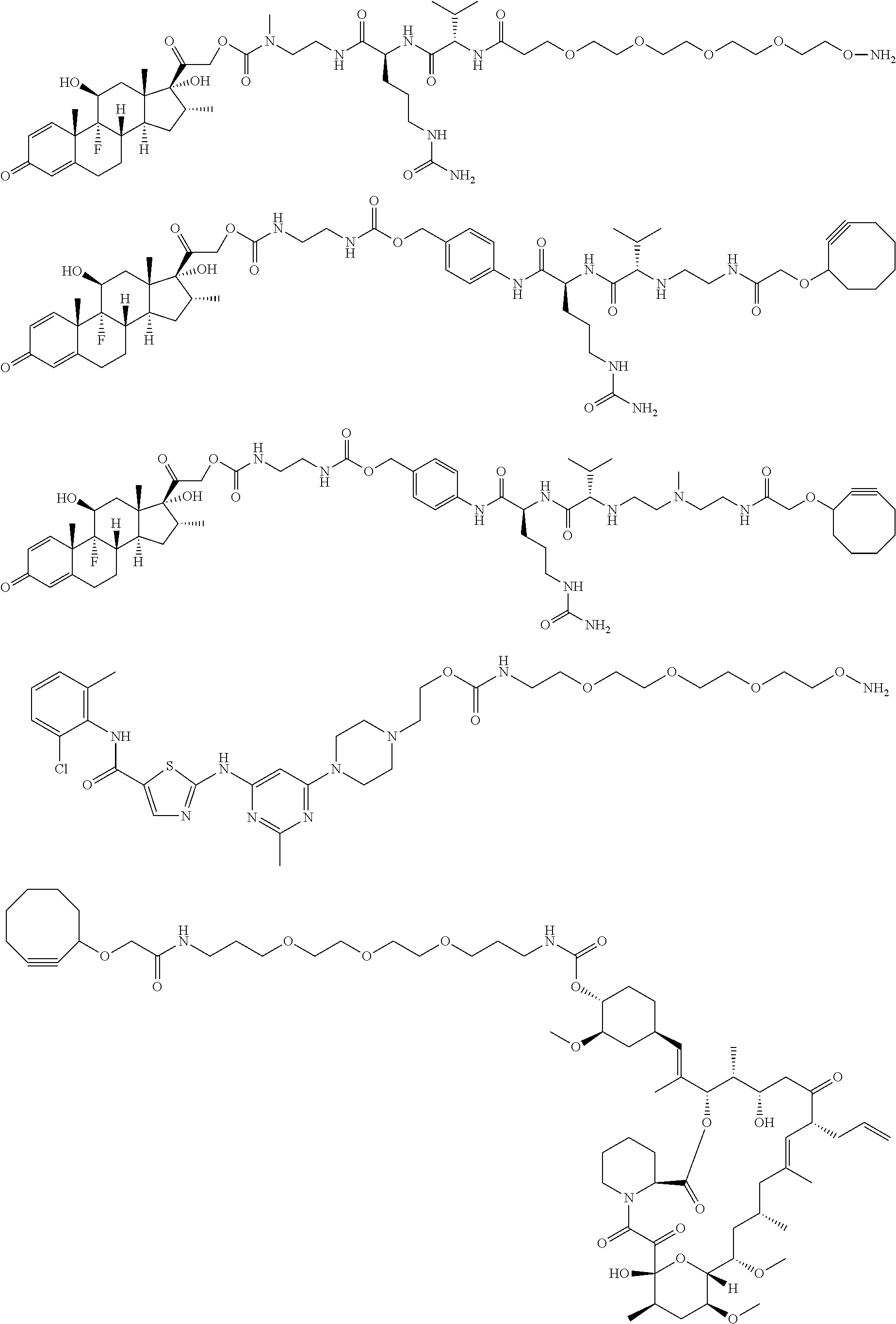

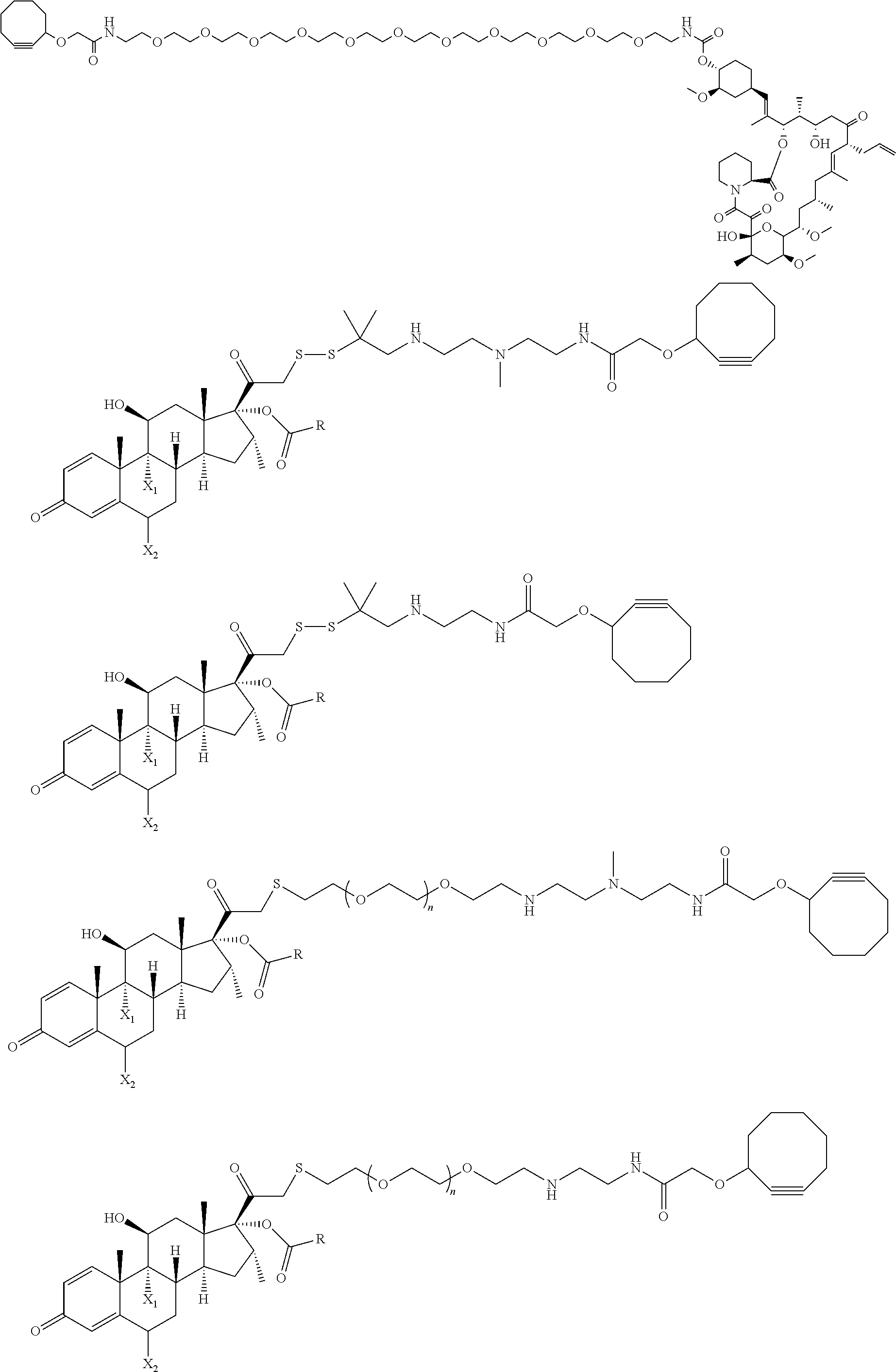

m=1-4

Non-Limiting Examples of NRL Conjugates Include:

For example, NRL linker of the present invention includes below used with dexamethasone. It can also be used with SAR and Dex analogs including, but not limited to, budesonide, mometasone furoate, and fluticasone furoate and these may be used in the treatment of a variety of conditions. An example of a linker of the present invention to be used in the treatment of a chronic immune disease:

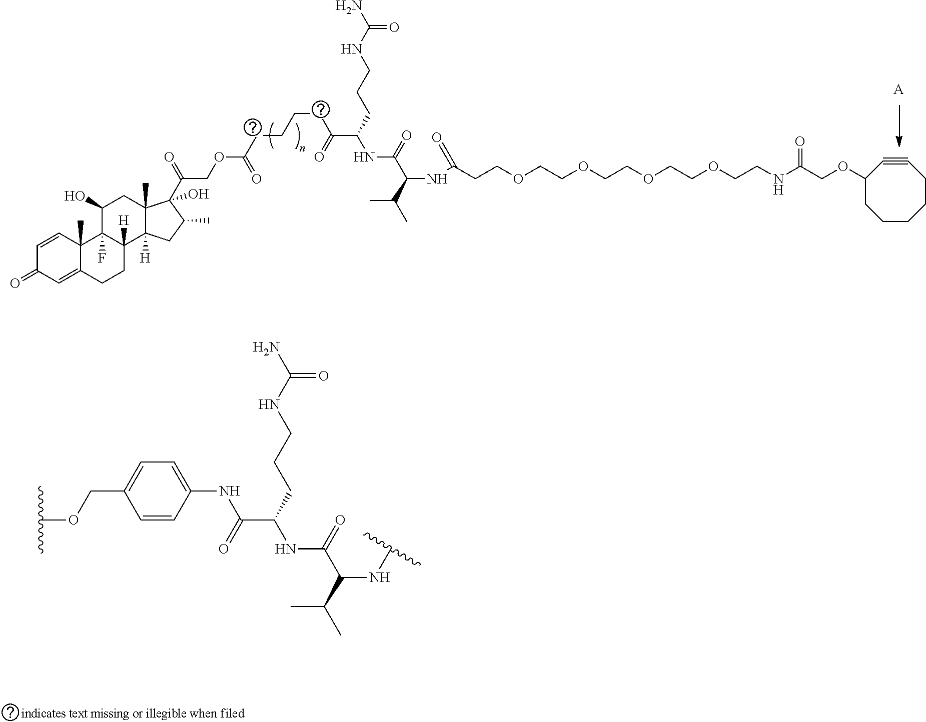

wherein A indicates where to avoid cyclooctatetraene.
For example, dexamethasone-hydroxylamine linker conjugation with pAF:

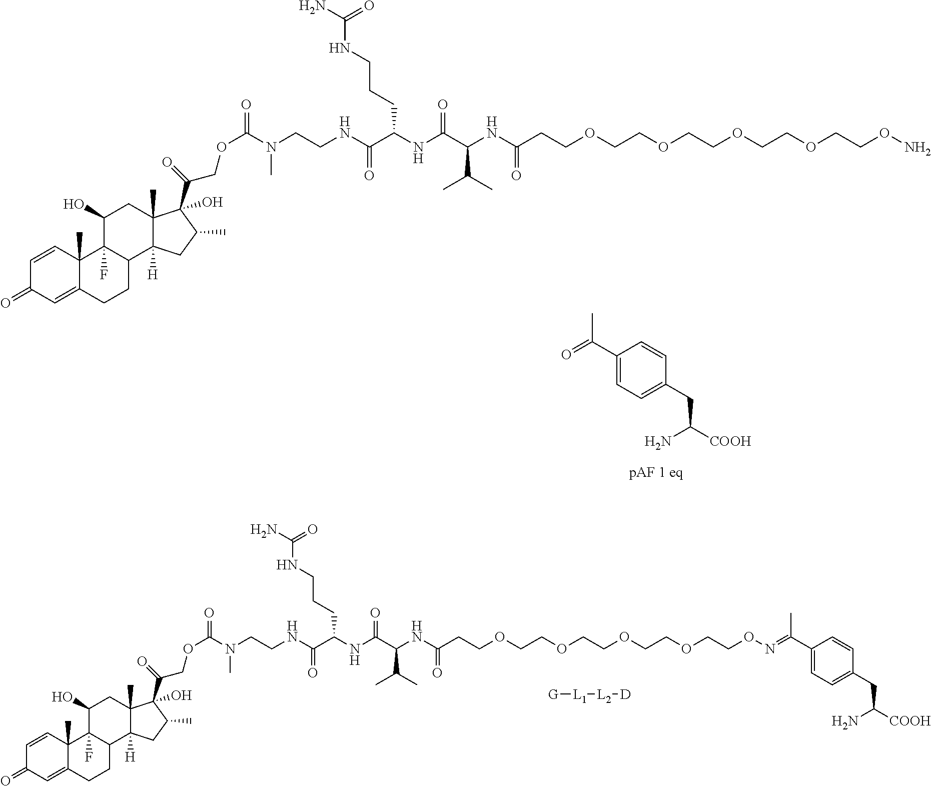

Also, by way of non-limiting example, dexamethasone and cleavable linkers with [2+3] chemistry:

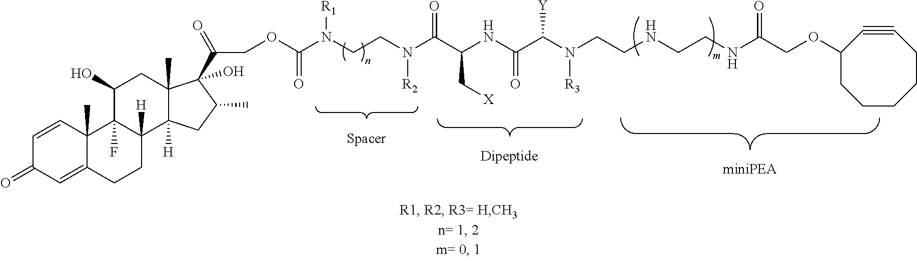

1. Evaluation of spacers: R1, R2=H, CH3

n=1,2

1st run examples:

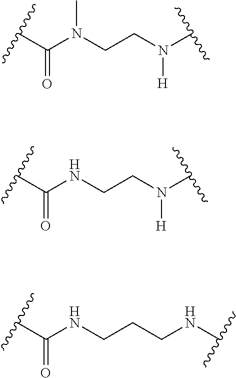

2. Evaluation of dipeptides with fixed miniPEA linker:

Val-Cit, ValPhe-Lys, Val-Glu, Val-Asp

1st run examples:

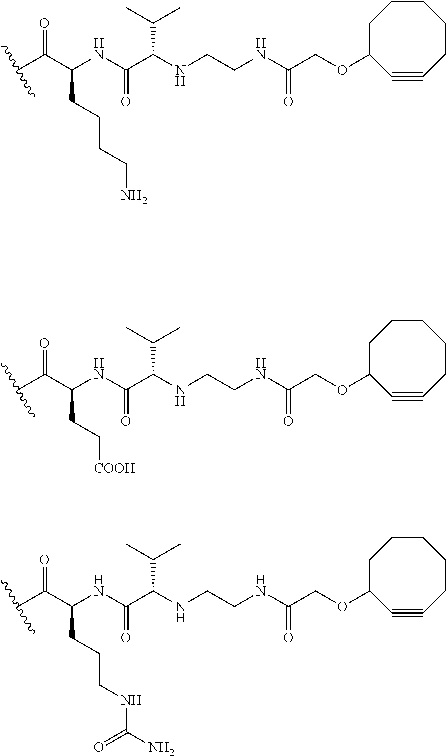 [SMILES: *C(=O)[C@H](CCCCN)NC(=O)[C@@H](NCCNC(=O)COC1C#CCCCCC1)C(C)C ; *C(=O)[C@H](CCC(=O)O)NC(=O)[C@@H](NCCNC(=O)COC1C#CCCCCC1)C(C)C ; *C(=O)[C@H](CCCNC(N)=O)NC(=O)[C@@H](NCCNC(=O)COC1C#CCCCCC1)C(C)C]

And new analogs and linkers based on dexamethasone derivative, mometasone furoate:

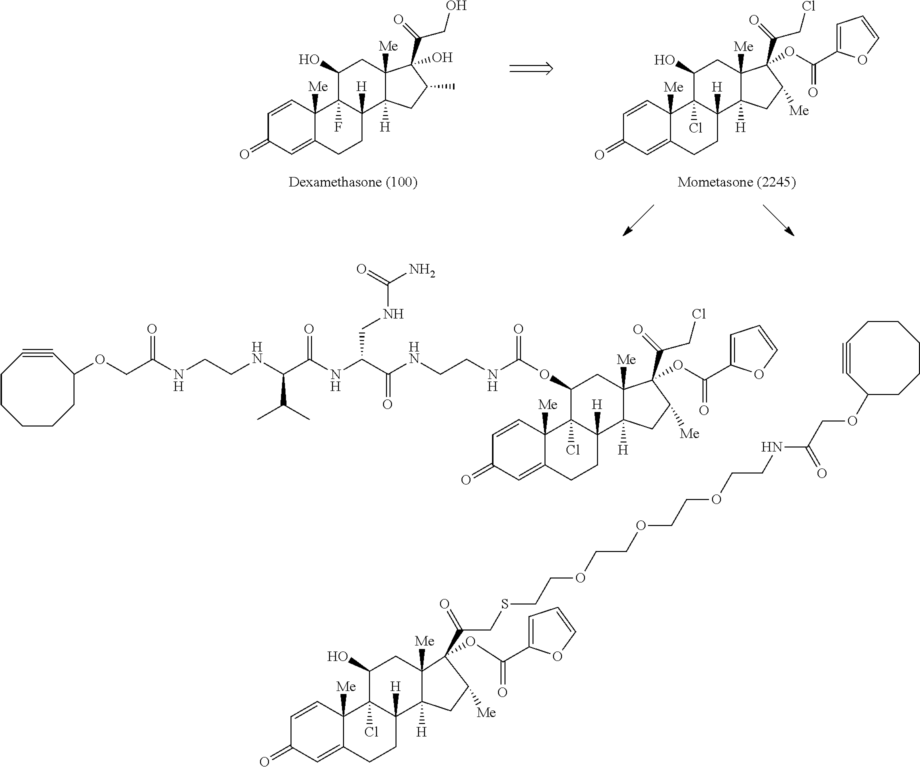

Non-limiting examples of antibody conjugated glucocorticoid receptor modulator linker derivatives, and/or antibody conjugated nuclear receptor ligand linker derivatives include:

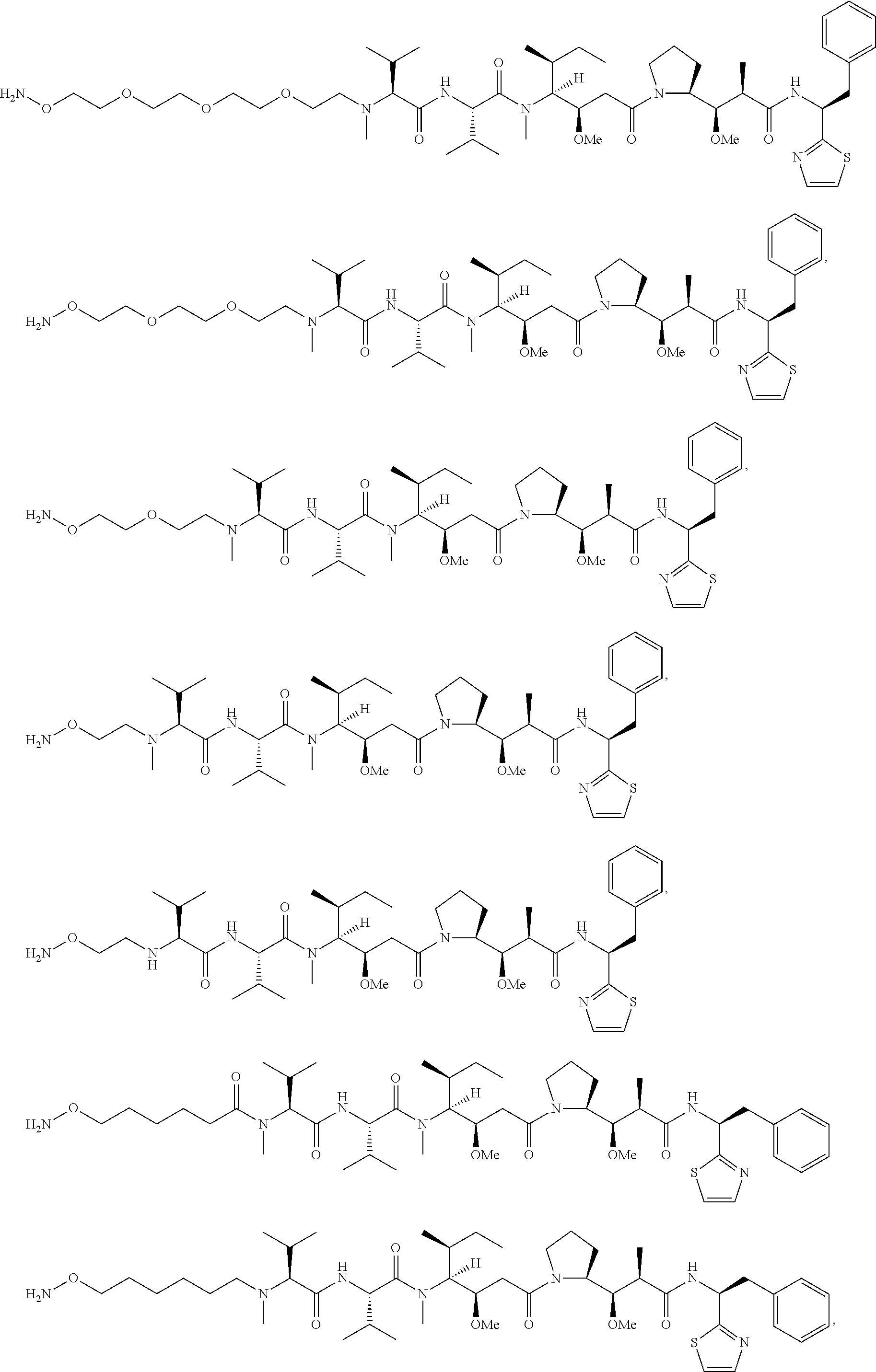

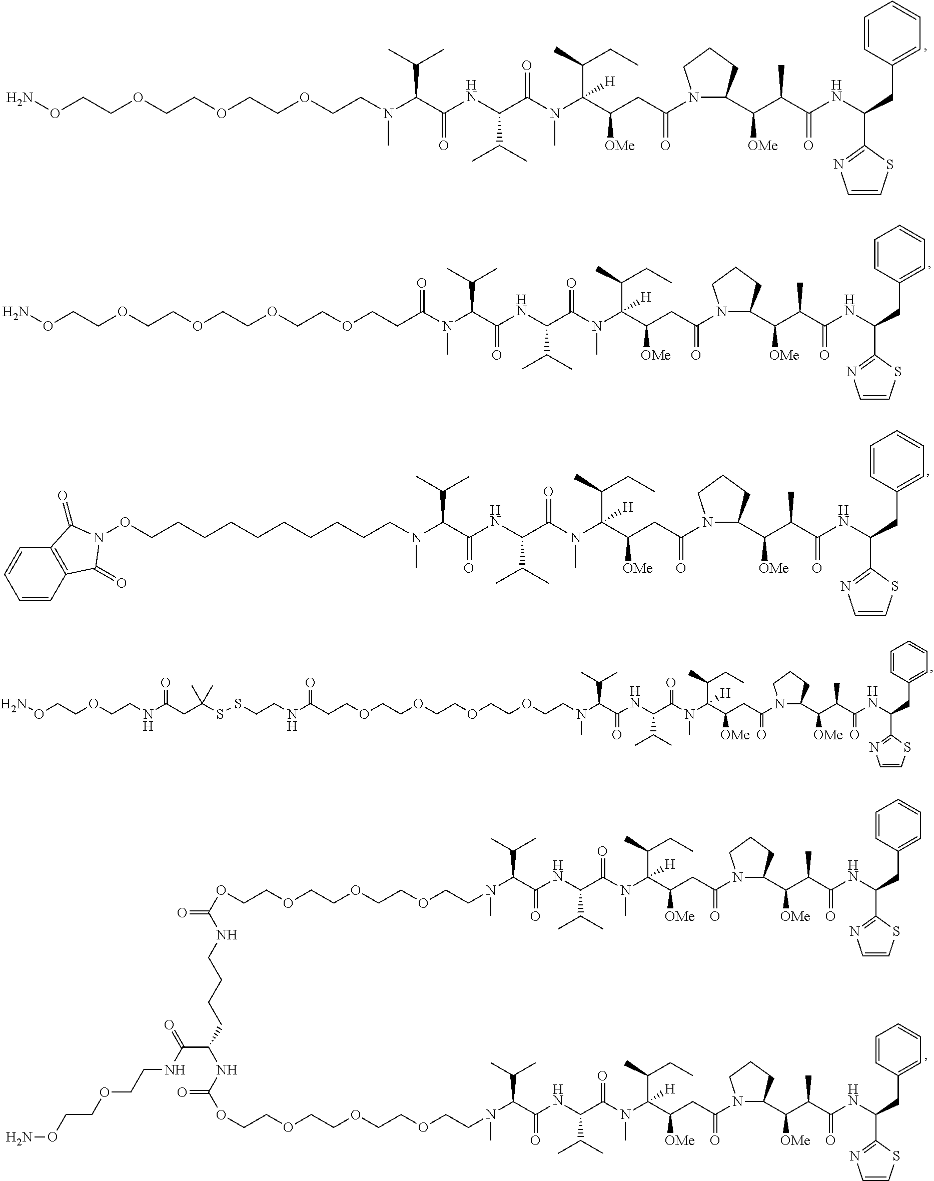

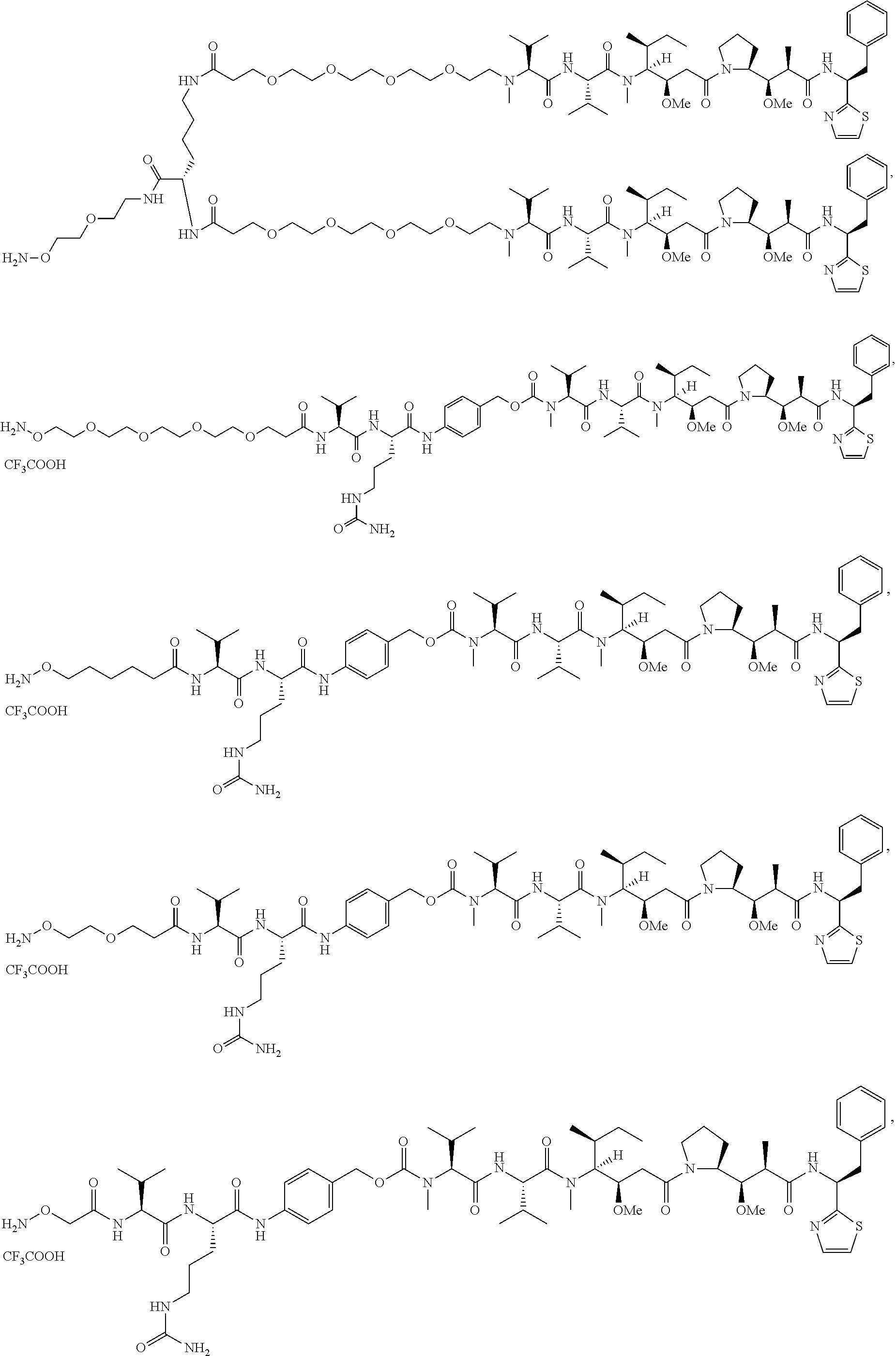

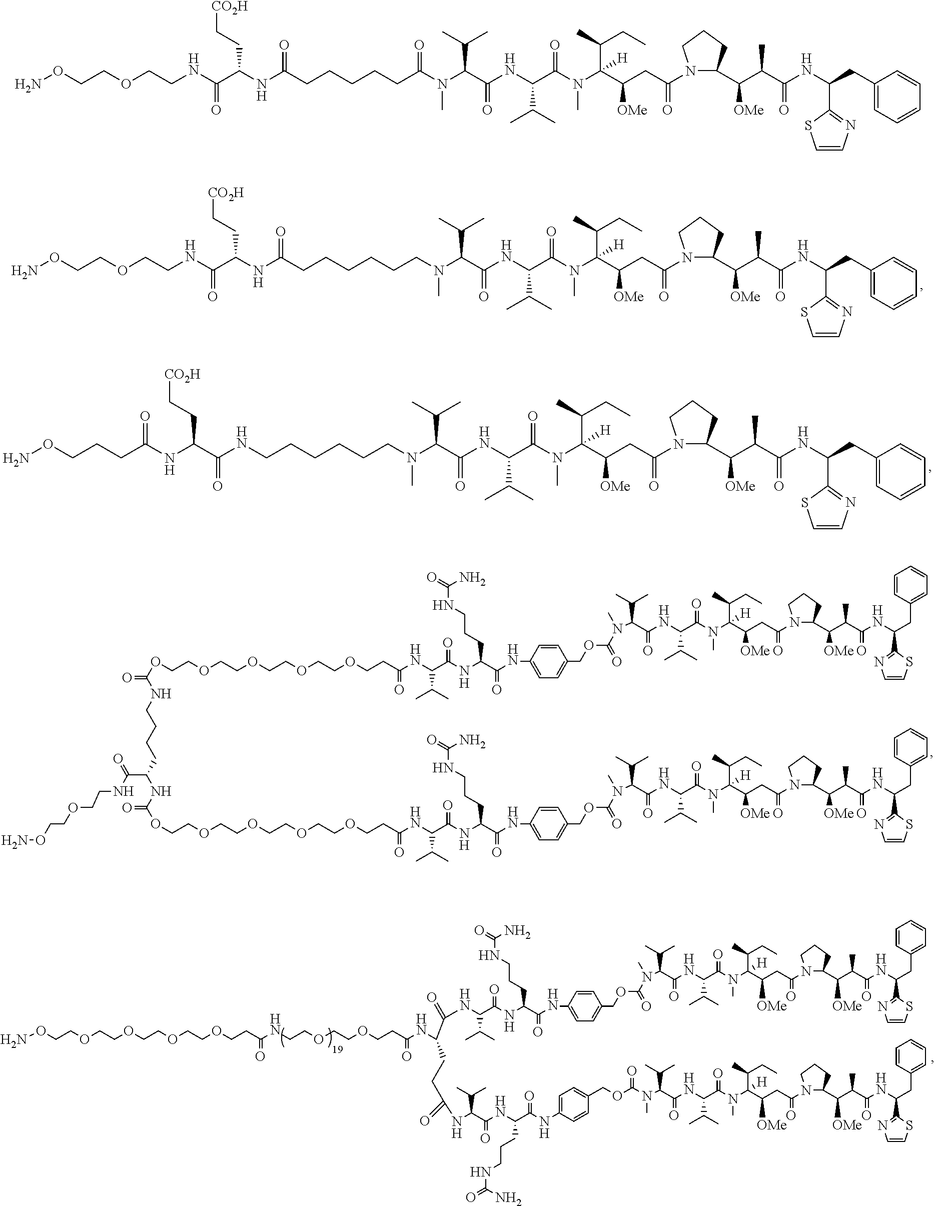

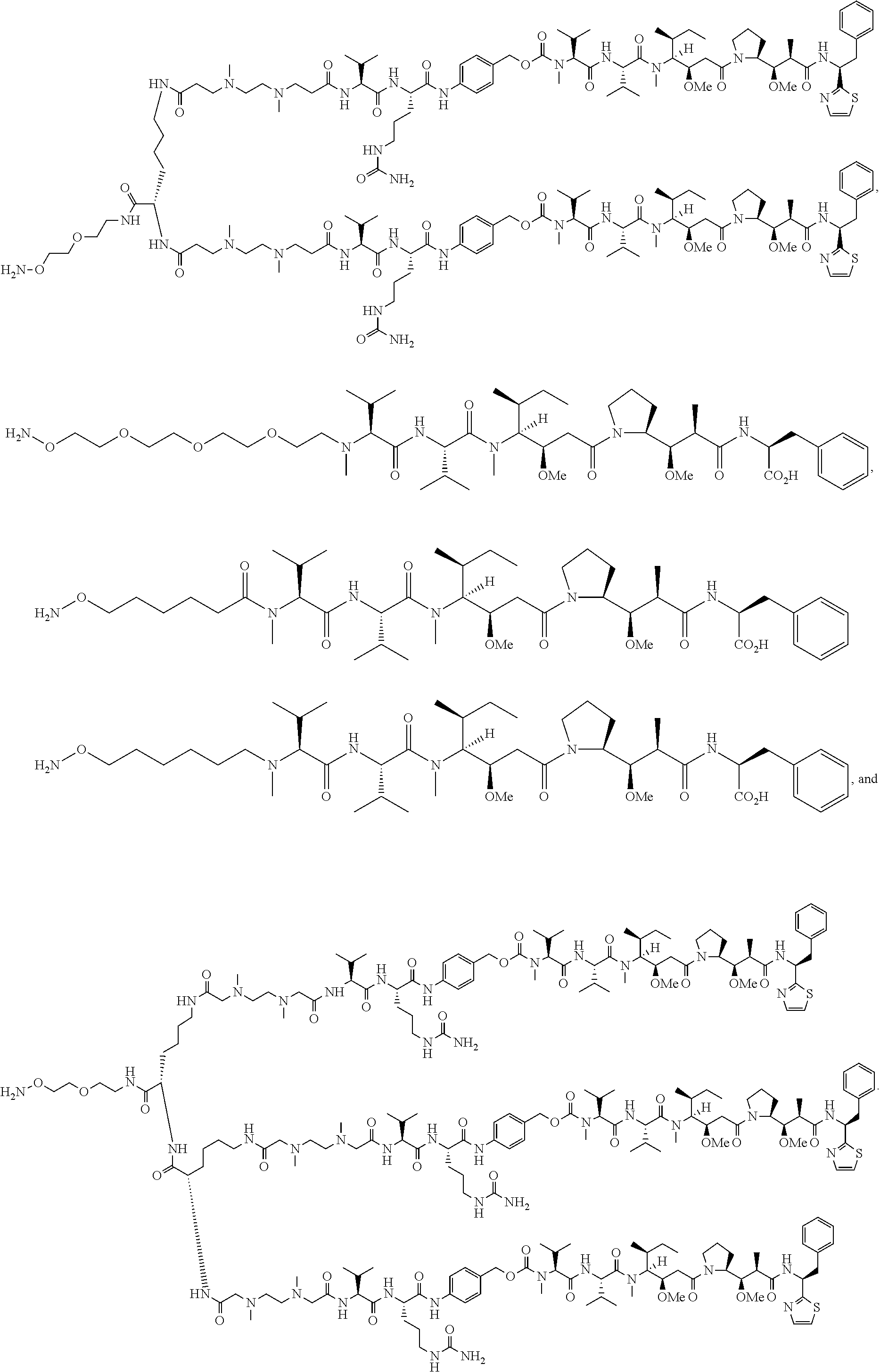

I. Non-Natural Amino Acid Derivatives

The non-natural amino acids used in the methods and compositions described herein have at least one of the following four properties: (1) at least one functional group on the sidechain of the non-natural amino acid has at least one characteristics and/or activity and/or reactivity orthogonal to the chemical reactivity of the 20 common, genetically-encoded amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), or at least orthogonal to the chemical reactivity of the naturally occurring amino acids present in the polypeptide that includes the non-natural amino acid; (2) the introduced non-natural amino acids are substantially chemically inert toward the 20 common, genetically-encoded amino acids; (3) the non-natural amino acid can be stably incorporated into a polypeptide, preferably with the stability commensurate with the naturally-occurring amino acids or under typical physiological conditions, and further preferably such incorporation can occur via an in vivo system; and (4) the non-natural amino acid includes an oxime functional group or a functional group that can be transformed into an oxime group by reacting with a reagent, preferably under conditions that do not destroy the biological properties of the polypeptide that includes the non-natural amino acid (unless of course such a destruction of biological properties is the purpose of the modification/transformation), or where the transformation can occur under aqueous conditions at a pH between about 4 and about 8, or where the reactive site on the non-natural amino acid is an electrophilic site. Any number of non-natural amino acids can be introduced into the polypeptide. Non-natural amino acids may also include protected or masked oximes or protected or masked groups that can be transformed into an oxime group after deprotection of the protected group or unmasking of the masked group. Non-natural amino acids may also include protected or masked carbonyl or dicarbonyl groups, which can be transformed into a carbonyl or dicarbonyl group after deprotection of the protected group or unmasking of the masked group and thereby are available to react with hydroxylamines or oximes to form oxime groups.

Non-natural amino acids that may be used in the methods and compositions described herein include, but are not limited to, amino acids comprising a amino acids with novel functional groups, amino acids that covalently or noncovalently interact with other molecules, glycosylated amino acids such as a sugar substituted serine, other carbohydrate modified amino acids, keto-containing amino acids, aldehyde-containing amino acids, amino acids comprising polyethylene glycol or other polyethers, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, and amino thioacid containing amino acids.

In some embodiments, non-natural amino acids comprise a saccharide moiety. Examples of such amino acids include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine and O-mannosaminyl-L-serine. Examples of such amino acids also include examples where the naturally-occurring N- or O-linkage between the amino acid and the saccharide is replaced by a covalent linkage not commonly found in nature including but not limited to, an alkene, an oxime, a thioether, an amide and the like. Examples of such amino acids also include saccharides that are not commonly found in naturally-occurring proteins such as 2-deoxy-glucose, 2-deoxygalactose and the like,

The chemical moieties incorporated into polypeptides via incorporation of non-natural amino acids into such polypeptides offer a variety of advantages and manipulations of polypeptides. For example, the unique reactivity of a carbonyl or dicarbonyl functional group (including a keto- or aldehyde-functional group) allows selective modification of proteins with any of a number of hydrazine- or hydroxylamine-containing reagents in vivo and in vitro. A heavy atom non-natural amino acid, for example, can be useful for phasing x-ray structure data. The site-specific introduction of heavy atoms using non-natural amino acids also provides selectivity and flexibility in choosing positions for heavy atoms. Photoreactive non-natural amino acids (including but not limited to, amino acids with benzophenone and arylazides (including but not limited to, phenylazide) side chains), for example, allow for efficient in vivo and in vitro photocrosslinking of polypeptides. Examples of photoreactive non-natural amino acids include, but are not limited to, p-azido-phenylalanine and p-benzoyl-phenylalanine. The polypeptide with the photoreactive non-natural amino acids may then be crosslinked at will by excitation of the photoreactive group-providing temporal control. In a non-limiting example, the methyl group of a non-natural amino can be substituted with an isotopically labeled, including but not limited to, with a methyl group, as a probe of local structure and dynamics, including but not limited to, with the use of nuclear magnetic resonance and vibrational spectroscopy.

A. Structure and Synthesis of Non-Natural Amino Acid Derivatives: Carbonyl, Carbonyl like, Masked Carbonyl, and Protected Carbonyl Groups

Amino acids with an electrophilic reactive group allow for a variety of reactions to link molecules via various chemical reactions, including, but not limited to, nucleophilic addition reactions. Such electrophilic reactive groups include a carbonyl- or dicarbonyl-group (including a keto- or aldehyde group), a carbonyl-like- or dicarbonyl-like-group (which has reactivity similar to a carbonyl- or dicarbonyl-group and is structurally similar to a carbonyl- or dicarbonyl-group), a masked carbonyl- or masked dicarbonyl-group (which can be readily converted into a carbonyl- or dicarbonyl-group), or a protected carbonyl- or protected dicarbonyl-group (which has reactivity similar to a carbonyl- or dicarbonyl-group upon deprotection). Such amino acids include amino acids having the structure of Formula (XXXVII):

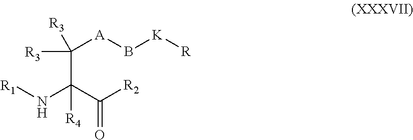

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —NS(O)₂—, —OS(O)₂—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
K is

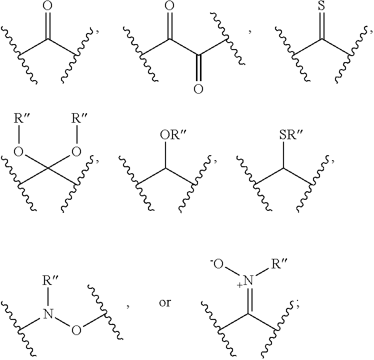

R is H, alkyl, substituted alkyl, cycloalkyl, or substituted cycloalkyl;
each R″ is independently II, alkyl, substituted alkyl, or a protecting group, or when more than one R″ group is present, two R″ optionally form a heterocycloalkyl;
R₁ is H, an amino protecting group, resin, amino acid, polypeptide, or polynucleotide; and
R₂ is OH, an ester protecting group, resin, amino acid, polypeptide, or polynucleotide;
each of R₃ and R₄ is independently H, halogen, lower alkyl, or substituted lower alkyl, or R₃ and R₄ or two R₃ groups optionally form a cycloalkyl or a heterocycloalkyl;
or the -A-B—K—R groups together form a bicyclic or tricyclic cycloalkyl or heterocycloalkyl comprising at least one carbonyl group, including a dicarbonyl group, protected carbonyl group, including a protected dicarbonyl group, or masked carbonyl group, including a masked dicarbonyl group;
or the —K—R group together forms a monocyclic or bicyclic cycloalkyl or heterocycloalkyl comprising at least one carbonyl group, including a dicarbonyl group, protected carbonyl group, including a protected dicarbonyl group, or masked carbonyl group, including a masked dicarbonyl group;
   with a proviso that when A is phenylene and each R₃ is H, B is present; and that when A is —(CH₂)₄— and each R₃ is H, B is not —NHC(O)(CH₂CH₂)—; and that when A and B are absent and each R₃ is H, R is not methyl. Such non-natural amino acids may be in the form of a salt, or may be incorporated into a non-natural amino acid polypeptide, polymer, polysaccharide, or a polynucleotide and optionally post translationally modified.

In certain embodiments, compounds of Formula (XXXVII) are stable in aqueous solution for at least 1 month under mildly acidic conditions. In certain embodiments, compounds of Formula (XXXVII) are stable for at least 2 weeks under mildly acidic conditions. In certain embodiments, compound of Formula (XXXVII) are stable for at least 5 days under mildly acidic conditions. In certain embodiments, such acidic conditions are pH 2 to 8.

In certain embodiments of compounds of Formula (XXXVII), B is lower alkylene, substituted lower alkylene, —O -(alkylene or substituted alkylene)-, —C(R′)═N—N(R′)—, —N(R′) CO—, —C(O)—, —C(R′)═N—, —C(O)-(alkylene or substituted alkylene)-, —CON(R′)-(alkylene or substituted alkylene)-, —S(alkylene or substituted alkylene)-, —S(O)(alkylene or substituted alkylene)-, or —S(O)₂(alkylene or substituted alkylene)-. In certain embodiments of compounds of Formula (XXXVII), B is —O(CH₂)—, —CH═N—, —CH═N—NH—, —NHCH₂—, —NHCO—, —C(O)—, —C(O)—(CH₂)—, —CONH—(CH₂)—, —SCH₂—, —S(═O)CH₂—, or —S(O)₂CH₂—. In certain embodiments of compounds of Formula (XXXVII), R is C₁₋₆ alkyl or cycloalkyl. In certain embodiments of compounds of Formula (XXXVII) R is —CH₃, —CH(CH₃)₂, or cyclopropyl. In certain embodiments of compounds of Formula (XXXVII), R₁ is H, tert-butyloxycarbonyl (Boc), 9-Fluorenylmethoxycarbonyl (Fmoc), N-acetyl, tetrafluoroacetyl (TFA), or benzyloxycarbonyl (Cbz). In certain embodiments of compounds of Formula (XXXVII), R₁ is a resin, amino acid, polypeptide, antibody, or polynucleotide. In certain embodiments of compounds of Formula (XXXVII), R₂ is OH, O-methyl, O-ethyl, or O-t-butyl. In certain embodiments of compounds of Formula (XXXVII), R₂ is a resin, amino acid, polypeptide, antibody, or polynucleotide. In certain embodiments of compounds of Formula (XXXVII), R₂ is a polynucleotide. In certain embodiments of compounds of Formula (XXXVII), R₂ is ribonucleic acid (RNA).

In certain embodiments of compounds of Formula (XXXVII),

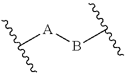

is selected from the group consisting of:
(i) A is substituted lower alkylene, C₄-arylene, substituted arylene, heteroarylene, substituted heteroarylene, alkarylene, substituted alkarylene, aralkylene, or substituted aralkylene;
B is optional, and when present is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS(O)₂—, —OS(O)₂—, —C(O)—, —C(O)-(alkylene or substituted alkylene)-, —C(S)—, —N(R′)—, —C(O)N(R′)—, —CON(R′)-(alkylene or substituted alkylene)-, —CSN(R′)—, —N(R′)CO-(alkylene or substituted alkylene)-, —N(R′)C(O)O—, —N(R′)C(S)—, —S(O)N(R′), —S(O)₂N(R′), —N(R′)C(O)N(R′)—, —N(R′)C(S)N(R′)—, —N(R′)S(O)N(R′)—, —N(R′)S(O)₂N(R′)—, —N(R′)—N═, —C(R′)═N—N(R′)—, —C(R′)═N—N═, —C(R′)₂—N═N—, and —C(R′)₂—N(R′)—N(R′)—;
(ii) A is optional, and when present is substituted lower alkylene, C₄-arylene, substituted arylene, heteroarylene, substituted heteroarylene, alkarylene, substituted alkarylene, aralkylene, or substituted aralkylene;
B is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS(O)₂—, —OS(O)₂—, —C(O)—, —C(O)-(alkylene or substituted alkylene)-, —C(S)—, —N(R′)—, —C(O)N(R′)—, —CON(R′)-(alkylene or substituted alkylene)-, —CSN(R′)—, —N(R′)CO-(alkylene or substituted alkylene)-, —N(R′)C(O)O—, —N(R′)C(S)—, —S(O)N(R′), —S(O)₂N(R′), —N(R′)C(O)N(R′)—, —N(R′)C(S)N(R′)—, —N(R′)S(O)N(R′)—, —N(R′)S(O)₂N(R′)—, —N(R′)—N═, —C(R′)═N—N(R′)—, —C(R′)═N—N═, —C(R′)₂—N═N—, and —C(R′)₂—N(R′)—N(R′)—;
(iii) A is lower alkylene;
B is optional, and when present is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS(O)₂—, —OS(O)₂—, —C(O)—, —C(O)-(alkylene or substituted alkylene)-, —C(S)—, —N(R′)—, —C(O)N(R′)—, —CSN(R′)—, —CON(R′)-(alkylene or substituted alkylene)-, —N(R′)C(O)O—, —N(R′)C(S)—, —S(O)N(R′), —S(O)₂N(R′), —N(R′)C(O)N(R′)—, —N(R′)C(S)N(R′)—, —N(R′)S(O)N(R′)—, —N(R′)S(O)₂N(R′)—, —N(R′)—N═, —C(R′)═N—N(R′)—, —C(R′)═N—N═, —C(R′)₂—N═N—, and —C(R′)₂—N(R′)—N(R′)—; and
(iv) A is phenylene;
B is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS(O)₂—, —OS(O)₂—, —C(O)—, —C(O)-(alkylene or substituted alkylene)-, —C(S)—, —N(R′)—, —C(O)N(R′)—, —CON(R′)-(alkylene or substituted alkylene)-, —CSN(R′)—, —N(R′)CO-(alkylene or substituted alkylene)-, —N(R′)C(O)O—, —N(R′)C(S)—, —S(O)N(R′), —S(O)₂N(R′), —N(R′)C(O)N(R′)—, —N(R′)C(S)N(R′)—, —N(R′)S(O)N(R′)—, —N(R′)S(O)₂N(R′)—, —N(R′)—N═, —C(R′)═N—N(R′)—, —C(R′)═N—N═, —C(R′)₂—N═N—, and —C(R′)₂—N(R′)—N(R′)—;
K is

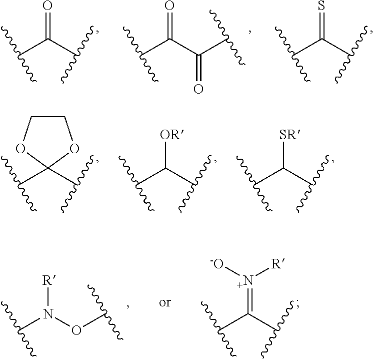

each R′ is independently H, alkyl, or substituted alkyl;
R₁ is optional, and when present, is H, an amino protecting group, resin, amino acid, polypeptide, or polynucleotide; and
R₂ is optional, and when present, is OH, an ester protecting group, resin, amino acid, polypeptide, or polynucleotide; and
each R₃ and R₄ is independently H, halogen, lower alkyl, or substituted lower alkyl;
R is H, alkyl, substituted alkyl, cycloalkyl, or substituted cycloalkyl;

In addition, amino acids having the structure of Formula (XXXVIII) are included:

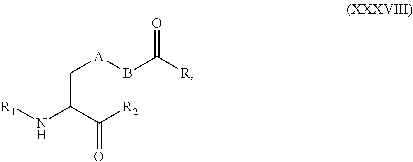

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —NS(O)₂—, —OS(O)₂—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is H, an amino protecting group, resin, at least one amino acid, polypeptide, or polynucleotide; and
R₂ is OH, an ester protecting group, resin, at least one amino acid, polypeptide, or polynucleotide;
   with a proviso that when A is phenylene, B is present; and that when A is —(CH₂)₄—, B is not —NHC(O)(CH₂CH₂)—; and that when A and B are absent, R is not methyl. Such non-natural amino acids may be in the form of a salt, or may be incorporated into a non-natural amino acid polypeptide, polymer, polysaccharide, or a polynucleotide and optionally post translationally modified.

In addition, amino acids having the structure of Formula (XXXIX) are included:

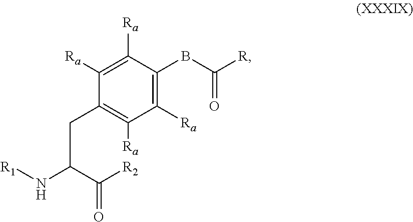

wherein:
B is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —NS(O)₂—, —OS(O)₂—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide; each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl. Such non-natural amino acids may be in the form of a salt, or may be incorporated into a non-natural amino acid polypeptide, polymer, polysaccharide, or a polynucleotide and optionally post translationally modified.

In addition, the following amino acids are included:

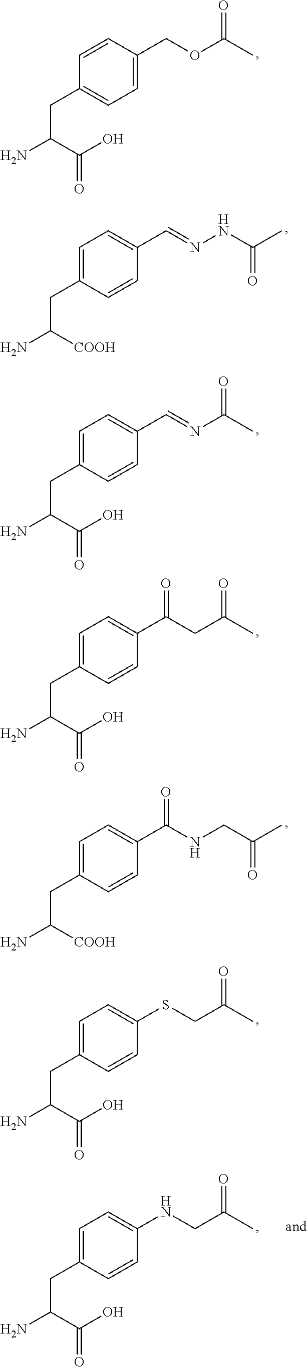

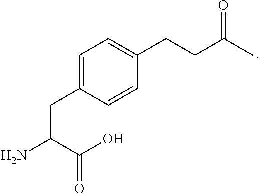 [SMILES: CC(=O)CCc1ccc(CC(N)C(=O)O)cc1]

Such non-natural amino acids may be are optionally amino protected group, carboxyl protected and/or in the form of a salt, or may be incorporated into a non-natural amino acid polypeptide, polymer, polysaccharide, or a polynucleotide and optionally post translationally modified.

In addition, the following amino acids having the structure of Formula (XXXX) are included:

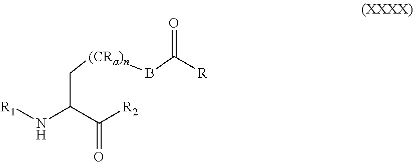

wherein
—NS(O)₂—, —OS(O)₂—, optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted allcyl;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl; and n is 0 to 8;
   with a proviso that when A is —(CH₂)₄—, B is not —NHC(O)(CH₂CH₂)—. Such non-natural amino acids may be in the form of a salt, or may be incorporated into a non-natural amino acid polypeptide, polymer, polysaccharide, or a polynucleotide and optionally post translationally modified.

In addition, the following amino acids are included:

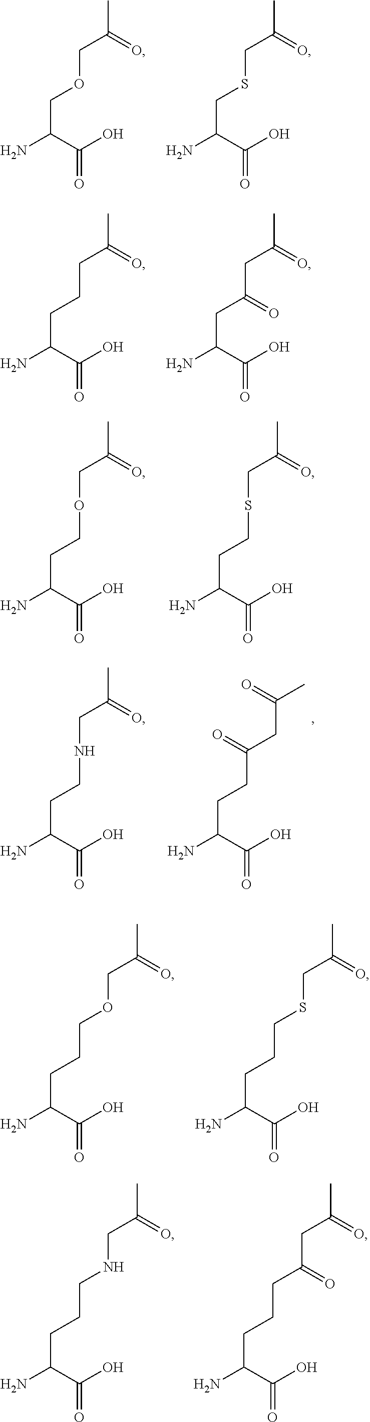

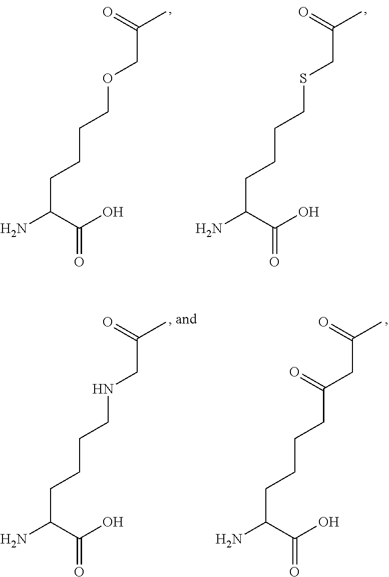

wherein such compounds are optionally amino protected, optionally carboxyl protected, optionally amino protected and carboxyl protected, or a salt thereof, or may be incorporated into a non-natural amino acid polypeptide, polymer, polysaccharide, or a polynucleotide and optionally post translationally modified.

In addition, the following amino acids having the structure of Formula (XXXXI) are included:

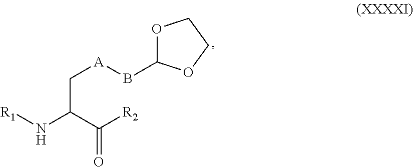

wherein,
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —NS(O)₂—, —OS(O)₂—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N═, —C(R′)₂—N═N—, and —C(R′)₂—N(R′)—N(R′)—, where each R′ is independently H, alkyl, or substituted alkyl;
R₁ is H, an amino protecting group, resin, amino acid, polypeptide, or polynucleotide; and
R₂ is OH, an ester protecting group, resin, amino acid, polypeptide, or polynucleotide.
   Such non-natural amino acids may be in the form of a salt, or may be incorporated into a non-natural amino acid polypeptide, polymer, polysaccharide, or a polynucleotide and optionally post translationally modified.

In addition, the following amino acids having the structure of Formula (XXXXII) are included:

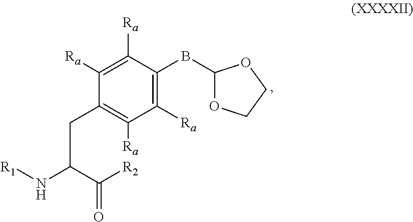

wherein,
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)₁ where k is 1, 2, or 3, —S(O)ₖ(alkylene or substituted alkylene)-, —C(O)—, —NS(O)₂—, —OS(O)₂—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide; wherein each Rₐ is independently selected from the group consisting of H, halogen, alkyl, substituted alkyl, —N(R′)₂, —C(O)ₖR′ where k is 1, 2, or 3, —C(O)N(R′)₂, —OR′, and S(O)ₖR′, where each R′ is independently H, alkyl, or substituted alkyl.
   Such non-natural amino acids may be in the form of a salt, or may be incorporated into a non-natural amino acid polypeptide, polymer, polysaccharide, or a polynucleotide and optionally post translationally modified.

In addition, the following amino acids are included:

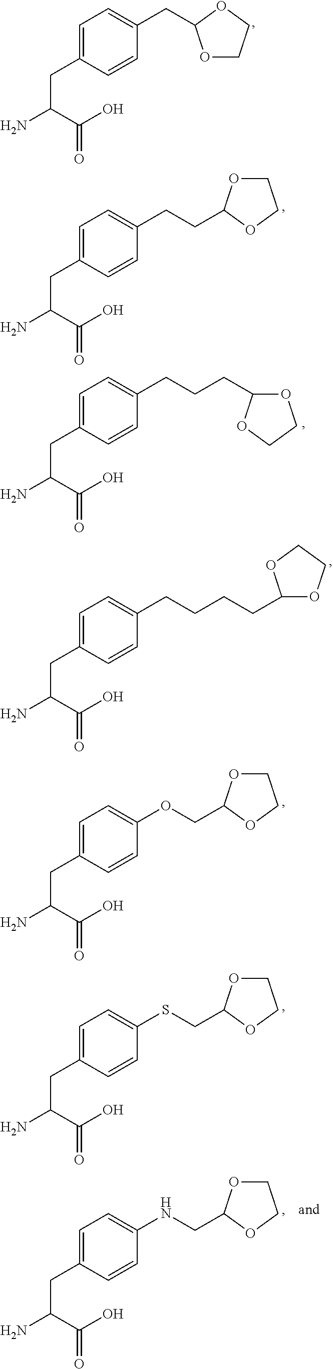

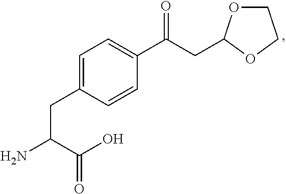 [SMILES: NC(Cc1ccc(C(=O)CC2OCCO2)cc1)C(=O)O]

wherein such compounds are optionally amino protected, optionally carboxyl protected, optionally amino protected and carboxyl protected, or a salt thereof, or may be incorporated into a non-natural amino acid polypeptide, polymer, polysaccharide, or a polynucleotide and optionally post translationally modified.

In addition, the following amino acids having the structure of Formula (XXXXIV) are included:

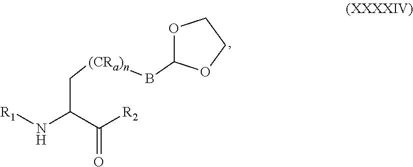

wherein,
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —NS(O)₂—, —OS(O)₂—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently alkyl, or substituted alkyl;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl; and n is 0 to 8.
   Such non-natural amino acids may be in the form of a salt, or may be incorporated into a non-natural amino acid polypeptide, polymer, polysaccharide, or a polynucleotide and optionally post translationally modified.

In addition, the following amino acids are included:

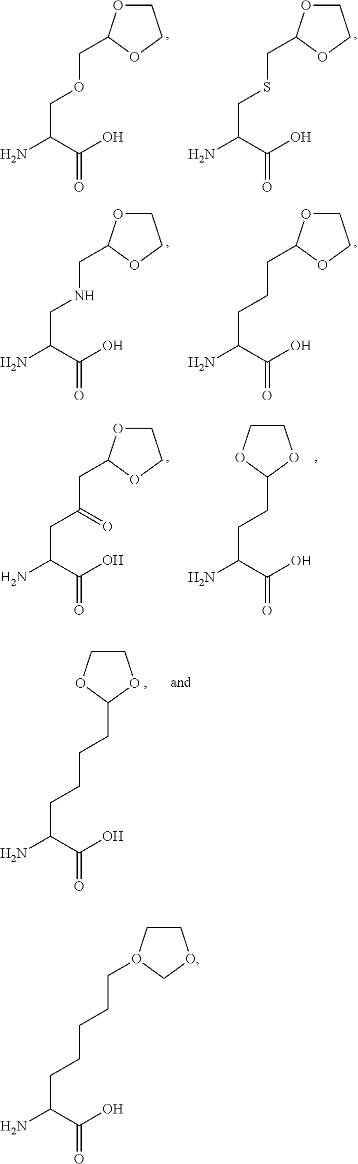

wherein such compounds are optionally amino protected, optionally carboxyl protected, optionally amino protected and carboxyl protected, or a salt thereof, or may be incorporated into a non-natural amino acid polypeptide, polymer, polysaccharide, or a polynucleotide and optionally post translationally modified.

In addition to monocarbonyl structures, the non-natural amino acids described herein may include groups such as dicarbonyl, dicarbonyl like, masked dicarbonyl and protected dicarbonyl groups.

For example, the following amino acids having the structure of Formula (XXXXV) are included:

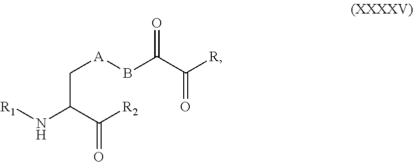

wherein,
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —NS(O)₂—, —OS(O)₂—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide.
   Such non-natural amino acids may be in the form of a salt, or may be incorporated into a non-natural amino acid polypeptide, polymer, polysaccharide, or a polynucleotide and optionally post translationally modified,

In addition, the following amino acids having the structure of Formula (XXXXVI) are included:

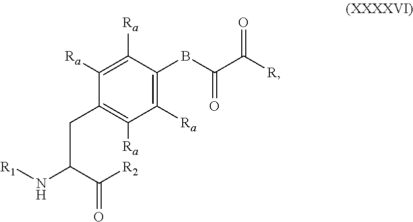

wherein,
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)k(alkylene or substituted alkylene)-, —C(O)—, —NS(O)₂—, —OS(O)₂—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;

wherein each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl.
Such non-natural amino acids may be in the form of a salt, or may be incorporated into a non-natural amino acid polypeptide, polymer, polysaccharide, or a polynucleotide and optionally post translationally modified.

In addition, the following amino acids are included:

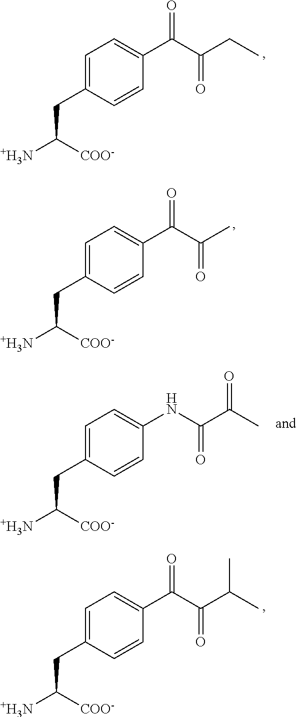

wherein such compounds are optionally amino protected and carboxyl protected, or a salt thereof. Such non-natural amino acids may be in the form of a salt, or may be incorporated into a non-natural amino acid polypeptide, polymer, polysaccharide, or a polynucleotide and optionally post translationally modified,

In addition, the following amino acids having the structure of Formula (XXXXVII) are included:

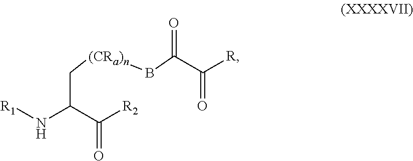

wherein,
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —NS(O)₂—, —OS(O)₂—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide; each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl; and n is 0 to 8. Such non-natural amino acids may be in the form of a salt, or may be incorporated into a non-natural amino acid polypeptide, polymer, polysaccharide, or a polynucleotide and optionally post translationally modified.

In addition, the following amino acids are included:

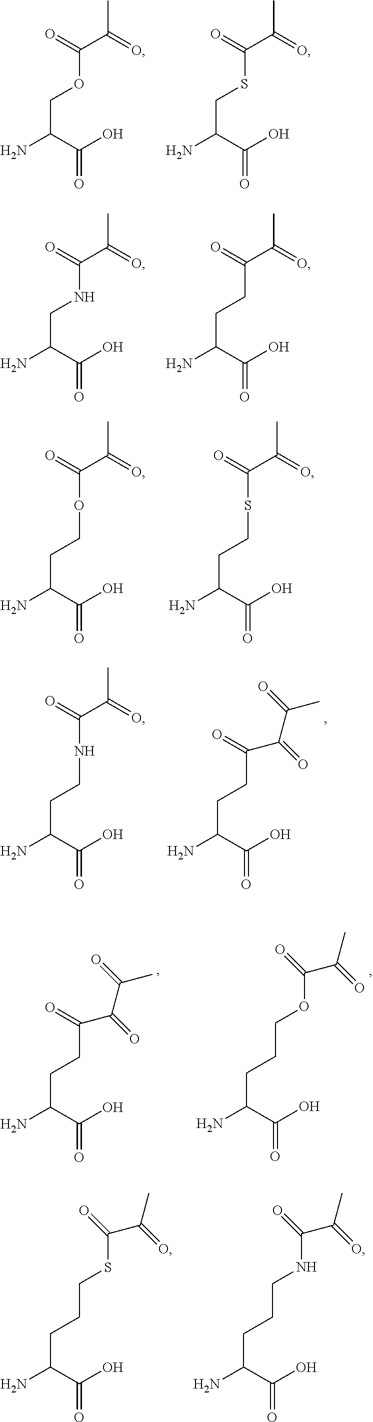

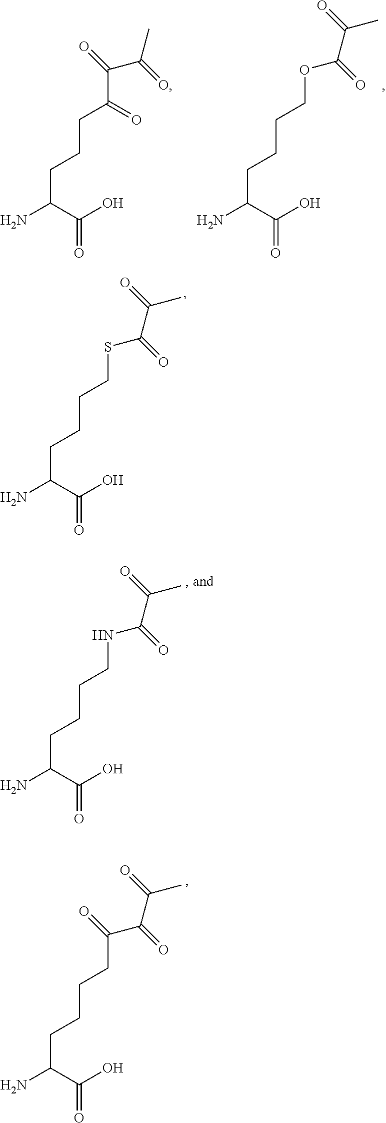 [SMILES: CC(=O)C(=O)C(=O)CCCC(N)C(=O)O ; CC(=O)C(=O)OCCCCC(N)C(=O)O ; CC(=O)C(=O)SCCCCC(N)C(=O)O ; CC(=O)C(=O)NCCCCC(N)C(=O)O ; CC(=O)C(=O)C(=O)CCCCC(N)C(=O)O]

wherein such compounds are optionally amino protected and carboxyl protected, or a salt thereof, or may be incorporated into a non-natural amino acid polypeptide, polymer, polysaccharide, or a polynucleotide and optionally post translationally modified.

In addition, the following amino acids having the structure of Formula (XXXXVIII) are included:

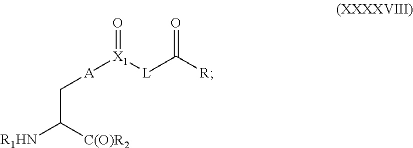

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
X₁ is C, S, or S(O); and L is alkylene, substituted alkylene, N(R′)(alkylene) or N(R′)(substituted alkylene), where R′ is H, alkyl, substituted alkyl, cycloalkyl, or substituted cycloalkyl.
   Such non-natural amino acids may be in the form of a salt, or may be incorporated into a non-natural amino acid polypeptide, polymer, polysaccharide, or a polynucleotide and optionally post translationally modified.

In addition, the following amino acids having the structure of Formula (XXXXIX) are included:

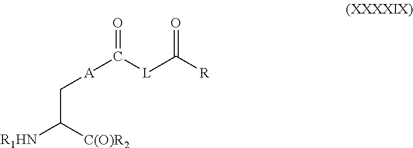

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
L is alkylene, substituted alkylene, N(R′)(alkylene) or N(R′)(substituted alkylene), where R′ is H, alkyl, substituted alkyl, cycloalkyl, or substituted cycloalkyl.
   Such non-natural amino acids may be in the form of a salt, or may be incorporated into a non-natural amino acid polypeptide, polymer, polysaccharide, or a polynucleotide and optionally post translationally modified.

In addition, the following amino acids having the structure of Formula (XXXXX) are included:

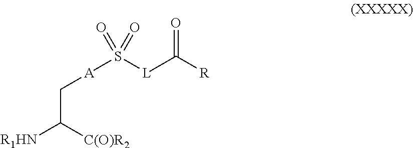

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;

R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
L is alkylene, substituted alkylene, N(R′)(alkylene) or N(R′)(substituted alkylene), where R′ is H, alkyl, substituted alkyl, cycloalkyl, or substituted cycloalkyl.
   Such non-natural amino acids may be in the form of a salt, or may be incorporated into a non-natural amino acid polypeptide, polymer, polysaccharide, or a polynucleotide and optionally post translationally modified.

In addition, the following amino acids having the structure of Formula (XXXXXI) are included:

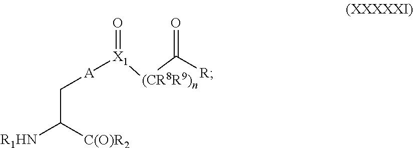

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
X₁ is C, S, or S(O); and n is 0, 1, 2, 3, 4, or 5; and each R⁸ and R⁹ on each CR⁸R⁹ group is independently selected from the group consisting of H, alkoxy, alkylamine, halogen, alkyl, aryl, or any R⁸ and R⁹ can together form ═O or a cycloalkyl, or any to adjacent R⁸ groups can together form a cycloalkyl.
   Such non-natural amino acids may be in the form of a salt, or may be incorporated into a non-natural amino acid polypeptide, polymer, polysaccharide, or a polynucleotide and optionally post translationally modified.

In addition, the following amino acids having the structure of Formula (XXXXXII) are included:

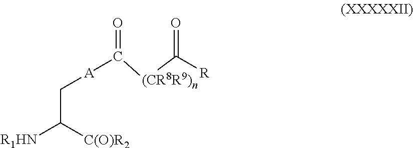

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is H, an amino protecting group, resin, amino acid, polypeptide, or polynucleotide; and

R₂ is OH, an ester protecting group, resin, amino acid, polypeptide, or polynucleotide; n is 0, 1, 2, 3, 4, or 5; and each R⁸ and R⁹ on each CR⁸R⁹ group is independently selected from the group consisting of H, alkoxy, alkylamine, halogen, alkyl, aryl, or any R⁸ and R⁹ can together form ═O or a cycloalkyl, or any to adjacent R⁸ groups can together form a cycloalkyl.
Such non-natural amino acids may be in the form of a salt, or may be incorporated into a non-natural amino acid polypeptide, polymer, polysaccharide, or a polynucleotide and optionally post translationally modified.

In addition, the following amino acids having the structure of Formula (XXXXXIII) are included:

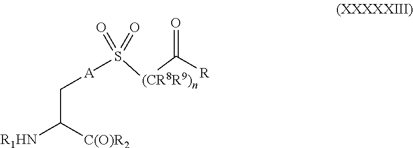

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide; n is 0, 1, 2, 3, 4, or 5; and each R⁸ and R⁹ on each CR⁸R⁹ group is independently selected from the group consisting of H, alkoxy, alkylamine, halogen, alkyl, aryl, or any R⁸ and R⁹ can together form ═O or a cycloalkyl, or any to adjacent R⁸ groups can together form a cycloalkyl.
   Such non-natural amino acids may be in the form of a salt, or may be incorporated into a non-natural amino acid polypeptide, polymer, polysaccharide, or a polynucleotide and optionally post translationally modified.

In addition, the following amino acids having the structure of Formula (XXXXXIV) are included:

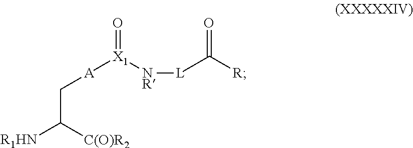

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;

R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
X₁ is C, S, or S(O); and L is alkylene, substituted alkylene, N(R′)(alkylene) or N(R′)(substituted alkylene), where R′ is H, alkyl, substituted alkyl, cycloalkyl, or substituted cycloalkyl.
   Such non-natural amino acids may be in the form of a salt, or may be incorporated into a non-natural amino acid polypeptide, polymer, polysaccharide, or a polynucleotide and optionally post translationally modified.

In addition, the following amino acids having the structure of Formula (XXXXXV) are included:

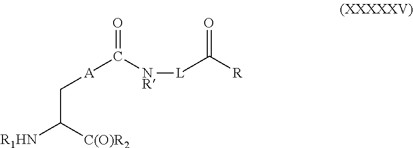

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
L is alkylene, substituted alkylene, N(R′)(alkylene) or N(R′)(substituted alkylene), where R′ is H, alkyl, substituted alkyl, cycloalkyl, or substituted cycloalkyl.
   Such non-natural amino acids may be in the form of a salt, or may be incorporated into a non-natural amino acid polypeptide, polymer, polysaccharide, or a polynucleotide and optionally post translationally modified.

In addition, the following amino acids having the structure of Formula (XXXXXVI) are included:

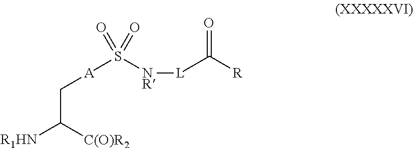

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
L is alkylene, substituted alkylene, N(R′)(alkylene) or N(R′)(substituted alkylene), where R′ is H, alkyl, substituted alkyl, cycloalkyl, or substituted cycloalkyl.
   Such non-natural amino acids may be in the form of a salt, or may be incorporated into a non-natural amino acid polypeptide, polymer, polysaccharide, or a polynucleotide and optionally post translationally modified.

In addition, amino acids having the structure of Formula (XXXXXVII) are included:

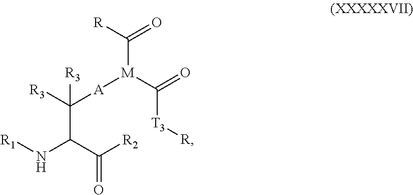

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;

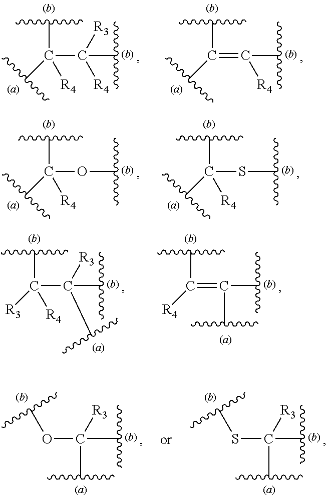

M is —C(R₃)—,
   where (a) indicates bonding to the A group and (b) indicates bonding to respective carbonyl groups, R₃ and R₄ are independently chosen from H, halogen, alkyl, substituted alkyl, cycloalkyl, or substituted cycloalkyl, or R₃ and R₄ or two R₃ groups or two R₄ groups optionally form a cycloalkyl or a heterocycloalkyl;
R is H, halogen, alkyl, substituted alkyl, cycloalkyl, or substituted cycloalkyl;
T₃ is a bond, C(R)(R), O, or S, and R is H, halogen, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide.
   Such non-natural amino acids may be in the form of a salt, or may be incorporated into a non-natural amino acid polypeptide, polymer, polysaccharide, or a polynucleotide and optionally post translationally modified.

In addition, amino acids having the structure of Formula (XXXXXVIII) are included:

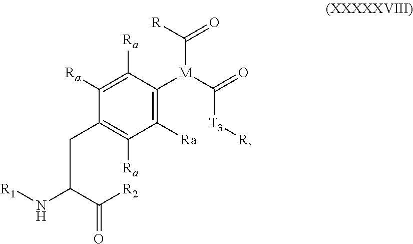

wherein:
M is —C(R₃)—,

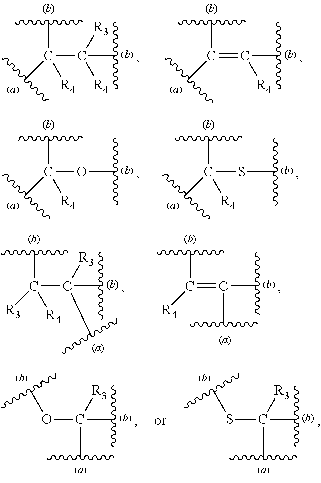

where (a) indicates bonding to the A group and (b) indicates bonding to respective carbonyl groups, R₃ and R₄ are independently chosen from H, halogen, alkyl, substituted alkyl, cycloalkyl, or substituted cycloalkyl, or R₃ and R₄ or two R₃ groups or two R₄ groups optionally form a cycloalkyl or a heterocycloalkyl;
R is H, halogen, alkyl, substituted alkyl, cycloalkyl, or substituted cycloalkyl;
T₃ is a bond, C(R)(R), O, or S, and R is H, halogen, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
   each Rₐ is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl,
   Such non-natural amino acids may be in the form of a salt, or may be incorporated into a non-natural amino acid polypeptide, polymer, polysaccharide, or a polynucleotide and optionally post translationally modified,

In addition, amino acids having the structure of Formula (XXXXXIX) are included:

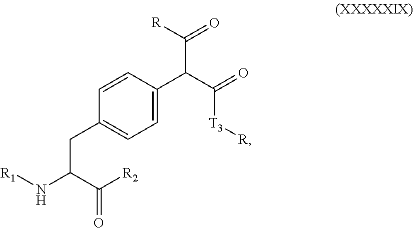

wherein:
R is H, halogen, alkyl, substituted alkyl, cycloalkyl, or substituted cycloalkyl; and
T₃ is O, or S,
   Such non-natural amino acids may be in the form of a salt, or may be incorporated into a non-natural amino acid polypeptide, polymer, polysaccharide, or a polynucleotide and optionally post translationally modified.

In addition, amino acids having the structure of Formula (XXXXXX) are included:

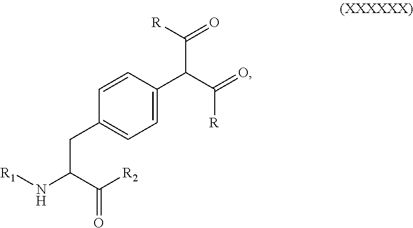

wherein:
R is H, halogen, alkyl, substituted alkyl, cycloalkyl, or substituted cycloalkyl.

In addition, the following amino acids having structures of Formula (XXXXXX) are included:

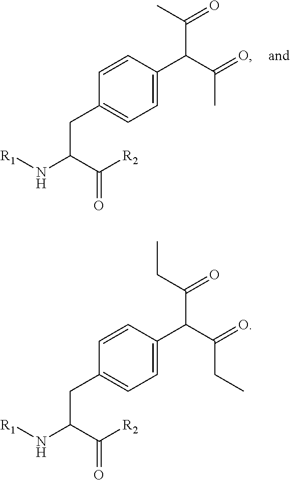

Such non-natural amino acids may be in the form of a salt, or may be incorporated into a non-natural amino acid polypeptide, polymer, polysaccharide, or a polynucleotide and optionally post translationally modified.

The carbonyl or dicarbonyl functionality can be reacted selectively with a hydroxylamine-containing reagent under mild conditions in aqueous solution to form the corresponding oxime linkage that is stable under physiological conditions. See, e.g., Jencks, W. P., J. Am. Chem. Soc. 81, 475-481 (1959); Shao, J. and Tam, J. P., J. Am, Chem. Soc. 117(14):3893-3899 (1995). Moreover, the unique reactivity of the carbonyl or dicarbonyl group allows for selective modification in the presence of the other amino acid side chains. See, e.g., Cornish, V. W., et al., J. Am. Chem. Soc. 118:8150-8151 (1996); Geoghegan, K. F. & Stroh, J. G., Bioconjug. Chem. 3:138-146 (1992); Mahal, L. K., et al., Science 276:1125-1128 (1997).

The synthesis of p-acetyl-(+/−)-phenylalanine and m-acetyl-(+/−)-phenylalanine is described in Zhang, Z., et al., Biochemistry 42: 6735-6746 (2003), incorporated by reference. Other carbonyl- or dicarbonyl-containing amino acids can be similarly prepared.

In some embodiments, a polypeptide comprising a non-natural amino acid is chemically modified to generate a reactive carbonyl or dicarbonyl functional group. For instance, an aldehyde functionality useful for conjugation reactions can be generated from a functionality having adjacent amino and hydroxyl groups. Where the biologically active molecule is a polypeptide, for example, an N-terminal serine or threonine (which may be normally present or may be exposed via chemical or enzymatic digestion) can be used to generate an aldehyde functionality under mild oxidative cleavage conditions using periodate. See, e.g., Gaertner, et. al., Bioconjug. Chem. 3: 262-268 (1992); Geoghegan, K. & Stroh, J., Bioconjug. Chem. 3:138-146 (1992); Gaertner et al., J. Biol. Chem. 269:7224-7230 (1994). However, methods known in the art are restricted to the amino acid at the N-terminus of the peptide or protein.

Additionally, by way of example a non-natural amino acid bearing adjacent hydroxyl and amino groups can be incorporated into a polypeptide as a “masked” aldehyde functionality. For example, 5-hydroxylysine bears a hydroxyl group adjacent to the epsilon amine. Reaction conditions for generating the aldehyde typically involve addition of molar excess of sodium metaperiodate under mild conditions to avoid oxidation at other sites within the polypeptide. The pH of the oxidation reaction is typically about 7.0. A typical reaction involves the addition of about 1.5 molar excess of sodium meta periodate to a buffered solution of the polypeptide, followed by incubation for about 10 minutes in the dark. See, e.g. U.S. Pat. No. 6,423,685.

### B. Structure And Synthesis of Non-Natural Amino Acids: Dicarbonyl, Dicarbonyl-Like, Masked Dicarbonyl, and Protected Dicarbonyl Groups

Amino acids with an electrophilic reactive group allow for a variety of reactions to link molecules via nucleophilic addition reactions among others, Such electrophilic reactive groups include a dicarbonyl group (including a diketone group, a ketoaldehyde group, a ketoacid group, a ketoester group, and a ketothioester group), a dicarbonyl-like group (which has reactivity similar to a dicarbonyl group and is structurally similar to a dicarbonyl group), a masked dicarbonyl group (which can be readily converted into a dicarbonyl group), or a protected dicarbonyl group (which has reactivity similar to a dicarbonyl group upon deprotection). Such amino acids include amino acids having the structure of Formula (XXXVII):

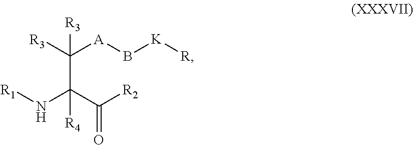

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker linked at one end to a diamine containing moiety, the linker selected from the group consisting of lower alkylene, substituted lower alkylene, lower alkenylene, substituted lower alkenylene, lower heteroalkylene, substituted lower heteroalkylene, —O-(alkylene or substituted alkylene)-, —S-(alkylene or substituted alkylene)-, —C(O)R″—, —S(O)ₖ(alkylene or substituted alkylene)-, where k is 1, 2, or 3, —C(O)-(alkylene or substituted alkylene)-, —C(S)-(alkylene or substituted alkylene)-, —NR″-(alkylene or substituted alkylene)-, —CON(R″)-(alkylene or substituted alkylene)-, —CSN(R″)-(alkylene or substituted alkylene)-, and —N(R″)CO-(alkylene or substituted alkylene)-, where each R″ is independently H, alkyl, or substituted alkyl;
K is

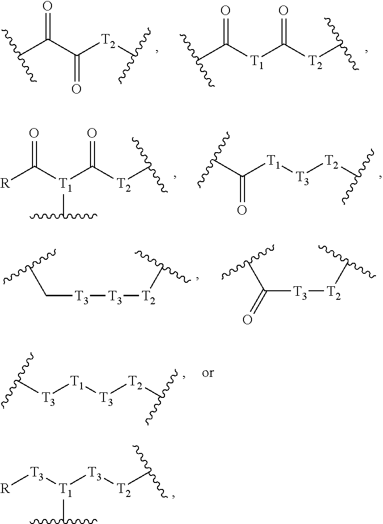

where,
T₁ is a bond, optionally substituted C₁-C₄ alkylene, optionally substituted C₁-C₄ alkenylene, or optionally substituted heteroalkyl;
wherein each optional substituents is independently selected from lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
T₂, is selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
T₃ is

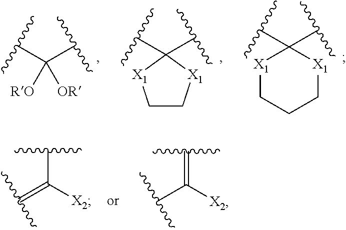

where each X₁ is independently selected from the group consisting of —O—, —S—, —N(H)—, —N(R)—, —N(Ac)-, and N(OMe)-; X₂ is OR, -OAc, —SR, —N(R)₂, —N(R)(Ac), —N(R)(OMe), or N₃, and where each R′ is independently H, alkyl, or substituted alkyl;
R is H, halogen, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
or the A-B—K—R groups together form a bicyclic or tricyclic cycloalkyl or heterocycloalkyl comprising at least one carbonyl group, including a dicarbonyl group, protected carbonyl group, including a protected dicarbonyl group, or masked carbonyl group, including a masked dicarbonyl group;
or the —K—R group together forms a monocyclic or bicyclic cycloalkyl or heterocycloalkyl comprising at least one carbonyl group, including a dicarbonyl group, protected carbonyl group, including a protected dicarbonyl group, or masked carbonyl group, including a masked dicarbonyl group.

Non-limiting example of dicarbonyl amino acids having the structure of Formula (XXXVII) include:

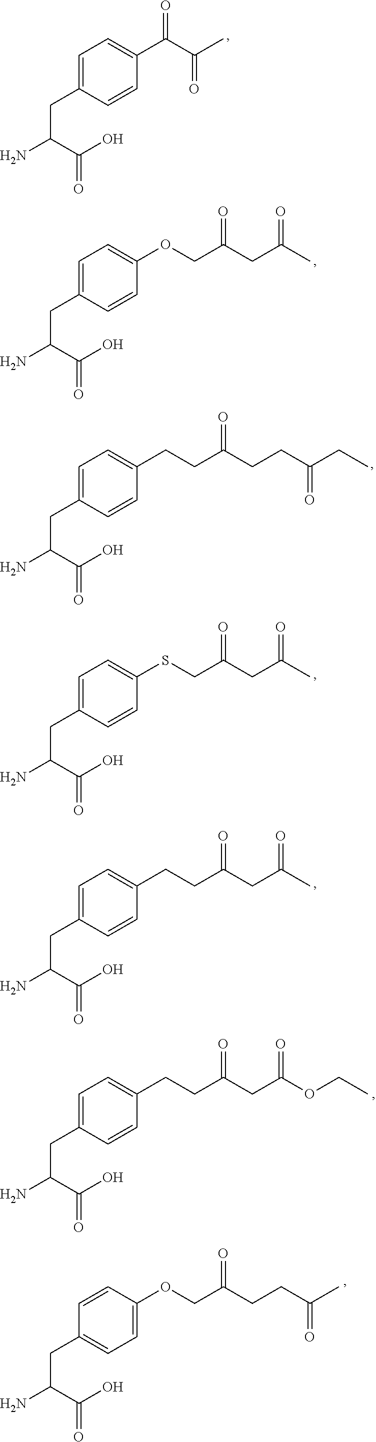

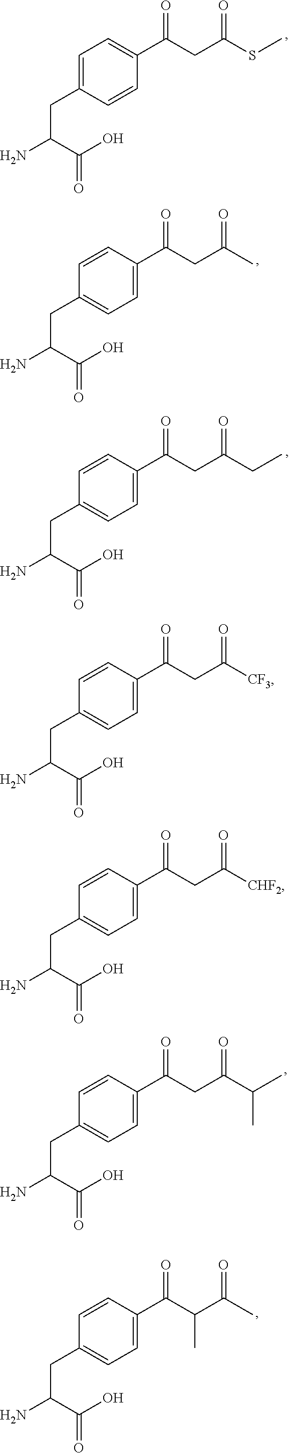

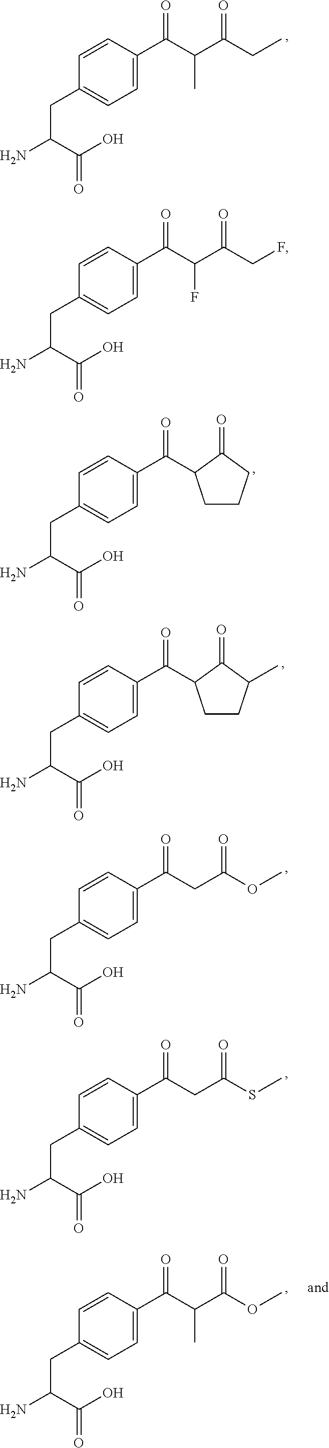

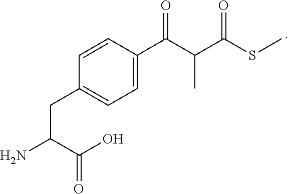 [SMILES: CSC(=O)C(C)C(=O)c1ccc(CC(N)C(=O)O)cc1]

The following amino acids having structures of Formula (XXXVII) are also included:

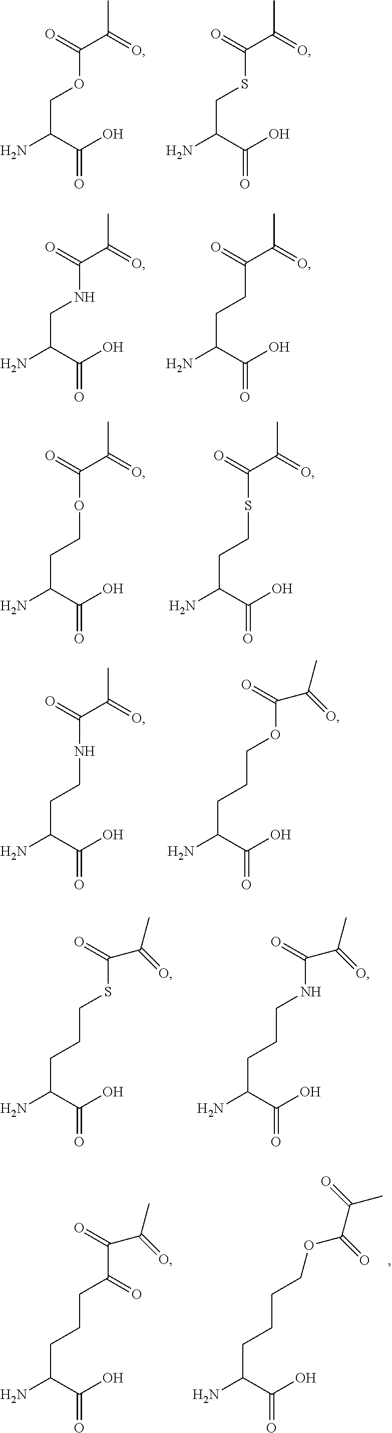

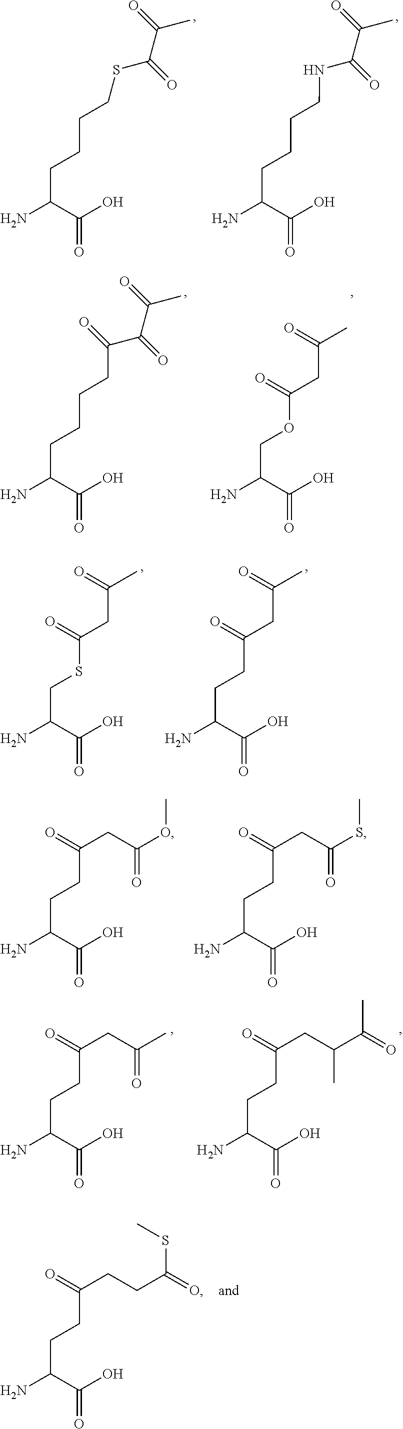

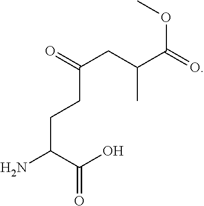 [SMILES: COC(=O)C(C)CC(=O)CCC(N)C(=O)O]

Such non-natural amino acids may be in the form of a salt, or may be incorporated into a non-natural amino acid polypeptide, polymer, polysaccharide, or a polynucleotide and optionally post translationally modified.

Structure and Synthesis of Non-Natural Amino Acids: Ketoalkyne, Ketoalkyne -like, Masked Ketoalkyne, Protected Ketoalkyne Groupk, Alkyne, and Cycloalkyne Groups

Amino acids containing reactive groups with dicarbonyl-like reactivity allow for the linking of molecules via nucleophilic addition reactions. Such electrophilic reactive groups include a ketoalkyne group, a ketoalkyne-like group (which has reactivity similar to a ketoalkyne group and is structurally similar to a ketoalkyne group), a masked ketoalkyne group (which can be readily converted into a ketoalkyne group), or a protected ketoalkyne group (which has reactivity similar to a ketoalkyne group upon deprotection). In some embodiments, amino acids containing reactive groups with a terminal alkyne, internal alkyne or cycloalkyne allow for linking of molecules via cycloaddition reactions (e.g., 1,3-dipolar cycloadditions, azide-alkyne Huisgen cycloaddition, etc.) Such amino acids include amino acids having the structure of Formula (XXXXXXI-A) or (XXXXXXI-B):

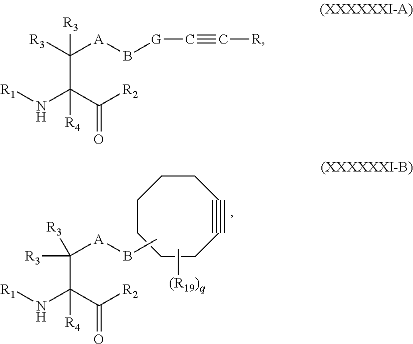

wherein;
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;

B is optional, and when present is a linker linked at one end to a diamine containing moiety, the linker selected from the group consisting of lower alkylene, substituted lower alkylene, lower alkenylene, substituted lower alkenylene, lower heteroalkylene, substituted lower heteroalkylene, —O-(alkylene or substituted alkylene)-, —S-(alkylene or substituted alkylene)-, —C(O)R″—, —S(O)ₖ(alkylene or substituted alkylene)-, where k is 1., 2, or 3, —C(O)-(alkylene or substituted alkylene)-, —C(S)-(alkylene or substituted alkylene)-, —NR″-(alkylene or substituted alkylene)-, —CON(R″)-(alkylene or substituted alkylene)-, —CSN(R″)-(alkylene or substituted alkylene)-, and —N(R″)CO-(alkylene or substituted alkylene)-, where each R″ is independently H, alkyl, or substituted alkyl;
G is optional, and when present is

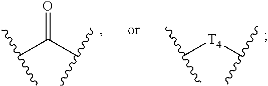

T₄ is a carbonyl protecting group including, but not limited to,

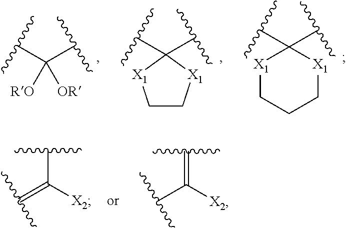

where each X₁ is independently selected from the group consisting of —O—, —S—, —N(H)—, —N(R)—, —N(Ac)-, and —N(OMe)-; X₂ is OR, -OAc, —SR, —N(R)₂, —N(R)(Ac), —N(R)(OMe), or N₃, and where each R′ is independently H, alkyl, or substituted alkyl;
R is H, halogen, alkyl, substituted alkyl, cycloalkyl, or substituted cycloalkyl;
R₁ is H, an amino protecting group, resin, amino acid, polypeptide, or polynucleotide;
R₂ is OH, an ester protecting group, resin, amino acid, polypeptide, or polynucleotide;
each of R₃ and R₄ is independently H, halogen, lower alkyl, or substituted lower alkyl, or R₃ and R₄ or two R₃ groups optionally form a cycloalkyl or a heterocycloalkyl;
each R₁₉ is independently selected from the group consisting of C₁-C₆ alkyl, C₁-C₆ alkoxy, ester, ether, thioether, aminoalkyl, halogen, alkyl ester, aryl ester, amide, aryl amide, alkyl halide, alkyl amine, alkyl sulfonic acid, alkyl nitro, thioester, sulfonyl ester, halosulfonyl, nitrile, alkyl nitrile, and nitro; and
q is 0, 1, 2, 3, 4, 5, 6, 7, 8, 9, 10 or 11.

### D. Structure and Synthesis of Non-Natural Amino Acids: Ketoamine, Ketoamine-like, Masked Ketoamine, and Protected Ketoamine Groups

Amino acids containing reactive groups with dicarbonyl-like reactivity allow for the linking of molecules via nucleophilic addition reactions. Such reactive groups include a ketoamine group, a ketoamine-like group (which has reactivity similar to a ketoamine group and is structurally similar to a ketoamine group), a masked ketoamine group (which can be readily converted into a ketoamine group), or a protected ketoamine group (which has reactivity similar to a ketoamine group upon deprotection). Such amino acids include amino acids having the structure of Formula (XXXXXXII):

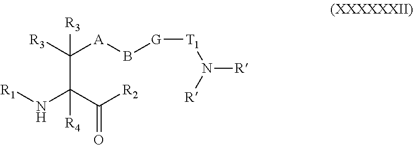

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker linked at one end to a diamine containing moiety, the linker selected from the group consisting of lower alkylene, substituted lower alkylene, lower alkenylene, substituted lower alkenylene, lower heteroalkylene, substituted lower heteroalkylene, —O-(alkylene or substituted alkylene)-, —S-(alkylene or substituted alkylene)-, —C(O)R″—, —S(O)ₖ(alkylene or substituted alkylene)-, where k is 1, 2, or 3, —C(O)-(alkylene or substituted alkylene)-, —C(S)-(alkylene or substituted alkylene)-, —NR″-(alkylene or substituted alkylene)-, —CON(R″)-(alkylene or substituted alkylene)-, —CSN(R″)-(alkylene or substituted alkylene)-, and —N(R″)CO-(alkylene or substituted alkylene)-, where each R″ is independently H, alkyl, or substituted alkyl;
G is

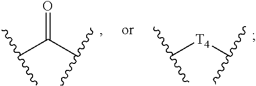

T₁ is an optionally substituted C₁-C₄ alkylene, an optionally substituted C₁-C₄ alkenylene, or an optionally substituted heteroalkyl;
T₄ is a carbonyl protecting group including, but not limited to,

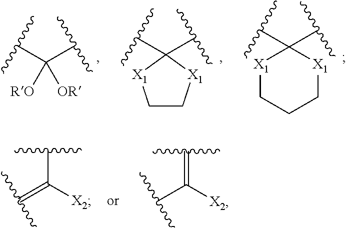

where each X₁ is independently selected from the group consisting of —O—, —S—, —N(H)—, —N(R′)—, —N(Ac)-, and —N(OMe)-; X₂ is OR, -OAc, —SR′, —N(R′)₂, —N(R′)(Ac), —N(R′)(OMe), or N₃, and where each R′ is independently H, alkyl, or substituted alkyl;
R is H, halogen, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
each of R₃ and R₄ is independently H, halogen, lower alkyl, or substituted lower alkyl, or R₃ and R₄ or two R₃ groups optionally form a cycloalkyl or a heterocycloalkyl.

Amino acids having the structure of Formula (XXXXXXII) include amino acids having the structure of Formula (XXXXXXIII) and Formula (XXXXXXIV):

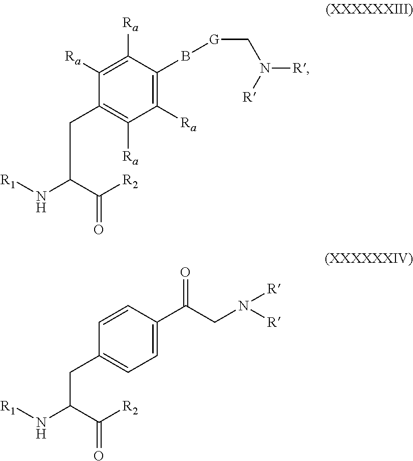

wherein each R, is independently selected from the group consisting of H, halogen, alkyl, substituted alkyl, —N(R′)₂, —C(O)ₖR′ where k is 1, 2, or 3, —C(O)N(R′)₂, —OR′, and —S(O)ₖR′, where each R′ is independently H, alkyl, or substituted alkyl.

### E. Structure and Synthesis of Non-Natural Amino Acids: Diamine, Diamine-like, Masked Diamine, Protected Amines and Azides

Amino acids with a nucleophilic reactive group allow for a variety of reactions to link molecules via electrophilic addition reactions among others. Such nucleophilic reactive groups include a diamine group (including a hydrazine group, an amidine group, an imine group, a 1,1-diamine group, a 1,2-diamine group, a 1,3-diamine group, and a 1,4-diamine group), a diamine-like group (which has reactivity similar to a diamine group and is structurally similar to a diamine group), a masked diamine group (which can be readily converted into a diamine group), or a protected diamine group (which has reactivity similar to a diamine group upon deprotection). In some embodiments, amino acids containing reactive groups with azides allow for linking of molecules via cycloaddition reactions (e.g., 1,3-dipolar cyclo additions, azide-alkyne Huisgen cycloaddition, etc.).

In another aspect are methods for the chemical synthesis of hydrazine-substituted molecules for the derivatization of carbonyl-substituted NRL derivatives. In one embodiment, the hydrazine-substituted molecule can NRL linked derivatives. In one embodiment are methods for the preparation of hydrazine-substituted molecules suitable for the derivatization of carbonyl-containing non-natural amino acid polypeptides, including by way of example only, ketone-, or aldehyde-containing non-natural amino acid polypeptides. In a further or additional embodiment, the non-natural amino acids are incorporated site-specifically during the in vivo translation of proteins. In a further or additional embodiment, the hydrazine-substituted NRL derivatives allow for the site-specific derivatization of carbonyl-containing non-natural amino acids via nucleophilic attack of each carbonyl group to form a heterocycle-derivatized polypeptide, including a nitrogen-containing heterocycle-derivatized polypeptide in a site-specific fashion. In a further or additional embodiment, the method for the preparation of hydrazine-substituted NRL derivatives provides access to a wide variety of site-specifically derivatized polypeptides. In a further or additional embodiment are methods for synthesizing hydrazine-functionalized polyethyleneglycol (PEG) linked NRL derivatives.

Such amino acids include amino acids having the structure of Formula (XXXVII-A) or (XXXVII-B):

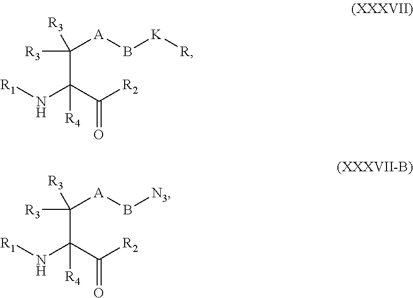

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker linked at one end to a diamine containing moiety, the linker selected from the group consisting of lower alkylene, substituted lower alkylene, lower alkenylene, substituted lower alkenylene, lower heteroalkylene, substituted lower heteroalkylene, —O-(alkylene or substituted alkylene)-, —S-(alkylene or substituted alkylene)-, —C(O)R″—, —C(O)R″—, —S(O)ₖ-(alkylene or substituted alkylene)-, where k is 1, 2, or 3, —C(O)-(alkylene or substituted alkylene)-, —C(S)-(alkylene or substituted alkylene)-, —NR″-(alkylene or substituted alkylene)-, —CON(R″)-(alkylene or substituted alkylene)-, —CSN(R″)-(alkylene or substituted alkylene)-, and —N(R″)CO-(alkylene or substituted alkylene)-, where each R″ is independently H, alkyl, or substituted alkyl;
K is

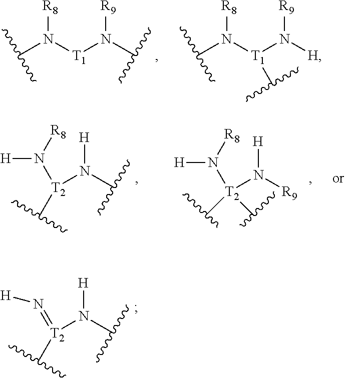

where:
R₈ and R₉ are independently selected from H, alkyl, substituted alkyl, cycloalkyl, substituted cycloalkyl, or amine protecting group;
T₁ is a bond, optionally substituted C₁-C₄ alkylene, optionally substituted C₁-C₄ alkenylene, or optionally substituted heteroalkyl;
T₂ is optionally substituted C₁-C₄ alkylene, optionally substituted C₁-C₄ alkenylene, optionally substituted heteroalkyl, optionally substituted aryl, or optionally substituted heteroaryl;
wherein each optional substituents is independently selected from lower alkyl, substituted lower alkyl, lower cycloalkyl, substituted lower cycloalkyl, lower alkenyl, substituted lower alkenyl, alkynyl, lower heteroalkyl, substituted heteroalkyl, lower heterocycloalkyl, substituted lower heterocycloalkyl, aryl, substituted aryl, heteroaryl, substituted heteroaryl, alkaryl, substituted alkaryl, aralkyl, or substituted aralkyl;
R is H, alkyl, substituted alkyl, cycloalkyl, or substituted cycloalkyl;
R₁ is H, an amino protecting group, resin, amino acid, polypeptide, or polynucleotide; and
R₂ is OH, an ester protecting group, resin, amino acid, polypeptide, or polynucleotide;
each of R₃ and R₄ is independently H, halogen, lower alkyl, or substituted lower alkyl, or R₃ and R₄ or two R₃ groups optionally form a cycloalkyl or a heterocycloalkyl;
or the A-B—K—R groups together form a bicyclic or tricyclic cycloalkyl or heterocycloalkyl comprising at least one diamine group, protected diamine group or masked diamine group;
or the B—K—R groups together form a bicyclic or tricyclic cycloalkyl or cycloaryl or heterocycloalkyl comprising at least one diamine group, protected diamine group or masked diamine group;
or the K—R group together forms a monocyclic or bicyclic cycloalkyl or heterocycloalkyl comprising at least one diamine group, protected diamine group or masked diamine group;
wherein at least one amine group on A-B—K—R is optionally a protected amine.

In one aspect are compounds comprising the structures 1 or 2:

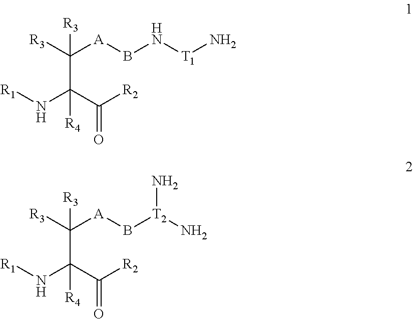

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker linked at one end to a diamine containing moiety, the linker selected from the group consisting of lower alkylene, substituted lower alkylene, lower alkenylene, substituted lower alkenylene, lower heteroalkylene, substituted lower heteroalkylene, —O-(alkylene or substituted alkylene)-, —S-(alkylene or substituted alkylene)-, —C(O)R″—, —S(O)ₖ (alkylene or substituted alkylene)-, where k is 1, 2, or 3, —C(O)-(alkylene or substituted alkylene)-, —C(S)-(alkylene or substituted alkylene)-, —NR″-(alkylene or substituted alkylene)-, —CON(R″)-(alkylene or substituted alkylene)-, —CSN(R″)-(alkylene or substituted alkylene)-, and —N(R″)CO-(alkylene or substituted alkylene)-, where each R″ is independently H, alkyl, or substituted alkyl;
T₁ is a bond or CH₂; and T₂ is CH;
wherein each optional substituents is independently selected from lower alkyl, substituted lower alkyl, lower cycloalkyl, substituted lower cycloalkyl, lower alkenyl, substituted lower alkenyl, alkynyl, lower heteroalkyl, substituted heteroalkyl, lower heterocycloalkyl, substituted lower heterocycloalkyl, aryl, substituted aryl, heteroaryl, substituted heteroaryl, alkaryl, substituted alkaryl, aralkyl, or substituted aralkyl;
R₁ is H, an amino protecting group, resin, amino acid, polypeptide, or polynucleotide; and
R₂ is OH, an ester protecting group, resin, amino acid, polypeptide, or polynucleotide;
each of R₃ and R₄ is independently H, halogen, lower alkyl, or substituted lower alkyl, or R₃ and R₄ or two R₃ groups optionally form a cycloalkyl or a heterocycloalkyl;
or the A-B-diamine containing moiety together form a bicyclic cycloalkyl or heterocycloalkyl comprising at least one diamine group, protected diamine group or masked diamine group;
or the B-diamine containing moiety groups together form a bicyclic or tricyclic cycloalkyl or cycloaryl or heterocycloalkyl comprising at least one diamine group, protected diamine group or masked diamine group;
wherein at least one amine group on A-B-diamine containing moiety is optionally a protected amine;
or an active metabolite, salt, or a pharmaceutically acceptable prodrug or solvate thereof.

The following non-limiting examples of amino acids having the structure of Formula (XXXVII) are included:

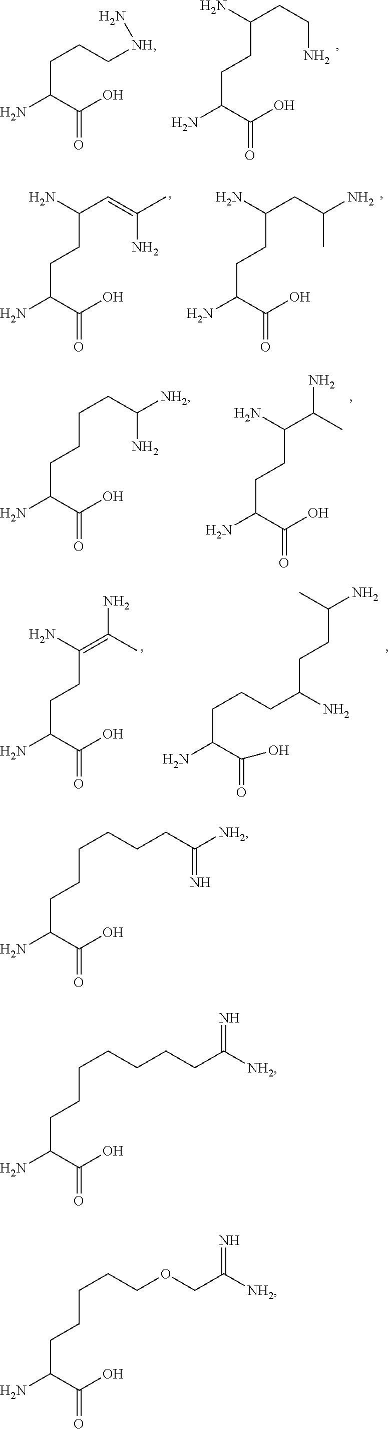

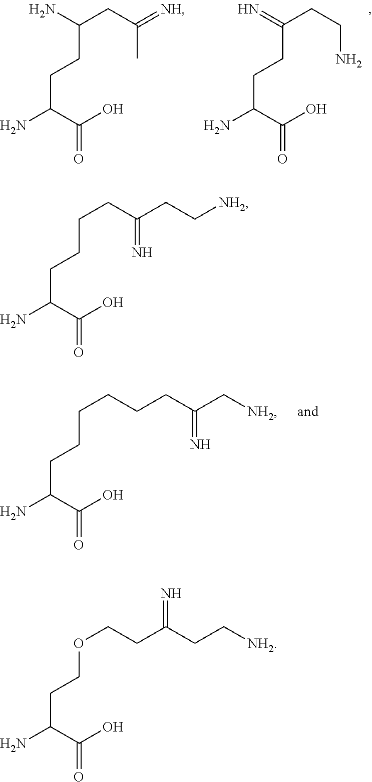

Such non-natural amino acids may also be in the form of a salt or may be incorporated into a non-natural amino acid polypeptide, polymer, polysaccharide, or a polynucleotide and/or optionally post translationally modified,

In certain embodiments, compounds of Formula (XXXVII) are stable in aqueous solution for at least 1 month under mildly acidic conditions. In certain embodiments, compounds of Formula (XXXVII) are stable for at least 2 weeks under mildly acidic conditions. In certain embodiments, compound of Formula (XXXVII) are stable for at least 5 days under mildly acidic conditions. In certain embodiments, such acidic conditions are pH about 2 to about 8,

In certain embodiments of compounds of Formula (XXXVII), B is lower alkylene, substituted lower alkylene, O-(alkylene or substituted alkylene)-, C(R′)═NN(R′)—, —N(R′)CO—, C(O)—, —C(R′)═N—, C(O)-(alkylene or substituted alkylene)-, CON(R′)(alkylene or substituted alkylene)-, —S(alkylene or substituted alkylene)-, —S(O)(alkylene or substituted alkylene)-, or —S(O)₂(alkylene or substituted alkylene)-. In certain embodiments of compounds of Formula (XXXVII), B is O(CH₂)—, —CH═N—, CH═NNH—, —NHCH₂—, —NHCO—, C(O)—, C(O)(CH₂)—, CONH(CH₂)—, —SCH₂—, —S(═O)CH₂—, or —S(O)₂CH₂—. In certain embodiments of compounds of Formula (XXXVII), R is C₁₋₆ alkyl or cycloalkyl. In certain embodiments of compounds of Formula (XXXVII) R is —CH₃, —CH(CH3)₂, or cyclopropyl. In certain embodiments of compounds of Formula (XXXVII), R₁ is H, tert-butyloxycarbonyl (Boc), 9-Fluorenylmethoxycarbonyl (Fmoc), N-acetyl, tetrafluoroacetyl (TFA), or benzyloxycarbonyl (Cbz), In certain embodiments of compounds of Formula (XXXVII), is a resin, amino acid, polypeptide, or polynucleotide. In certain embodiments of compounds of Formula (XXXVII), R₁ is an αPSMA antibody, antibody fragment or monoclonal antibody. In certain embodiments of compounds of Formula (XXXVII), R₂ is OH, O-methyl, O-ethyl, or O-t-butyl. In certain embodiments of compounds of Formula (XXXVII), R₂ is a resin, at least one amino acid, polypeptide, or polynucleotide. In certain embodiments of compounds of Formula (XXXVII), R₂ is an αPSMA antibody, antibody fragment or monoclonal antibody.

The following non-limiting examples of amino acids having the structure of Formula (XXXVII) are also included:

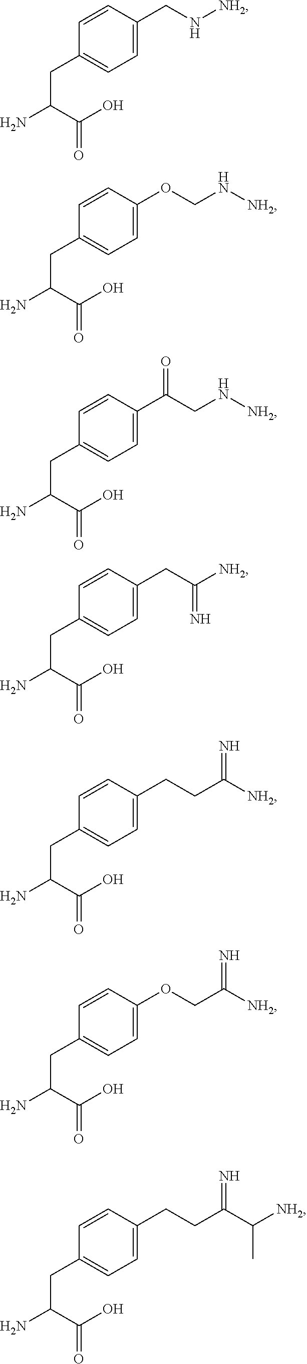

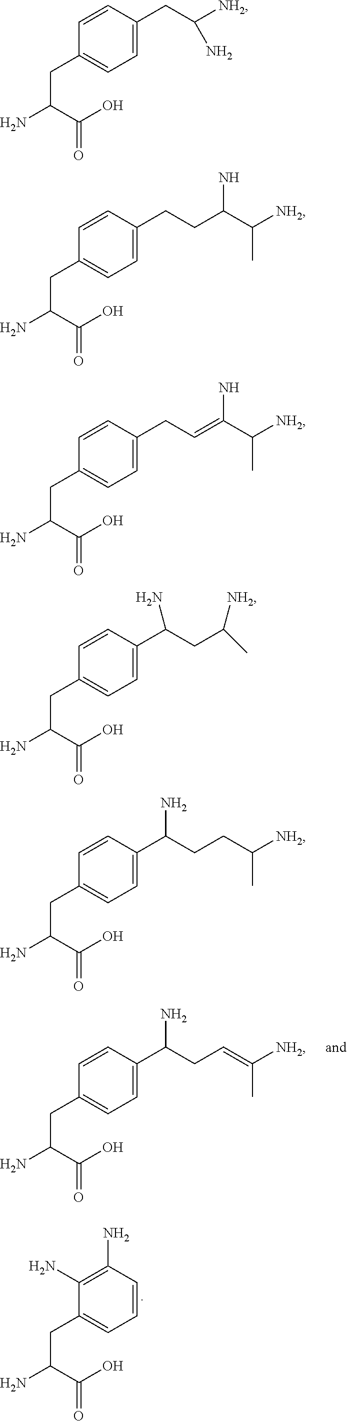

Non-limiting examples of protected amino acids having the structure of Formula (XXXVII) include:

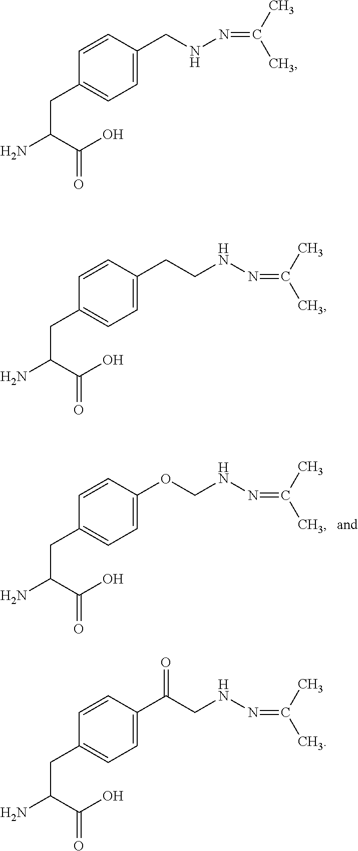 [SMILES: CC(C)=NNCc1ccc(CC(N)C(=O)O)cc1 ; CC(C)=NNCCc1ccc(CC(N)C(=O)O)cc1 ; CC(C)=NNCOc1ccc(CC(N)C(=O)O)cc1 ; CC(C)=NNCC(=O)c1ccc(CC(N)C(=O)O)cc1]

### F. Structure and Synthesis of Non-Natural Amino Acids: Aromatic Amines

Non-natural amino acids with nucleophilic reactive groups, such as, by way of example only, an aromatic amine group (including secondary and tertiary amine groups), a masked aromatic amine group (which can be readily converted into a aromatic amine group), or a protected aromatic amine group (which has reactivity similar to an aromatic amine group upon deprotection) allow for a variety of reactions to link molecules via various reactions, including but not limited to, reductive alkylation reactions with aldehyde containing NRL conjugates. Such aromatic amine containing non-natural amino acids include amino acids having the structure of Formula (XXXXXXV):

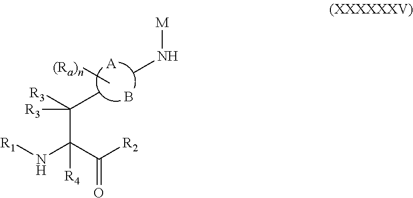

wherein:

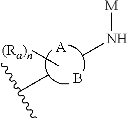

is selected from the group consisting of a monocyclic aryl ring, a bicyclic aryl ring, a multicyclic aryl ring, a monocyclic heteroaryl ring, a bicyclic heteroaryl ring, and a multicyclic heteroaryl ring;
A is independently CRₐ, or N;
B is independently CRₐ, N, O, or S;
each Rₐ is independently selected from the group consisting of H, halogen, alkyl, —NO₂, —CN, substituted alkyl, —N(R′)₂, —C(O)ₖR′, —C(O)N(R′)₂, —OR′, and —S(O)ₖR′, where k is 1, 2, or 3; and n is 0, 1, 2, 3, 4, 5, or 6;
R₁ is H, an amino protecting group, resin, at least one amino acid, polypeptide, or polynucleotide; and
R₂ is OH, an ester protecting group, resin, at least one amino acid, polypeptide, or polynucleotide;
each of R₃ and R₄ is independently H, halogen, lower alkyl, or substituted lower alkyl, or R₃ and R₄ or two R₃ groups optionally form a cycloalkyl or a heterocycloalkyl;
M is H or —CH₂R₅; or the M-N—C(R₅) moiety may form a 4 to 7 membered ring structure;
R₅ is alkyl, substituted alkyl, alkenyl, substituted alkenyl, alkynyl, substituted alkynyl, alkoxy, substituted alkoxy, alkylalkoxy, substituted alkylalkoxy, polyalkylene oxide, substituted polyalkylene oxide, cycloalkyl, substituted cycloalkyl, aryl, substituted aryl, heteroaryl, substituted heteroaryl, heterocycle, substituted heterocycle, alkaryl, substituted alkaryl, aralkyl, substituted aralkyl, —C(O)R″, —C(O)OR″, —C(O)N(R″)₂, —C(O)NHCH(R″)₂, -(alkylene or substituted alkylene)-N(R″)₂, -(alkenylene or substituted alkenylene)-N(R″)₂, -(alkylene or substituted alkylene)-(aryl or substituted aryl), -(alkenylene or substituted alkenylene)-(aryl or substituted aryl), -(alkylene or substituted alkylene)-ON(R″)₂, -(alkylene or substituted alkylene)-C(O)SR″, -(alkylene or substituted alkylene)-S—S-(aryl or substituted aryl), wherein each R″ is independently hydrogen, alkyl, substituted alkyl, alkenyl, substituted alkenyl, alkoxy, substituted alkoxy, aryl, substituted aryl, heteroaryl, substituted heteroaryl, heterocycle, substituted heterocycle, alkaryl, substituted alkaryl, aralkyl, substituted aralkyl, or —C(O)OR′;
or two R₅ groups optionally form a cycloalkyl or a heterocycloalkyl;
or R₅ and any Rₐ optionally form a cycloalkyl or a heterocycloalkyl; and
each R′ is independently H, alkyl, or substituted alkyl.
   Such non-natural amino acids may also be in the form of a salt, or may be incorporated into a non-natural amino acid polypeptide, polymer, polysaccharide, or a polynucleotide and optionally reductively alkylated.
   The structure

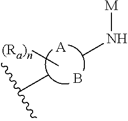

(as presented in all examples herein) does not present the relative orientations of “A,” “B,” “NH-M” and “Rₐ”; rather these four features of this structure may be oriented in any chemically-sound manner (along with other features of this structure), as illustrated by example herein,

Non-natural amino acids containing an aromatic amine moiety having the structure of Formula (A) include non-natural amino acids having the structures:

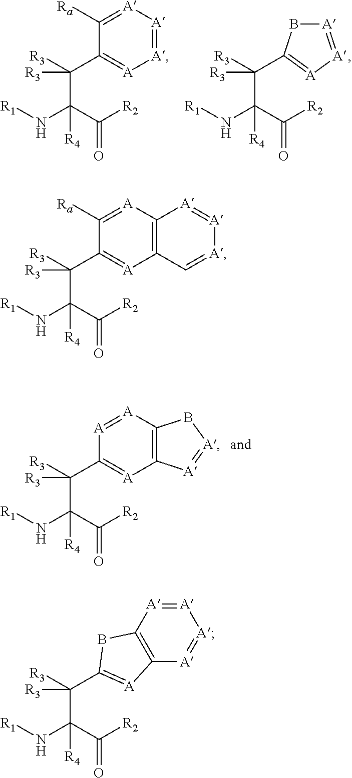

wherein, each A′ is independently selected from CRₐ, N, or

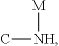

### and up to two A′ may be

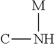

with the remaining A′ selected from CRₐ, or N,
Such non-natural amino acids may also be in the form of a salt, or may be incorporated into a non-natural amino acid polypeptide, polymer, polysaccharide, or a polynucleotide and optionally reductively alkylated.

Non-limiting examples of non-natural amino acids containing an aromatic amine moiety having the structure of Formula (XXXXXXV) include non-natural amino acids having the structure of Formula (XXXXXXVI), and Formula (XXXXXXVII),

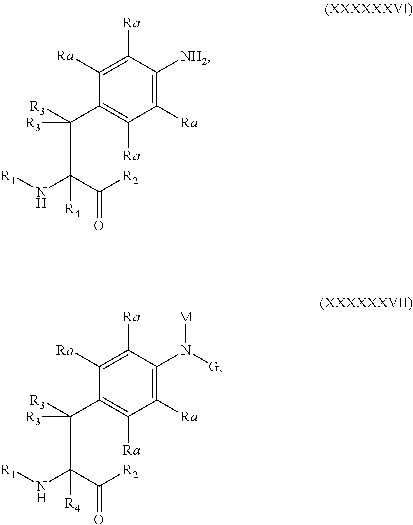

wherein; G is an amine protecting group, including, but not limited to,

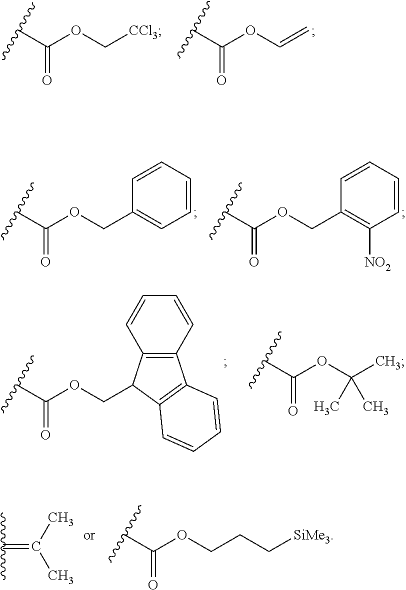

Such non-natural amino acids may be in the form of a salt, or may be incorporated into a non-natural amino acid polypeptide, polymer, polysaccharide, or a polynucleotide and optionally reductively alkylated.

Non-natural amino acids containing an aromatic amine moiety have the following structures:

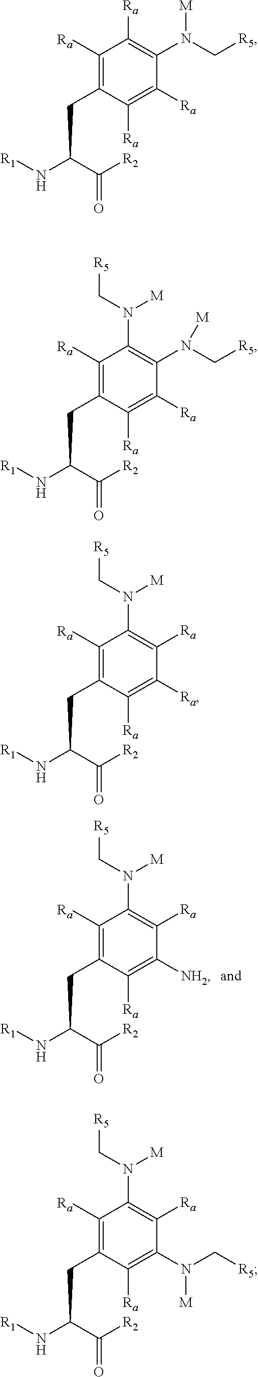

wherein each R, is independently selected from the group consisting of H, halogen, alkyl, —NO₂, —CN, substituted alkyl, —N(R′)₂, —C(O)ₖR′, —C(O)N(R′)₂, —OR′, and —S(O)ₖR′, where k is 1, 2, or 3;
M is H or —CH₂R₅; or the M-N—C(R₅) moiety may form a 4 to 7 membered ring structure;
R₁ is H, an amino protecting group, resin, amino acid, polypeptide, or polynucleotide;
R₂ is OH, an ester protecting group, resin, amino acid, polypeptide, or polynucleotide;
R₅ is alkyl, substituted alkyl, alkenyl, substituted alkenyl, alkynyl, substituted alkynyl, alkoxy, substituted alkoxy, alkylalkoxy, substituted alkylalkoxy, polyalkylene oxide, substituted polyalkylene oxide, cycloalkyl, substituted cycloalkyl, aryl, substituted aryl, heteroaryl, substituted heteroaryl, heterocycle, substituted heterocycle, alkaryl, substituted alkaryl, aralkyl, substituted aralkyl, —C(O)R″, —C(O)OR″, —C(O)N(R″)₂, —C(O)NHCH(R″)₂, -(alkylene or substituted alkylene)-N(R″)₂, -(alkenylene or substituted alkenylene)-N(R″)₂, -(alkylene or substituted alkylene)-(aryl or substituted aryl), -(alkenylene or substituted alkenylene)-(aryl or substituted aryl), -(alkylene or substituted alkylene)-ON(R″)₂, -(alkylene or substituted alkylene)-C(O)SR″, -(alkylene or substituted alkylene)-S—S-(aryl or substituted aryl), wherein each R″ is independently hydrogen, alkyl, substituted alkyl, alkenyl, substituted alkenyl, alkoxy, substituted alkoxy, aryl, substituted aryl, heteroaryl, substituted heteroaryl, heterocycle, substituted heterocycle, alkaryl, substituted alkaryl, aralkyl, substituted aralkyl, or —C(O)OR′;
or R₅ and any R optionally form a cycloalkyl or a heterocycloalkyl; and each R′ is independently H, alkyl, or substituted alkyl. Such non-natural amino acids may also be in the form of a salt, or may be incorporated into a non-natural amino acid polypeptide, polymer, polysaccharide, or a polynucleotide.

Such non-natural amino acids of Formula (XXXXXXV) may be formed by reduction of protected or masked amine moieties on the aromatic moiety of a non-natural amino acid. Such protected or masked amine moieties include, but are not limited to, imines, hydrazines, nitro, or azide substituents. The reducing agents used to reduce such protected or masked amine moieties include, but are not limited to, TCEP, Na₂S, Na₂S₂O₄, LiAlH₄, NaBH₄ or NaBCNH₃.

II. Non-Natural Amino Acid Linked Nuclear Receptor Ligand Conjugates

In another aspect described herein are methods, strategies and techniques for incorporating at least one such NRL conjugate into a non-natural amino acid. Also included with this aspect are methods for producing, purifying, characterizing and using such NRL conjugates containing at least one such non-natural amino acid. Also included with this aspect are compositions of and methods for producing, purifying, characterizing and using oligonucleotides (including DNA and RNA) that can be used to produce, at least in part, a NRL conjugate containing at least one non-natural amino acid. Also included with this aspect are compositions of and methods for producing, purifying, characterizing and using cells that can express such oligonucleotides that can be used to produce, at least in part, a nuclear receptor ligand linker derivative containing at least one non-natural amino acid.

Thus, nuclear receptor ligand linker derivatives comprising at least one non-natural amino acid or modified non-natural amino acid with a carbonyl, dicarbonyl, alkyne, cycloalkyne, azide, oxime or hydroxylamine group are provided and described herein. In certain embodiments, NRL conjugates with at least one non-natural amino acid or modified non-natural amino acid with a carbonyl, dicarbonyl, alkyne, cycloalkyne, azide, oxime or hydroxylamine group include at least one post-translational modification at some position on the polypeptide. In some embodiments the co-translational or post-translational modification occurs via the cellular machinery (e.g., glycosylation, acetylation, acylation, lipid-modification, palmitoylation, palmitate addition, phosphorylation, glycolipid-linkage modification, and the like), in many instances, such cellular-machinery-based co-translational or post-translational modifications occur at the naturally occurring amino acid sites on the polypeptide, however, in certain embodiments, the cellular-machinery-based co-translational or post-translational modifications occur on the non-natural amino acid site(s) on the polypeptide.

In other embodiments, the post-translational modification does not utilize the cellular machinery, but the functionality is instead provided by attachment of a molecule (a polymer; a water-soluble polymer; a derivative of polyethylene glycol; a second protein or polypeptide or polypeptide analog; an antibody or antibody fragment; and any combination thereof) comprising a second reactive group to the at least one non-natural amino acid comprising a first reactive group (including but not limited to, non-natural amino acid containing a ketone, aldehyde, acetal, hemiacetal, alkyne, cycloalkyne, azide, oxime, or hydroxylamine functional group) utilizing chemistry methodology described herein, or others suitable for the particular reactive groups. In certain embodiments, the co-translational or post-translational modification is made in vivo in a eukaryotic cell or in a non-eukaryotic cell. In certain embodiments, the post-translational modification is made in vitro not utilizing the cellular machinery. Also included with this aspect are methods for producing, purifying, characterizing and using such NRL conjugates containing at least one such co-translationally or post-translationally modified non-natural amino acids.

Also included within the scope of the methods, compositions, strategies and techniques described herein are reagents capable of reacting with a NRL conjugate (containing a carbonyl or dicarbonyl group, oxime group, alkyne, cycloalkyne, azide, hydroxylamine group, or masked or protected forms thereof) that is part of a polypeptide so as to produce any of the aforementioned post-translational modifications. In certain embodiments, the resulting post-translationally modified NRL conjugate will contain at least one oxime group; the resulting modified oxime-containing NRL linker derivative may undergo subsequent modification reactions. Also included with this aspect are methods for producing, purifying, characterizing and using such reagents that are capable of any such post-translational modifications of such NRL linker derivative(s).

In certain embodiments, the polypeptide or non-natural amino acid linked NRL derivative includes at least one co-translational or post-translational modification that is made in vivo by one host cell, where the post-translational modification is not normally made by another host cell type. In certain embodiments, the polypeptide includes at least one co-translational or post-translational modification that is made in vivo by a eukaryotic cell, where the co-translational or post-translational modification is not normally made by a non-eukaryotic cell. Examples of such co-translational or post-translational modifications include, but are not limited to, glycosylation, acetylation, acylation, lipid-modification, palmitoylation, palmitate addition, phosphorylation, glycolipid-linkage modification, and the like. In one embodiment, the co-translational or post-translational modification comprises attachment of an oligosaccharide to an asparagine by a GlcNAc-asparagine linkage (including but not limited to, where the oligosaccharide comprises (GlcNAc-Man)₂-Man-GlcNAc-GlcNAc, and the like). In another embodiment, the co-translational or post-translational modification comprises attachment of an oligosaccharide (including but not limited to, Gal-GalNAc, Gal-GlcNAc, etc.) to a serine or threonine by a GalNAc-serine, a GalNAc-threonine, a GlcNAc-serine, or a GlcNAc-threonine linkage. In certain embodiments, a protein or polypeptide can comprise a secretion or localization sequence, an epitope tag, a FLAG tag, a polyhistidine tag, a GST fusion, and/or the like. Also included with this aspect are methods for producing, purifying, characterizing and using such polypeptides containing at least one such co-translational or post-translational modification. In other embodiments, the glycosylated non-natural amino acid polypeptide is produced in a non-glycosylated form. Such a non-glycosylated form of a glycosylated non-natural amino acid may be produced by methods that include chemical or enzymatic removal of oligosaccharide groups from an isolated or substantially purified or unpurified glycosylated non-natural amino acid polypeptide; production of the non-natural amino acid in a host that does not glycosylate such a non-natural amino acid polypeptide (such a host including, prokaryotes or eukaryotes engineered or mutated to not glycosylate such a polypeptide), the introduction of a glycosylation inhibitor into the cell culture medium in which such a non-natural amino acid polypeptide is being produced by a eukaryote that normally would glycosylate such a polypeptide, or a combination of any such methods. Also described herein are such non-glycosylated forms of normally-glycosylated non-natural amino acid polypeptides (by normally-glycosylated is meant a polypeptide that would be glycosylated when produced under conditions in which naturally-occurring polypeptides are glycosylated). Of course, such non-glycosylated forms of normally-glycosylated non-natural amino acid polypeptides (or indeed any polypeptide described herein) may be in an unpurified form, a substantially purified form, or in an isolated form.

In certain embodiments, the non-natural amino acid polypeptide includes at least one post-translational modification that is made in the presence of an accelerant, wherein the post-translational modification is stoichiometric, stoichiometric-like, or near-stoichiometric. In other embodiments the polypeptide is contacted with a reagent of Formula (XIX) in the presence of an accelerant. In other embodiments the accelerant is selected from the group consisting of:

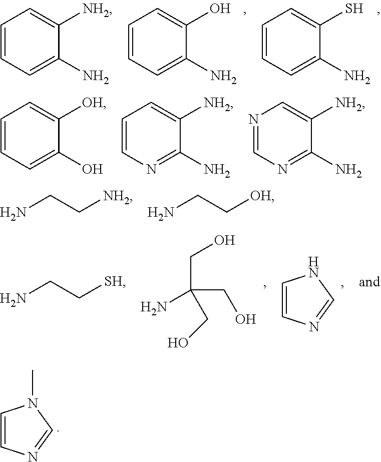

Chemical Synthesis of Non Natural Amino Acid Linked Nuclear Receptor Ligand Derivatives: Oxime-Containing Linked Nuclear Receptor Ligand Derivatives

Non-natural amino acid NRL linked derivatives containing an oxime group allow for reaction with a variety of reagents that contain certain reactive carbonyl- or dicarbonyl-groups (including but not limited to, ketones, aldehydes, or other groups with similar reactivity) to form new non-natural amino acids comprising a new oxime group. Such an oxime exchange reaction allows for the further functionalization of NRL linked derivatives. Further, the original NRL linked derivative containing an oxime group may be useful in their own right as long as the oxime linkage is stable under conditions necessary to incorporate the amino acid into a polypeptide (e.g., the in vivo, in vitro and chemical synthetic methods described herein).

Thus, in certain embodiments described herein are non-natural amino acid NRL linked derivatives with sidechains comprising an oxime group, an oxime-like group (which has reactivity similar to an oxime group and is structurally similar to an oxime group), a masked oxime group (which can be readily converted into an oxime group), or a protected oxime group (which has reactivity similar to an oxime group upon deprotection),

Such non-natural amino acid NRL linked derivatives include NRL linked derivatives having the structure of Formula (VIII) or (IX) wherein NRL is any nuclear receptor ligand:

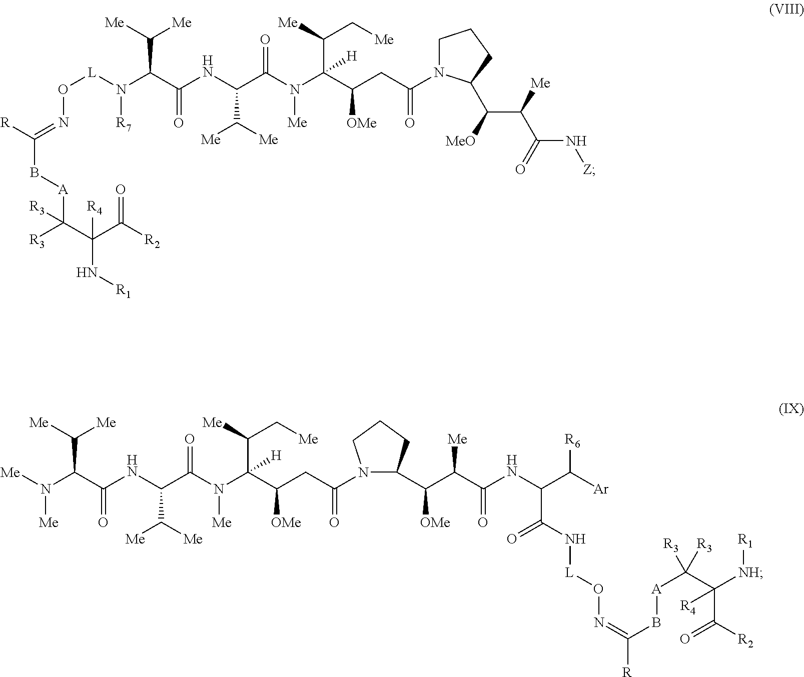

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —N(R′)—N═, —C(R′)═N—, —C(R′)═N—N(R′)—, —C(R′)═N—N═, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is H, an amino protecting group, resin, at least one amino acid, polypeptide, or polynucleotide;
R₂ is OH, an ester protecting group, resin, at least one amino acid, polypeptide, or polynucleotide;
R₃ and R₄ are each independently H, halogen, lower alkyl, or substituted lower alkyl, or R₃ and R₄ or two R₃ groups optionally form a cycloalkyl or a heterocycloalkyl;
L is a linker selected from the group consisting of -alkylene-, -alkylene-C(O)—, -(alkylene-O)ₙ-alkylene-, -(alkylene-O)ₙ-alkylene-C(O)—, -(alkylene-O)ₙ—(CH₂)_{n′}—NHC(O)—(CH₂)_{n″}—C(Me)₂-S—S—CH₂)_{n′″}—NHC(O)-(alkylene-O)_{n″″}--alkylene-, -(alkylene-O)ₙ-alkylene-W—, -alkylene-C(O)—W—, -(alkylene-O)ₙ-alkylene-U-alkylene-C(O)—, and -(alkylene-O)ₙ-alkylene-U-alkylene-;
   W has the structure of:

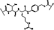 [SMILES: *C(=O)N[C@H](C(=O)N[C@@H](CCCNC(N)=O)C(=O)Nc1ccc(COC(*)=O)cc1)C(C)C]

U has the structure of:

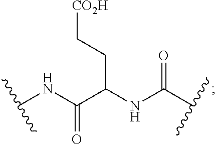 [SMILES: *NC(=O)C(CCC(=O)O)NC(*)=O]

and
each n, n′, n″, n′″ and n″″ are independently integers greater than or equal to one;

or an active metabolite, or a pharmaceutically acceptable prodrug or solvate thereof

In certain embodiments of compounds of Formula (VIII) and (IX), n is an integer from 0 to 20. In certain embodiments of compounds of Formula (VIII) and (IX), n is an integer from 0 to 10. In certain embodiments of compounds of Formula (VIII) and (IX), n is an integer from 0 to 5. In certain embodiments of Formula (VIII) and (IX), alkylene is methylene, ethylene, propylene, butylenes, pentylene, hexylene, or heptylene.

In certain embodiments of compounds of Formula (VIII) and (IX), each L is independently a cleavable linker or non-cleavable linker. In certain embodiments of compounds of Formula (VIII) and (IX), each L is independently a oligo(ethylene glycol) derivatized linker.

In certain embodiments of compounds of Formula (VIII) and (IX), each alkylene, alkylene′, alkylene“, and alkylene”′ independently is —CH₂—, —CH₂CH₂, CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —CH₂CH₂CH₂CH₂CH₂CH₂CH₂CH₂CH₂—, —CH₂CH₂CH₂CH₂CH₂CH₂CH₂CH₂CH₂CH₂—, —CH₂CH₂CH₂CH₂CH₂CH₂CH₂CH₂CH₂CH₂CH₂—, or —CH₂CH₂CH₂CH₂CH₂CH₂CH₂CH₂CH₂CH₂CH₂CH₂—. In certain embodiments of compounds of Formula (VIII) and (IX), alkylene is methylene, ethylene, propylene, butylenes, pentylene, hexylene, or heptylene.

In certain embodiments of compounds of Formula (VIII) and (IX), each n, n′, n″, n′″, and n″″ independently is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In certain embodiments of compounds of Formula (VIII) or (IX), R₁ is a polypeptide. In certain embodiments of compounds of Formula (VIII) or (IX), R₂ is a polypeptide. In certain embodiments of compounds of Formula (VIII) or (IX), the polypeptide is an αPSMA antibody.

In certain embodiments, compounds of Formula (X), (XI), (XII) or (XIII) are stable in aqueous solution for at least 1 month under mildly acidic conditions. In certain embodiments, compounds of Formula (X), (XI), (XII) or (XIII) are stable for at least 2 weeks under mildly acidic conditions. In certain embodiments, compound of Formula (X), (XI), (XII) or (XIII) are stable for at least 5 days under mildly acidic conditions. In certain embodiments, such acidic conditions are pH 2 to 8. Such non-natural amino acids may be in the form of a salt, or may be incorporated into a non-natural amino acid polypeptide, polymer, polysaccharide, or a polynucleotide and optionally post translationally modified.

Oxime-based non-natural amino acids may be synthesized by methods already described in the art, or by methods described herein, including: (a) reaction of a hydroxylamine-containing non-natural amino acid with a carbonyl- or dicarbonyl-containing reagent; (b) reaction of a carbonyl- or dicarbonyl-containing non-natural amino acid with a hydroxylamine-containing reagent; or (c) reaction of an oxime-containing non-natural amino acid with certain carbonyl- or dicarbonyl-containing reagents.

Chemical Structure and Synthesis of Non-Natural Amino Acid Linked Nuclear Receptor Ligand Derivatives: Alkylated Aromatic Amine Linked Nuclear Receptor Ligand Derivatives

In one aspect are NRL linker derivatives for the chemical derivatization of non-natural amino acids based upon the reactivity of an aromatic amine group. In further or additional embodiments, at least one of the aforementioned non-natural amino acids is incorporated into an NRL linker derivative, that is, such embodiments are non-natural amino acid linked NRL derivatives. In further or additional embodiments, the NRL linker derivatives are functionalized on their sidechains such that their reaction with a derivatizing non-natural amino acid generates an amine linkage. In further or additional embodiments, the NRL linker derivatives are selected from NRL linker derivatives having aromatic amine sidechains. In further or additional embodiments, the NRL linker derivatives comprise a masked sidechain, including a masked aromatic amine group. In further or additional embodiments, the non-natural amino acids are selected from amino acids having aromatic amine sidechains. In further or additional embodiments, the non-natural amino acids comprise a masked sidechain, including a masked aromatic amine group.

In another aspect are carbonyl-substituted NRL linker derivatives such as, by way of example, aldehydes, and ketones, for the production of derivatized non-natural amino acid polypeptides based upon an amine linkage. In a further embodiment are aldehyde-substituted NRL linker derivatives used to derivatize aromatic amine-containing non-natural amino acid polypeptides via the formation of an amine linkage between the derivatizing NRL linker and the aromatic amine-containing non-natural amino acid polypeptide.

In further or additional embodiments, the non-natural amino acids comprise aromatic amine sidechains where the aromatic amine is selected from an aryl amine or a heteroaryl amine. In a further or additional embodiment, the non-natural amino acids resemble a natural amino acid in structure but contain aromatic amine groups. In another or further embodiment the non-natural amino acids resemble phenylalanine or tyrosine (aromatic amino acids). In one embodiment, the non-natural amino acids have properties that are distinct from those of the natural amino acids. In one embodiment, such distinct properties are the chemical reactivity of the sidechain; in a further embodiment this distinct chemical reactivity permits the sidechain of the non-natural amino acid to undergo a reaction while being a unit of a polypeptide even though the sidechains of the naturally-occurring amino acid units in the same polypeptide do not undergo the aforementioned reaction. In a further embodiment, the sidechain of the non-natural amino acid has a chemistry orthogonal to those of the naturally-occurring amino acids. In a further embodiment, the sidechain of the non-natural amino acid comprises a nucleophile-containing moiety; in a further embodiment, the nucleophile-containing moiety on the sidechain of the non-natural amino acid can undergo a reaction to generate an amine-linked derivatized NRL. In a further embodiment, the sidechain of the non-natural amino acid comprises an electrophile-containing moiety; in a further embodiment, the electrophile-containing moiety on the sidechain of the non-natural amino acid can undergo nucleophilic attack to generate an amine-linked derivatized NRL. In any of the aforementioned embodiments in this paragraph, the non-natural amino acid may exist as a separate molecule or may be incorporated into a polypeptide of any length; if the latter, then the polypeptide may further incorporate naturally-occurring or non-natural amino acids.

Modification of non-natural amino acids described herein using reductive alkylation or reductive amination reactions have any or all of the following advantages. First, aromatic amines can be reductively alkylated with carbonyl-containing compounds, including aldehydes, and ketones, in a pH range of about 4 to about 10 (and in certain embodiments in a pH range of about 4 to about 7) to generate substituted amine, including secondary and tertiary amine, linkages. Second, under these reaction conditions the chemistry is selective for non-natural amino acids as the sidechains of naturally occurring amino acids are unreactive. This allows for site-specific derivatization of polypeptides which have incorporated non-natural amino acids containing aromatic amine moieties or protected aldehyde moieties, including, by way of example, recombinant proteins. Such derivatized polypeptides and proteins can thereby be prepared as defined homogeneous products. Third, the mild conditions needed to effect the reaction of an aromatic amine moiety on an amino acid, which has been incorporated into a polypeptide, with an aldehyde-containing reagent generally do not irreversibly destroy the tertiary structure of the polypeptide (excepting, of course, where the purpose of the reaction is to destroy such tertiary structure). Similarly, the mild conditions needed to effect the reaction of an aldehyde moiety on an amino acid, which has been incorporated into a polypeptide and deprotected, with an aromatic amine-containing reagent generally do not irreversibly destroy the tertiary structure of the polypeptide (excepting, of course, where the purpose of the reaction is to destroy such tertiary structure). Fourth, the reaction occurs rapidly at room temperature, which allows the use of many types of polypeptides or reagents that would otherwise be unstable at higher temperatures. Fifth, the reaction occurs readily is aqueous conditions, again allowing use of polypeptides and reagents incompatible (to any extent) with non-aqueous solutions. Six, the reaction occurs readily even when the ratio of polypeptide or amino acid to reagent is stoichiometric, stoichiometric-like, or near-stoichiometric, so that it is unnecessary to add excess reagent or polypeptide to obtain a useful amount of reaction product. Seventh, the resulting amine can be produced regioselectively and/or regiospecifically, depending upon the design of the amine and carbonyl portions of the reactants. Finally, the reductive alkylation of aromatic amines with aldehyde-containing reagents, and the reductive amination of aldehydes with aromatic amine containing reagents, generates amine, including secondary and tertiary amine, linkages which are stable under biological conditions.

Non-natural amino acids with nucleophilic reactive groups, such as, by way of example only, an aromatic amine group (including secondary and tertiary amine groups), a masked aromatic amine group (which can be readily converted into a aromatic amine group), or a protected aromatic amine group (which has reactivity similar to a aromatic amine group upon deprotection) allow for a variety of reactions to link molecules via various reactions, including but not limited to, reductive alkylation reactions with aldehyde containing NRL linked derivatives.

### Chemical Synthesis of Non-Natural Amino Acid Linked Nuclear Receptor Ligand Conjugates: Heteroaryl-Containing Nuclear Receptor Ligand Conjugates

In one aspect are non-natural amino acids for the chemical derivatization of NRL linked derivatives based upon the reactivity of a dicarbonyl group, including a group containing at least one ketone group, and/or at least one aldehyde groups, and/or at least one ester group, and/or at least one carboxylic acid, and/or at least one thioester group, and wherein the dicarbonyl group can be a 1,2-dicarbonyl group, a 1,3-dicarbonyl group, or a 1,4-dicarbonyl group. In further or additional aspects are non-natural amino acids for the chemical derivatization of NRL linked derivatives based upon the reactivity of a diamine group, including a hydrazine group, an amidine group, an imine group, a 1,1 diamine group, a 1,2-diamine group, a 1,3-diamine group, and a 1,4-diamine group. In further or additional embodiments, at least one of the aforementioned non-natural amino acids is incorporated into a NRL linked derivative, that is, such embodiments are non-natural amino acid linked NRL derivatives. In further or additional embodiments, the non-natural amino acids are functionalized on their sidechains such that their reaction with a derivatizing molecule generates a linkage, including a heterocyclic-based linkage, including a nitrogen-containing heterocycle, and/or an aldol-based linkage. In further or additional embodiments are non-natural amino acid polypeptides that can react with a derivatizing NRL linker to generate a non-natural amino acid linked NRL derivatives containing a linkage, including a heterocyclic-based linkage, including a nitrogen-containing heterocycle, and/or an aldol-based linkage. In further or additional embodiments, the non-natural amino acids are selected from amino acids having dicarbonyl and/or diamine sidechains. In further or additional embodiments, the non-natural amino acids comprise a masked sidechain, including a masked diamine group and/or a masked dicarbonyl group. In further or additional embodiments, the non-natural amino acids comprise a group selected from: keto-amine (i.e., a group containing both a ketone and an amine); keto-alkyne (i.e., a group containing both a ketone and an alkyne); and an ene-dione (i.e., a group containing a dicarbonyl group and an alkene).

In further or additional embodiments, the non-natural amino acids comprise dicarbonyl sidechains where the carbonyl is selected from a ketone, an aldehyde, a carboxylic acid, or an ester, including a thioester. In another embodiment are non-natural amino acids containing a functional group that is capable of forming a heterocycle, including a nitrogen-containing heterocycle, upon treatment with an appropriately functionalized reagent. In a further or additional embodiment, the non-natural amino acids resemble a natural amino acid in structure but contain one of the aforementioned functional groups. In another or further embodiment the non-natural amino acids resemble phenylalanine or tyrosine (aromatic amino acids); while in a separate embodiment, the non-natural amino acids resemble alanine and leucine (hydrophobic amino acids). In one embodiment, the non-natural amino acids have properties that are distinct from those of the natural amino acids. In one embodiment, such distinct properties are the chemical reactivity of the sidechain, in a further embodiment this distinct chemical reactivity permits the sidechain of the non-natural amino acid to undergo a reaction while being a unit of a polypeptide even though the sidechains of the naturally-occurring amino acid units in the same polypeptide do not undergo the aforementioned reaction. In a further embodiment, the sidechain of the non-natural amino acid has a chemistry orthogonal to those of the naturally-occurring amino acids. In a further embodiment, the sidechain of the non-natural amino acid comprises an electrophile-containing moiety; in a further embodiment, the electrophile-containing moiety on the sidechain of the non-natural amino acid can undergo nucleophilic attack to generate a heterocycle-derivatized protein, including a nitrogen-containing heterocycle-derivatized protein. In any of the aforementioned embodiments in this paragraph, the non-natural amino acid may exist as a separate molecule or may be incorporated into a polypeptide of any length; if the latter, then the polypeptide may further incorporate naturally-occurring or non-natural amino acids.

In another aspect are diamine-substituted molecules, wherein the diamine group is selected from a hydrazine, an amidine, an imine, a 1,1-diamine, a 1,2-diamine, a 1,3-diamine and a 1,4-diamine group, for the production of derivatized non-natural amino acid linked NRL derivatives based upon a heterocycle, including a nitrogen-containing heterocycle, linkage. In a further embodiment are diamine-substituted NRL derivatives used to derivatize dicarbonyl-containing non-natural amino acid polypeptides via the formation of a heterocycle, including a nitrogen-containing heterocycle, linkage between the derivatizing molecule and the dicarbonyl-containing non-natural amino acid polypeptide. In further embodiments the aforementioned dicarbonyl-containing non-natural amino acid polypeptides are diketone-containing non-natural amino acid polypeptides. In further or additional embodiments, the dicarbonyl-containing non-natural amino acids comprise sidechains where the carbonyl is selected from a ketone, an aldehyde, a carboxylic acid, or an ester, including a thioester. In further or additional embodiments, the diamine-substituted molecules comprise a group selected from a desired functionality. In a further embodiment, the sidechain of the non-natural amino acid has a chemistry orthogonal to those of the naturally-occurring amino acids that allows the non-natural amino acid to react selectively with the diamine-substituted molecules. In a further embodiment, the sidechain of the non-natural amino acid comprises an electrophile-containing moiety that reacts selectively with the diamine-containing molecule; in a further embodiment, the electrophile-containing moiety on the sidechain of the non-natural amino acid can undergo nucleophilic attack to generate a heterocycle-derivatized protein, including a nitrogen-containing heterocycle-derivatized protein. In a further aspect related to the embodiments described in this paragraph are the modified non-natural amino acid polypeptides that result from the reaction of the derivatizing molecule with the non-natural amino acid polypeptides. Further embodiments include any further modifications of the already modified non-natural amino acid polypeptides.

In another aspect are dicarbonyl-substituted molecules for the production of derivatized non-natural amino acid polypeptides based upon a heterocycle, including a nitrogen-containing heterocycle, linkage. In a further embodiment are dicarbonyl-substituted molecules used to derivatize diamine-containing non-natural amino acid polypeptides via the formation of a heterocycle, including a nitrogen-containing heterocycle group. In a further embodiment are dicarbonyl-substituted molecules that can form such heterocycle, including a nitrogen-containing heterocycle groups with a diamine-containing non-natural amino acid polypeptide in a pH range between about 4 and about 8. In a further embodiment are dicarbonyl-substituted molecules used to derivatize diamine-containing non-natural amino acid polypeptides via the formation of a heterocycle, including a nitrogen-containing heterocycle, linkage between the derivatizing molecule and the diamine-containing non-natural amino acid polypeptides. In a further embodiment the dicarbonyl-substituted molecules are diketone-substituted molecules, in other aspects ketoaldehyde-substituted molecules, in other aspects ketoacid-substituted molecules, in other aspects ketoester-substituted molecules, including ketothioester-substituted molecules. In further embodiments, the dicarbonyl-substituted molecules comprise a group selected from a desired functionality. In further or additional embodiments, the aldehyde-substituted molecules are aldehyde-substituted polyethylene glycol (PEG) molecules. In a further embodiment, the sidechain of the non-natural amino acid has a chemistry orthogonal to those of the naturally-occurring amino acids that allows the non-natural amino acid to react selectively with the carbonyl-substituted molecules. In a further embodiment, the sidechain of the non-natural amino acid comprises a moiety (e.g., diamine group) that reacts selectively with the dicarbonyl-containing molecule; in a further embodiment, the nucleophilic moiety on the sidechain of the non-natural amino acid can undergo electrophilic attack to generate a heterocyclic-derivatized protein, including a nitrogen-containing heterocycle-derivatized protein. In a further aspect related to the embodiments described in this paragraph are the modified non-natural amino acid polypeptides that result from the reaction of the derivatizing molecule with the non-natural amino acid polypeptides. Further embodiments include any further modifications of the already modified non-natural amino acid polypeptides.

In one aspect are methods to derivatize proteins via the reaction of carbonyl and hydrazine reactants to generate a heterocycle-derivatized protein, including a nitrogen-containing heterocycle-derivatized NRL. Included within this aspect are methods for the derivatization of NRL conjugates based upon the condensation of carbonyl- and hydrazine-containing reactants to generate a heterocycle-derivatized NRL, including a nitrogen-containing heterocycle-derivatized NRL. In additional or further embodiments are methods to derivatize ketone-containing NRL derivatives or aldehyde-containing NRL derivatives with hydrazine-functionalized non-natural amino acids. In yet additional or further aspects, the hydrazine-substituted molecule can include proteins, other polymers, and small molecules.

In another aspect are methods for the chemical synthesis of hydrazine-substituted molecules for the derivatization of carbonyl-substituted NRL conjugates. In one embodiment, the hydrazine-substituted molecule is a NRL conjugate suitable for the derivatization of carbonyl-containing non-natural amino acid polypeptides, including by way of example only, ketone-, or aldehyde-containing non-natural amino acid polypeptides.

In one aspect are non-natural amino acids for the chemical derivatization of NRL analogs based upon a quinoxaline or phenazine linkage. In further or additional embodiments, the non-natural amino acids are functionalized on their sidechains such that their reaction with a derivatizing NRL linker generates a quinoxaline or phenazine linkage. In further or additional embodiments, the non-natural amino acids are selected from amino acids having 1,2-dicarbonyl or 1,2-aryldiamine sidechains. In further or additional embodiments, the non-natural amino acids are selected from amino acids having protected or masked 1,2-dicarbonyl or 1,2-aryldiamine sidechains. Further included are equivalents to 1,2-dicarbonyl sidechains, or protected or masked equivalents to 1,2-dicarbonyl sidechains.

In another aspect are derivatizing molecules for the production of derivatized non-natural amino acid polypeptides based upon quinoxaline or phenazine linkages. In one embodiment are 1,2-dicarbonyl substituted NRL linker derivatives used to derivatize 1,2-aryldiamine containing non-natural amino acid polypeptides to form quinoxaline or phenazine linkages. In another embodiment are 1,2-aryldiamine substituted NRL linker derivatives used to derivatize 1,2-dicarbonyl containing non-natural amino acid polypeptides to form quinoxaline or phenazine linkages. In a further aspect related to the above embodiments are the modified non-natural amino acid polypeptides that result from the reaction of the derivatizing NRL linker with the non-natural amino acid polypeptides. In one embodiment are 1,2-aryldiamine containing non-natural amino acid polypeptides derivatized with 1,2-dicarbonyl substituted NRL linker derivative to form quinoxaline or phenazine linkages. In another embodiment are 1,2-dicarbonyl containing non-natural amino acid polypeptides derivatized with 1,2-aryldiamine substituted NRL linker derivatives to form quinoxaline or phenazine linkages.

Provided herein in certain embodiments are derivatizing molecules for the production of compounds comprising non-natural amino acid polypeptides based upon triazole linkages. In some embodiments, the reaction between the first and second reactive groups can proceed via a dipolarophile reaction. In certain embodiments, the first reactive group can be an azide and the second reactive group can be an alkyne. In further or alternative embodiments, the first reactive group can be an alkyne and the second reactive group can be an azide. In some embodiments, the Huisgen cycloaddition reaction (see, e.g., Huisgen, in 1,3-DIPOLAR CYCLOADDITION CHEMISTRY, (ed. Padwa, A., 1984), p. 1-176) provides for the incorporation of non-naturally encoded amino acids bearing azide and alkyne-containing side chains permits the resultant polypeptides to be modified with extremely high selectivity. In certain embodiments, both the azide and the alkyne functional groups are inert toward the twenty common amino acids found in naturally-occurring polypeptides. When brought into close proximity, however, the “spring-loaded” nature of the azide and alkyne groups is revealed and they react selectively and efficiently via Huisgen[3 2] cycloaddition reaction to generate the corresponding triazole. See, e.g., Chin et al., Science 301:964-7 (2003); Wang et al., J. Am, Chem. Soc., 125, 3192-3193 (2003); Chin et al., J. Am. Chem. Soc., 124:9026-9027 (2002). Cycloaddition reaction involving azide or alkyne-containing polypeptides can be carried out at room temperature under aqueous conditions by the addition of Cu(II) (e.g., in the form of a catalytic amount of CuSO₄) in the presence of a reducing agent for reducing Cu(II) to Cu(I), in situ, in catalytic amount. See, e.g., Wang et al., J. Am, Chem. Soc. 125, 3192-3193 (2003); Tornoe et al., J. Org. Chem. 67:3057-3064 (2002); Rostovtsev, Angew. Chem. Int, Ed. 41:2596-2599 (2002). Preferred reducing agents include ascorbate, metallic copper, quinine, hydroquinone, vitamin K, glutathione, cysteine, Fe², Co², and an applied electric potential.

Modification of NRL linked derivatives described herein with such reactions have any or all of the following advantages. First, diamines undergo condensation with dicarbonyl-containing compounds in a pH range of about 5 to about 8 (and in further embodiments in a pH range of about 4 to about 10, in other embodiments in a pH range of about 3 to about 8, in other embodiments in a pH range of about 4 to about 9, and in further embodiments a pH range of about 4 to about 9, in other embodiments a pH of about 4, and in yet another embodiment a pH of about 8) to generate heterocycle, including a nitrogen-containing heterocycle, linkages. Under these conditions, the sidechains of the naturally occurring amino acids are unreactive. Second, such selective chemistry makes possible the site-specific derivatization of recombinant proteins: derivatized proteins can now be prepared as defined homogeneous products. Third, the mild conditions needed to effect the reaction of the diamines described herein with the dicarbonyl-containing polypeptides described herein generally do not irreversibly destroy the tertiary structure of the polypeptide (excepting, of course, where the purpose of the reaction is to destroy such tertiary structure). Fourth, the reaction occurs rapidly at room temperature, which allows the use of many types of polypeptides or reagents that would be unstable at higher temperatures. Fifth, the reaction occurs readily is aqueous conditions, again allowing use of polypeptides and reagents incompatible (to any extent) with non-aqueous solutions. Six, the reaction occurs readily even when the ratio of polypeptide or amino acid to reagent is stoichiometric, near stoichiometric, or stoichiometric-like, so that it is unnecessary to add excess reagent or polypeptide to obtain a useful amount of reaction product, Seventh, the resulting heterocycle can be produced regioselectively and/or regiospecifically, depending upon the design of the diamine and dicarbonyl portions of the reactants, Finally, the condensation of diamines with dicarbonyl-containing molecules generates heterocycle, including a nitrogen-containing heterocycle, linkages which are stable under biological conditions.

Location of Non-Natural Amino Acids in Nuclear Receptor Ligand Linker Derivatives

The methods and compositions described herein include incorporation of one or more non-natural amino acids into a NRL linker derivative. One or more non-natural amino acids may be incorporated at one or more particular positions which do not disrupt activity of the NRL linker derivative. This can be achieved by making “conservative” substitutions, including but not limited to, substituting hydrophobic amino acids with non-natural or natural hydrophobic amino acids, bulky amino acids with non-natural or natural bulky amino acids, hydrophilic amino acids with non-natural or natural hydrophilic amino acids) and/or inserting the non-natural amino acid in a location that is not required for activity.

A variety of biochemical and structural approaches can be employed to select the desired sites for substitution with a non-natural amino acid within the NRL linker derivative. In some embodiments, the non-natural amino acid is linked at the C-terminus of the NRL derivative. In other embodiments, the non-natural amino acid is linked at the N-terminus of the NRL derivative, Any position of the NRL linker derivative is suitable for selection to incorporate a non-natural amino acid, and selection may be based on rational design or by random selection for any or no particular desired purpose. Selection of desired sites may be based on producing a non-natural amino acid polypeptide (which may be further modified or remain unmodified) having any desired property or activity, including but not limited to a receptor binding modulators, receptor activity modulators, modulators of binding to binder partners, binding partner activity modulators, binding partner conformation modulators, dimer or multimer formation, no change to activity or property compared to the native molecule, or manipulating any physical or chemical property of the polypeptide such as solubility, aggregation, or stability. Alternatively, the sites identified as critical to biological activity may also be good candidates for substitution with a non-natural amino acid, again depending on the desired activity sought for the polypeptide. Another alternative would be to simply make serial substitutions in each position on the polypeptide chain with a non-natural amino acid and observe the effect on the activities of the polypeptide, Any means, technique, or method for selecting a position for substitution with a non-natural amino acid into any polypeptide is suitable for use in the methods, techniques and compositions described herein.

The structure and activity of naturally-occurring mutants of a polypeptide that contain deletions can also be examined to determine regions of the protein that are likely to be tolerant of substitution with a non-natural amino acid. Once residues that are likely to be intolerant to substitution with non-natural amino acids have been eliminated, the impact of proposed substitutions at each of the remaining positions can be examined using methods including, but not limited to, the three-dimensional structure of the relevant polypeptide, and any associated ligands or binding proteins. X-ray crystallographic and NMR structures of many polypeptides are available in the Protein Data Bank, a centralized database containing three-dimensional structural data of large molecules of proteins and nucleic acids, one can be used to identify amino acid positions that can be substituted with non-natural amino acids. In addition, models may be made investigating the secondary and tertiary structure of polypeptides, if three-dimensional structural data is not available, Thus, the identity of amino acid positions that can be substituted with non-natural amino acids can be readily obtained.

Exemplary sites of incorporation of a non-natural amino acid include, but are not limited to, those that are excluded from potential receptor binding regions, or regions for binding to binding proteins or ligands may be fully or partially solvent exposed, have minimal or no hydrogen-bonding interactions with nearby residues, may be minimally exposed to nearby reactive residues, and/or may be in regions that are highly flexible as predicted by the three-dimensional crystal structure of a particular polypeptide with its associated receptor, ligand or binding proteins.

A wide variety of non-natural amino acids can be substituted for, or incorporated into, a given position in a polypeptide. By way of example, a particular non-natural amino acid may be selected for incorporation based on an examination of the three dimensional crystal structure of a polypeptide with its associated ligand, receptor and/or binding proteins, a preference for conservative substitutions

In one embodiment, the methods described herein include incorporating into the NRL linker derivative, where the NRL linker derivative comprises a first reactive group; and contacting the NRL linker derivative with a molecule (including but not limited to a second protein or polypeptide or polypeptide analog; an ccPSMA antibody or antibody fragment; and any combination thereof) that comprises a second reactive group. In certain embodiments, the first reactive group is a hydroxylamine moiety and the second reactive group is a carbonyl or dicarbonyl moiety, whereby an oxime linkage is formed. In certain embodiments, the first reactive group is a carbonyl or dicarbonyl moiety and the second reactive group is a hydroxylamine moiety, whereby an oxime linkage is formed. In certain embodiments, the first reactive group is a carbonyl or dicarbonyl moiety and the second reactive group is an oxime moiety, whereby an oxime exchange reaction occurs. In certain embodiments, the first reactive group is an oxime moiety and the second reactive group is carbonyl or dicarbonyl moiety, whereby an oxime exchange reaction occurs.

In some cases, the NRL linker derivative incorporation(s) will be combined with other additions, substitutions, or deletions within the polypeptide to affect other chemical, physical, pharmacologic and/or biological traits. In some cases, the other additions, substitutions or deletions may increase the stability (including but not limited to, resistance to proteolytic degradation) of the polypeptide or increase affinity of the polypeptide for its appropriate receptor, ligand and/or binding proteins. In some cases, the other additions, substitutions or deletions may increase the solubility (including but not limited to, when expressed in E. coli or other host cells) of the polypeptide. In some embodiments sites are selected for substitution with a naturally encoded or non-natural amino acid in addition to another site for incorporation of a non-natural amino acid for the purpose of increasing the polypeptide solubility following expression in E. coli, or other recombinant host cells. In some embodiments, the polypeptides comprise another addition, substitution, or deletion that modulates affinity for the associated ligand, binding proteins, and/or receptor, modulates (including but not limited to, increases or decreases) receptor dimerization, stabilizes receptor dimers, modulates circulating half-life, modulates release or bio-availability, facilitates purification, or improves or alters a particular route of administration. Similarly, the non-natural amino acid polypeptide can comprise chemical or enzyme cleavage sequences, protease cleavage sequences, reactive groups, antibody-binding domains (including but not limited to, FLAG or poly-His) or other affinity based sequences (including but not limited to, FLAG, poly-His, GST, etc.) or linked molecules (including but not limited to, biotin) that improve detection (including but not limited to, GFP), purification, transport thru tissues or cell membranes, prodrug release or activation, size reduction, or other traits of the polypeptide.

### Additional Synthetic Methodology

The non-natural amino acids described herein may be synthesized using methodologies described in the art or using the techniques described herein or by a combination thereof. As an aid, the following table provides various starting electrophiles and nucleophiles which may be combined to create a desired functional group. The information provided is meant to be illustrative and not limiting to the synthetic techniques described herein.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Non-limiting examples of carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other non-limiting examples of carbon nucleophiles include phosphorus yields, enol and enolate reagents; these carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile.

Non-limiting examples of non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), wherein X is a hetereoatom, including, but not limited to, oxygen, sulfur, or nitrogen.

The present disclosures provide targeting moieties conjugated with NRLs. In some aspects, the NRLs are capable of acting at nuclear receptors involved in metabolism or glucose homeostasis, and the conjugate provides superior biological effects on metabolism or glucose homeostasis compared to the peptide alone or the NRL alone. Without being bound by a theory of the invention, the targeting moieties may serve to target the NRLs to particular types of cells or tissues; or alternatively the NRLs may serve to target an antibody or enhance its transport into the cell, e.g. through binding of peptide to a receptor that internalizes the conjugate.

The targeting moiety—NRL conjugates of the invention can be represented by the following formula:

Ab-L-Y

wherein Ab is a targeting moiety, Y is a NRL, and L is a linking group or a bond,

The targeting moiety (Ab) in some embodiments is a molecule that binds to a defined soluble molecular target. The targeting moiety may bind a receptor, a cytokine, a hormone, a drug, or other soluble molecule. Antibody is used throughout the specification as a protypical example of a targeting moiety.

In the present disclosures relating to Ab-L-Y conjugates, Y is a ligand that acts at any nuclear receptor, including any one of the “nuclear hormone receptor superfamily” (NHR superfamily) set forth in Table 1, or a separate nuclear receptor class or subgroup thereof. This NITR superfamily is composed of structurally related proteins found within the interior of cells that regulate the transcription of genes. These proteins include receptors for steroid and thyroid hormones, vitamins, and other “orphan” proteins for which no ligands have been found. Nuclear hormone receptors generally include at least one of a C4-type zinc finger DNA-Binding Domain (DBD) and/or a Ligand Binding Domain (LBD). The DBD functions to bind DNA in the vicinity of target genes, and the LBD binds and responds to its cognate hormone. “Classical Nuclear Hormone Receptors” possess both a DBD and a LBD (e.g. Estrogen receptor alpha), while other nuclear hormone receptors possess only a DBD (e.g. Knirps, ORD) or only a LBD (e.g. Short Heterodimer Partner (SHP)).

### Antibodies (Ab)

Exemplary antibodies include α-PSMA antibodies having affinity and selectivity for PSMA.

Other exemplary parent antibodies include those selected from, and without limitation, anti-estrogen receptor antibody, anti-progesterone receptor antibody, anti-p53 antibody, anti-HER-2/neu antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9p24 antibody, anti-CD10 antibody, anti-CD11c antibody, anti-CD13 antibody, anti-CD14 antibody, anti-CD15 antibody, anti-CD19 antibody, anti-CD20 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, anti-CD41 antibody, anti-LCACD45 antibody, anti-CD45RO antibody, anti-CD45RA antibody, anti-CD39 antibody, anti-CD100 antibody, anti-CD95/Fas antibody, anti-CD99 antibody, anti-CD106 antibody, anti-ubiquitin antibody, anti-CD71 antibody, anti-c-myc antibody, anti-cytokeratins antibody, anti-vimentins antibody, anti-HPV proteins antibody, anti-kappa light chains antibody, anti-lambda light chains antibody, anti-melanosomes antibody, anti-prostate specific antigen antibody, anti-S-100 antibody, anti-tau antigen antibody, anti-fibrin antibody, anti-keratins antibody and anti-Tn-antigen antibody.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An antibody “which binds” a molecular target or an antigen of interest (non-limiting examples include PSMA, CD45, CD70, and CD74), is one capable of binding that antigen with sufficient affinity such that the antibody is useful in targeting a cell expressing the antigen. Where the antibody is one which binds, for example, PSMA, CD45, CD70, or CD74, it may be one which does not significantly cross-react with other proteins.

Molecular targets for antibodies encompassed by the present invention include prostate-specific membrane antigen, CD proteins and their ligands, such as, but not limited to: (i) CD3, CD4, CD8, CD19, CD20, CD22, CD34, CD40, CD45, CD70, CD74, CD79.alpha. (CD79a), and CD79.beta. (CD79b); (ii) members of the ErbB receptor family such as the EGF receptor, HER2, HER3 or HER4 receptor; (iii) cell adhesion molecules such as LFA-1, Mac 1, p150,95, VLA-4, ICAM-1, VCAM and .alpha.v.beta.3 integrin, including either alpha or beta subunits thereof (e.g. anti-CD11a, anti-CD18 or anti-CD11b antibodies); (iv) growth factors such as VEGF; IgE; blood group antigens; flk2/flt3 receptor; obesity (OB) receptor; mpl receptor; CTLA-4; protein C, BR3, c-met, tissue factor, ,beta.7 etc; and (v) cell surface and transmembrane tumor-associated antigens (TAA).

In one embodiment of the invention the target cell specific protein or peptide is selected from prostate cell, anti-A33, C595, 4D5, trastuzumab (Herceptin), egf/R3, humanized h-R3, C225 (Erbitux), BrE-3, murine A7, C50, humanized MN-14, anti-A33, MSN-1, bivatuzumab, U36, KIS1, HuM195, anti-CD45, anti-CD19, TXU(anti-CD7)-pokeweed antiviral protein, M195, anti-CD23, apolizumab (Hu 1D10), Campath-1H, N901, Ep2, somatostatin analogues (e.g. octreotide), tositumomab (Bexxar), ibritumomab tiuxetan (Zevalin), HB22.7, anti-CD40, OC125, PAM4 and J591.

### Anti-PSMA Antibody

Anti prostate-specific membrane antigen (αPSMA) antibodies known in the art are suitable for use in the present invention. For example, sequences for αPSMA J591 antibody are given in U.S. Pat. No. 7,666,425; αPSMA antibodies and antigen-binding fragments are given in U.S. Pat. No. 8,114,965; each incorporated herein by reference. Other U.S. patents disclosing αPSMA antibody sequences and/or PSMA binding agents, all of which are herein incorporated by reference, include U.S. Pat. No. 7,910,693; U.S. Pat. No. 7,875,278; U.S. Pat. No. 7,850,971; U.S. Pat. No. 7,514,078; U.S. Pat. No. 7,476,513; U.S. Pat. No. 7,381,407; U.S. Pat. No. 7,201,900; U.S. Pat. No. 7,192,586; U.S. Pat. No. 7,045,605; U.S. Pat. No. 6,962,981; U.S. Pat. No. 6,387,888; and U.S. Pat. No. 6,150,508.

### Anti-CD45 Antibody

CD45 is a hematopoietic cell-specific transmembrane protein tyrosine phosphatase essential for T and B cell antigen receptor-mediated signaling and also plays a important role in cytokine receptor signaling, chemokine and cytokine response and apoptosis regulation in multiple different leukocyte cell subsets (T cells, B cells, NK cells, myeloid cells, granulocytes, and dendritic cells). CD45 constitutes nearly 10% of T and B cell surface protein. The protein includes a large extracellular domain, and a phosphatase containing cytosolic domain. CD45 may act as both a positive and negative regulator depending on the nature of the stimulus and the cell type involved. CD45 RNA transcripts are alternatively spliced at the N-terminus, which results in extracellular domains of various sizes. The protein controls the activity of Src-family kinases, which if left unregulated, can cause cancer and autoimmunity. Mice and humans lacking CD45 expression have been shown to be immunodeficient. Multiple human or rodent mutations that result in altered CD45 expression or functional activity are associated with distinct malignancies, including autoimmunity, immunodeficiency, overt activation of T cells, susceptibility to infection, type I or type II associated immune disorders, and haemotologic malignancies (reviewed in Tchilian and Beverly, Trends in Immunology, 2006).

One embodiment of the present invention comprises administering to a patient in need of such treatment, an effective immunosuppressive amount of at least one compound which binds specifically to a CD45 leukocyte antigen present on T-cells conjugated to a nuclear receptor ligand. For example, the method of the present invention can be used to treat a patient undergoing transplant rejection, including graft-versus host disease or afflicted with an autoimmune disease. Preferably, the Ab binds to the CD45RB receptor. The present invention additionally provides pharmaceutical compositions comprising an effective immunosuppressive amount of at least one compound which specifically binds to a CD45 antigen in combination with a pharmaceutically acceptable carrier. In some embodiments of the present invention, the compound of the present method is an antibody. In still other embodiments, the antibody administered will be capable of binding to the CD45RB leukocyte antigen, the CD45RO leukocyte antigen, the CD45RA leukocyte antigen or the CD45RC leukocyte antigen. Most preferably, the antibody is capable of binding to the CD45RB or CD45RO leukocyte antigen.

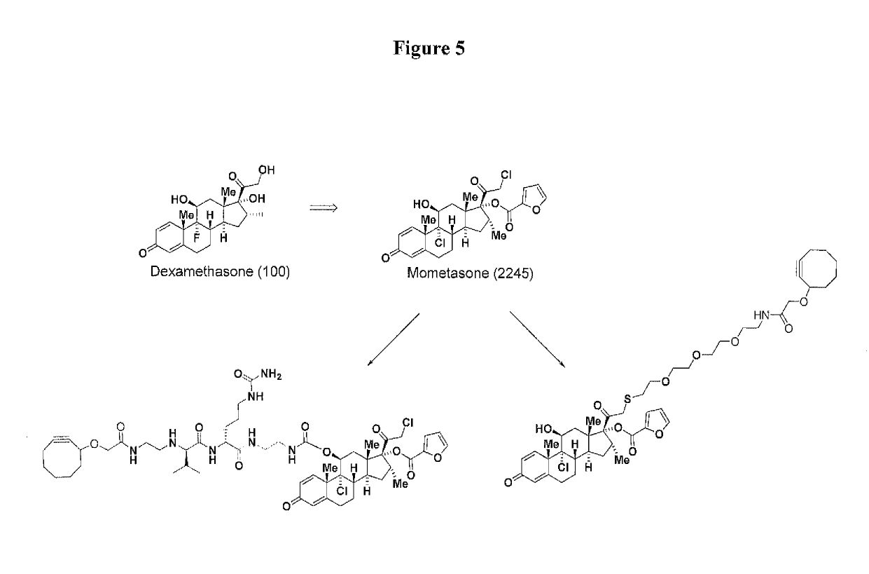

By “CD45” as used herein is meant a CD45 mRNA, protein, peptide, or polypeptide. The term “CD45” is also known in the art as PTPRC (protein tyrosine phosphatase, receptor type, C), B220, GP 180, LCA, LYS, and T200. The sequence of human CD45 cDNA is recorded at GenBank Accession No. NM.sub.--002838.2 (version dated Jan. 13, 2008) (see FIGS. 5A and 5B). Other human CD45 sequences are recorded at GenBank Accession Nos. NM.sub.--080921.2, NM.sub.--080922.2, NM.sub.--080923.2, Y00062.1, Y00638.1, BC014239.2, BC017863.1, BC031525.1, BC121086.1, BC121087.1, BC127656.1, BC127657.1, AY429565.1, AY567999.1, AK130573.1, DA670254.1, DA948670.1, AY429566.1, and CR621867.1. Mouse CD45 mRNA sequences are found at GenBank Accession Nos, NM.sub.--011210.2, AK054056.1, AK088215.1, AK154893.1, AK171802.1, BC028512.1, EF101553.1, L36091.1, M11934.1, M14342.1, M14343.1, M15174.1, M17320.1, and M92933.1. Rhesus monkey CD45 mRNA sequence are found at GenBank Accession No. XR.sub.--012672.1.

### Anti-CD70 Antibody

CD70 is a member of the tumor necrosis factor (TNF) family of cell membrane-bound and secreted molecules that are expressed by a variety of normal and malignant cell types. The primary amino acid (AA) sequence of CD70 predicts a transmembrane type II protein with its carboxyl terminus exposed to the outside of cells and its amino terminus found in the cytosolic side of the plasma membrane (Bowman et al., 1994, J. Immunol. 152:1756-61; Goodwin et al., 1993, Cell 73:447-56). Human CD70 is composed of a 20 AA cytoplasmic domain, an 18 AA transmembrane domain, and a 155 AA extracytoplasmic domain with two potential N-linked glycosylation sites (Bowman et al., supra; Goodwin et al., supra). Specific immunoprecipitation of radioisotope-labeled CD70-expressing cells by anti-CD70 antibodies yields polypeptides of 29 and 50 kDa (Goodwin et al., supra; Hintzen et al., 1994, J. Immunol. 152:1762-73). Based on its homology to TNF-alpha and TNF-beta, especially in structural strands C, D, H and I, a trimeric structure is predicted for CD70 (Petsch et al., 1995, Mol. Immunol. 32:761-72).

Original immunohistological studies revealed that CD70 is expressed on germinal center B cells and rare T cells in tonsils, skin, and gut (Hintzen et al., 1994, Int. Immunol. 6:477-80), Subsequently, CD70 was reported to be expressed on the cell surface of recently antigen-activated T and B lymphocytes, and its expression wanes after the removal of antigenic stimulation (Lens et al., 1996, Eur. J. Immunol. 26:2964-71; Lens et al., 1997, Immunology 90:38-45), Within the lymphoid system, activated natural killer cells (Orengo et al., 1997, Clin. Exp. Immunol, 107:608-13) and mouse mature peripheral dendritic cells (Akiba et al., 2000, J. Exp. Med. 191:375-80) also express CD70. In non-lymphoid lineages, CD70 has been detected on thymic medullar epithelial cells (Hintzen et al., 1994, supra; Hishima et al., 2000, Am. J. Surg. Pathol. 24:742-46).

CD70 is not expressed on normal non-hematopoietic cells, CD70 expression is mostly restricted to recently antigen-activated T and B cells under physiological conditions, and its expression is down-regulated when antigenic stimulation ceases. Evidence from animal models suggests that CD70 may contribute to immunological disorders such as, e.g., rheumatoid arthritis (Brugnoni et al., 1997, Immunol. Lett, 55:99-104), psoriatic arthritis (Brugnoni et al., 1997, Immunol. Lett. 55:99-104), and lupus (Oelke et al., 2004, Arthritis Rheum. 50:1850-60). In addition to its potential role in inflammatory responses, CD70 is also expressed on a variety of transformed cells including lymphoma B cells, Hodgkin's and Reed-Sternberg cells, malignant cells of neural origin, and a number of carcinomas.

In one embodiment of the present invention, anti-CD70 antibodies conjugated to a nuclear receptor ligand are provided. In some embodiments of the present invention, anti-CD70 antibodies conjugated to glucocorticoid receptor modulators is provided. In some embodiments, the anti-CD70 antibody includes at least one effector domain mediating at least an ADCC, ADCP or CDC response in the subject. In some embodiments, the binding agent exerts a cytostatic, cytotoxic or immunomodulatory effect in the absence of conjugation to a therapeutic agent. In some embodiments, the binding agent is conjugated to a therapeutic agent that exerts a cytotoxic, cytostatic or immunodulatory effect. The antibody can compete for binding to CD70 with monoclonal antibody 1F6 or 2F2.

In another aspect, a method of treating a CD70-expressing cancer in a subject is provided. The method generally includes administering to the subject an effective amount of a conjugated CD70 antibody. In some embodiments, the binding agent includes at least one effector domain mediating at least an ADCC, ADCP or CDC response in the subject. In some embodiments, the antibody exerts a cytostatic, cytotoxic or immunomodulatory effect in the absence of conjugation to a therapeutic agent. In some embodiments, the binding agent is conjugated to a therapeutic agent that exerts a cytotoxic, cytostatic or immunodulatory effect.

The anti-CD70 antibody can include, for example, an effector domain of a human IgM or IgG antibody. The IgG antibody can be, for example, a human IgG1 or IgG3 subtype. In some embodiments, the antibody includes a human constant region. In some embodiments, the CD70 binding agent competes for binding to CD70 with monoclonal antibody 1F6 or 2F2. In other embodiments, the antibody is a humanized 1F6. In other embodiments, the antibody is a humanized 2F2. The antibody can be, for example, monovalent, divalent or multivalent.

The CD70-expressing cancer can be, a kidney tumor, a B cell lymphoma, a colon carcinoma, Hodgkin's Disease, multiple myeloma, Waldenstrom's macroglobulinemia, non-Hodgkin's lymphoma, a mantle cell lymphoma, chronic lymphocytic leukemia, acute lymphocytic leukemia, a nasopharyngeal carcinoma, brain tumor or a thymic carcinoma. The kidney tumor can be, for example, a renal cell carcinoma. The brain tumor can be, for example, a glioma, a glioblastoma, an astrocytoma or a meningioma. The subject can be, for example, a mammal, such as a human being.

In another aspect, a method for treating an immunological disorder is provided. The method includes administering to a subject an effective amount of a CD70 binding agent. In some embodiments, the binding agent includes at least one effector domain mediating at least an ADCC, ADCP or CDC response in the subject. In some embodiments, the binding agent exerts a cytostatic, cytotoxic or immunomodulatory effect in the absence of conjugation to a therapeutic agent. In some embodiments, the binding agent is conjugated to a therapeutic agent that exerts a cytotoxic, cytostatic or immunodulatory effect. The CD70 binding agent can be, for example, an antibody. The antibody can include, for example, an effector domain of a human IgM or IgG antibody. The IgG antibody can be, for example, a human IgG₁ or IgG₃ subtype. In some embodiments, the antibody includes a human constant region.

The immunological disorder can be, for example, a T cell-mediated immunological disorder. In some embodiments, the T cell mediated immunogical disorder comprises activated T cells expressing CD70. In some embodiments, resting T cells are not substantially depleted by administration of the antibody-drug conjugate. The T cell-mediated immunological disorder also can be, for example, rheumatoid arthritis, psoriatic arthritis, systemic lupus erythematosus (SLE), Type I diabetes, asthma, atopic dermatitis, allergic rhinitis, thrombocytopenic purpura, multiple sclerosis, psoriasis, Sjogren's syndrome, Hashimoto's thyroiditis, Graves' disease, primary biliary cirrhosis, Wegener's granulomatosis, tuberculosis, or graft versus host disease. In other embodiments, the immunological disorder is an activated B-lymphocyte disorder. The subject can be, for example, a mammal, such as a human being.

The anti-CD70 antibody can be a monoclonal, chimeric or humanized antibody, or a fragment or derivative thereof. In some embodiments, the anti-CD70 antibody includes an antibody constant region or domain. The antibody constant region or domain can be, for example, of the IgG subtype. In an exemplary embodiment, the anti-CD70 antibody, fragment or derivatives thereof, competes with the murine monoclonal antibody (mAb) 1F6 or 2F2 for binding to CD70 and comprises human antibody constant region sequences. In another exemplary embodiment, the anti-CD70 antibody, or fragment or derivative thereof, has an effector domain (e.g., an Fe portion) that can interact with effector cells or complement to mediate a cytotoxic, cytostatic, and/or immunomodulatory effect that results in the depletion or inhibition of the proliferation of CD70-expressing cells. In another exemplary embodiment, the anti-CD70 antibody lacks effector function. In another exemplary embodiment, the anti-CD70 antibody is conjugated to a therapeutic agent. Also included are kits and articles of manufacture comprising a CD70 binding agent (e.g., a humanized anti-CD70 antibody).

### Anti-CD74 Antibody

The human leukocyte antigen-DR(HLA-DR) is one of three polymorphic isotypes of the class II major histocompatibility complex (MHC) antigen. Because HLA-DR is expressed at high levels on a range of hematologic malignancies, there has been considerable interest in its development as a target for antibody-based lymphoma therapy. However, safety concerns have been raised regarding the clinical use of HLA-DR-directed antibodies, because the antigen is expressed on normal as well as tumor cells, (Dechant et al., 2003, Semin Oncol 30:465-75) HLA-DR is constitutively expressed on normal B cells, monocytesmacrophages, dendritic cells, and thymic epithelial cells. In addition, interferon-gamma may induce HLA class II expression on other cell types, including activated T and endothelial cells (Dechant et al., 2003). The most widely recognized function of HLA molecules is the presentation of antigen in the form of short peptides to the antigen receptor of T lymphocytes. In addition, signals delivered via HLA-DR molecules contribute to the functioning of the immune system by up-regulating the activity of adhesion molecules, inducing T-cell antigen counterreceptors, and initiating the synthesis of cytokines. (Nagy and Mooney, 2003, J Mol Med 81:757-65; Scholl et al., 1994, Immunol Today 15:418-22)

The CD74 antigen is an epitope of the major histocompatibility complex (MHC) class II antigen invariant chain, Ii, present on the cell surface and taken up in large amounts of up to 8.times.10.sup.6 molecules per cell per day (Hansen et al., 1996, Biochem. J., 320: 293-300). CD74 is present on the cell surface of B-lymphocytes, monocytes and histocytes, human B-lymphoma cell lines, melanomas, T-cell lymphomas and a variety of other tumor cell types. (Hansen et al., 1996, Biochem. J., 320: 293-300) CD74 associates with α/β chain MHC II heterodimers to form MHC II αβIi complexes that are involved in antigen processing and presentation to T cells (Dixon et al., 2006, Biochemistry 45:5228-34; Loss et al., 1993, J Immunol 150:3187-97; Cresswell et al., 1996; Cell 84:505-7).

CD74 plays a role in cell proliferation and survival. Binding of the CD74 ligand, macrophage migration inhibitory factor (MIF), to CD74 activates the MAP kinase cascade and promotes cell proliferation (Leng et al., 2003, J Exp Med 197:1467-76), Binding of MIF to CD74 also enhances cell survival through activation of NF-.kappa.B and Bcl-2 (Lantner et al., 2007, Blood 110:4303-11),

Antibodies against CD74 and/or HLA-DR have been reported to show efficacy against cancer cells. Such anti-cancer antibodies include the anti-CD74 hLL1 antibody (milatuzumab) and the anti-HLA-DR antibody hL243 (also known as IMMU-114) (Berkova et al., Expert Opin. Investig. Drugs 19:141-49; Burton et al., 2004, Clin Cancer Res 10:6605-11; Chang et al., 2005, Blood 106:4308-14; Griffiths et al., 2003, Clin Cancer Res 9:6567-71; Stein et al., 2007, Clin Cancer Res 13:5556s-63s; Stein et al., 2010, Blood 115:5180-90). In some embodiments, an anti-CD74 antibody conjugated to a glucocorticoid receptor modulator via a non-naturally encoded amino acid is provided. In other embodiments of the present invention, an anti-CD74 antibody is conjugated to an interferon gamma via a non-naturally encoded amino acid. In other embodiments, the conjugated anti-CD74 antibody will be administered to a patient in need thereof. In some embodiments, the administration of interferon-gamma increases the expression of CD74 and enhances the sensitivity of cancer cells, autoimmune disease cells or immune dysfunction cells to the cytotoxic effects of anti-CD74 antibodies,

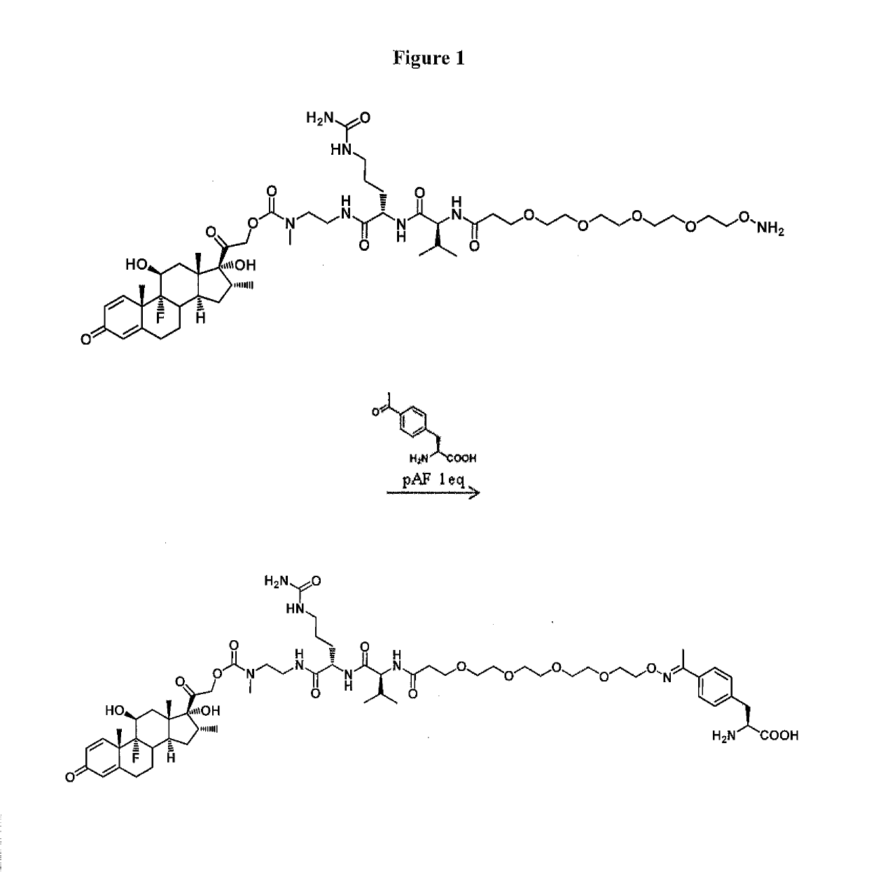

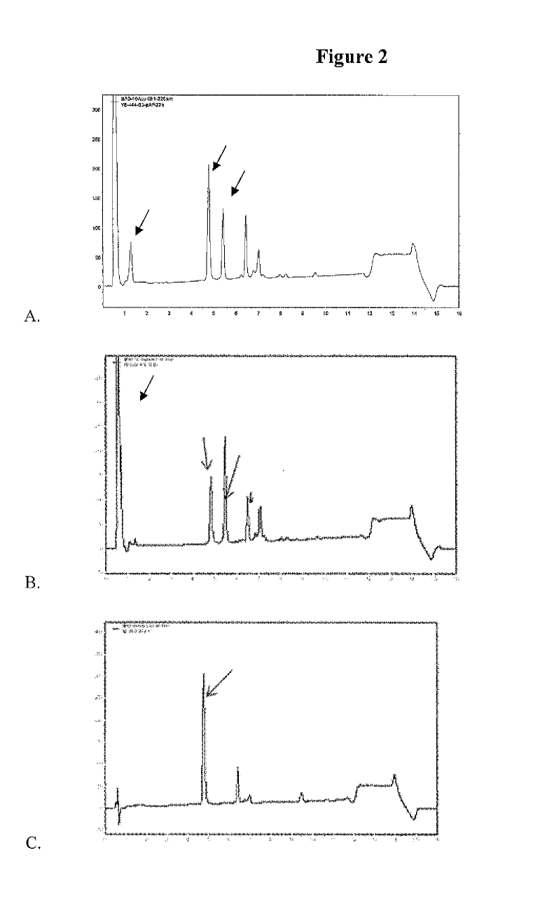

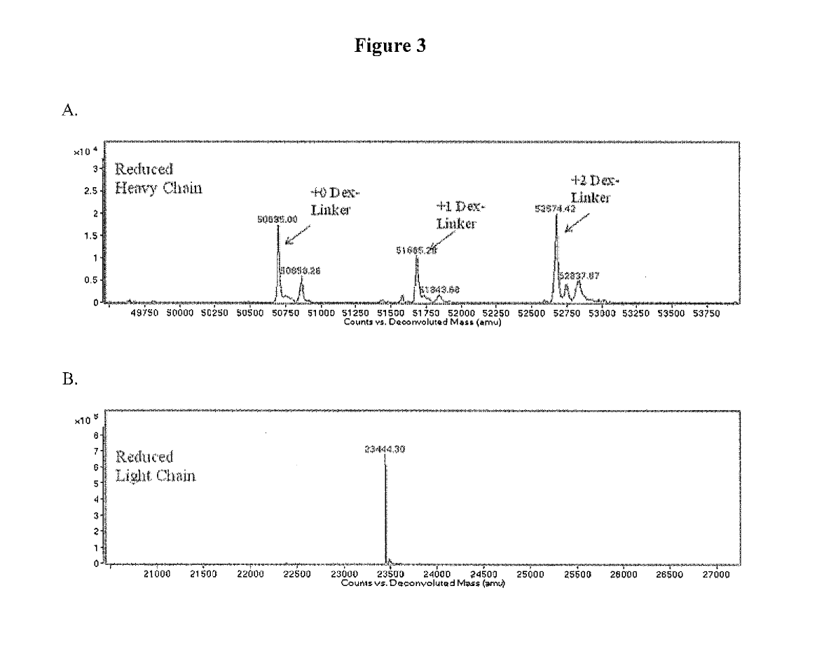

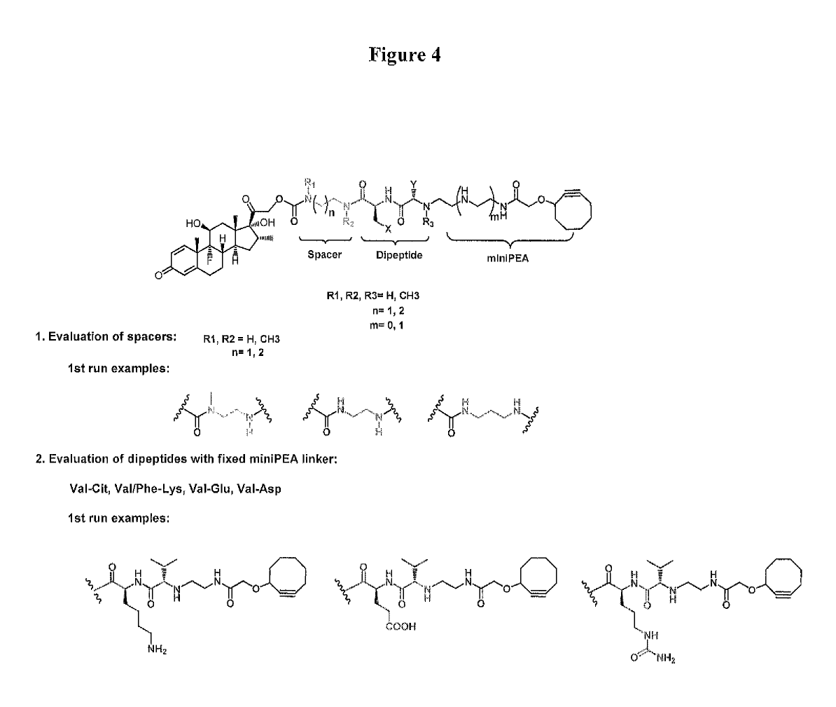

Many examples of anti-CD74 antibodies are known in the art and any such known antibody or fragment thereof may be utilized. In a preferred embodiment, the anti-CD74 antibody is an hLL1 antibody (also known as milatuzumab) that comprises the light chain complementarity-determining region (CDR) sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6). A humanized LL1 (hLL1) anti-CD74 antibody suitable for use is disclosed in U.S. Pat. No. 7,312,318, incorporated herein by reference from Col. 35, line 1 through Col. 42, line 27 and FIG. 1 through FIG. 4. However, in alternative embodiments, other known anti-CD74 antibodies may be utilized, such as LS-B1963, LS-B2594, LS-B1859, LS-B2598, LS-05525, LS-C44929, etc. (LSBio, Seattle, Wash.); LN2 (BIOLEGEND®, San Diego, Calif.); PIN.1, SPM523, LN3, CerCLIP.1 (ABCAM®, Cambridge, Mass.); At14/19, Bu45 (SEROTEC®, Raleigh, N.C.); 1D1 (ABNOVA®, Taipei City, Taiwan); 5-329 (EBIOSCIENCE®, San Diego, Calif.); and any other anti-CD74 antibody known in the art.

The anti-CD74 antibody may be selected such that it competes with or blocks binding to CD74 of an LL1 antibody comprising the light chain CDR sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6). Alternatively, the anti-CD74 antibody may bind to the same epitope of CD74 as an LL1 antibody. In still other alternatives, the anti-CD74 antibody may exhibit a functional characteristic such as internalization by Raji lymphoma cells in culture or inducing apoptosis of Raji cells in cell culture when cross-linked. These embodiments include anti-CD74 antibodies comprising a non-naturally encoded amino acid. These embodiments also include anti-CD74 antibodies comprising more than one non-naturally encoded amino acids.

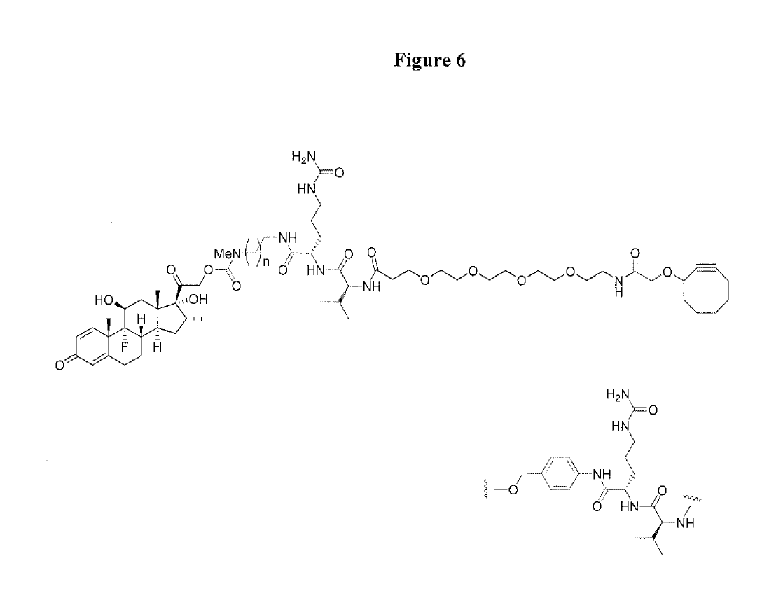

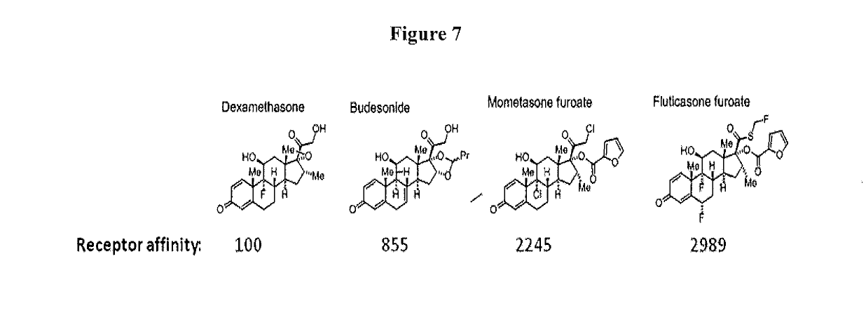

Alternative embodiments may involve use of anti-HLA-DR antibodies or fragments thereof and treatment with interferon-gamma to increase expression of HLA-DR and enhance sensitivity of cancer or autoimmune disease cells to anti-HLA-DR antibodies. Many examples of anti-HLA-DR antibodies are known in the art and any such known antibody or fragment thereof may be utilized. In a preferred embodiment, the anti-HLA-DR antibody is an hL243 antibody (also known as IMMU-114) that comprises the heavy chain CDR sequences CDR1 (NYGMN, SEQ ID NO:7), CDR2 (WINTYTREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12). A humanized L243 anti-HLA-DR antibody suitable for use is disclosed in U.S. Pat. No. 7,612,180, incorporated herein by reference in its entirety, as well as specific reference to the disclosure from Col. 46, line 45 through Col. 60, line 50 and FIG. 1 through FIG. 6. However, in alternative embodiments, other known anti-HLA-DR antibodies may be utilized, such as 1D10 (apolizumab) (Kostelny et al., 2001, Int J Cancer 93:556-65); MS-GPC-1, MS-GPC-6, MS-GPC-8, MS-GPC-10, etc, (U.S. Pat. No. 7,521,047); Lym-1, TAL 8.1, 520B, ML11C11, SPM289, MEM-267, TAL 15.1, TAL 1B5, G-7, 4D12, Bra30, etc. (Santa Cruz Biotechnology, Inc., Santa Cruz, Calif.); TAL 16.1, TU36, C120 (ABCAM®, Cambridge, Mass.); and any other anti-HLA-DR antibody known in the art.

The anti-HLA-DR antibody may be selected such that it competes with or blocks binding to HLA-DR of an L243 antibody comprising the heavy chain CDR sequences CDR1 (NYGMN, SEQ ID NO:7), CDR2 (WINTYTREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12). Alternatively, the anti-HLA-DR antibody may bind to the same epitope of HLA-DR as an L243 antibody.

The anti-CD74 and/or anti-HLA-DR antibodies or fragments thereof may be used as naked antibodies, alone or in combination with one or more therapeutic agents. Alternatively, the antibodies or fragments may be utilized as immunoconjugates, attached to one or more therapeutic agents. (For methods of making immunoconjugates, see, e.g., U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240, the Examples section of each incorporated herein by reference.) Therapeutic agents may be selected from the group consisting of a radionuclide, an enzyme, an immunomodulator, an anti-angiogenic agent, a pro-apoptotic agent, a cytokine, a hormone, an oligonucleotide molecule (e.g., an antisense molecule or a gene) or a second antibody or fragment thereof. Antisense molecules may include antisense molecules that correspond to bcl-2 or p53. However, other antisense molecules are known in the art, as described below, and any such known antisense molecule may be used. Second antibodies or fragments thereof may bind to an antigen selected from the group consisting of carbonic anhydrase IX, CCCL19, CCCL21, CSAp, CD1, CD1a, CD2, CD3, CD4, CD5, CD8, CD11A, CD14, CD15, CD16, CD18, CD19, IGF-1R, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD74, CD79a, CD80, CD83, CD95, CD126, CD133, CD138, CD147, CD154, CXCR4, CXCR7, CXCL12, HIF-1.alpha., AFP, PSMA, CEACAM5, CEACAM6, B7, ED-B of fibronectin, Factor H, FHL-1, Flt-3, folate receptor, GROB, HMGB-1, hypoxia inducible factor (HIF), HM1.24, insulin-like growth factor-1 (IGF-1), IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, IP-10, MAGE, mCRP, MCP-1, MIP-1A, MIP-1B, MIF, MUC1, MUC2, MUC3, MUC4, MUC5, NCA-95, NCA-90, Ia, HM1.24, EGP-1, EGP-2, HLA-DR, tenascin, Le(y), RANTES, T101, TAC, Tn antigen, Thomson-Friedenreich antigens, tumor necrosis antigens, TNF-.alpha., TRAIL receptor (R1 and R2), VEGFR, EGFR, P1GF, complement factors C3, C3a, C3b, C5a, C5, and an oncogene product.

The therapeutic agent may be selected from the group consisting of aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLRl, DNase I, Staphylococcal enterotoxin-A, pokeweed antiviral protein, gelonin, diphtheria toxin, Pseudomonas exotoxin, and Pseudomonas endotoxin,

The therapeutic agent may be an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

An immunomodulator of use may be selected from the group consisting of a cytokine, a stem cell growth factor, a lymphotoxin, a hematopoietic factor, a colony stimulating factor (CSF), an interferon (IFN), erythropoietin, thrombopoietin and combinations thereof. Exemplary immunomodulators may include IL-1, IL-2, IL-3, IL-6, IL-10, IL-12, IL-18, IL-21, interferons, interferon-β, interferon-γ, G-CSF; GM-CSF, and mixtures thereof.

Exemplary anti-angiogenic agents may include angiostatin, endostatin, basculostatin, canstatin, maspin, anti-VEGF binding molecules, anti-placental growth factor binding molecules, or anti-vascular growth factor binding molecules.

In certain embodiments of the present invention, the anti-CD74 or anti-HLA-DR complex may be formed by a technique known as dock-and-lock (DNL) (see, e.g., U.S. Pat. Nos. 7,521,056; 7,527,787; 7,534,866; 7,550,143 and U.S. Patent Publ. No. 20090060862, filed Oct. 26, 2007, the Examples section of each of which is incorporated herein by reference.) Generally, the DNL technique takes advantage of the specific and high-affinity binding interaction between a dimerization and docking domain (DDD) sequence derived from cAMP-dependent protein kinase and an anchor domain (AD) sequence derived from any of a variety of AKAP proteins. The DDD and AD peptides may be attached to any protein, peptide or other molecule. Because the DDD sequences spontaneously dimerize and bind to the AD sequence, the DNL technique allows the formation of complexes between any selected molecules that may be attached to DDD or AD sequences. Although the standard DNL complex comprises a trimer with two DDD-linked molecules attached to one AD-linked molecule, variations in complex structure allow the formation of dimers, trimers, tetramers, pentamers, hexamers and other multimers. In some embodiments, the DNL complex may comprise two or more antibodies, antibody fragments or fusion proteins which bind to the same antigenic determinant or to two or more different antigens. The DNL complex may also comprise one or more other effectors, such as a cytokine or PEG moiety.

Also disclosed is a method for treating and/or diagnosing a disease or disorder that includes administering to a patient a therapeutic and/or diagnostic composition that includes any of the aforementioned antibodies or fragments thereof. Typically, the composition is administered to the patient intravenously, intramuscularly or subcutaneously at a dose of 20-5000 mg.

In some embodiments of the present invention, the disease or disorder is associated with CD74- and/or HLA-DR-expressing cells and may be a cancer, an immune dysregulation disease, an autoimmune disease, an organ-graft rejection, a graft-versus-host disease, a solid tumor, non-Hodgkin's lymphoma, Hodgkin's lymphoma, multiple myeloma, a B-cell malignancy, or a T-cell malignancy. A B-cell malignancy may-include indolent forms of B-cell lymphomas, aggressive forms of B-cell lymphomas, chronic lymphatic leukemias, acute lymphatic leukemias, and/or multiple myeloma. Solid tumors may include melanomas, carcinomas, sarcomas, and/or gliomas. A carcinoma may include renal carcinoma, lung carcinoma, intestinal carcinoma, stomach carcinoma, breast carcinoma, prostate cancer, ovarian cancer, and/or melanoma.

Exemplary autoimmune diseases include acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositisdermatomyositis, polychondritis, pemphigus vulgaris, Wegener's granulomatosis, membranous nephropathy, amyotrophic lateral sclerosis, tabes dorsalis, giant cell arteritispolymyalgia, pernicious anemia, rapidly progressive glomerulonephritis, psoriasis, or fibrosing alveolitis. However, the skilled artisan will realize that any disease or condition characterized by expression of CD74 and/or HLA-DR may be treated using the claimed compositions and methods.

Table 2 presents a list of human CD antigen designations, antibodies to which may be used as targeting moieties in the present invention.

TABLE 2

| CD | | Molecular | | Other | Family |
|---|---|---|---|---|---|
| antigen | Cellular expression | weight | Functions | names | relationships |
| CD1a | Cortical thymocytes, | 43-49 | MHC class I- | | Ig superfamily |
| CD1b | Langerhans cells, | | like molecule, | | (IgSF) |
| CD1c | Dendritic cells, B cells | | associated with | | |
| CD1d | (CD1c), Intestinal | | b2- | | |
| | epithelium (CD1d) | | microglobulin. | | |
| | | | May have | | |
| | | | specialised role | | |
| | | | in antigen | | |
| | | | presentation | | |
| CD2 | T cells, thymocytes, | 45-58 | Adhesion | T11, LFA-2 | IgSF |
| | NK cells | | molecule, | | |
| | | | binding CD58 | | |
| | | | (LFA-3). Can | | |
| | | | activate T cells | | |
| CD2R | Activated T cells | 45-58 | activation- | T11-3 | IgSF |
| | | | dependent | | |
| | | | conformational | | |
| | | | form of CD2 | | |
| CD3 | Thymocytes, T cells | g: 25-28 | Associated with | T3 | IgSF (gde) z/h |
| | | d: 20 | the T cell | | related to FcR g |
| | | e: 20 | antigen receptor. | | chain |
| | | z: 16 | Required for cell | | |
| | | h: 22 | surface | | |
| | | | expression of | | |
| | | | and signal | | |
| | | | transduction by | | |
| | | | TCR. | | |
| CD4 | Thymocyte subsets, | 55 | Coreceptor for | T4, L3T4 | IgSF |
| | helper and | | MHC class II | | |
| | inflammatory T cells | | molecules. | | |
| | (about two thirds of | | Binds lck on | | |
| | peripheral T cells), | | cytoplasmic face | | |
| | monocytes, | | of membrane. | | |
| | macrophages | | Receptor for | | |
| | | | HIV-I and HIV- | | |
| | | | 2 gp120. | | |
| CD5 | Thymocytes, T cells, | 67 | Binds to CD72 | T1, Ly1 | Scavenger |
| | subset of B cells | | | | receptor |
| CD6 | Thymocytes, T cells, B | 100-130 | unknown. | T12 | Scavenger |
| | cell CLL | | | | receptor |
| CD7 | Pluripotential | 40 | unknown. | | IgSF |
| | hematopoietic cells, | | Marker for T | | |
| | thymocytes, T cells | | cell ALL and | | |
| | | | pluripotential | | |
| | | | stem cell | | |
| | | | leukemias | | |
| CD8 | Thymocyte subsets, | a: 32-34 | Coreceptor for | T8, Lyt2, 3 | IgSF |
| | cytotoxic T cells (about | b: 32-34 | MHC class I | | |
| | one third of peripheral | | molecules. | | |
| | T cells) | | Binds lck on | | |
| | | | cytoplasmic face | | |
| | | | of membrane | | |
| CD9 | Pre-B cells, | 22-27 | possible role in | | tetraspanning |
| | eosinophils, basophils, | | platelet | | membrane protein |
| | platelets | | aggregation and | | |
| | | | activation | | |
| CD10 | B and T cell precursors, | 100 | zinc | Neutral | |
| | bone marrow stromal | | metalloproteinase, | endopeptidase, | |
| | cells | | marker for pre | Common | |
| | | | B ALL | Acute | |
| | | | | Lymphocytic | |
| | | | | Leukemia | |
| | | | | Antigen | |
| | | | | (CALLA) | |
| CD11a | lymphocytes, | 180 | aL subunit of | LFA-1 | |
| | granulocytes, | | integrin LFA-1 | | |
| | monocytes and | | (associated with | | |
| | macrophages | | CD18); binds to | | |
| | | | CD54 (ICAM- | | |
| | | | 1), ICAM-2 and | | |
| | | | ICAM-3 | | |
| CD11b | myeloid and natural | 170 | aM subunit of | Mac-1 | |
| | killer cells | | integrin CR3 | | |
| | | | (associated with | | |
| | | | CD18); binds | | |
| | | | CD54, | | |
| | | | complement | | |
| | | | component iC3b | | |
| | | | and extracellular | | |
| | | | matrix proteins | | |
| CD11c | myeloid cells | 150 | aX subunit of | CR4, | |
| | | | integrin CR4 | p150, 95 | |
| | | | (associated with | | |
| | | | CD18); binds | | |
| | | | fibrinogen | | |
| CDw12 | monocytes, | 90-120 | unknown | | |
| | granulocytes, platelets | | | | |
| CD13 | myelomonocytic cells | 150-170 | zinc | aminopeptidase N | |
| | | | metalloproteinase | | |
| CD14 | myelomonocytic cells | 53-55 | receptor for | | |
| | | | complex of LPS | | |
| | | | and LPS binding | | |
| | | | protein (LBP) | | |
| CD15 | neutrophils, | | branched | Lewsi-x | |
| & CD15S | eosinophils, monocytes | | pentasaccharide, | (Lex) | |
| | | | expressed on | | |
| | | | glycolipids and | | |
| | | | many cell | | |
| | | | surface | | |
| | | | glycoproteins; | | |
| | | | the sialylated | | |
| | | | form is a ligand | | |
| | | | for CD62E | | |
| | | | (ELAM) | | |
| CD15u | sulphated CD15 | | | | |
| CD16 | neutrophils, NK cells, | 50-80 | component of | FcgRIII | IgSF |
| a & b | macrophages | | low affinity Fc | | |
| | | | receptor, | | |
| | | | FcgRIII, | | |
| | | | mediates | | |
| | | | phagocytosis | | |
| | | | and ADCC. | | |
| CDw17 | neutrophils, monocytes, | | lactosyl | | |
| | platelets | | ceramide, a cell | | |
| | | | surface | | |
| | | | glycosphingolipid | | |
| CD18 | Leukocytes | 95 | integrin b2 | | |
| | | | subunit, | | |
| | | | associates with | | |
| | | | CD11a,b and c. | | |
| CD19 | B cells | 95 | forms complex | | IgSF |
| | | | with CD21 | | |
| | | | (CR2)and CD81 | | |
| | | | (TAPA-1); | | |
| | | | coreceptor for B | | |
| | | | cells | | |
| CD20 | B cells | 33-37 | possible role in | | tetraspanning |
| | | | regulating B cell | | membrane protein |
| | | | activation | | |
| CD21 | mature B cells, FDC | 145 | receptor for | CR2 | complement |
| | | | complement | | control protein |
| | | | component C3d, | | (CCP) |
| | | | EBV. With | | superfamily |
| | | | CD19 and CD81 | | |
| | | | forms coreceptor | | |
| | | | for B cells | | |
| CD22 | mature B cells | a: 130 | Adhesion of B | BL-CAM | IgSF |
| | | b: 140 | cells to | | |
| | | | monocytes, T | | |
| | | | cells | | |
| CD23 | mature B cells, | 45 | low affinity | FceRII | C-type lectin |
| | activated macrophages, | | receptor for IgE, | | |
| | eosinophils, follicular | | ligand for | | |
| | dendritic cells, platelets | | CD19:CD21:CD81 | | |
| | | | coreceptor | | |
| CD24 | B cells, granulocytes | 35-45 | unknown | possible | |
| | | | | human | |
| | | | | homologue | |
| | | | | of mouse | |
| | | | | Heat Stable | |
| | | | | Antigen | |
| | | | | (HSA) or | |
| | | | | J11d. | |
| CD25 | activated T cells, B | a: 55 | IL-2 receptor | Tac | a: CCP b: cytokine |
| | cells, monocytes | :b75 | | | receptor |
| | | | | | superfamily |
| CD26 | Activated B and T | 110 | protease. | Dipeptidyl | |
| | cells, macrophages | | Recently | peptidase IV | |
| | | | implicated in | | |
| | | | HIV entry into | | |
| | | | cells. | | |
| CD27 | Medullary thymocytes, | 50-55 | unknown | | NGF receptor |
| | T cells | | | | superfamily |
| CD28 | T cell subsets, activated | 44 | Activation of | Tp44 | IgSF |
| | B cells | | naive T cells, | | |
| | | | receptor for | | |
| | | | costimulatory | | |
| | | | signal (signal 2) | | |
| | | | binds CD80 | | |
| | | | (B7-1) and B7-2 | | |
| CD29 | Leukocytes | 130 | Integrin b1 | | |
| | | | subunit, | | |
| | | | associates with | | |
| | | | CD49a in VLA- | | |
| | | | 1 integrin | | |
| CD30 | Activated B and T cells | 105-120 | unknown | Ki-1 | NGF receptor |
| | | | | | superfamily |
| CD31 | monocytes, platelets, | 130-140 | Possibly an | PECAM-1 | IgSF |
| | granulocytes, B cells, | | adhesion | | |
| | endothelial cells | | molecule | | |
| CDw32 | Monocytes, | 40 | low affinity Fc | FcgRII | IgSF |
| | granulocytes, B cells | | receptor for | | |
| | eosinophils | | aggregated | | |
| | | | Ig/immune | | |
| | | | complexes | | |
| CD33 | myeloid progenitor | 67 | unknown | | IgSF |
| | cells, monocytes | | | | |
| CD34 | hematopoietic | 105-120 | Ligand for | | |
| | precursors, capillary | | CD62 (L- | | |
| | endothelium | | selectin) | | |
| CD35 | Erythrocytes, B cells, | 250 | Complement | CR1 | CCP superfamily |
| | monocytes, neutrophils, | | receptor 1, binds | | |
| | eosinophils, FDC | | C3B and C4b, | | |
| | | | mediates | | |
| | | | phagocytosis | | |
| CD36 | platelets, monocytes | 88 | unknown | platelet | |
| | | | | GPIV | |
| CD37 | mature B cells, mature | 40-52 | unknown | | tetraspanning |
| | T cells, myeloid cells | | | | membrane protein |
| CD38 | early B and T cells, | 45 | unknown | | |
| | activated T cells, | | | | |
| | germinal centre B cells, | | | | |
| | plasma cells | | | | |
| CD40 | B cells, monocytes, | 50 | receptor for | | NGF receptor |
| | dendritic cells | | costimulatory | | superfamily |
| | | | signal for B | | |
| | | | cells, binds | | |
| | | | CD40 Ligand | | |
| | | | (T-BAM), | | |
| CD40L | activated CD4 T cells | 39 | ligand for CD40, | T-BAM | TNF-like |
| (CD154) | | | | | |
| CD41 | platelets, | 125/22 | aIIb integrin, | | |
| | megakaryocytes | dimer | associates with | | |
| | | | CD61 to form | | |
| | | | GPIIb, binds | | |
| | | | fibrinogen, | | |
| | | | fibronectin, von | | |
| | | | Willebrand | | |
| | | | factor and | | |
| | | | thrombospondin | | |
| CD42a, | platelets, | a: 23 | binds von | a: GPIX | |
| b, | megakaryocytes | b: 135, 23 | Willebrand | b: GPIba | |
| c, d | | c: 22 | factor, thrombin; | c: GPIbb | |
| | | d: 85 | essential for | d: GPV | |
| | | | platelet adhesion | | |
| | | | at sites of injury | | |
| CD43 | leukocytes, except | 115-135 | binds CD54 | leukosialin, | |
| | resting B cells | (neutrophils) | (ICAM-1) | sialophorin | |
| | | 95-115 (T | | | |
| | | cells) | | | |
| CD44 | leukocytes, | 80-95 | binds hyaluronic | Hermes | |
| | erythrocytes | | acid, mediates | antigen, | |
| | | | adhesion of | Pgp-1 | |
| | | | leukocytes | | |
| CD45 | leukocytes | 180-240 | tyrosine | Leukocyte | |
| | | | phosphatase, | common | |
| | | | augments | antigen | |
| | | | signalling | (LCA), | |
| | | | through antigen | T200, B220 | |
| | | | receptor of B | | |
| | | | and T cells, | | |
| | | | multiple | | |
| | | | isoforms result | | |
| | | | from alternative | | |
| | | | splicing (see | | |
| | | | below) | | |
| CD45RO | T cell subsets, B cell | 180 | isoform of CD45 | | |
| | subsets, monocytes, | | containing none | | |
| | macrophages | | of the A, B and | | |
| | | | C exons | | |
| CD45RA | B cells, T cell subsets | 205-220 | isoforms of | | |
| | (naive T cells) | | CD45 | | |
| | monocytes | | containing the A | | |
| | | | exon | | |
| CD45RB | T cell subsets, B cells, | 190-220 | isoforms of | T200 | |
| | monocytes, | | CD45 | | |
| | macrophages, | | containing the B | | |
| | granulocytes | | exon | | |
| CD45RC | Restricted T200 | | | | |
| CD46 | hematopoietic and non- | 56/66 (splice | membrane | MCP | CCP superfamily |
| | hematopoietic | variants) | cofactor protein, | | |
| | nucleated cells | | binds to C3b and | | |
| | | | C4b to permit | | |
| | | | their degradation | | |
| | | | by Factor I | | |
| CD47 | all cells | 47-52 | unknown, | | |
| | | | associated with | | |
| | | | Rh blood group | | |
| CD47R | previously CDw149 | | | | |
| CD48 | leukocytes | 40-47 | unknown | Blast-1 | IgSF |
| CD49a | activated T cells, | 210 | a1 integrin, | VLA-1 | |
| | monocytes | | associates with | | |
| | | | CD29, binds | | |
| | | | collagen, | | |
| | | | laminin | | |
| CD49b | B cells, monocyte, | 165 | a2 integrin, | VLA-2 | |
| | platelets | | associates with | | |
| | | | CD29, binds | | |
| | | | collagen, | | |
| | | | laminin | | |
| CD49c | B cells | 125 | a3 integrin, | VLA-3 | |
| | | | associates with | | |
| | | | CD29, | | |
| | | | bindslaminin, | | |
| | | | fibronectin | | |
| CD49d | B cells, thymocytes | 150 | a4 integrin, | VLA-4 | |
| | | | associates with | | |
| | | | CD29, binds | | |
| | | | fibronectin, | | |
| | | | PeyerÕs Patch | | |
| | | | HEV, VCAM-1 | | |
| CD49e | memory T cells, | 135, 25 | a5 integrin, | VLA-5 | |
| | monocytes, platelets | dimer | associates with | | |
| | | | CD29, binds | | |
| | | | fibronectin | | |
| CD49f | memory T cells, | 120, 25 | a6integrin, | VLA-6 | |
| | thymocytes, monocytes | dimer | associates with | | |
| | | | CD29, binds | | |
| | | | laminin | | |
| CD50 | thymocytes, T cells, B | 130 | unknown | ICAM3 | |
| | cells, monocytes, | | | | |
| | granulocytes | | | | |
| CD51 | platelets, | 125, 24 | av integrin, | vitronectin | |
| | megakaryocytes | dimer | associates with | receptor | |
| | | | CD61, binds | | |
| | | | vitronectin, von | | |
| | | | Willebrand | | |
| | | | factor, | | |
| | | | fibrinogen and | | |
| | | | thrombospondin | | |
| CD52 | thymocytes, T cells, B | 21-28 | unknown, target | CAMPATH-1 | |
| | cells (not plasma cells), | | for antibodies | | |
| | monocytes, | | used | | |
| | granulocytes | | therapeutically | | |
| | | | to deplete T | | |
| | | | cells | | |
| CD53 | leukocytes | 35-42 | unknown | MRC OX44 | tetraspanning |
| | | | | | membrane protein |
| CD54 | hematopoietic and non- | 85-110 | InterCellular | ICAM-1 | IgSF |
| | hematopoietic cells | | Adhesion | | |
| | | | Molecule, | | |
| | | | (ICAM)-1 binds | | |
| | | | CD11a/CD18 | | |
| | | | (LFA-1) and | | |
| | | | CD11b/CD18 | | |
| | | | (Mac-1) | | |
| | | | integrins, | | |
| | | | receptor for | | |
| | | | rhinovirus | | |
| CD55 | hematopoietic and non- | 60-70 | Decay | DAF | CCP superfamily |
| | hematopoietic cells | | Accelerating | | |
| | | | Factor (DAF), | | |
| | | | binds C3b, | | |
| | | | disassembles | | |
| | | | C3/C5 | | |
| | | | convertase | | |
| CD56 | NK cells | 175-185 | isoform of | NKH-1 | IgSF |
| | | | Neural Cell | | |
| | | | Adhesion | | |
| | | | Molecule | | |
| | | | (NCAM), | | |
| | | | adhesion | | |
| | | | molecule | | |
| CD57 | NK cells subsets of T | | oligosaccharide, | HNK-1, | |
| | cells, B cells and | | found on many | Leu-7 | |
| | monocytes | | cell surface | | |
| | | | glycoproteins | | |
| CD58 | hematopoietic and non- | 55-70 | Leukocyte | LFA-3 | IgSF |
| | hematopoietic cells | | Function- | | |
| | | | associated | | |
| | | | Antigen-3 | | |
| | | | (LFA-3), binds | | |
| | | | CD2, adhesion | | |
| | | | molecule | | |
| CD59 | hematopoietic and non- | 19 | binds | Protectin, | |
| | hematopoietic cells | | comlement | Mac | |
| | | | components C8 | inhibitor | |
| | | | and C9, blocks | | |
| | | | assembly of | | |
| | | | membrane | | |
| | | | attack complex | | |
| CD60 | T cell subsets, platelets, | | oligosaccharide | | |
| | monocytes | | present on | | |
| | | | gangliosides | | |
| CD60a | GD3 | | | | |
| CD60b | 9-O-acetyl-GD3 | | | | |
| CD60c | 7-O-acetyl-GD3 | | | | |
| CD61 | platelets, | 105 | integrin b3 | | |
| | megakaryocytes, | | subunit, | | |
| | macrophages | | associates with | | |
| | | | CD41 | | |
| | | | (GPIIb/IIIa) or | | |
| | | | CD51 | | |
| | | | (vitronectin | | |
| | | | receptor) | | |
| CD62E | endothelium | 140 | endothelium | ELAM-1, E- | C type lectin |
| | | | leukocyte | selectin | |
| | | | adhesion | | |
| | | | molecule | | |
| | | | (ELAM), | | |
| | | | bindssialyl- | | |
| | | | Lewis x, | | |
| | | | mediates rolling | | |
| | | | interaction of | | |
| | | | neutrophils on | | |
| | | | endothelium | | |
| CD62L | B cells, T cells, | 150 | leukocyte | LAM-1, L- | C type lectin |
| | monocytes, NK cells | | adhesion | selectin, | |
| | | | molecule | LECAM-1 | |
| | | | (LAM), binds | | |
| | | | CD34, | | |
| | | | GlyCAM, | | |
| | | | mediates rolling | | |
| | | | interactions with | | |
| | | | endothelium | | |
| CD62P | platelets, | 140 | adhesion | P-selectin, | C type lectin |
| | megakaryocytes, | | molecule, binds | PADGEM | |
| | endothelium | | sialyl Lewis x, | | |
| | | | mediates | | |
| | | | interaction of | | |
| | | | platelets with | | |
| | | | neutrophils, | | |
| | | | monocytes and | | |
| | | | rolling | | |
| | | | interaction of | | |
| | | | neutrophils on | | |
| | | | endothelium | | |
| CD63 | activated platelets, | 53 | unknown, is | | tetraspanning |
| | monocytes, | | lysosomal | | membrane protein |
| | macrophages | | membrane | | |
| | | | protein | | |
| | | | translocated to | | |
| | | | cell surface after | | |
| | | | activation | | |
| CD64 | monocytes, | 72 | hign affinity | FcgRI | IgSF |
| | macrophages | | receptor for IgG | | |
| | myeloid cells | | oligosaccharide | | |
| | | | component of a | | |
| | | | ceramide | | |
| | | | dodecasaccharide | | |
| CD65 | Ceramide | | | | |
| | dodecasaccharide 4c | | | | |
| CD66a | neutrophils | 160-180 | unknown, | biliary | IgSF |
| | | | member of | glycoprotein- | |
| | | | carcinoembryonic | 1 (BGP-1) | |
| | | | antigen (CEA) | | |
| | | | family (see | | |
| | | | below) | | |
| CD66b | granulocytes | 95-100 | unknown, | previously | IgSF |
| | | | member of | CD67 | |
| | | | carcinoembryonic | | |
| | | | antigen (CEA) | | |
| | | | family | | |
| CD66c | neutrophils, colon | 90 | unknown, | Nonspecific | IgSF |
| | carcinoma | | member of | Crossreacting | |
| | | | carcinoembryonic | Antigen | |
| | | | antigen (CEA) | (NCA) | |
| | | | family | | |
| CD66d | neutrophils | 30 | unknown, | | IgSF |
| | | | member of | | |
| | | | carcinoembryonic | | |
| | | | antigen (CEA) | | |
| | | | family | | |
| CD66e | adult colon epithelium, | 180-200 | unknown, | CarcinoEmbryonic | IgSF |
| | colon carcinoma | | member of | Antigen | |
| | | | carcinoembryonic | (CEA) | |
| | | | antigen (CEA) | | |
| | | | family | | |
| CD66f | Pregnancy specific | | | | |
| | glycoprotein | | | | |
| CD68 | monocytes, | 110 | unknown | macrosialin | |
| | macrophages, | | | | |
| | neutrophils, basophils, | | | | |
| | large lymphocytes | | | | |
| CD69 | activated B cells, | 28, 32 | unknown, early | Activation | |
| | activated T cells, | homodimer | activation | Inducer | |
| | activated macrophages, | | antigen | Molecule | |
| | activated NK cells | | | (AIM) | |
| CD70 | activated B cells, | 75, 95, 170 | unknown | Ki-24 | |
| | activated T cells, | | | | |
| | macrophages | | | | |
| CD71 | activated leukocytes | 90-95 | transferrin | T9 | |
| | | homodimer | receptor | | |
| CD72 | B cells | 42 | unknown, ligand | Lyb-2 | C type lectin |
| | | homodimer | for CD5 | | |
| CD73 | B cell subsets, T cell | 69 | ecto-5O- | | |
| | subsets | | nucleotidase, | | |
| | | | dephosphorylates | | |
| | | | nucleotides to | | |
| | | | allow nucleoside | | |
| | | | uptake | | |
| CD74 | B cells, macrophages, | 33, 35, 41, 43 | MHC class II | Ii, Ig | |
| | monocytes, MHC class | (alternate | associated | | |
| | II positive cells | initiation | Invariant chain | | |
| | | and splicing) | | | |
| CD75 | mature B cells, T cell | | unknown, | | |
| | subsets | | possibly | | |
| | | | oligosaccharide, | | |
| | | | dependent on | | |
| | | | sialylation | | |
| CDw76 | alpha-2,6-sialylated | | unknown, | | |
| CD75s | lactosamines | | possibly | | |
| | | | oligosaccharide, | | |
| | | | dependent on | | |
| | | | sialylation | | |
| CD77 | germinal center B cells | | unknown, | Globotriaocylceramide | |
| | | | | (Gb3), Pk | |
| | | | | blood group | |
| CDw78 | B cells | | unknown | Ba | |
| (deleted) | | | | | |
| CD79a, | B cells | a: 32-33 | components of B | Iga, Igb | IgSF |
| CD79b | | b: 37-39 | cell antigen | | |
| | | | receptor | | |
| | | | analogous to | | |
| | | | CD3, required | | |
| | | | for cell surface | | |
| | | | expression and | | |
| | | | signal | | |
| | | | transduction | | |
| CD80 | B cell subset | 60 | costimulator, | B7 (now B7- | IgSF |
| | | | ligand for CD28 | 1), BB1 | |
| | | | and CTLA-4 | | |
| CD81 | lymphocytes | 26 | associates with | Target of | tetraspanning |
| | | | CD19, CD21 to | AntiProliferative | membrane protein |
| | | | form B cell | Antibody | |
| | | | coreceptor | (TAPA-1) | |
| CD82 | leukocytes | 50-53 | unknown | R2 | tetraspanning |
| | | | | | membrane protein |
| CD83 | Activated B cells, | 43 | | HB15 | |
| | activated T cells, | | | | |
| | circulating dendritic | | | | |
| | cells (veil cells) | | | | |
| CDw84 | monocytes, platelets, | 73 | | GR6 | |
| | circulating B cells | | | | |
| CD85 | ILT/LIR family | | | | |
| CD86 | monocytes, activated B | 80 | | FUN-1, | |
| | cells | | | GR65 | |
| CD87 | granulocytes, | 50-65 | Urokinase | UPA-R | |
| | monocytes, | | plasminogen | | |
| | macrophages, activated | | activator | | |
| | T cells | | receptor | | |
| CD88 | polymorphonuclear | 40 | Receptor for | C5aR | rhodopsin |
| | leukocytes, | | complement | | superfamily |
| | macrophages, mast | | component C5a | | |
| | cells | | | | |
| CD89 | monocytes, | 50-70 | IgA receptor | FcaR | IgSF |
| | macrophages, | | | | |
| | granulocytes, | | | | |
| | neutrophils, B cell | | | | |
| | subsets, T cell subsets | | | | |
| CD90 | CD34+ prothymocytes | 18 | unknown | Thy-1 | IgSF |
| | (human) thymocytes, T | | | | |
| | cells (mouse) | | | | |
| CD91 | monocytes | 600 | a2 | unknown | |
| | | | macroglobulin | | |
| | | | receptor see | | |
| | | | lipoprotein | | |
| CDw92 | neutrophils, monocytes, | 70 | unknown | GR9 | |
| | platelets, endothelium | | | | |
| CD93 | neutrophils, monocytes, | 120 | unknown | GR11 | |
| | endothelium | | | | |
| CD94 | T cell subsets, NK cells | 43 | unknown | KP43 | |
| CD95 | wide variety of cell | 43 | binds TNF-like | Apo-1, Fas | NGF receptor |
| | lines in vivodistribution | | ligand, induces | | superfamily |
| | uncertain | | apoptosis | | |
| CD96 | activated T cells | 160 | Unknown | T cell | |
| | | | | ACTivation | |
| | | | | Increased | |
| | | | | Late | |
| | | | | Expression | |
| | | | | (TACTILE) | |
| CD97 | activated cells | 74, 80, 90 | unknown | GR1 | |
| CD98 | T cells, B cells, NK | 80, 40 | unknown | 4F2 | |
| | cells, granulocytes, all | heterodimer | | | |
| | human cell lines | | | | |
| CD99 | Peripheral blood | 32 | Unknown | MIC2, E2 | |
| | lymphocytes, | | | | |
| | thymocytes | | | | |
| CD100 | Broad expression on | 150 | unknown | GR3 | |
| | haematopoietic cells | | | | |
| CD101 | Granulocytes, | 140 | Unknown | BPC#4, BB27; | |
| | macrophages | | | BA27 | |
| CD102 | Resting lymphocytes, | 55-65 | binds | ICAM-2 | IgSF |
| | monocytes, strongest | | CD11a/CD18 | | |
| | on vascular endothelial | | (LEA-1) but not | | |
| | cells | | CD11b/CD18 | | |
| | | | (Mac-1) | | |
| CD103 | Intraepithelial | 150, 25 | aE integrin | HML-1, a6, | |
| | lymphocytes, 2-6% of | | | aE integrin | |
| | peripheral blood | | | | |
| | lymphocytes | | | | |
| CD104 | Epithelia, Schwann | 220 | b4 integrin | b4, b4 | |
| | cells, some tumour | | | integrin | |
| | cells | | | | |
| CD105 | Endothelial cells, bone | 95, | unknown, | Endoglin | |
| | marrow cell subset, in | homodimer | possibly ligand | | |
| | vitro activated | | for an integrin | | |
| | macrophages | | | | |
| CD106 | Endothelial cells | 100, 110 | Adhesion | VCAM-1 | IgSF |
| | | | molecule, ligand | | |
| | | | for VLA-4 | | |
| CD107a | Activated platelets | 110 | Unknown, is | Lysosomal | |
| | | | lysosomal | Associated | |
| | | | membrane | Membrane | |
| | | | protein | Protein-1 | |
| | | | translocated to | (LAMP-1) | |
| | | | the cell surface | | |
| | | | after activation | | |
| CD107b | activated platelets | 120 | Unknown, is | LAMP-2 | |
| | | | lysosomal | | |
| | | | membrane | | |
| | | | protein | | |
| | | | translocated to | | |
| | | | the cell surface | | |
| | | | after activation | | |
| CD108 | Activated T cells in | 80 | Unknown | GR2 | |
| | spleen, some stromal | | | | |
| | cells | | | | |
| CD109 | Activated T cells, | 170/50 | Unknown | Platelet | |
| | platelets, endothelial | | | activation | |
| | cells | | | factor, GR56 | |
| CD110 | Platelets | | | NPL, TPO R | |
| CD111 | Myeloid Cells | | | PRR1/Nectin1 | |
| CD112 | Myeloid Cells | | | PRR2 | |
| CD113 | For Many of the upper antibodies, between CD114 to CD130 please also see our | | | | |
| | Interleukin & Growth Factor Receptor Data Pack | | | | |
| CD114 | G-CSF receptor | | | | |
| CD115 | monocytes, | 150 | Macrophage | M-CSFR, c- | IgSF |
| | macrophages | | Colony | fms | |
| | | | Stimulating | | |
| | | | Factor (M-CSF) | | |
| | | | receptor | | |
| CD116 | monocytes, | 70-85 | Granulocyte, | GM-CSFRa | cytokine receptor |
| | neutrophils, eosinophils, | | Macrophage | | superfamily |
| | endothelium | | Colony | | |
| | | | Stimulating | | |
| | | | Factor (GM- | | |
| | | | CSF) receptor a | | |
| | | | chain | | |
| CD117 | hematopoietic | 145 | Stem Cell Factor | c-kit | IgSF tyrosine |
| | progenitors | | (SCF) receptor | | kinase |
| CD118 | Broad cellular | | Interferon a, b | IFNa, bR | |
| | expression | | receptor | | |
| CD119 | marophages, | 90-100 | Interferon g | IFNgR | |
| | monocytes, B cells, | | receptor | | |
| | endothelium | | | | |
| CD120a | hematopoietic and non- | 50-60 | TNF receptor, | TNFR-I | NFG receptor |
| | hematopoietic cells, | | binds both TNFa | | superfamily |
| | highest on epithelial | | and TNFb | | |
| | cells | | | | |
| CD120b | hematopoietic and non- | 75-85 | TNF receptor, | TNFR-II | NFG receptor |
| | hematopoietic cells, | | binds both TNFa | | superfamily |
| | highest on myeloid | | and TNFb | | |
| | cells | | | | |
| CD121a | thymocytes, T cells | 80 | type I | IL-1R type I | IgSF |
| | | | interleukin 1 | | |
| | | | receptor, binds | | |
| | | | IL-1a and IL-1b | | |
| CDw121b | B cells, macrophages, | 60-70 | type II | IL-1R, ytpe | IgSF |
| | monocytes | | interleukin 1 | II | |
| | | | receptor, binds | | |
| | | | IL-1a and IL-1b | | |
| CD122 | Natural killer cells, | 75 | IL-2 receptor b | IL-2Rb | |
| | resting T cell | | chain | | |
| | subpopulation, some B | | | | |
| | cell lines | | | | |
| CDw123 | Bone marrow stem | 70 | IL-3 receptor a | IL-3R | cytokine receptor |
| | cells, granulocytes, | | chain | | superfamily |
| | monocytes, | | | | fibronectin type |
| | megakaryocytes | | | | III superfamily |
| CD124 | Mature B and T cells, | 130-150 | IL-4 receptor | IL-4R | cytokine receptor |
| | haematopoietic | | | | superfamily |
| | precursor cells | | | | fibronectin type |
| | | | | | III superfamily |
| CDw125 | Eosinophils, basophils | 55-60 | IL-5 receptor | IL-5R | cytokine receptor |
| | | | | | superfamily |
| | | | | | fibronectin type |
| | | | | | III superfamily |
| CD126 | Activated B cells and | 80 | IL-6 receptor a | IL-6R | IgSF cytokine |
| | plsama cells, weak on | | subunit | | receptor |
| | most leukocytes | | | | superfamily |
| | | | | | fibronectin type |
| | | | | | III superfamily |
| CD127 | Bone marrow lymphoid | 68-79, | IL-7 receptor | IL-7R | fibronectin type |
| | precursors, pro-B cells, | possibly | | | III superfamily |
| | mature T | forms | | | |
| | cells, monocytes | homodimers | | | |
| CDw128 | Neutrophils, basophils, | 58-67 | IL-8 receptor | IL-8R | rhodopsin |
| | T cell subset | | | | superfamily |
| CD129 | not yet assigned | | | | |
| CDw130 | Activated B cells and | 130 | IL-6 receptor b | IL-6rb | IgSF |
| | plasma cells; weak on | | subunit | | cytokine receptor |
| | most leukocytes; | | | | superfamily |
| | endothelial cels | | | | fibronectin type |
| | | | | | III superfamily |
| CDw131 | monocytes, | 95-120 | B and T cell | IL-3R | |
| | granulocytes, | | growth | common | |
| | eosinophils, B cells, | | | beta chain | |
| CD132 | T and B cells, | 64 | B and T cell | common | |
| | fibroblasts | | growth | gamma | |
| | haematopoietic | | | chain | |
| | precursors | | | | |
| CD133 | stem/progenitor cells | | | AC133 | |
| CD134 | activated T cells | 48-50 | adhesion | OX-40 | |
| | | | activated T | | |
| | | | cells; ligand for | | |
| | | | gp34 | | |
| CD135 | CD34 cells, carcinoma | 130-150 | receptor tyrosine | FLT3/FLK2 | |
| | cells | | kinase | | |
| CDw136 | | 180 | receptor tyrosine | MSP-R | |
| | | | kinase | | |
| CDw137 | T cells | 30 | co-stimulatory T | 4-1BB | |
| | | | cell activation | | |
| CD138 | plasma cells; syndecan 1 | | ligand for | syndecan-1 | |
| | | | collagen type 1 | | |
| CD139 | | | | | |
| CD140a | | 180 | PDGFRa | | |
| CD140b | | 180 | PDGFRb | | |
| CD141 | myeloid cells, | 100 | myeloid | Thrombomodulin | |
| | endothelial cells, | | thrombin | | |
| | smooth muscle cells | | receptor; | | |
| | | | regulation of | | |
| | | | coagulation | | |
| CD142 | monocytes, endothelial | 45 | receptor for | Tissue | |
| | cells | | clotting factor | Factor | |
| | | | VII, inhibits | | |
| | | | clotting | | |
| CD143 | endothelial and | 170 | peptidyl- | Angiotensin | |
| | epithelial cells | | peptidase, ACE | converting | |
| | | | | enzyme | |
| | | | | (ACE) | |
| CD144 | endothelial cells | 135 | adhesion | VE- | |
| | | | molecule; role in | Cadherin | |
| | | | cell-cell | | |
| | | | adhesion | | |
| CDw145 | endothelial cells | | | | |
| CD146 | endothelial cells, | 113-118 | homing of | MUC18, S- | |
| | melanoma cells, | | activated T cells | endo | |
| | follicular dendritic cell | | | | |
| CD147 | endothelial cells, | | | Neurothelin, | |
| | myeloid cells, | | | basoglin | |
| | lymphocytes | | | | |
| CD148 | | | | HPTP-eta, | |
| | | | | p260 | |
| CDw149 | | | | MEM-133 | |
| (now | | | | | |
| CD47R) | | | | | |
| CD150 | T and B cells | 75-95 | signalling | SLAM(surface | |
| | | | molecule | lymphocyte | |
| | | | | activation | |
| | | | | marker) | |
| CD151 | | | PETA-3 | | |
| CD152 | T cells | 44 | neg. regul. for T | CTLA-4 | |
| | | | cells co | | |
| | | | stimul; ligand | | |
| | | | CD80,86 | | |
| CD153 | T cells | 40 | co-stimulatory | CD30L | |
| | | | for T cells; | | |
| | | | ligand for CD30 | | |
| CD154 | T cells | 32-39 | co-stimulatory | CD40L, T- | |
| | | | molecule; ligand | BAM | |
| | | | for CD40 | | |
| CD155 | | | Polio virus | | |
| | | | receptor (PVR) | | |
| CD156a | | | ADAM8 | | |
| CD156b | | | | ADAM17/TACE | |
| CD157 | | | MO-5, BST-1 | | |
| CD158a | T cells | 58/50 | inhibition of | p58.1 | |
| | | | cytotox; class-I | | |
| | | | specific NK- | | |
| | | | receptor | | |
| CD158b | T cells | 58/50 | inhibition of | p58.2 | |
| | | | cytotox; class-I | | |
| | | | specific NK- | | |
| | | | receptor | | |
| CD158c | T cells | 55-58 | activating | p58.3 | |
| | | | molecule; member | | |
| | | | of Ig | | |
| | | | superfamily | | |
| CD159a | NK cells | | | NKG2A | |
| CD160 | T cells | | | BY55 | |
| CD161 | NK cells | 60 | regulation of NK | NKRP-1A | |
| | | | cell-mediated | | |
| | | | cytotoxicity | | |
| CD162 | monocytes, | 240 | adhesion | P-selectin | |
| | granulocytes, T cells, | | molecule; leucocyte | glycoprotein | |
| | subset of B cells | | rolling; | ligand-1 | |
| | | | ligand P-selectin | (PSGL-1) | |
| CD162R | NK cells | | PEN5 | | |
| CD163 | | | GHI/61, M130 | | |
| CD164 | myeloid cells, T cells, | 80 | adhesion | MGC-24 | |
| | epithelial cells, bone | | molecule haem | | |
| | marrow stroma cells | | progenitor cells | | |
| | | | to stroma | | |
| CD165 | T cells, NK cells, | 37 | adhesion | AD2, gp37, | |
| | platelets, thymocytes, | | molecule | A108 | |
| | thymic epithelium | | thymocytes/thymic | | |
| | | | epithelium | | |
| CD166 | T cells, NK cells, | 100 | adhesion | ALCAM | |
| | platelets, thymocytes, | | molecule; ligand | | |
| | activated B and T cells, | | for CD6 | | |
| | eosinophils, fibroblasts, | | | | |
| | endothelial cells, | | | | |
| | keratinocytes | | | | |
| CD167a | | | | Discoidin | |
| | | | | domain R | |
| | | | | DDR1 | |
| CD168 | | | | RHAMM | |
| CD169 | | | | sialoadhesion | |
| CD170 | | | | Siglec-5 | |
| CD171 | | | | L1 | |
| CD172a | | | | SIRP Alpha | |
| CD173 | | | | Blood group | |
| | | | | H type 2 | |
| CD174 | | | | Lewis y | |
| CD175 | | | | Tn | |
| CD175s | | | | Sialyl-Tn | |
| CD176 | | | | TF | |
| CD177 | | | | NB1 | |
| CD178 | | | | Fas ligand | |
| CD179a | | | | Vpre-B | |
| CD179b | | | | Lambda 5 | |
| CD180 | | | | RP105/Bgp95 | |
| CD183 | | | | CXCR3 | |
| CD184 | | | | CXCR4 | |
| CD195 | | | | CCR5 | |
| CDw197 | | | | CCR7 | |
| CD200 | | | | OX2 | |
| CD201 | | | | EPC R | |
| CD202b | | | | Tie2/Tek | |
| CD203c | | | | NPP3/PDNP3 | |
| CD204 | | | | macrophage | |
| | | | | scavenger | |
| | | | | receptor | |
| CD205 | | | | DEC205 | |
| CD206 | | | | macrophage | |
| | | | | mannose | |
| | | | | receptor | |
| CD207 | | | | Langerin | |
| CD208 | | | | DC-Lamp | |
| CD209 | | | | DC-SIGN | |
| CD210 | | | | IL-10R | |
| CD212 | | | | IL-12R | |
| CD213a1 | | | | IL- | |
| | | | | 13Ralpha1 | |
| CD213a2 | | | | IL-13R | |
| | | | | alpha 2 | |
| CD217 | | | | IL-17R | |
| CD220 | | | insulin data | insulin R | |
| | | | pack | | |
| CD221 | | | insulin data | IGF1R | |
| | | | pack | | |
| CD222 | | | | mannose-6- | |
| | | | | phosphate/ | |
| | | | | IGF2 | |
| | | | | receptor | |
| CD223 | | | | LAG-3 | |
| CD224 | | | | gamma- | |
| | | | | glutmyl | |
| | | | | transferase | |
| CD225 | | | | Leu13 | |
| CD226 | | | | DNAM-1 | |
| CD227 | | | | Muc.1 | |
| CD228 | | | | Melanotransferrin | |
| CD229 | | | | Ly9 | |
| CD230 | | | | prion protein | |
| CD231 | | | | TALLA- | |
| | | | | 1/A15 | |
| CD232 | | | | VESP R | |
| CD233 | | | | Band 3 | |
| CD234 | | | | DARC | |
| CD235a | | | | Glycophorin A | |
| CD235b | | | | Glycophorin B | |
| CD236 | | | | Glycophorin | |
| | | | | C/D | |
| CD236R | | | | Glycophorin C | |
| CD238 | | | | Kell | |
| CD239 | | | | B-CAM | |
| CD240CE | | | | Rh30CE | |
| CD240D | | | | Rh30D | |
| CD241 | | | | RhAg | |
| CD242 | | | | ICAM-4 | |
| CD243 | | | | MDR-1 | |
| CD244 | | | | 2B4 | |
| CD245 | | | | p220/240 | |
| CD246 | | | | Anaplatic | |
| | | | | lymphoma | |
| | | | | kinase | |
| CD247 | | | | zeta chain | |

### Nuclear Receptors

Nuclear receptors are a superfamily of regulatory proteins that are structurally and functionally related and are receptors for, e.g., steroids, retinoids, vitamin D and thyroid hormones (see, e.g., Evans (1988) Science 240:889-895). These proteins bind to cis-acting elements in the promoters of their target genes and modulate gene expression in response to ligands for the receptors.

Nuclear receptors can be classified based on their DNA binding properties (see, e.g., Evans, supra and Glass (1994) Endocr. Rev. 15:391-407). For example, one class of nuclear receptors includes the glucocorticoid, estrogen, androgen, progestin and mineralocorticoid receptors which bind as homodimers to hormone response elements (HREs) organized as inverted repeats (see, e.g., Glass, supra). A second class of receptors, including those activated by retinoic acid, thyroid hormone, vitamin D.sub.3, fatty acidsperoxisome proliferators (i.e., peroxisome proliferator activated receptor (PPAR)) and ecdysone, bind to HREs as heterodimers with a common partner, the retinoid X receptors (i.e., RXRs, also known as the 9-cis retinoic acid receptors; see, e.g., Levin et al. (1992) Nature 355:359-361 and Heyman et al. (1992) Cell 68:397-406).

RXRs are unique among the nuclear receptors in that they bind DNA as a homodimer and are required as a heterodimeric partner for a number of additional nuclear receptors to bind DNA (see, e.g., Mangelsdorf et al. (1995) Cell 83:841-850). The latter receptors, termed the class II nuclear receptor subfamily, include many which are established or implicated as important regulators of gene expression. There are three RXR genes (see, e.g., Mangelsdorf et al, (1992) Genes Dev. 6:329-344), coding for RXRa, -.beta., and -.gamma., all of which are able to heterodimerize with any of the class II receptors, although there appear to be preferences for distinct RXR subtypes by partner receptors in vivo (see, e.g., Chiba et al. (1997) Mol, Cell. Biol, 17:3013-3020). In the adult liver, RXRa is the most abundant of the three RXRs (see, e.g., Mangelsdorf et al. (1992) Genes Dev. 6:329-344), suggesting that it might have a prominent role in hepatic functions that involve regulation by class II nuclear receptors. See also, Wan et al, (2000) Mol, Cell. Biol 20:4436-4444.

### Orphan Nuclear Receptors

Included in the nuclear receptor superfamily of regulatory proteins are nuclear receptors for whom the ligand is known and those which lack known ligands. Nuclear receptors falling in the latter category are referred to as orphan nuclear receptors. The search for activators for orphan receptors has led to the discovery of previously unknown signaling pathways (see, e.g., Levin et al., (1992), supra and Heyman et al., (1992), supra). For example, it has been reported that bile acids, which are involved in physiological processes such as cholesterol catabolism, are ligands for the farnesoid X receptor (infra).

Since it is known that products of intermediary metabolism act as transcriptional regulators in bacteria and yeast, such molecules may serve similar functions in higher organisms (see, e.g., Tomkins (1975) Science 189:760-763 and O'Malley (1989) Endocrinology 125:1119-1120). For example, one biosynthetic pathway in higher eukaryotes is the mevalonate pathway, which leads to the synthesis of cholesterol, bile acids, porphyrin, dolichol, ubiquinone, carotenoids, retinoids, vitamin D, steroid hormones and farnesylated proteins,

### Farnesoid X Receptor

The farnesoid X receptor (originally isolated as RIP14 (retinoid X receptor-interacting protein-14), see, e.g., Seol et al. (1995) Mol. Endocrinol. 9:72-85) is a member of the nuclear hormone receptor superfamily and is primarily expressed in the liver, kidney and intestine (see, e.g., Seol et al., supra and Forman et al. (1995) Cell 81:687-693). It functions as a heterodimer with the retinoid X receptor (RXR) and binds to response elements in the promoters of target genes to regulate gene transcription. The farnesoid X receptor-RXR heterodimer binds with highest affinity to an inverted repeat-1 (IR-1) response element, in which consensus receptor-binding hexamers are separated by one nucleotide. The farnesoid X receptor is part of an interrelated process, in that the receptor is activated by bile acids (the end product of cholesterol metabolism) (see, e.g., Makishima et al. (1999) Science 284:1362-1365, Parks et al. (1999) Science 284:1365-1368, Wang et al. (1999) Mol. Cell. 3:543-553), which serve to inhibit cholesterol catabolism. See also, Urizar et al. (2000) J. Biol. Chem. 275:39313-39317.

### Nuclear Receptors and Disease

Nuclear receptor activity, including the farnesoid X receptor and/or orphan nuclear receptor activity, has been implicated in a variety of diseases and disorders, including, but not limited to, hyperlipidemia and hypercholesterolemia, and complications thereof, including without limitation coronary artery disease, angina pectoris, carotid artery disease, strokes, cerebral arteriosclerosis and xanthoma, (see, e.g., International Patent Application Publication No. WO 0057915), osteoporosis and vitamin deficiency (see, e.g., U.S. Pat. No. 6,316,5103), hyperlipoproteinemia (see, e.g., International Patent Application Publication No. WO 0160818), hypertriglyceridemia, lipodystrophy, peripheral occlusive disease, ischemic stroke, hyperglycemia and diabetes mellitus (see, e.g., International Patent Application Publication No. WO 0182917), disorders related to insulin resistance including the cluster of disease states, conditions or disorders that make up “Syndrome X” such as glucose intolerance, an increase in plasma triglyceride and a decrease in high-density lipoprotein cholesterol concentrations, hypertension, hyperuricemia, smaller denser low-density lipoprotein particles, and higher circulating levels of plasminogen activator inhibitor-1, atherosclerosis and gallstones (see, e.g., International Patent Application Publication No. WO 00/37077), disorders of the skin and mucous membranes (see, e.g., U.S. Pat. Nos. 6,184,215 and 6,187,814, and International Patent Application Publication No. WO 9832444), obesity, acne (see, e.g., International Patent Application Publication No. WO 0049992), and cancer, cholestasis, Parkinson's disease and Alzheimer's disease (see, e.g., International Patent Application Publication No. WO 00/7334).

The activity of nuclear receptors, including the farnesoid X receptor and/or orphan nuclear receptors, has been implicated in physiological processes including, but not limited to, triglyceride metabolism, catabolism, transport or absorption, bile acid metabolism, catabolism, transport, absorption, re-absorption or bile pool composition, cholesterol metabolism, catabolism, transport, absorption, or re-absorption. The modulation of cholesterol 7,alpha,-hydroxylase gene (CYP7A1) transcription (see, e.g., Chiang et al. (2000) J. Biol. Chem. 275:10918-10924), HDL metabolism (see, e.g., Urizar et al. (2000) J. Chem. 275:39313-39317), hyperlipidemia, cholestasis, and increased cholesterol efflux and increased expression of ATP binding cassette transporter protein (ABC1) (see, e.g., International Patent Application Publication No. WO 0078972) are also modulated or otherwise affected by the farnesoid X receptor.

### Nuclear Receptor Ligands (NRLs)

Nuclear hormone receptors can be divided into four mechanistic classes: Type I, Type II, Type III, and Type IV. Ligand binding to Type I receptors (NR3 Group) results in the dissociation of heat shock proteins (HSP) from the receptor, homodimerization of the receptor, translocation from the cytoplasm into the cell nucleus, and binding to inverted repeat hormone response elements (HRE's) of DNA. The nuclear receptor/DNA complex then recruits other proteins which transcribe DNA downstream from the HRE into messenger RNA. Type II receptors (NR1 Group) are retained in the nucleus and bind as heterodimers, usually with Retinoid X Receptors (RXR), to DNA. Type II nuclear hormone receptors are often complexed with corepressor proteins. Ligand binding to the Type II receptor causes dissociation of the corepressor and recruitment of coactivator proteins. Additional proteins are recruited to the nuclear receptor/DNA complex, which transcribe DNA into messenger RNA. Type III nuclear hormone receptors (NR2 Group) are orphan receptors that bind to direct repeat HRE's of DNA as homodimers. Type IV nuclear hormone receptors bind to DNA either as monomers or dimers, Type IV receptors are unique because a single DNA binding domain of the receptor binds to a single half site HRE. The NHR ligand can be a ligand that acts at any one or more of the Type 1, Type II, Type III or Type IV nuclear hormone receptors (e.g. as an agonist or antagonist).

TABLE 1
Nuclear Receptors

| Group | Genes | Trivial Names | Accession |
|---|---|---|---|
| 1A | NR1A1 | TRa, c-erbA-1, THRA | M24748 |
| | NR1A2 | TRb, c-erbA-2, THRB | X04707 |
| 1B | NR1B1 | RARa | X06538 |
| | NR1B2 | RARb, HAP | Y00291 |
| | NR1B3 | RARg, RARD | M57707 |
| 1C | NR1C1 | PPARa | L02932 |
| | NR1C2 | PPARb, NUC1, PPARd, | L07592 |
| | | FAAR | |
| | NR1C3 | PPARg | L40904 |
| 1D | NR1D1 | REVERBa, EAR1, | M24898 |
| | | EAR1A | |
| | NR1D2 | REVERBb, EAR1b, | L31785 |
| | | BD73, RVR, HZF2 | |
| 1F | NR1F1 | RORa, RZRa | U04897 |
| | NR1F2 | RORb, RZRb | Y08639 |
| | NR1F3 | RORg, TOR | U16997 |
| 1H | NR1H2 | UR, OR-1, NER1, | U07132 |
| | | RIP15, LXRb | |
| | NR1H3 | RLD1, LXR, LXRa | U22662 |
| | NR1H4 | FXR, RIP14, HRR1 | U09416 |
| 1I | NR1I1 | VDR | J03258 |
| | NR1I2 | ONR1, PXR, SXR, | X75163 |
| | | BXR | |
| | NR1I3 | MB67, CAR1, CARa | Z30425 |
| | | CAR3 (splice variant) | |
| 2A | NR2A1 | HNF4 | X76930 |
| | NR2A2 | HNF4G | Z49826 |
| 2B | NR2B1 | RXRA | X52773 |
| | NR2B2 | RXRB, H-2RIIBP, | M84820 |
| | | RCoR-1 | |
| | NR2B3 | RXRG | X66225 |
| | NR2B4 | USP, Ultraspiracle, | X52591 |
| | | 2C1, CF1, RXR1, | |
| | | RXR2 | |
| 2C | NR2C1 | TR2, TR2-11 | M29960 |
| 2E | NR2E1 | TLL, TLX, XTLL | S72373 |
| | NR2E3 | PNR | AF121129 |
| 2F | NR2F1 | COUP-TFI, COUPTFA, | X12795 |
| | | EAR3, SVP44 | |
| | NR2F2 | COUP-TFII, | M64497 |
| | | COUPTFB, ARP1, | |
| | | SVP40 | |
| | NR2F6 | EAR2 | X12794 |
| 3A | NR3A1 | Era | X03635 |
| | NR3A2 | ERb | U57439 |
| 3B | NR3B1 | ERR1, ERRa | X51416 |
| | NR3B2 | ERR2, ERRb | X51417 |
| | NR3B3 | ERR3, ERRg | AF094318 |
| 3C | NR3C1 | GR | X03225 |
| | NR3C2 | MR | M16801 |
| | NR3C3 | PR | M15716 |
| | NR3C4 | AR | M20132 |
| 4A | NR4A1 | NGFIB, TR3, N10, | L13740 |
| | | NUR77, NAK1 | |
| | NR4A2 | NURR1, NOT, RNR1, | X75918 |
| | | HZF-3, TINOR | |
| | NR4A3 | NOR1, MINOR | D38530 |
| 5A | NR5A1 | SF1, ELP, FTZ-F1, | D88155 |
| | | AD4BP | |
| | NR5A2 | LRH1, xFF1rA, | U93553 |
| | | xFF1rB, FFLR, PHR, | |
| | | FTF | |
| 6A | NR6A1 | GCNF1, RTR | U14666 |
| 0B | NR0B1 | DAX1, AHCH | S74720 |
| | NR0B2 | SHP | L76571 |

In some embodiments, Y is an antagonist that acts by competing with or blocking binding of native or non-native ligand to the active site. In some embodiments, the NRL is an antiandrogenic compound. In certain embodiments, the antiandrogenic NRL is selected from the group consisting of antiandrogens; alpha-substituted steroids; carbonylamino-benzimidazole; 17-hydroxy 4-aza androstan-3-ones; antiandrogenic biphenyls; goserelin; nilutamid; decursin; flutamide; p,p′-DDE; vinclozolin; cyproterone acetate; linuron. In certain embodiments, the antiandrogenic NRL is selected from the group consisting of fluorinated 4-azasteroids; fluorinated 4-azasteroids derivatives; antiandrogens; alpha-substituted steroids; carbonylamino-benzimidazole; 17-hydroxy 4-aza androstan-3-ones; antiandrogenic biphenyls; goserelin; nilutamid; decursin; flutamide; p,p′-DDE; vinclozolin; cyproterone acetate; and linuron. In other embodiments, the NRL is an antagonist that acts by binding to the active site or an allosteric site and preventing activation of, or de-activating, the NR.

In some embodiments, Y exhibits an ECso for nuclear receptor activation (or in the case of an antagonist, an IC50) of about 10 mM or less, or 1 mM (1000 μM) or less (e.g., about 750 μM or less, about 500 μM or less, about 250 μM or less, about 100 μM or less, about 75 μM or less, about 50 μM or less, about 25 μM or less, about 10 μM or less, about 7.5 μM or less, about 6 μM or less, about 5 μM or less, about 4 μM or less, about 3 μM or less, about 2 μM or less or about 1 μM or less). In some embodiments, Y exhibits an EC50 or IC50 at a nuclear hormone receptor of about 1000 nM or less (e.g., about 750 nM or less, about 500 nM or less, about 250 nM or less, about 100 nM or less, about 75 nM or less, about 50 nM or less, about 25 nM or less, about 10 nM or less, about 7.5 nM or less, about 6 nM or less, about 5 nM or less, about 4 nM or less, about 3 nM or less, about 2 nM or less or about 1 nM or less). In some embodiments, Y has an EC₈₀ or IC₅₀ at a nuclear hormone receptor which is in the picomolar range. Accordingly, in some embodiments, Y exhibits an EC₈₀ or IC₅₀ at a nuclear hormone receptor of about 1000 pM or less (e.g., about 750 pM or less, about 500 pM or less, about 250 pM or less, about 100 pM or less, about 75 pM or less, about 50 pM or less, about 25 pM or less, about 10 pM or less, about 7.5 pM or less, about 6 pM or less, about 5 pM or less, about 4 pM or less, about 3 pM or less, about 2 pM or less or about 1 pM or less).

In some embodiments, Y exhibits an EC₈₀ or IC₅₀ at a nuclear hormone receptor that is about 0.001 pM or more, about 0.01 pM or more, or about 0.1 pM or more. Nuclear hormone receptor activation (nuclear hormone receptor activity) can be measured in vitro by any assay known in the art. For example, the activity at the nuclear hormone receptor can be measured by expressing the receptor in yeast cells also harboring a reporter gene (e.g., lacZ which encodes β-galactosidase) under the control of a hormone-responsive promoter. Thus, in the presence of a ligand that acts at the receptor, the reporter gene is expressed and the activity of the reporter gene product can be measured (e.g., by measuring the activity of β-galactosidase in breaking down a chromogenic substrate, such as chlorophenol red--D-galactopyranoside (CPRG), which is initially yellow, into a red product that can be measured by absorbance). See, e.g., Jungbauer and Beck, J. Chromatog. B, 77: 167-178 (2002); Routledge and Sumpter, J. Biol. Chem, 272: 3280-3288 (1997); Liu et al., J. Biol. Chem., 274: 26654-26660 (1999). Binding of the NHR ligand to the nuclear hormone receptor can be determined using any binding assay known in the art such as, for example, fluorescence polarization or a radioactive assay. See, e.g., Ranamoorthy et al., 138(4): 1520-1527 (1997).

In some embodiments, Y exhibits about 0.001% or more, about 0.01% or more, about 0.1% or more, about 0.5% or more, about 1% or more, about 5% or more, about 10% or more, about 20% or more, about 30% or more, about 40% or more, about 50% or more, about 60% or more, about 75% or more, about 100% or more, about 125% or more, about 150% or more, about 175% or more, about 200% or more, about 250% or more, about 300% or more, about 350% or more, about 400% or more, about 450% or more, or about 500% or higher activity at the nuclear hormone receptor relative to the native nuclear hormone (nuclear hormone potency). In some embodiments, Y exhibits about 5000% or less or about 10,000% or less activity at the nuclear hormone receptor relative to native nuclear hormone. The activity of Y at a receptor relative to a native ligand of the receptor is calculated as the inverse ratio of EC50S for Y versus the native ligand. In some embodiments, Y is the native ligand of the receptor.

The NRL of the invention (Y) is partly or wholly non-peptidic and is hydrophobic or lipophilic. In some embodiments, the NHR ligand has a molecular weight that is about 5000 daltons or less, or about 4000 daltons or less, or about 3000 daltons or less, or about 2000 daltons or less, or about 1750 daltons or less, or about 1500 daltons or less, or about 1250 daltons or less, or about 1000 daltons or less, or about 750 daltons or less, or about 500 daltons or less, or about 250 daltons or less. The structure of Y can be in accordance with any of the teachings disclosed herein.

In the embodiments described herein, Y is conjugated to L (e.g. when L is a linking group) or Ab (e.g. when L is a bond) at any position of Y that is capable of reacting with Ab or L. One skilled in the art could readily determine the position and means of conjugation in view of general knowledge and the disclosure provided herein.

In any of the embodiments described herein wherein Y comprises a tetracyclic skeleton having three 6-membered rings joined to one 5-membered ring or a variation thereof (e.g. a Y that acts at the vitamin D receptor), the carbon atoms of the skeleton are referred to by position number, as shown below:

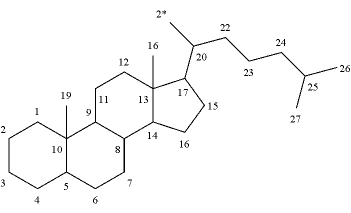 [SMILES: CC(C)CCCC(C)C1CCC2C3CCC4CCCCC4(C)C3CCC12C]

For example, a modification having a ketone at position-6 refers to the following structure:

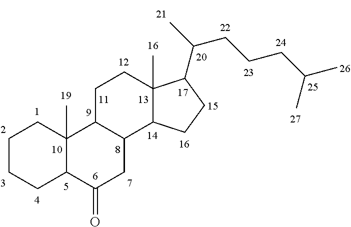 [SMILES: CC(C)CCCC(C)C1CCC2C3CC(=O)C4CCCCC4(C)C3CCC12C]

In some embodiments of the invention, the NRL (Y) acts on a Type I nuclear hormone receptor. In some embodiments, Y can have any structure that permits or promotes agonist activity upon binding of the ligand to a Type I nuclear hormone receptor, while in other embodiments Y is an antagonist of the Type I nuclear hormone receptor.

In some embodiments of the invention, the NHR ligand (Y) acts on a Type I nuclear hormone receptor. In some embodiments, Y can have any structure that permits or promotes agonist activity upon binding of the ligand to a Type I nuclear hormone receptor, while in other embodiments Y is an antagonist of the Type I nuclear hormone receptor.

In exemplary embodiments, Y comprises a structure as shown in Formula A:

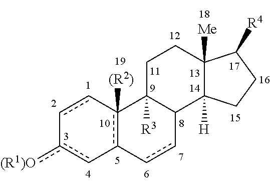

wherein R1 and R2, when present, are independently moieties that permit or promote agonist or antagonist activity upon binding of the compound of Formula A to the Type I nuclear hormone receptor; R³ and R⁴ are independently moieties that permit or promote agonist or antagonist activity upon binding of the compound of Formula A to the Type I nuclear hormone receptor; and each dashed line represents an optional double bond. Formula A may further comprise one or more substituents at one or more of positions 1, 2, 3, 4, 5, 6, 7, 8, 9, 11, 12, 14, 15, 16, 17, 18, and 19. Contemplated optional substituents include, but are not limited to, OH, NH₂, ketone, and C1-C18 alkyl groups.

In some embodiments, Y comprises a structure of Formula A wherein

R¹ is present and is hydrogen, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OCi_Ci₈ alkyl, (C₀-C₈ alkyl)C(O)OC₂-Ci₈ alkenyl, (C₀-C₈ alkyl)C(O)OC₂-Ci₈ alkynyl, (C₀-C₈ alkyl)C(O)OH, (C₀-C₈ alkyl)C(O)O aryl, (C₀-C₈ alkyl)C(O)0 heteroaryl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, or SO₃H;

R² is present and is hydrogen, (Co—C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl)NR²⁴C₁-C₈ alkyl, (C₀-C₈ alkyl)NR²⁴C₂-C₁₈ alkenyl, (C₀-C₈ alkyl)NR²⁴C₂-C₁₈ alkynyl, (Co—C₈ alkyl)NR²⁴H₂, (C₀-C₈ alkyl)C(O)C₁-C₁₈ alkyl, (C₀-C₈ alkyl) C(0)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(0)heteroaryl, (C₀-C₈ alkyl)C(0)OC₁-C₁₈ alkyl, (C₀-C₈ alkyl)C(O)OC₂-C₁₈ alkenyl, (C₀-C₈ alkyl)C(0)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₁₈ alkyl, (C₀-C₈ alkyl)OC(O)C₂-C₁₈ alkenyl, (C₀-C₈ alkyl)OC(O)C₂-Ci₈ alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl) NR²⁴C(O)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴(O)OC₁-C₁₈ alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(0)OH;

R³ is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl)NR²⁴C₁-C₁₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₁₈ alkyl, (C₀-C₈ alkyl)OC(O)C₂-C₁₈ alkenyl, (C₀-C₈ alkyl)OC(O)C₂-C₁₈ alkynyl, (C₀-C₈ alkyl)C(0)NR²⁴C₁-C₁₈ alkyl, (C₀-C₈ alkyl)C(0)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C_{R} alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(0)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR¹⁴(O)OC₁-C₁ alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(O)OH;

R⁴ is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl(NR²⁴C₁-C₁₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₁₈ alkyl, (C₀-C₈ alkyl)OC(O)C₂-C₁₈ alkenyl, (C₀-C₈ alkyl)OC(O)C₂-C₁₈ alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(O)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴(O)OC₁-C₁₈ alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(O)OH; and

R24 is hydrogen or C₁-C₁₈

In some embodiments, Y comprises a structure of Formula A

wherein R¹ is present and is hydrogen, C1-C7 alkyl; (C₀-C₃ alkyl)C(O)C₁-C₇ alkyl, (C₀-C₃ alkyl)C(O)aryl, or SO₃H;

R is present and is hydrogen, halo, OH, or Ci-C₇ alkyl;

R is hydrogen, halo, OH, or C₁-C₇ alkyl;

R⁴ is hydrogen, (C₀-C₈ alkyl)halo, C₁-C₈ alkyl, C₂-C₈ alkenyl, C₂₋₁₈ alkynyl, heteroalkyl, (C₀-C₈ alkyl)aryl, (C₀-C₈ alkyl)heteroaryl, (C₀-C₈ alkyl)OC₁-C₈ alkyl, (C₀-C₈ alkyl)OC₂-C₈ alkenyl, (C₀-C₈ alkyl)OC₂-C₈ alkynyl, (C₀-C₈ alkyl)OH, (C₀-C₈ alkyl)SH, (C₀-C₈ alkyl)NR²⁴C₁-C₈ alkyl, (C₀-C₈ alkyl)NR²⁴C₂-C₈ alkenyl, (C₀-C₈ alkyl)NR²⁴C₂-C₈ alkynyl, (C₀-C₈ alkyl)NR²⁴H₂, (C₀-C₈ alkyl)C(O)C₁-C₈ alkyl, (C₀-C₈ alkyl)C(O)C₂-C₈ alkenyl, (C₀-C₈ alkyl)C(O)C₂-C₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₈ alkyl, (C₀-C₈ alkyl)C(O)OC₂-C₈ alkenyl, (C₀-C₈ alkyl)C(O)OC₂-C₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₈ alkyl, (C₀-C₈ alkyl)OC(O)C₂-C₈ alkenyl, (C₀-C₈ alkyl)OC(O)C₂-C₁₈ alkynyl, (C₀-C₈ alkyl)C(O)NR²⁴C₁-C₈ alkyl, (C₀-C₈ alkyl)C(O)NR²⁴C₂-C₈ alkenyl, (C₀-C₈ alkyl)C(O)NR²⁴C₂-C₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₈ alkyl, (C₀-C₈ alkyl)NR²⁴C(O)C₂-C₈ alkenyl, or (C₀-C₈ alkyl)NR²⁴C(O)C₂-C₈ alkynyl, (C₀-C₈ alkyl)NR²⁴C(O)OH, (C₀-C₈ alkyl)OC(O)OC₁-C₈ alkyl, (C₀-C₈ alkyl)OC(O)OC₂-C₈ alkenyl, (C₀-C₈ alkyl)OC(O)OC₂-C₈ alkynyl, (C₀-C₈ alkyl)OC(O)OH, (C₀-C₈ alkyl)OC(O)NR²⁴C₁-C₈ alkyl, (C₀-C₈ alkyl)OC(O)NR²⁴C₂-C₈ alkenyl, (C₀-C₈ alkyl)OC(O)NR²⁴C₂-C₈ alkynyl, (C₀-C₈ alkyl)OC(O)NR²⁴H₂, (C₀-C₈ alkyl)NR²⁴(O)OC₁-C₈ alkyl, (C₀-C₈ alkyl)NR²⁴(O)OC₂-C₈ alkenyl, (C₀-C₈ alkyl)NR²⁴O)OC₂-C₈ alkynyl, or (C₀-C₈ alkyl)NR²⁴(O)OH; and,

R²⁴ is hydrogen or C1-C7 alkyl.

In some embodiments, R¹ is hydrogen, propionate, acetate, benzoate, or sulfate; R² is hydrogen or methyl; R³ is hydrogen or methyl; and R⁴ is acetate, cypionate, hemisucciniate, enanthate, or propionate.

In embodiments wherein Y comprises a structure of Formula A, Y is conjugated to L (e.g. when L is a linking group) or Ab (e.g. when L is a bond) at any position of Formula A that is capable of reacting with Ab or L. One skilled in the art could readily determine the position of conjugation on Formula A and means of conjugation of Formula A to Ab or L in view of general knowledge and the disclosure provided herein. In some embodiments, Formula A is conjugated to L or Ab at any of positions 1, 2, 3, 4, 5, 6, 7, 8, 9, 10, 11, 12, 13, 14, 15, 16, 17, 18, 19, or 20 of Formula A. In some embodiments, Formula A is conjugated to L or Ab at position 1, 3, 6, 7, 12, 10, 13, 16, 17, or 19 of Formula A.

In some embodiments, Y acts at an estrogen receptor (e.g. ERa, ERJ3). In some embodiments, Y permits or promotes agonist activity at the estrogen receptor, while in other embodiments Y is an antagonist of ER. In exemplary embodiments, Y can have a structure of Formula B:

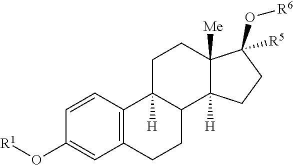

wherein R¹, R⁵ and R⁶ are moieties that permit or promote agonist or antagonist activity upon binding of the compound of Formula B to the estrogen receptor. In some embodiments, Formula B further comprises one or more substitutents at one or more of positions 1, 2, 4, 6, 7, 8, 9, 11, 12, 14, 15, and 16 (e.g. a ketone at position-6).

In some embodiments when Y comprises a structure of Formula B, wherein

R¹ is hydrogen, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, C₀-C₈ alkyl)C(O)O aryl, (C₀-C₈ alkyl)C(O)O heteroaryl, (C₀-C₈ alkyl)C(O)NR²⁴C₁-C₁₈ alkyl, (C₀-C₈ alkyl)C(O)NR²⁴C₂-Ci₈ alkenyl, (C₀-C₈ alkyl)C(O)NR²⁴C₂-Ci₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, or SO₃H;

R⁵ is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl)NR²⁴C₁-C₁₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OCi_Ci₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₁₈ alkyl, (C₀-C₈ alkyl)OC(O)C₂-C₁₈ alkenyl, (C₀-C₈ alkyl)OC(O)C₂-C₁₈ alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(O)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴(O)OC₁-C₁₈ alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(O)OH; (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)OC₁-C₁₈ alkyl, (C₀-C₈ alkyl)C(O)OC₂-Cig alkenyl, (C₀-C₈ alkyl)C(O)OC₂-C₁₈ alkynyl, (C₀-C₈ alkyl)C(O)OH, (C₀-C₈ alkyl)OC(O)C₁-C₁₈ alkyl, (C₀-C₈ alkyl)OC(O)C₂-C₁₈ alkenyl, (C₀-C₈ alkyl)OC(O)C₂-C₁ g alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(O)C₂-C₁₈ alkynyl, or (C₀-C₈ alkyl)NR²⁴C(O)OH;

R⁶ is hydrogen, C₁-C₁₈ alkyl, C₂-C₁₈ alkenyl, C₂-C₁₈ alkynyl, heteroalkyl, (Co—C₈ alkyl)aryl, (C₀-C₈ alkyl)heteroaryl, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, C₀-C₈ alkyl)C(O)O aryl, (C₀-C₈ alkyl)C(O)O heteroaryl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, or SO₃H; and,

R24 is hydrogen or C₁-C₁₈ alkyl.

In some embodiments, Y comprises a structure of Formula B, wherein

R¹ is hydrogen, C₁-C₇ alkyl; (C₀-C₃ alkyl)C(O)C₁-C₇ alkyl, (C₀-C₃ alkyl)C(O)aryl, or SO₃H;

R⁵ is hydrogen, (C₀-C₈ alkyl)halo, C₁-C₈ alkyl, C₂-C₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₈ alkyl, (C₀-C₈ alkyl)OC₂-C₈ alkenyl, (C₀-C₈ alkyl)OC₂-C₈ alkynyl, (C₀-C₈ alkyl)OH, (C₀-C₈ alkyl)SH, (C₀-C₈ alkyl)NR²⁴C₁-C₈ alkyl, (C₀-C₈ alkyl)NR²⁴C₂-C₈ alkenyl, (C₀-C₈ alkyl)NR²⁴C₂-C₈ alkynyl, (C₀-C₈ alkyl)NR²⁴H₂, (C₀-C₈ alkyl)C(O)C₁-C₈ alkyl, (C₀-C₈ alkyl)C(O)C₂-C₈ alkenyl, (C₀-C₈ alkyl)C(O)C₂-C₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₈ alkyl, (C₀-C₈ alkyl)C(O)OC₂-C₈ alkenyl, (C₀-C₈ alkyl)C(O)OC₂-C₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₈ alkyl, (C₀-C₈ alkyl)OC(O)C₂-C₈ alkenyl, (C₀-C₈ alkyl)OC(O)C₂-C₁₈ alkynyl, (C₀-C₈ alkyl)C(O)NR²⁴C₁-C₈ alkyl, (C₀-C₈ alkyl)C(O)NR²⁴C₂-C₈ alkenyl, (C₀-C₈ alkyl)C(O)NR²⁴C₂-C₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₈ alkyl, (C₀-C₈ alkyl)NR²⁴C(O)C₂-C₈ alkenyl, or (C₀-C₈ alkyl)NR²⁴C(O)C₂-C₈ alkynyl, (C₀-C₈ alkyl)NR²⁴C(O)OH, (C₀-C₈ alkyl)OC(O)OC₁-C₈ alkyl, (C₀-C₈ alkyl)OC(O)OC₂-C₈ alkenyl, (C₀-C₈ alkyl)OC(O)OC₂-C₈ alkynyl, (C₀-C₈ alkyl)OC(O)OH, (C₀-C₈ alkyl)OC(O)NR²⁴C₁-C₈ alkyl, (C₀-C₈ alkyl)OC(O)NR²⁴C₂-C₈ alkenyl, (C₀-C₈ alkyl)OC(O)NR²⁴C₂-C₈ alkynyl, (C₀-C₈ alkyl)OC(O)NR²⁴H₂, (C₀-C₈ alkyl)NR²⁴(O)OC₁-C₈ alkyl, (C₀-C₈ alkyl)NR²⁴(O)OC₂-C₈ alkenyl, (C₀-C₈ alkyl)NR²⁴(O)OC₂-C₈ alkynyl, or (C₀-C₈ alkyl)NR²⁴(O)OH;

R⁶ is hydrogen, C₁-C₈ alkyl, C₂-C₈ alkenyl, C₂-C₈ alkynyl, heteroalkyl, (C₀-C₈ alkyl)aryl, (C₀-C₈ alkyl)heteroaryl, (C₀-C₈ alkyl)C(O)C₁-C₈ alkyl, (C₀-C₈ alkyl)C(O)C₂-C₈ alkenyl, (C₀-C₈ alkyl)C(O)C₂-C₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₈ alkyl, (C₀-C₈ alkyl)C(O)OC₂-C₈ alkenyl, (C₀-C₈ alkyl)C(O)OC₂-C₈ alkynyl, (C₀-C₈ alkyl)C(O)OH, C₀-C₈ alkyl)C(O)O aryl, (C₀-C₈ alkyl)C(O)O heteroaryl, (C₀-C₈ alkyl)C(O)NR²⁴C₁-C₈ alkyl, (C₀-C₈ alkyl)C(O)NR²⁴C₂-C₈ alkenyl, (C₀-C₈ alkyl)C(O)NR²⁴C₂-C₈ alkynyl, (C₀-C₈ alkyl)C(O)NR²⁴H₂, (C₀-C₈ alkyl)C(O)NR²⁴aryl, or (C₀-C₈ alkyl)C(O)N²⁴-heteroaryl; and

R²⁴ is hydrogen or C₁-C₇ alkyl.

For example, R¹ is hydrogen, propionate, acetate, benzoate, or sulfate; R⁵ is hydrogen, ethynyl, hydroxyl; and R⁶ is acetate, cypionate, hemisucciniate, enanthate, or propionate.

Nonlimiting examples of the compound of Formula B include 17β-estradiol, modified forms of estradiol such as β-estradiol 17-acetate, β-estradiol 17-cypionate, β-estradiol 17-enanthate, β-estradiol 17-valerate, β-estradiol 3,17-diacetate, β-estradiol 3,17-dipropionate, β-estradiol 3-benzoate, β-estradiol 3-benzoate 17-n-butyrate, β-estradiol 3-glycidyl ether, β-estradiol 3-methyl ether, β-estradiol 6-one, β-estradiol 3-glycidyl, β-estradiol 6-one 6-(O-carboxymethyloxime), 16-epiestriol, 17-epiestriol, 2-methoxy estradiol, 4-methoxy estradiol, estradiol 7-phenylpropionate, and 17β-estradril 2-methyl ether, 17a-ethynylestradiol, megestrol acetate, estriol, and derivatives thereof. In some embodiments, carbon 17 has a ketone substitutent and R⁵ and R⁶ are absent (e.g. estrone). Some of the aforementioned compounds of Formula B are shown below:

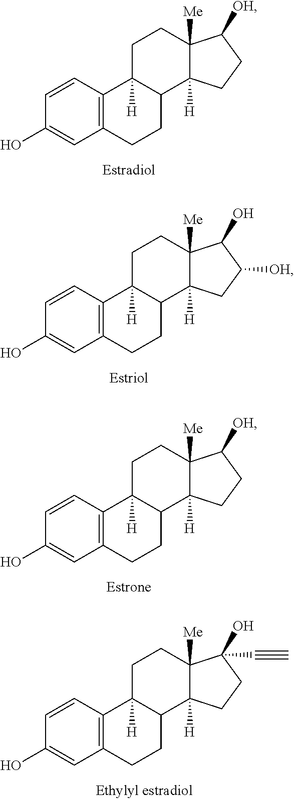

In embodiments wherein Y comprises a structure of Formula B, Y is conjugated to L (e.g. when L is a linking group) or Ab (e.g. when L is a bond) at any position of Formula B that is capable of reacting with Ab or L. One skilled in the art could readily determine the position of conjugation on Formula B and means of conjugation of Formula B to Ab or L in view of general knowledge and the disclosure provided herein. In some embodiments, Formula B is conjugated to L or Ab at any of positions 1, 2, 3, 4, 5, 6, 7, 8, 9, 10, 11, 12, 13, 14, 15, 16, 17, 18, 19, or 20 of Formula B. In some embodiments, Formula B is conjugated to L or Ab at position 3 or 17 of Formula B.

In other embodiments, Y acts at an estrogen receptor but is not encompassed by Formula B. Nonlimiting examples of ligands that act at an estrogen receptor that are not encompassed by Formula B are shown below:

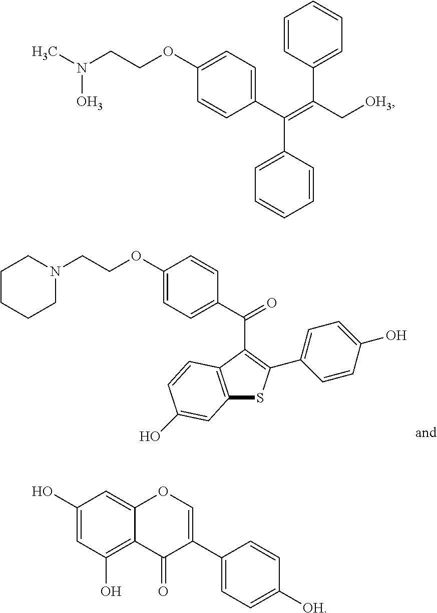

In some embodiments, Y acts at a glucocorticoid receptor (GR). In some embodiments, Y comprises any structure that permits or promotes agonist activity at the GR, while in other embodiments Y is an antagonist of GR. In exemplary embodiments, Y comprises a structure of Formula C:

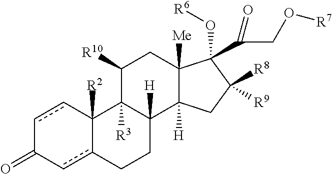

wherein R², R³, R⁶, R⁷, R⁸, R⁹, and R¹⁰ are each independently moieties that permit or promote agonist or antagonist activity upon the binding of the compound of Formula C to the GR; and each dash respresents an optional double bond. In some embodiments, Formula C further comprises one or more substituents at one or more of positions 1, 2, 4, 5, 6, 7, 8, 9, 11, 12, 14, and 15 (e.g. hydroxyl or ketone at position-11).

In some embodiments, Y comprises a structure of Formula C wherein

R² is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl)NR²⁴C₁-C₁₈ alkyl, (C₀-C₈ alkyl)NR²⁴C₂-C₁₈, alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₁₈ alkyl, (C₀-C₃ alkyl)OC(O)C₂-C₁₈ alkenyl, (C₀-C₈ alkyl)OC(O)C₂-C₁₈ alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(O)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴(O)OC₁-C₁₈, alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(O)OH;

R³ is hydrogen, (C₀-C₈ alkyl)halo, C₁-C₁₈ alkyl, C₂-C₁₃ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl)NR²⁴C₁-C₁₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₁₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₁₈ alkyl, (C₀-C₈ alkyl)OC(O)C₂-C₁₈ alkenyl, (C₀-C₈ alkyl)OC(O)C₂-C₁₈ alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(0)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴(O)OC₁-C₁₈ alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(O)OH;

R⁶ is hydrogen, C₁-C₁₈ alkyl, C₂-C₁₈ alkenyl, C₂-C₁₈ alkynyl, heteroalkyl, (Co—C₈ alkyl)aryl, (C₀-C₈ alkyl)heteroaryl, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, C₀-C₈ alkyl)C(O)O aryl, (C₀-C₈ alkyl)C(O)O heteroaryl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, or (C₀-C₈ alkyl)C(O)NR²⁴heteroaryl;

R⁷ is hydrogen, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, C₀-C₈ alkyl)C(O)O aryl, (C₀-C₈ alkyl)C(O)O heteroaryl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, or (C₀-C₈ alkyl)C(O)NR²⁴heteroaryl;

R⁸ is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl;

R⁹ is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl;

R¹⁰ is hydrogen, (C₀-C₈ alkyl)halo, C₁-C₁₈ alkyl, or (C₀-C₈ alkyl)OH; and

R²⁴ is hydrogen or C₁-C₁₈ alkyl.

In some embodiments, Y comprises a structure of Formula C, wherein

R² is hydrogen, halo, OH, or C₁-C₇ alkyl;

R³ is hydrogen, halo, OH, or C₁-C₇ alkyl;

R⁶ is hydrogen, C₁-C₈ alkyl, C₂-C₈ alkenyl, C₂-C₈ alkynyl, heteroalkyl, (C₀-C₈ alkyl)aryl, (C₀-C₈ alkyl)heteroaryl, (C₀-C₈ alkyl)C(O)C₁-C₈ alkyl, (C₀-C₈ alkyl)C(O)C₂-C₈ alkenyl, (C₀-C₈ alkyl)C(O)C₂-C₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₈ alkyl, (C₀-C₈ alkyl)C(O)OC₂-C₈ alkenyl, (C₀-C₈ alkyl)C(O)OC₂-C₈ alkynyl, (C₀-C₈ alkyl)C(O)OH, C₀-C₈ alkyl)C(O)O aryl, (C₀-C₈ alkyl)C(O)O heteroaryl, (C₀-C₈ alkyl)C(O)NR²⁴C₁-C₈ alkyl, (C₀-C₈ alkyl)C(O)NR²⁴C₂-C₈ alkenyl, (C₀-C₈ alkyl)C(O)NR²⁴C₂-C₈ alkynyl, (C₀-C₈ alkyl)C(O)NR²⁴H₂, (C₀-C₈ alkyl)C(O)NR²⁴aryl, or (C₀-C₈ alkyl)C(O)NR²⁴heteroaryl;

R⁷ is hydrogen, C₁-C₈ alkyl, C₂-C₈ alkenyl, C₂-C₈ alkynyl, heteroalkyl, (C₀-C₈ alkyl)aryl, (C₀-C₈ alkyl)heteroaryl, (C₀ alkyl)C(O)C₁-C₈ alkyl, (Co alkyl)C(O)C₂-C₈ alkenyl, (C₀ alkyl)C(O)C₂-C₈ alkynyl, (C₀)C(O)aryl, (C₀)C(O)heteroaryl, (C₀)C(O)OC₁-C₈ alkyl, (C₀ alkyl)C(O)OC₂-C₈ alkenyl, (C₀ alkyl)C(O)OC₂-C₈ alkynyl, or (C₀ alkyl)C(O)OH;

R⁸ is hydrogen or C₁-C₇ alkyl;

R⁹ is hydrogen or C₁-C₇ alkyl;

R¹⁰ hydrogen or OH; and

R²⁴ is hydrogen or C₁-C₇ alkyl.

For example, R² is hydrogen or methyl; R³ is hydrogen, fluoro, chloro, or methyl; R⁶ is hydrogen or C(O) C₁-C₇ alkyl; R⁷ is hydrogen, C(O)CH₃, or C(O)CH₂CH₃; R⁸ is hydrogen or methyl; R⁹ is hydrogen or methyl; and R¹⁰ is hydroxyl.

Nonlimiting examples of structures of Formula C include:

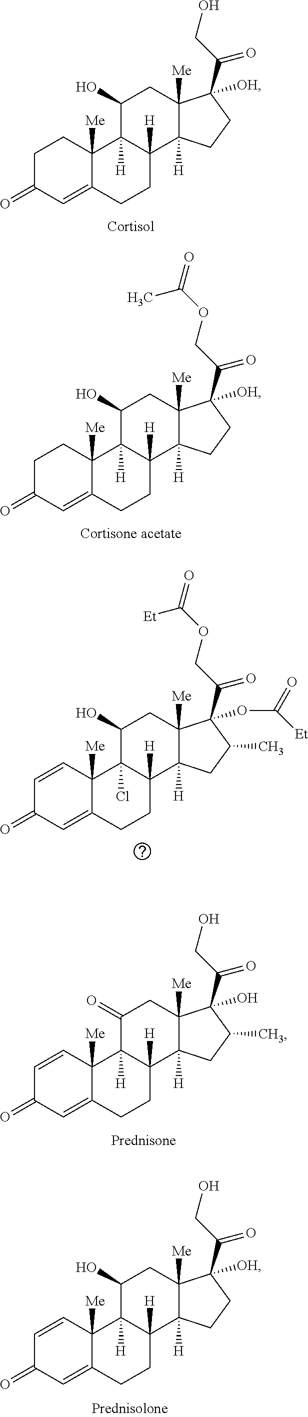

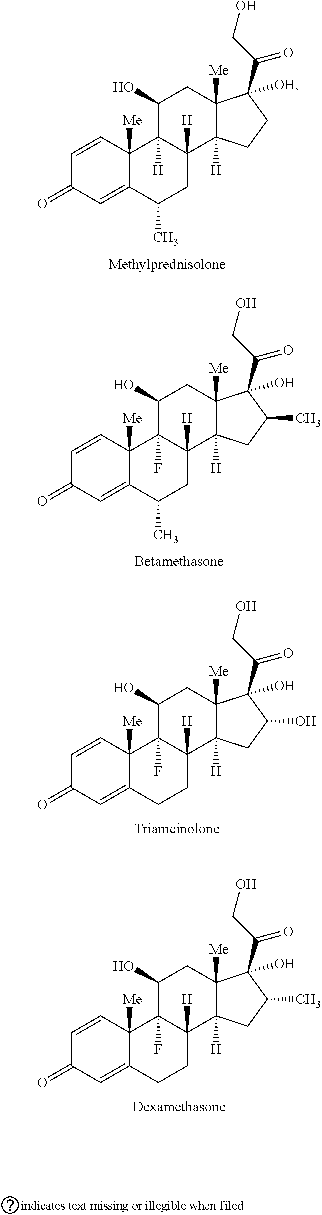

and derivatives thereof.

In embodiments wherein Y comprises a structure of Formula C, Y is conjugated to L (e.g. when L is a linking group) or Ab (e.g. when L is a bond) at any position of Formula C that is capable of reacting with Ab or L. One skilled in the art could readily determine the position of conjugation on Formula C and means of conjugation of Formula C to Ab or L in view of general knowledge and the disclosure provided herein. In some embodiments, Formula C is conjugated to L or Ab at any of positions 1, 2, 3, 4, 5, 6, 7, 8, 9, 10, 11, 12, 13, 14, 15, 16, 17, 18, 19, 20, 21, 22, or 23 of Formula C. In some embodiments, Formula C is conjugated to L or Ab at position 3, 10, 16 or 17 of Formula C.

In some embodiments, Y acts at a mineralcorticoid receptor (MR), In some embodiments, Y comprises any structure that permits or promotes agonist activity at the MR, while in other embodiments Y is an antagonist of MR. In exemplary embodiments, Y comprises a structure of Formula D:

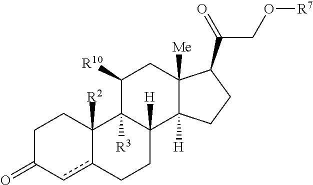

wherein R², R³, R⁷ and R¹⁰ are each independently a moiety that permits or promotes agonist or antagonist activity upon binding of the compound of Formula D to the MR; and the dashed line indicates an optional double bond. In some embodiments, Formula D further comprises one or more substituents at one or more of positions 1, 2, 4, 5, 6, 7, 8, 11, 12, 14, 15, 16, and 17.

In some embodiments, Y comprises a structure of Formula D wherein

R² is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl)NR²⁴C₁-C₁₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₁₈ (C₀-C₈ alkyl)OC(O)C₂-C₁₈ alkenyl, (C₀-C₈ alkyl)OC(O)C₂-C₁₈ alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(O)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁₈ alkenyl, (C₀-C₁₈ alkyl)OC(O)OC₂-C₁₈ alkynyl, (C₀-C₁₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴(O)OC₁-C₁₈ alkyl, (C₀-C₈ alkyl)NR²⁴(O)OC₂-C₁₈ alkenyl, (C₀-C₁₈ alkyl)NR²⁴(O)OC₂-C₁₈ alkynyl, or (C₀-C₈ alkyl)NR²⁴(O)OH;

R³ is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl)NR²⁴C₁-C₁₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₁₈ alkyl, (C₀-C₈ alkyl)OC(O)C₂-C₁₈ alkenyl, (C₀-C₈ alkyl)OC(O)C₂-C₁₈ alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(O)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴(O)OC₁-C₁₈ alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(O)OH;

R⁷ is hydrogen, C₁-C₁₈ alkyl, C₂-C₁₈ alkenyl, C₂-C₁₈ alkynyl, heteroalkyl, (Co—C₈ alkyl)aryl, (C₀-C₈ alkyl)heteroaryl, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, C₀-C₈ alkyl)C(O)O aryl, (C₀-C₈ alkyl)C(O)O heteroaryl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, or (C₀-C₈ alkyl)C(O)NR²⁴heteroaryl;

R¹⁰ is hydrogen, (C₀-C₈ alkyl)halo, C₁-C₁₈ alkyl, or (C₀-C₈ alkyl)OH; and

R²⁴ is hydrogen or C₁-C₁₈ alkyl.

In some embodiments, Y comprises a structure of Formula D, wherein

R is hydrogen, halo, OH, or C₁-C₇ alkyl;

R is hydrogen, halo, OH, or C₁-C₇ alkyl;

R is hydrogen, C₁-C₈ alkyl, C₂-C₈ alkenyl, C₂-C₈ alkynyl, heteroalkyl, (C₀-C₈ alkyl)aryl, (Co—C₈ alkyl)heteroaryl, (C₀ alkyl)C(O)C₁-C₈ alkyl, (C₀ alkyl)C(O)C₂-C₈ alkenyl, (C₀ alkyl)C(O)C₂-C₈ alkynyl, (C₀)C(O)aryl, (C₀)C(O)heteroaryl, (C₀)C(O)OC₁-C₈ alkyl, (Co alkyl)C(O)OC₂-C₈ alkenyl, (C₀ alkyl)C(O)OC₂.C₈ alkynyl, or (C₀ alkyl)C(O)OH;

R¹⁰ is hydrogen or OH; and

R²⁴ is hydrogen or C₁-C₇ alkyl.

For example, R is hydrogen or methyl; R is hydrogen, fluoro, chloro, or methyl; R is hydrogen, C(0)CH₃, or C(0)CH₂CH₃; and R¹⁰ is hydroxyl. Nonlimiting examples of compounds of Formula D include:

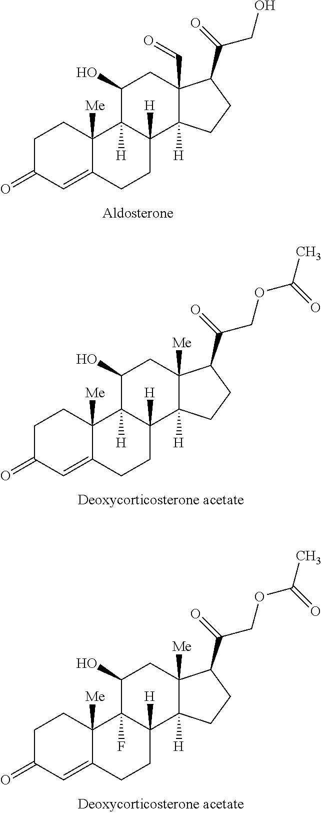

and derivatives thereof.

In embodiments wherein Y comprises a structure of Formula D, Y is conjugated to L (e.g. when L is a linking group) or Ab (e.g. when L is a bond) at any position of Formula D that is capable of reacting with Ab or L. One skilled in the art could readily determine the position of conjugation on Formula D and means of conjugation of Formula D to Ab or L in view of general knowledge and the disclosure provided herein. In some embodiments, Formula D is conjugated to L or Ab at any of positions 1, 2, 3, 4, 5, 6, 7, 8, 9, 10, 11, 12, 13, 14, 15, 16, 17, 18, 19, 20, 21, 22, 23, or 24 of Formula D. In some embodiments, Formula D is conjugated to L or Ab at position 3, 10, 13, or 17 of Formula D.

In some embodiments, Y acts at a progesterone receptor (PR). In some embodiments, Y comprises any structure that permits or promotes agonist activity at the PR, while in other embodiments Y is an antagonist of PR. In exemplary embodiments, Y comprises a structure of Formula E:

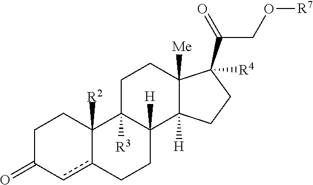

wherein R², R³, R⁴, and R⁷ are each independently moieties that permit or promote agonist or antagonist activity upon binding of the compound of Formula E to the PR; and the dashed line indicates an optional double bond. In some embodiments, Formula E further comprises one or more substituents at one or more of positions 1, 2, 4, 5, 6, 7, 8, 11, 12, 14, 15, 16, and 17 (e.g. a methyl group at position 6).

In some embodiments, Y comprises a structure of Formula E wherein

R² is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁ alkynyl, (C₀-C₈ alkyl)OH, (C₀-C₈ alkyl)SH, (C₀-C₈ alkyl)NR²⁴C₁-C₁₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₁₈ alkyl, (C₀-C₈ alkyl)OC(O)C₂-C₁₈ alkenyl, (C₀-C₈ alkyl)OC(O)C₂-C₁₈ alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(O)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴(O)OC₁-C₁₈ alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(O)OH; R²⁴ is hydrogen or C₁-C₁₈ alkyl,

R³ is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl)NR²⁴C₁-C₁₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₁₈ alkyl, (C₀-C₈ alkyl)OC(O)C₂-C₁₈ alkenyl, (C₀-C₈ alkyl)OC(O)C₂-C₁₈ alkynyl, (C₀-C₈ alkyl)C(O)NR²⁴C₂-C₁₈ alkyl, (C₀-C₈ alkyl)C(O)NR²⁴C₂-C₁₁ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(O)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴(O)OC₁-C₁₈ alkyl, (C₀-C₈ alkyl)NR²⁴ (O)OC₂-C₁₈ alkenyl, (C₀-C₈ alkyl)NR²⁴(O)OC₂-C₁₈ alkynyl, or (C₀-C₈ alkyl)NR²⁴(O)OH;

R⁴ is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl)NR²⁴C₁-C₁₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(0)H, (C₀-C₈ alkyl)C(O)aryl, (C₀-C₈ alkyl)C(O)heteroaryl, (C₀-C₈ alkyl)C(O)OCi_Ci₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)Ci_Ci₈ alkyl, (C₀-C₈ alkyl)OC(O)C₂-C₁₈ alkenyl, (C₀-C₈ alkyl)OC(O)C₂-C₁₈ alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(O)C₂-C₁₈ alkynyl, (C₀-C₈ alkyl)NR²⁴C(O)OH, (C₀-C₈ alkyl)OC(O)NR²⁴C₁-C₁₈ alkyl, (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴ (O)OC₁-C₁₈ alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(O)OH;

R⁷ is hydrogen, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl) C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, C₀-C₈ alkyl)C(O)O aryl, (C₀-C₈ alkyl)C(O)O heteroaryl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, or (C₀-C₈ alkyl)C(O)NR²⁴heteroaryl; and

R²⁴ is hydrogen or C₁-C₁₈ alkyl.

In some embodiments, Y comprises a structure of Formula E, wherein

R² is hydrogen, halo, OH, or C₁-C₇ alkyl;

R³ is hydrogen, halo, OH, or C₁-C₇ alkyl;

R⁴ is hydrogen, (C₀-C₈ alkyl)halo, C₁-C₈ alkyl, C₂-C₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₈ alkyl, (C₀-C₈ alkyl)OC₂-C₈ alkenyl, (C₀-C₈ alkyl)OC₂-C₈ alkynyl, (C₀-C₈ alkyl)OH, (C₀-C₈ alkyl)SH, (C₀-C₈ alkyl)NR²⁴C₁-C₈ alkyl, (C₀-C₈ alkyl)NR²⁴C₂OC₈ alkenyl, (C₀-C₈ alkyl)NR²⁴C₂-C₈ alkynyl, (C₀-C₈ alkyl)NR²⁴H₂, (C₀-C₈ alkyl)C(O)C₁-C₈ alkyl, (C₀-C₈ alkyl)C(O)C₂OC₈ alkenyl, (C₀-C₈ alkyl)C(O)C₂-C₈ alkynyl, (C₀-C₈ alkyl)C(O)H, (C₀-C₈ alkyl)C(O)aryl, (C₀-C₈ alkyl) C(O)heteroaryl, (C₀-C₈ alkyl)C(O)OC₁-C₈ alkyl, (C₀-C₈ alkyl)C(O)OC₂-C₈ alkenyl, (C₀-C₈ alkyl)C(O)OC₂-C₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₈ alkyl, (C₀-C₈ alkyl)OC(O)C₂-C₈ alkenyl, (C₀-C₈ alkyl)OC(O)C₂-C₁₈ alkynyl, (C₀-C₈ alkyl)C(O)NR²⁴C₁-C₈ alkyl, (C₀-C₈ alkyl)C(O)NR²⁴C₂-C₈ alkenyl, (C₀-C₈ alkyl)C(O)NR²⁴C₂-C₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₈ alkyl, (C₀-C₈ alkyl)NR²⁴C(O)C₂-C₈ alkenyl, or (C₀-C₈ alkyl)NR²⁴C(O)C₂-C₈ alkynyl, (C₀-C₈ alkyl)NR²⁴C(O)OH, (C₀-C₈ alkyl)OC(O)OC₁-C₈ alkyl, (C₀-C₈ alkyl)OC(O)OC₂-C₈ alkenyl, (C₀-C₈ alkyl)OC(O)OC₂-C₈ alkynyl, (C₀-C₈ alkyl)OC(O)OH, (C₀-C₈ alkyl)OC(O)NR²⁴C₁-C₈ alkyl, (C₀-C₈ alkyl)OC(O)NR²⁴C₂-C₈ alkenyl, (C₀-C₈ alkyl)OC(O)NR²⁴C₂-C₈ alkynyl, (C₀-C₈ alkyl)OC(O)NR²⁴H₂, (C₀-C₈ alkyl)NR²⁴(O)OC₁-C₈ alkyl, (C₀-C₈ alkyl)NR²⁴(O)OC₂-C₈ alkenyl, (C₀-C₈ alkyl)NR²⁴(O)OC₂-C₈ alkynyl, or (C₀-C₈ alkyl)NR²⁴(O)OH;

R⁷ is hydrogen, C₁-C₈ alkyl, C₂-C₈ alkenyl, C₂-C₈ alkynyl, heteroalkyl, (C₀-C₈ alkyl)aryl, (C₀-C₈ alkyl)heteroaryl, (C₀ alkyl)C(O)C₁-C₈ alkyl, (C₀ alkyl)C(O)C₂-C₈ alkenyl, (Co alkyl)C(O)C₂-C₈ alkynyl, (C₀)C(O)aryl, (C₀)C(O)heteroaryl, (C₀)C(O)OC₁-C₈ alkyl, (C₀ alkyl)C(O)OC₂-C₈ alkenyl, (C₀ alkyl)C(O)OC₂-C₈ alkynyl, or (C₀ alkyl)C(O)OH; and

R²⁴ is hydrogen or C₁-C₇ alkyl,

For example, R² is hydrogen or methyl; R³ is hydrogen or methyl; R⁴ is (C₁ alkyl)C(O)C₁-C4 alkyl, acetate, cypionate, hemisucciniate, enanthate, or propionate; and R⁷ is hydrogen, C(O)CH₃, or C(O)CH₂CH₃,

Nonlimiting examples of compounds of Formula E include:

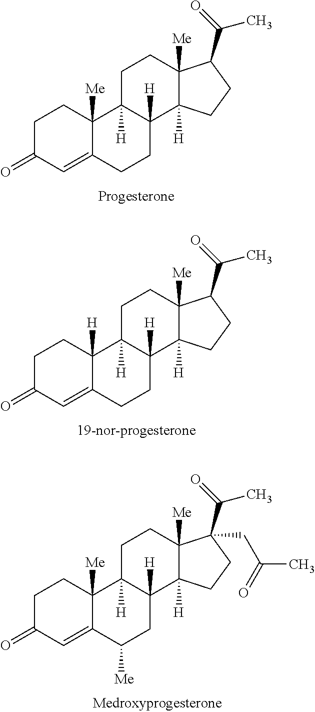

and derivatives thereof.

In embodiments wherein Y comprises a structure of Formula E, Y is conjugated to L (e.g. when L is a linking group) or Ab (e.g. when L is a bond) at any position of Formula E that is capable of reacting with Ab or L. One skilled in the art could readily determine the position of conjugation on Formula E and means of conjugation of Formula E to Ab or L in view of general knowledge and the disclosure provided herein. In some embodiments, Formula E is conjugated to L or Ab at any of positions 1, 2, 3, 4, 5, 6, 7, 8, 9, 10, 11, 12, 13, 14, 15, 16, 17, 18, 19, 20, 21, 22, 23, or 24 of Formula E. In some embodiments, Formula E is conjugated to L or Ab through position 3 or 17 of Formula E.

In other embodiments, Y acts at a progesterone receptor but is not is not encompassed by Formula E. For example, V can comprise the below structure and analogs thereof:

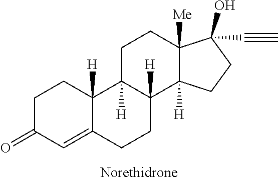

In some embodiments, Y acts at an androgen receptor (AR). In some embodiments, Y comprises any structure that permits or promotes agonist activity at the AR, while in other embodiments Y is an antagonist of AR. In exemplary embodiments, Y comprises a structure of Formula F:

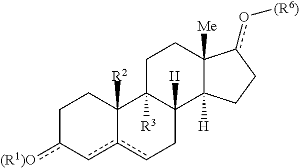

wherein R¹, when present, R², R³ and R⁶ are each independently a moiety that permits or promotes agonist or antagonist activity upon binding of the compound of Formula F to the AR; and each dashed line represents an optional double bond, with the proviso that no more than one of the optional carbon-carbon double bond is present at position 5. In some embodiments, Formula F further comprises one or more substituents at one or more of positions 1, 2, 4, 5, 6, 7, 8, 11, 12, 14, 15, 16, and 17.

In some embodiments, Y comprises a structure of Formula F wherein

R² is hydrogen, (C₀-C₈ alkyl)halo, C₁-C₁₈ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl)NR²⁴C₁-C₁₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-C₁₈ alkyl, (C₀-C₈ alkyl)OC(O)C₂-C₁₈ alkenyl, (C₀-C₈ alkyl)OC(O)C₂-C₁₈ alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, (C₀-C₈ alkyl)NR²⁴C(O)C₁-C₁₈ alkyl, (C₀-C₈ alkyl)NR²⁴C(O)C₂-C₁₈ alkenyl, or (C₀-C₈ alkyl)NR²⁴C(O)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴(O)OC₁-C₁₈ alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(O)OH;

R³ is hydrogen, (C₀-C₈ alkyl)halo, C₁-C₁₅ alkyl, C₂-C₁₈ alkenyl, C₂-C₁₈ alkynyl, heteroalkyl, (C₀-C₈ alkyl)aryl, (C₀-C₈ alkyl)heteroaryl, (C₀-C₈ alkyl)OC₁-C₁₈ alkyl, (C₀-C₈ alkyl)OC₂-C₁₈ alkenyl, (C₀-C₈ alkyl)OC₂-C₁₈ alkynyl, (C₀-C₈ alkyl)OH, (C₀-C₈ alkyl)SH, (C₀-C₈ alkyl)NR²⁴C₁-C₁₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)NR²⁴H₂, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(0)OH, (C₀-C₈ alkyl)C(O)O aryl, (C₀-C₈ alkyl)C(O)O heteroaryl, (C₀-C₈ alkyl)OC(O)C₁-C₁₈ alkyl, (C₀-C₈ alkyl)OC(O)C₂-C₁₈ alkenyl, (C₀-C₈ alkyl)OC(O)C₂-C₁₈ alkynyl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl) C(O)NR²⁴heteroaryl, (C₀-C₈ alkyl)NR²⁴C(O)C₁-C₁₈ alkyl, (C₀-C₈ alkyl)NR²⁴C(O)C₂-C₈ alkenyl, or (C₀-C₈ alkyl)NR²⁴C(0)C₂-C₁₈ alkynyl, (C₀-C₈ alkyl)NR²⁴C(O)OH, (C₀-C₈ alkyl)OC(O)OC₁-C₁₈ alkyl, (C₀-C₈ alkyl)OC(O)OC₂-C₁ₛ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₁-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴H₂, (C₀-C₈ alkyl)NR²⁴(O)OC₁-C₁₈ alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(O)OH;

R⁶ is hydrogen, C₁-C₁₈ alkyl, C₂-C₁₈ alkenyl, C₂-C₁₈ alkynyl, heteroalkyl, (Co—C₈ alkyl)aryl, (C₀-C₈ alkyl)heteroaryl, (C₀-C₈ alkyl)C(O)C₁-C₁₈ alkyl, (C₀-C₈ alkyl)C(O)C₂-C₁₈ alkenyl, (C₀-C₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, C₀-C₈ alkyl)C(O)O aryl, (C₀-C₈ alkyl)C(O)O heteroaryl, (C₀-C₈ alkyl)C(O)NR²⁴C₁-C₁₈ alkyl, (C₀-C₈ alkyl)C(O)NR²⁴C₂-C₁₈ alkenyl, (C₀-C₈ alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C(O)NR²⁴heteroaryl, or SO₃H; and

R²⁴ is hydrogen or C₁-C₁₈ alkyl.

In some embodiments, Y comprises a structure of Formula E,
wherein R¹ is hydrogen, C₁-C₇ alkyl; (C₀-C₃ alkyl)C(O)C₁-C₇ alkyl, (C₀-C₃ alkyl)C(O)aryl, or SO₃H;
R is hydrogen, halo, OH, or C₁-C₇ alkyl;
R is hydrogen, halo, OH, or C₁-C₇ alkyl;
R⁶ is hydrogen, C₁-C₈ alkyl, C₂-C₈ alkenyl, C₂-C₈ alkynyl, heteroalkyl, (C₀-C₈ alkyl)aryl, (C₀-C₈ alkyl)heteroaryl, (C₀-C₈ alkyl)C(O)C₁-C₈ alkyl, (C₀-C₈ alkyl)C(O)C₂-C₈ alkenyl, (C₀-C₈ alkyl)C(O)C₂-C₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₈ alkyl, (C₀-C₈ alkyl)C(O)OC₂-C₈ alkenyl, (C₀-C₈ alkyl)C(O)OC₂-C₈ alkynyl, (C₀-C₈ alkyl)C(O)OH, C₀-C₈ alkyl)C(O)O aryl, (C₀-C₈ alkyl)C(O)O heteroaryl, (C₀-C₈ alkyl)C(O)NR²⁴C₁-C₈ alkyl, (C₀-C₈ alkyl)C(O)NR²⁴C₂-C₈ alkenyl, (C₀-C₈ alkyl)C(O)NR²⁴C₂-C₈ alkynyl, (C₀-C₈ alkyl)C(O)NR²⁴H₂, (C₀-C₈ alkyl)C(O)NR²⁴aryl, or (C₀-C₈ alkyl)C(O)NR²⁴heteroaryl; and
R²⁴ is hydrogen or C₁-C₇ alkyl.

For example, R¹ is hydrogen or absent; R² is hydrogen or methyl; R³ is hydrogen or methyl; and R⁶ is H or absent.

Nonlimiting examples of compounds of Formula F include:

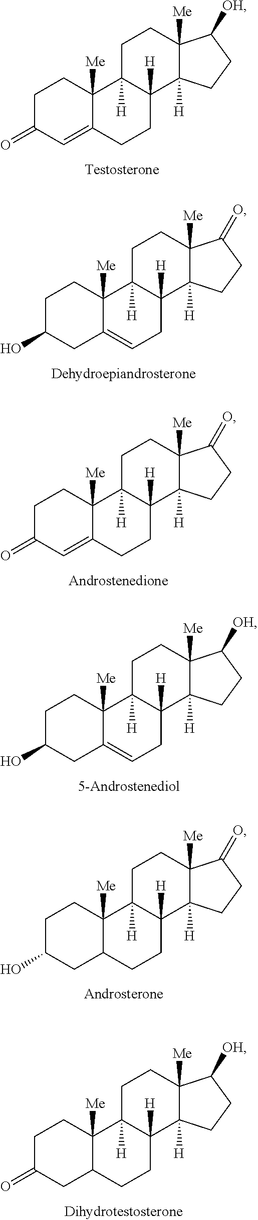

and derivatives thereof.

In embodiments wherein Y comprises a structure of Formula F, Y is conjugated to L (e.g. when L is a linking group) or Ab (e.g. when L is a bond) at any position of Formula F that is capable of reacting with Ab or L. One skilled in the art could readily determine the position of conjugation on Formula F and means of conjugation of Formula F to Ab or L in view of general knowledge and the disclosure provided herein. In some embodiments, Formula F is conjugated to L or Ab at any of positions 1, 2, 3, 4, 5, 6, 7, 8, 9, 10, 11, 12, 13, 14, 15, 16, 17, 18, 19, 20, 21, or 22 of Formula F. In some embodiments, Formula F is conjugated to L or Ab at position 3 or 17 of Formula F.

In some embodiments, the binding of the NRL to the Type I nuclear hormone receptor results in agonist activity (or antagonist activity) in some but not all cells or tissues expressing the Type I nuclear hormone receptor.

In some embodiments of the invention, the NRL (Y) acts on a Type II nuclear hormone receptor. In some embodiments, Y can have any structure that permits or promotes agonist activity upon binding of the ligand to a Type II nuclear hormone receptor, while in other embodiments Y is an antagonist of the Type II nuclear hormone receptor. In exemplary embodiments, Y exhibits agonist (or antagonist) activity at a thyroid hormone receptor (TR), retinoic acid receptor (RAR), peroxisome proliferator activated receptor (PPAR), Liver X Receptor (LXR), farnesoid X receptor (FXR), vitamin D receptor (VDR), and/or pregnane X receptor (PXR).

In some embodiments, Y acts at a thyroid hormone receptor (e.g. TRa, TR). In some embodiments, Y comprises any structure that permits or promotes agonist activity at the TR, while in other embodiments Y is an antagonist of TR. Nonlimiting examples of Y include the following compounds:

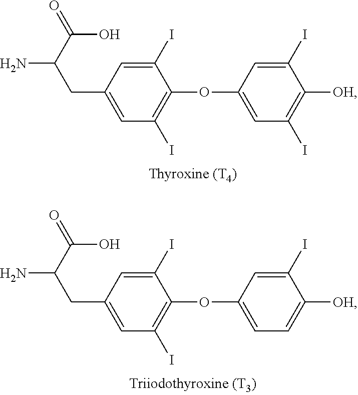 [SMILES: NC(Cc1cc(I)c(Oc2cc(I)c(O)c(I)c2)c(I)c1)C(=O)O ; NC(Cc1cc(I)c(Oc2ccc(O)c(I)c2)c(I)c1)C(=O)O]

and derivatives thereof.

In embodiments wherein Y comprises a structure that permits or promotes agonist or antagonist activity at a TR, Y is conjugated to L (e.g. when L is a linking group) or Ab (e.g. when L is a bond) at any position of Y that is capable of reacting with Ab or L. One skilled in the art could readily determine the position of conjugation on Y and means of conjugation of Y to Ab or L in view of general knowledge and the disclosure provided herein. In some embodiments, Y is conjugated to L or Ab through any position of Y. In some embodiments, I(is conjugated to L or Ab through the carboxylic acid or alcohol moieties, as indicated below:

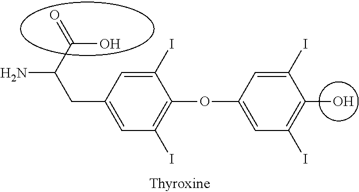 [SMILES: NC(Cc1cc(I)c(Oc2cc(I)c(O)c(I)c2)c(I)c1)C(=O)O]

In some embodiments, Y acts at a retinoic acid receptor (e.g. RARα, RARβ, RARγ). In some embodiments, Y comprises any structure that permits or promotes agonist activity at the RAR, while in other embodiments Y is an antagonist of RAR. In exemplary embodiments, Y comprises a structure of Formula G:

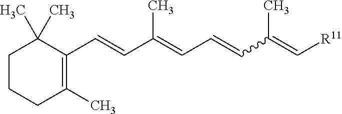

wherein R¹¹ is a moiety that permits or promotes agonist or antagonist activity upon the binding of the compound of Formula G to a RAR, and [symbol] represents either E or Z stereochemistry.

In some embodiments, Y comprises a structure of Formula G wherein R¹¹ is C(O)OH, CH₂OH, or C(O)H. In some embodiments, Y comprises a structure of Formula G wherein R¹¹ is a carboxylic acid derivative (e.g. acyl chloride, anhydride, and ester).

Nonlimiting examples of the compound of Formula G include:

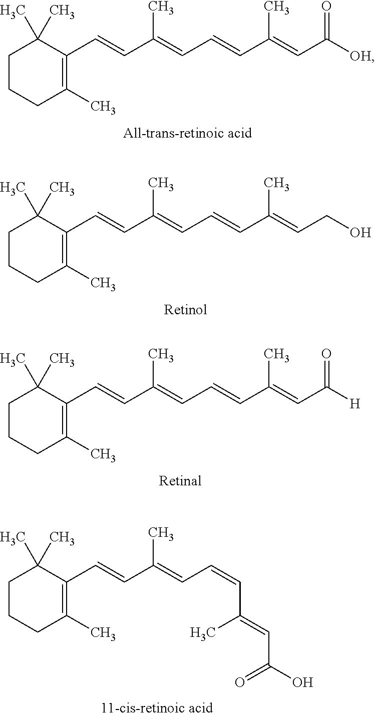 [SMILES: CC1=C(/C=C/C(C)=C/C=C/C(C)=C/C(=O)O)C(C)(C)CCC1 ; CC1=C(/C=C/C(C)=C/C=C/C(C)=C/CO)C(C)(C)CCC1 ; CC1=C(/C=C/C(C)=C/C=C/C(C)=C/C=O)C(C)(C)CCC1 ; CC1=C(/C=C/C(C)=C/C=C\C(C)=C\C(=O)O)C(C)(C)CCC1]

In embodiments wherein Y comprises a structure of Formula G, Y is conjugated to L (e.g. when L is a linking group) or Ab (e.g. when L is a bond) at any position of Formula G that is capable of reacting with Ab or L. One skilled in the art could readily determine the position of conjugation on Y and means of conjugation of Y to Ab or L in view of general knowledge and the disclosure provided herein. In some embodiments, Y is conjugated to L or Ab through any position of Y, In some embodiments, Formula G is conjugated to L or Ab at R¹¹.

In some embodiments, Y acts at a peroxisome proliferator activated receptor (e.g. PPARα, PPARβ/δ, PPARγ). In some embodiments, Y comprises any structure that permits or promotes agonist activity at the PPAR, while in other embodiments Y is an antagonist of PPAR. In some embodiments, Y is a saturated or unsaturated, halogenated or nonhalogenated free fatty acid (FFA) as described by Formula H:

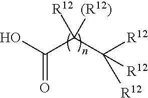

wherein n is 0-26 and each R¹², when present, is independently a moiety that permits or promotes agonist or antagonist activity upon binding of the compound of Formula H to a PPAR.

In some embodiments, Y comprises a structure of Formula H, wherein n is 0-26 and each R¹², when present, is independently hydrogen, C₁-C₇ alkyl, or halogen. In some embodiments Formula B is saturated such as, for example, formic acid, acetic acid, n-caproic acid, heptanoic acid, caprylic acid, nonanoic acid, capric acid, undecanoic acid, lauric acid, tridecanoic acid, myristic acid, pentadeconoic acid, palmitic acid, heptadecanoic acid, stearic acid, nonadecanoic acid, arachidic acid, heneicosanoic acid, behenic acid, tricosanoic acid, perfluorononanoic acid (see below), perfluorooctanoic acid (see below), and derivatives thereof.

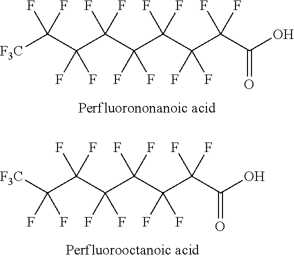 [SMILES: O=C(O)C(F)(F)C(F)(F)C(F)(F)C(F)(F)C(F)(F)C(F)(F)C(F)(F)C(F)(F)F ; O=C(O)C(F)(F)C(F)(F)C(F)(F)C(F)(F)C(F)(F)C(F)(F)C(F)(F)F]

In some embodiments Formula H is unsaturated with either cis or trans stereochemistry such as, for example, mead acid, myristoleic acid, palmitoleic acid, sapienic acid, oleic acid, linoleic acid, a-linolenic acid, elaidic acid, petroselinic acid, arachidonic acid, dihydroxyeicosatetraenoic acid (DiHETE), octadecynoic acid, eicosatriynoic acid, eicosadienoic acid, eicosatrienoic acid, eicosapentaenoic acid, erucic acid, dihomolinolenic acid, docosatrienoic acid, docosapentaenoic acid, docosahexaenoic acid, adrenic acid, and derivatives thereof.

In embodiments wherein Y comprises a structure of Formula H, Y is conjugated to L (e.g. when L is a linking group) or Ab (e.g. when L is a bond) at any position of Formula H that is capable of reacting with Ab or L, One skilled in the art could readily determine the position of conjugation on Formula H and means of conjugation of Formula H to Ab or L in view of general knowledge and the disclosure provided herein. In some embodiments, Formula H is conjugated to L or Ab at any position on Formula H. In some embodiments, Formula H is conjugated to L or Ab through the terminal carboxylic acid moiety.

In some of these embodiments, Y is an eiconsanoid. In specific embodiments, Y is a prostaglandin or a leukotriene. In some exemplary embodiments, Y is a prostaglandin having a structure as described by Formulae J1-J6:

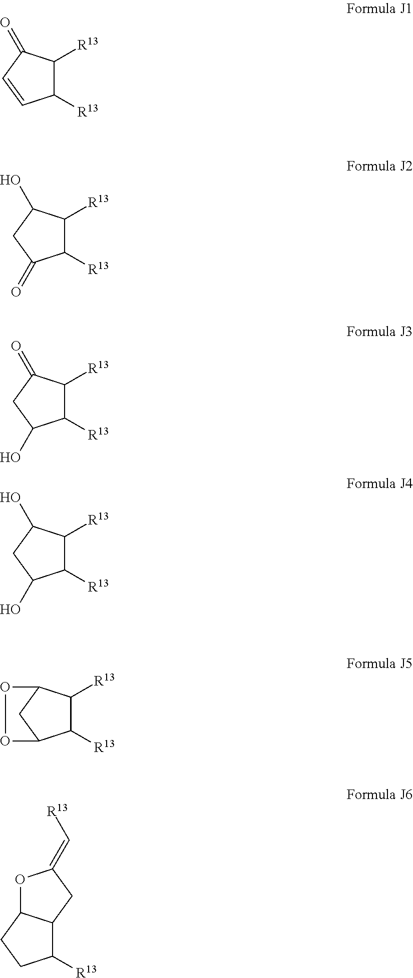

wherein each R13 is independently a moiety that permits or promotes agonist or antagonist activity upon the binding of the compound of Formula J to a PPAR (e.g. PGJ2 as shown below):

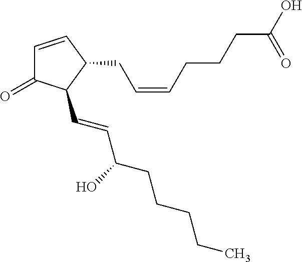 [SMILES: CCCCC[C@H](O)/C=C/[C@H]1C(=O)C=C[C@@H]1C/C=C\CCCC(=O)O]

In some embodiments when Y comprises a structure of any one of Formulae J1-J6, each R¹³ is independently C₇-C₈ alkyl, C₇-C₈ alkenyl, C₇-C₈ alkynyl, or heteroalkyl.

In embodiments wherein Y is an eicosanoid, Y is conjugated to L (e.g. when L is a linking group) or Ab (e.g. when Ab is a bond) at any position of the eicosanoid that is capable of reacting with Ab or L. One skilled in the art could readily determine the position of conjugation on Y and means of conjugation of Y to Ab or L in view of general knowledge and the disclosure provided herein. In some embodiments, Y is conjugated to L or Ab through any position of Y. In some embodiments, the eicosanoid is conjugated to L or Ab through a terminal carboxylic acid moiety or through a pendant alcohol moiety.

In some exemplary embodiments, Y is a leukotriene having a structure as described by Formula K or a derivatized form of Formula K:

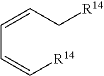

wherein each R is independently a moiety that permits or promotes agonist or antagonist activity upon the binding of the compound of Formula K to a PPAR (e.g. leukotriene B4 as shown below):

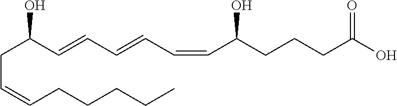 [SMILES: CCCCC/C=C\C[C@@H](O)/C=C/C=C/C=C\[C@@H](O)CCCC(=O)O]

In some embodiments when Y comprises a structure of Formula K, each R is independently C₃-C₁₃ alkyl, C₃-C₁₃ alkenyl, C3-C₁₃ alkynyl, or heteroalkyl.

In embodiments wherein Y comprises a structure of Formula K, Y is conjugated to L (e.g. when L is a linking group) or. Ab (e.g. when L is a bond) at any position of Formula K that is capable of reacting with Ab or L. One skilled in the art could readily determine the position of conjugation on Formula K and means of conjugation of Formula K to Ab or L in view of general knowledge and the disclosure provided herein. In some embodiments, Formula K is conjugated to L or Ab at any position on Formula K. In some embodiments,

Formula K is conjugated to L or Ab through the terminal carboxylic acid moiety or through a pendant alcohol moiety.

In some exemplary embodiments, Y is a thiazolidinedione comprising a structure as described by Formula L:

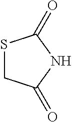

Nonlimiting examples of the compound of Formula L include:

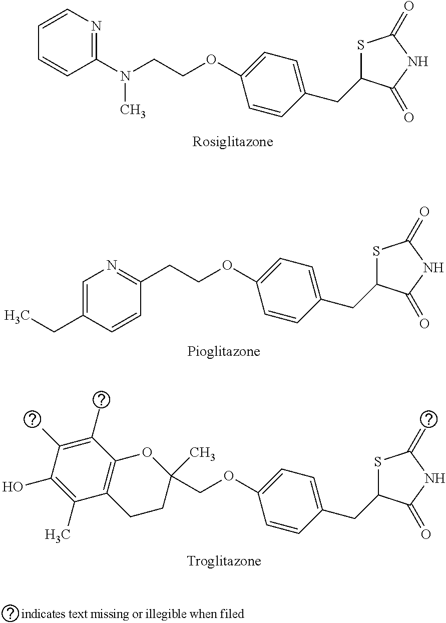 [SMILES: CN(CCOc1ccc(CC2SC(=O)NC2=O)cc1)c1ccccn1 ; CCc1ccc(CCOc2ccc(CC3SC(=O)NC3=O)cc2)nc1 ; C=C1NC(=O)C(Cc2ccc(OCC3(C)CCc4c(C)c(O)c(C)c(C)c4O3)cc2)S1]

and derivatives thereof

In embodiments wherein Y comprises a structure of Formula L, Y is conjugated to L (e.g. when L is a linking group) or Ab (e.g. when L is a bond) at any position of Formula L that is capable of reacting with Ab or L. One skilled in the art could readily determine the position of conjugation on Formula L and means of conjugation of Formula L to Ab or L in view of general knowledge and the disclosure provided herein. In some embodiments, Formula L is conjugated to L or Ab at any position on Formula L, such as, for example, a pendant alcohol moiety, or through an aromatic substituent.

In some embodiments, Y acts at a RAR-related orphan receptor (e.g. RORα, RORβ, RORγ). In some embodiments, Y comprises any structure that permits or promotes agonist activity at the ROR, while in other embodiments Y is an antagonist of ROR.

Nonlimiting examples of Y include:

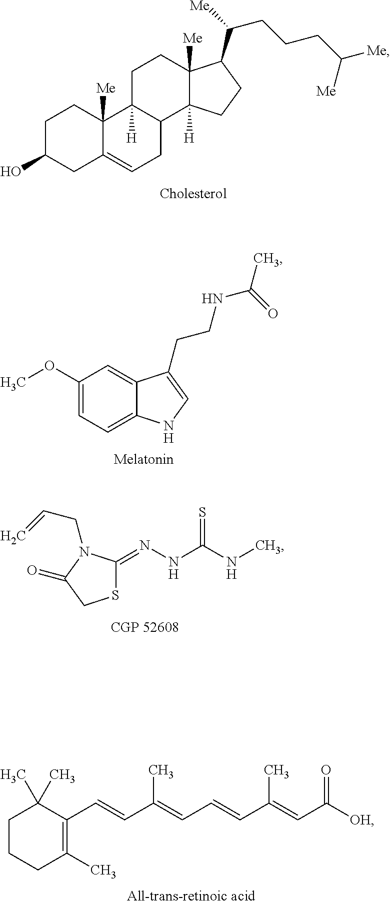

and derivatives thereof.

In embodiments wherein Y acts at a ROR, Y is conjugated to L (e.g, when L is a linking group) or Ab (e.g. when L is a bond) at any position of Y that is capable of reacting with Ab or L. One skilled in the art could readily determine the position of conjugation on Y and means of conjugation of Y to Ab or L in view of general knowledge and the disclosure provided herein. In some embodiments, Y is conjugated to L or Ab through any position of Y, such as, for example, any of the positions previously described herein.

In some embodiments, Y acts at a liver X receptor (LXRa, LXR). In some embodiments, Y comprises any structure that permits or promotes agonist activity at the LXR, while in other embodiments Y is an antagonist of LXR. In exemplary embodiments, Y is an oxysterol (i.e. oxygenated derivative of cholesterol). Nonlimiting examples of Y in these embodiments include 22(R)-hydroxycholesterol (see below), 24(S)-hydroxycholesterol (see below), 27-hydroxycholesterol, cholestenoic acid, and derivatives thereof.

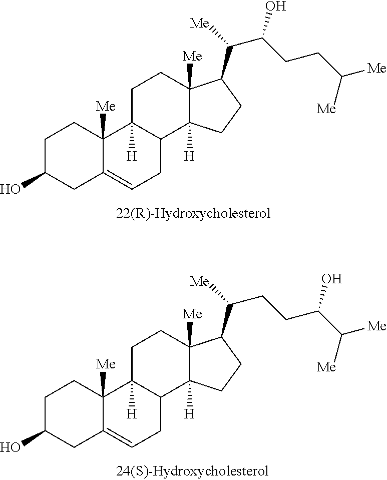

In embodiments wherein Y acts at a LXR, Y is conjugated to L (e.g. when L is a linking group) or Ab (e.g. when L is a bond) at any position of Y that is capable of reacting with Ab or L. One skilled in the art could readily determine the position of conjugation on Y and means of conjugation of Y to Ab or L in view of general knowledge and the disclosure provided herein. In some embodiments, Y is conjugated to L or Ab at any of positions 1, 2, 3, 4, 5, 6, 7, 8, 9, 10, 11, 12, 13, 14, 15, 16, 17, 18, 19, 20, 21, 22, 23, 24, 25, or 26 of Formula F. In some embodiments, Formula F is conjugated to L or Ab at position 3 or 17 of Formula F.

In some embodiments, Y acts at the farnesoid X receptor (FXR). In some embodiments, Y comprises any structure that permits or promotes agonist activity at the FXR, while in other embodiments Y is an antagonist of FXR. In some of these embodiments, Y is a bile acid. In exemplary embodiments, Y has a structure of Formula M:

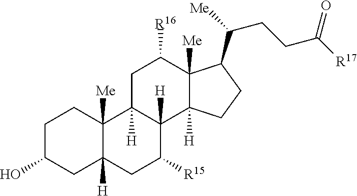

wherein each of R¹⁵, R¹⁶, and R¹⁷ are independently moieties that permit or promote agonist or antagonist activity upon binding of the compound of Formula M to a FXR.

In some embodiments when Y comprises a structure of Formula M, each of R¹⁵ and R¹⁶ are independently hydrogen, (C₀-C₈ alkyl)halo, C₁-C₁₈ alkyl, C₂-C₁₈ alkenyl, C₂-C₁₈ alkynyl, heteroalkyl, or (C₀-C₈ alkyl)OH; and R¹⁷ is OH, (C₀-C₈ alkyl)NH(C₁-C₄ alkyl)SO₃H, or (C₀-C₈ alkyl)NH(C₁-C₄ alkyl)COOH.

In some embodiments when Y comprises a structure of Formula M, each of R¹⁵ and R¹⁶ are independently hydrogen or OH; and R¹⁷ is OH, NH(C₁-C₂ alkyl)SO₃H, or NH(C₁-C₂ alkyl)COOH.

Nonlimiting examples of the compound of Formula M include:

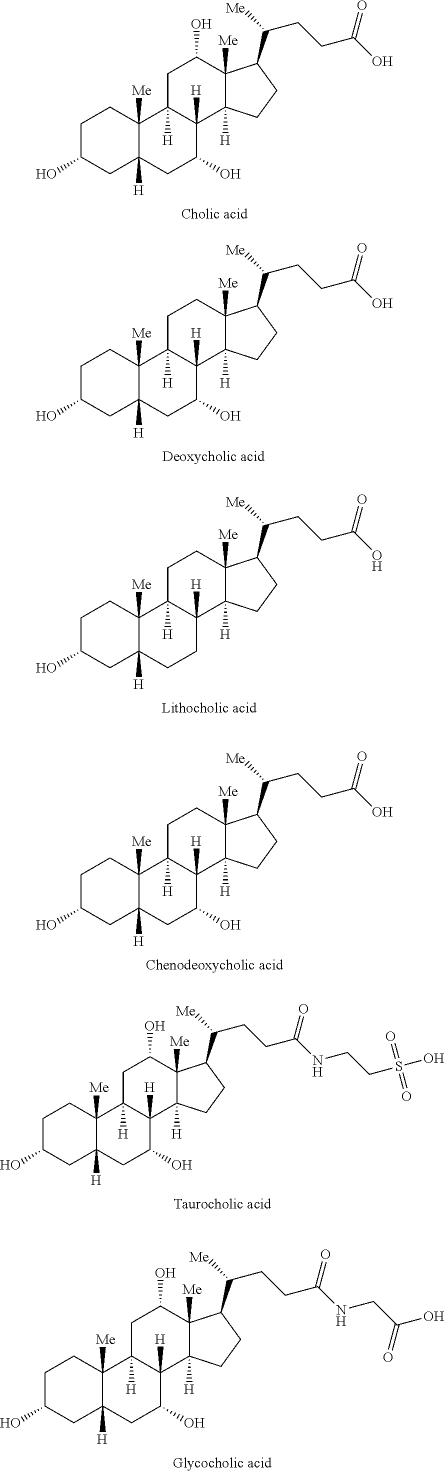

and derivatives thereof.

In embodiments wherein Y comprises a structure of Formula M, Y is conjugated to L (e.g. when L is a linking group) or Ab (e.g. when L is a bond) at any position of Formula M that is capable of reacting with Ab or L. One skilled in the art could readily determine the position of conjugation on Formula M and means of conjugation of Formula M to Ab or L in view of general knowledge and the disclosure provided herein. In some embodiments, Formula M is conjugated to L or Ab at any of positions 1, 2, 3, 4, 5, 6, 7, 8, 9, 10, 11, 12, 13, 14, 15, 16, 17, 18, 19, 20, 21, 22, 23, 24, or 25 of Formula M. In some embodiments, Formula M is conjugated to L or Ab at position 3, 7, 12 or 17 of Formula M.

In some embodiments, Y acts at the vitamin D receptor (VDR), In some embodiments, Y comprises any structure that permits or promotes agonist activity at the VDR, while in other embodiments Y is an antagonist of VDR. In exemplary embodiments, Y has a structure of Formula N:

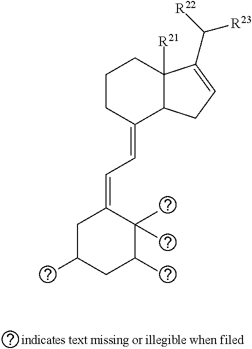

wherein each of R¹⁸, R¹⁹, R²⁰, R²¹, R²², and R²³ are moieties that permit or promote agonist or antagonist activity upon binding of the compound of Formula N to the VDR such as, for example, any of the vitamin D compounds found in Bouillon et al., Endocrine Reviews, 16(2):200-257 (1995).

In some embodiments wherein Y comprises a structure of Formula N,

R¹⁸ and R¹⁹ are each independently hydrogen, (C₀-C₈ alkyl)halo, (C₀-C₈ alkyl)heteroaryl, or (C₀-C₈ alkyl)OH;

both of R²⁰ are hydrogen or both of R²⁰ are taken together to form CH2;

each of R²¹ and R²² are independently C₁-C4 alkyl; and

R²³ is C₄-C₁₈ alkyl, C₄-C₁₈ alkenyl, C₄-C₁₈ alkynyl, heteroalkyl, (C₄-C₁₈ alkyl)aryl, (C₄-C₁₈ alkyl)heteroaryl, (C₀-C₈ alkyl)OC₁-C₁₈ alkyl, (C₀-C₈ alkenyl)OC₁-C₁₈ alkyl, (C₀-C₈ alkynyl)OC₁-C₁₈ alkyl, (C₀-C₈ alkyl)OC₂-C₁₈ alkenyl, (C₀-C₈ alkyl)OC₂-C₁₈ alkynyl, (C₆-C₁₈ alkyl)OH, (C₆-C₁₈ alkyl)SH, (C₆-C₁₈ alkenyl)OH, (C₆-C₁₈ alkynyl)OH, (C₀-C₈ alkyl)NR²⁴C₁-C₁₈ alkyl, (C₀-C₈ alkenyl)NR²⁴C₁-C₁₈ alkyl, (C₀-C₈ alkynyl)NR²⁴C₁-C₃₈ alkyl, (C₀-C₈ alkyl)NR²⁴C₂-C₁₈ alkenyl, (C₀-C₈ alkyl)NR²⁴C₂-C₁₈ alkynyl, (C₀-C₈ alkyl)C(O)C₁-C₁₈ alkyl, (C₀-C₈ alkyl)C(O)C₂-C₁₈ alkenyl, (C₀-C₁₈ alkyl)C(O)C₂-C₁₈ alkynyl, (C₀-C₈ alkyl)C(O)H, (C₀-C₈ alkyl)C(O)aryl, (C₀-C₈ alkyl)C(O)heteroaryl, (C₀-C₈ alkyl)C(O)OC₁-C₁₈ alkyl, (C₀-C₈ alkyl)C(O)OC₂-C₁₈ alkenyl, (C₀-C₈ alkyl)C(O)OC₂-C₁₈ alkynyl, (C₀-C₈ alkyl)C(O)OH, (C₀-C₈ alkyl)C(O)O aryl, (C₀-C₈ alkyl)C(O)O heteroaryl, (C₀-C₈ alkyl)OC(O)C₁-Ci₈ alkyl, (C₀-C₈ alkyl)OC(O)C₂-C₁₈ alkenyl, (C₀-C₈ alkyl)OC(O)C₂-C₁₈ alkynyl, (C₂-C₈ alkyl)C(O)NR²⁴C₁-C₁₈ alkyl, (C₀-C₈ alkyl)C(O)NR²⁴C₂-C₁₈ alkenyl, (C₀-C₈-alkyl)C(O)NR²⁴C₂-C₁₈ alkynyl, (C₀-C₈ alkyl)C(O)NR²⁴H₂, (C₀-C₈ alkyl)C(O)NR²⁴aryl, (C₀-C₈ alkyl)C-(O)NR²⁴heteroaryl, (C₀-C₈ alkyl)NR²⁴C(O)C₂-C₁₈ alkyl, (C₀-C₈ alkyl)NR²⁴C(O)C₂-C₈ alkenyl, or (C₀-C₈ alkyl)NR²⁴C(0)C₂-C₁₈ alkynyl, (C₀-C₈ alkyl)NR²⁴C(O)OH, (C₀-C₈ alkyl)OC(O)OC₁-C₁₈ (C₀-C₈ alkyl)OC(O)OC₂-C₁₈ alkenyl, (C₀-C₈ alkyl)OC(O)OC₂-C₁₈ alkynyl, (C₀-C₈ alkyl)OC(O)OH, (C₀-C₈ alkyl)OC(O)NR²⁴C₂-C₁₈ alkyl, (C₀-C₈ alkyl)OC(O)NR²⁴C₂-C₁₈ alkenyl, (C₀-C₈ alkyl)OC(O)NR²⁴C₂-C₁₈ alkynyl, (C₀-C₈ alkyl)OC(O)NR²⁴C₂, (C₀-C₈ alkyl)NR²⁴(O)OC₁-C₁₈ alkyl, (C₀-C₈ alkyl)NR²⁴(O)OC₂-C₁₈ alkenyl, (C₀-C₈ alkyl)NR²⁴(O)OC₂-C₁₈ alkynyl, or (C₀-C₈ alkyl)NR²⁴(O)OH; and

R²⁴ is hydrogen or C₁-C₁₈ alkyl,

Nonlimiting examples of the compound of Formula N include:

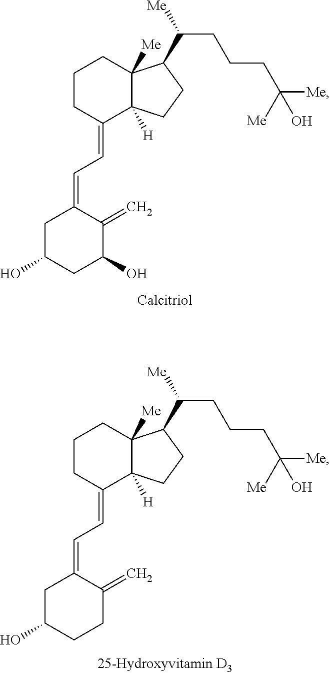 [SMILES: C=C1/C(=C\C=C2/CCC[C@]3(C)[C@@H]([C@H](C)CCCC(C)(C)O)CC[C@@H]23)C[C@@H](O)C[C@@H]1O ; C=C1CC[C@H](O)C/C1=C/C=C1\CCC[C@]2(C)[C@@H]([C@H](C)CCCC(C)(C)O)CC[C@@H]12]

and derivatives thereof.

In embodiments wherein Y comprises a structure of Formula N, Y is conjugated to L (e.g. when L is a linking group) or Ab (e.g. when L is a bond) at any position of Formula N that is capable of reacting with Ab or L. One skilled in the art could readily determine the position of conjugation on Formula N and means of conjugation of Formula N to Ab or L in view of general knowledge and the disclosure provided herein. In some embodiments, Formula N is conjugated to L or Ab at any of positions 1, 2, 3, 4, 5, 6, 7, 8, 9, 10, 11, 12, 13, 14, 15, 16, 17, 18, 19, 20, 21, 22, 23, 24, 25, or 26 of Formula N. In some embodiments. Formula N is conjugated to L or Ab at position 1, 3, 19, or 25 of Formula N.

In some embodiments, Y acts at the pregnane X receptor (PXR). In some embodiments, Y comprises any structure that permits or promotes agonist activity at the PXR, while in other embodiments Y is an antagonist of PXR. In some embodiments, Y is a steroid, antibiotic, antimycotic, bile acid, hyperforin, or a herbal compound. In exemplary embodiments, V is compound that is able to induce CYP3A4, such as dexamethasone and rifampicin. In embodiments wherein V comprises a structure that acts at the PXR. V is conjugated to L (e.g. when L is a linking group) or Ab (e.g. when L is a bond) at any position of Y that is capable of reacting with Ab or L. One skilled in the art could readily determine the position of conjugation on V and means of conjugation of V to Ab or L in view of general knowledge and the disclosure provided herein. In some embodiments, V is conjugated to L or Ab at any of positions on Y.

In some embodiments, the NM., is derivatized or otherwise chemically modified to comprise a reactive moiety that is capable of reacting with the glucagon superfamily peptide (Ab) or the linking group (N. In the embodiments described herein, V is derivatized at any position of Y that is capable of reacting with Ab or L. The position of derivatization on Y is apparent to one skilled in the art and depends on the type of NRL used and the activity that is desired. For example, in embodiments wherein NT has a structure comprising a tetracyclic skeleton having three 6-membered rings joined to one 5-membered ring or a variation thereof, Y can be derivatized at any of positions 1, 2, 3, 4, 5, 6, 7, 8, 9, 10, 11, 12, 13, 14, 15, 16, 17, 18, 19, 20, 21, 22, 23, 24, or 25. Other positions of derivatization can be as previously described herein.

The NRL can be derivatized using any agent known to one skilled in the art or described herein (e.g. see The Linking Group section and the Chemical Modification of Ab and/or Y subsection). For example, estradiol can be derivatized with succinic acid, succinic anhydride, benzoic acid, ethyl 2-bromoacetate, or iodoacetic acid to form the below derivatives of estradiol that are capable of conjugating to Ab or L.

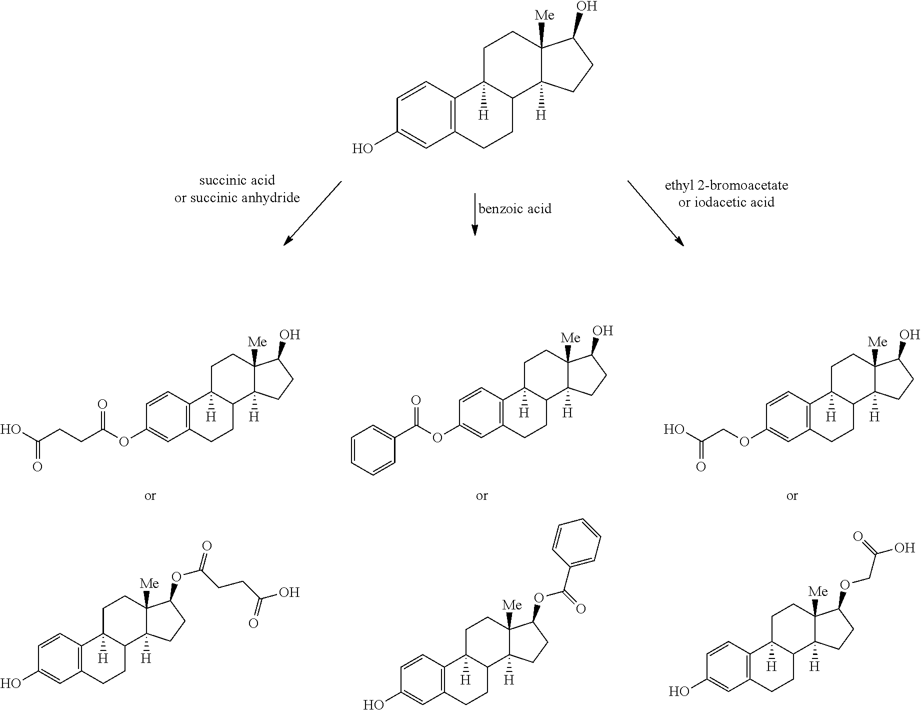

Similarly, any of the aforementioned NRL can be derivatized by methods known in the art. Additionally, certain derivatized ligands are commercially available and can be purchased from chemical companies such as Sigma-Aldrich.

### Conjugates

In some embodiments, the peptides and antibodies (Ab) described herein are glycosylated, amidated, carboxylated, phosphorylated, esterified, N-acylated, cyclized via, e.g., a disulfide bridge, or converted into a salt (e.g., an acid addition salt, a basic addition salt), and/or optionally dimerized, multimerized, or polymerized, or conjugated. As described herein. Ab can be a glucagon superfamily peptide, glucagon related peptide, including a Class 1, 2, 3, 4 or 5 glucagon related peptide, or osteocalcin, calcitonin, amylin, or an analog, derivative or conjugate thereof.

The present disclosure also encompasses conjugates in which Ab of Ab-L-Y is further linked to a heterologous moiety. The conjugation between Ab and the heterologous moiety can be through covalent bonding, non-covalent bonding (e.g. electrostatic interactions, hydrogen bonds, van der Wools interactions, salt bridges, hydrophobic interactions, and the like), or both types of bonding. A variety of non-covalent coupling systems may be used, including biotin-avidin, ligand/receptor, enzyme/substrate, nucleic acid/nucleic acid binding protein, binding protein, cellular adhesion molecule partners; or any binding partners or fragments thereof which have affinity for each other. In some aspects, the covalent bonds are peptide bonds. The conjugation of Ab to the heterologous moiety may be indirect or direct conjugation, the former of which may involve a linker or spacer. Suitable linkers and spacers are known in the art and include, but not limited to, any of the linkers or spacers described herein.

As used herein, the term “heterologous moiety” is synonymous with the term “conjugate moiety” and refers to any molecule (chemical or biochemical, naturally-occurring or non-coded) which is different from Ab to which it is attached. Exemplary conjugate moieties that can be linked to Ab include but are not limited to a heterologous peptide or polypeptide (including for example, a plasma protein), a targeting agent, an immunoglobulin or portion thereof (e.g., variable region, CDR, or Fe region), a diagnostic label such as a radioisotope, fluorophore or enzymatic label, a polymer including water soluble polymers, or other therapeutic or diagnostic agents. In some embodiments a conjugate is provided comprising Ab and a plasma protein, wherein the plasma protein is selected from the group consisting, of albumin, transferin, fibrinogen and globulins. In some embodiments the plasma protein moiety of the conjugate is albumin or transferin. The conjugate in some embodiments comprises Ab and one or more of a polypeptide, at nucleic acid molecule, an antibody or fragment thereof, a polymer, a quantum dot, a small molecule, a diagnostic agent, a carbohydrate, an amino acid.

### Hydrophilic Heterologous Moiety

In some embodiments, Ab described herein is covalently bonded to a hydrophilic moiety. As described herein, Ab can be a glucagon superfamily peptide, glucagon related peptide, including a Class 1, 2, 3, 4 or 5 glucagon related peptide, or osteocalcin, calcitonin, amylin, or an analog, derivative or conjugate thereof. Hydrophilic moieties can be attached to Ab under any suitable conditions used to react a protein with an activated polymer molecule. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, a-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., alpha-iodo acetic acid, alpha-bromoacetic acid, alpha-chloroacetic acid). If attached to the peptide by reductive alkylation, the polymer selected should have a single reactive aldehyde so that the degree of polymerization is controlled. See, for example, Kinstier et al., Adv. Drug. Delivery Rev, 54: 477-485 (2002); Roberts et at, Adv. Drug Delivery Rev. 54: 459-476 (2002); and Zalipsky et al., Adv. Drug Delivery Rey. 16: 157-182 (1995).

Further activating groups which can be used to link the hydrophilic moiety (water soluble polymer) to a protein include an alpha-halogenated acyl group (e.g., alpha-iodo acetic acid, alpha-bromoacetic acid, alpha-chloroacetic acid). In specific aspects, an amino acid residue of the peptide having a thiol is modified with a hydrophilic moiety such as PEG. In some embodiments, an amino acid on Ab comprising a thiol is modified with maleimide-activated PEG in a Michael addition reaction to result in a PEGylated peptide comprising the thioether linkage shown below:

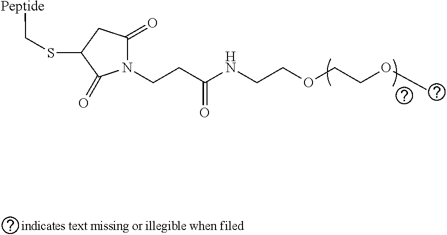

In some embodiments, the thiol of an amino acid of Ab is modified with a haloacetyl-activated PEG in a nucleophilic substitution reaction to result in a PEGylated peptide comprising the thioether linkage shown below:

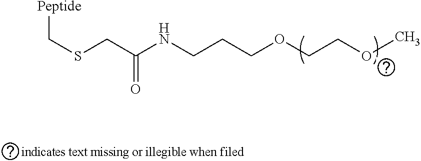

Suitable hydrophilic moieties include polyethylene glycol (PEG), polypropylene glycol, polyoxyethylated polyols (e.g., FOG), polyoxyethylated sorbitol, polyoxyethylated glucose, polyoxyethylated glycerol (POG), polyoxyalkylenes, polyethylene glycol propionaldehyde, copolymers of ethylene glycolpropylene glycol, monomethoxy-polyethylene glycol, mono-(C1-C10) alkoxy- or aryloxy-polyethylene carboxymethylcellulose, polyacetals, polyvinyl alcohol (PVA), polyvinyl pyrrolidone, poly-1, 3-dioxolane, poly-1,3,6-trioxane, ethylenemaleic anhydride copolymer, poly (.beta.-amino acids) (either homopolymers or random copolymers), poly(n-vinyl pyrrohdone)polyethylene glycol, propropylene glycol homopolymers (PPG) and other polyakylene oxides, polypropylene oxide/ethylene oxide copolymers, colonic acids or other polysaccharide polymers, Ficoll or dextran and mixtures thereof. Dextrans are polysaccharide polymers of glucose subunits, predominantly linked by (41-6 linkages. Dextran is available in many molecular weight ranges, e.g., about 1 kD to about 100 kD, or from about 5, 10, 15 or 20 kD to about 20, 30, 40, 50, 60, 70, 80 or 90 kD.

The hydrophilic moiety, e.g., polyethylene glycol chain, in accordance with some embodiments has a molecular weight selected from the range of about 500 to about 40,000 Daltons. In some embodiments the polyethylene glycol chain has a molecular weight selected from the range of about 500 to about 5,000 Daltons, or about 1,000 to about 5,000 Daltons. In another embodiment the hydrophilic moiety, e.g., polyethylene glycol chain, has a molecular weight of about 10,000 to about 20,000 Daltons. In yet other exemplary embodiments the hydrophilic moiety, e.g. polyethylene glycol chain, has a molecular weight of about 20,000 to about 40,000 Daltons.

Linear or branched hydrophilic polymers are contemplated. Resulting preparations of conjugates may be essentially monodisperse or polydisperse, and may have about 0.5, 0.7, 1, 1.2, 1.5 or 2 polymer moieties per peptide.

In some embodiments, the native amino acid of the peptide is substituted with an amino acid having a side chain suitable for crosslinking with hydrophilic moieties, to facilitate linkage of the hydrophilic moiety to the peptide. Exemplary amino acids include Cys, Lys, Orn, homo-Cys, or acetyl phenylalanine (Ac-Phe). In other embodiments, an amino acid modified to comprise a hydrophilic group is added to the peptide at the C-terminus.

In some embodiments, the peptide of the conjugate is conjugated to a hydrophilic moiety. (e.g. PEG, via covalent linkage between a side chain of an amino acid of the peptide and the hydrophilic moiety. In some embodiments, where Ab is a Class 1, 2, 3, 4 or 5 glucagon-related peptide, the peptide is conjugated to a hydrophilic moiety via the side chain of an amino acid at position 16, 17, 21, 24, 29, 40, a position within a C-terminal extension, or the C-terminal amino acid, or a combination of these positions. In some aspects, the amino acid covalently linked to a hydrophilic moiety (e.g., the amino acid comprising a hydrophilic moiety) is a Cys, Lys, Orn, homo-Cys, or Ac-Phe, and the side chain of the amino acid is covalently bonded to a hydrophilic moiety (e.g., PEG).

### The Linking Group (L)

As described herein, the present disclosures provide glucagon superfamily peptides conjugated with NIIR ligands having the formula Ab-L-V, wherein L is a linking group or a chemical bond. In some embodiments. L is stable in vivo. In some embodiments. L is hydrolyzable in vivo. In some embodiments, L is metastable in vivo.

Ab and V can be linked together through L using standard linking agents and procedures known to those skilled in the art. In some aspects, Ab and Y are fused directly and L is a bond. In other aspects, Ab and V are fused through a linking group L. For example, in some embodiments, Ab and Y are linked together via a peptide bond, optionally through a peptide or amino acid spacer. In some embodiments. Ab and Y are linked together through chemical conjugation, optionally through a linking group (L). In some embodiments. L is directly conjugated to each of Ab and V.

Chemical conjugation can occur by reacting a nucleophilic reactive group of one compound to an electrophilic reactive group of another compound. In some embodiments when L is a bond, Ab is conjugated to Y either by reacting to nucleophilic reactive moiety on Ab with an electrophilic reactive moiety on Y, or by reacting an electrophilic reactive moiety on Ab with a nucleophilic reactive moiety on Y. In embodiments when L is a group that links Ab and Y together, Ab and/or Y can be conjugated to L either by reacting, a nucleophilic reactive moiety on Ab and/or V with an electrophilic reactive moiety on L, or by reacting an electrophilic reactive moiety on Ab and/or Y with a nucleophilic reactive moiety on L. Nonlimiting examples of nucleophilic reactive groups include amino, thiol, and hydroxyl. Nonlimiting examples of electrophilic reactive groups include carboxyl, acyl chloride, anhydride, ester, succinimide ester, alkyl halide, sulfonate ester, maleimido, haloacetyl, and isocyanate. In embodiments where Ab and Y are conjugated together by reacting a carboxylic acid with an amine, an activating agent can be used to form an activated ester of the carboxylic acid.

The activated ester of the carboxylic acid can be, for example, N-hydroxysuccinimide (NHS), tosylate (Tos), mesylate, triflate, t carbodiimide, or a hexafluorophosphate. In some embodiments, the carbodiimide is 1,3-dicyclohexylearbodiimide (DCC), 1,1′ carbonyldiimidazole. (CDI), 1-ethyl-3-(3-dimethylaminopropyl)carbodiimide hydrochloride (EDC), or 1,3-diisopropylcarbodiimide (DICD). In some embodiments, the hexafluorophosphate is selected from a group consisting of hexafluorophosphate benzo triazol-1-yl -oxy-tris(dimethylamino)phosphonium hexafluorophosphate (BOP), benzotriazol-1-yl-oxytripyrrolidinophosphonium hexafluorophosphate (PyBOP), 2-(1H-7-azabenzottiazol-1-yl)-1,1,3,3-tetramethyl uranium hexafluorophosphate (HAM), and o-benzotriazole-N,N,N′,N′-tetramethyl -uronium -hexafluoro-phosphate (HBTU).

In some embodiments, Ab comprises a nucleophilic reactive group (e.g. the amino group, thiol group, or hydroxyl group of the side chain of lysine, cysteine or serine) that is capable of conjugating to an electrophilic reactive group on V or L. In some embodiments, Ab comprises an electrophilic reactive group (e.g. the carboxylase group of the side chain of Asp or Glu) that is capable of conjugating to a nucleophilic reactive group on Y or L. In some embodiments, Ab is chemically modified to comprise a reactive group that is capable of conjugating directly to V or to L. In some embodiments, Ab is modified at the C-terminal to comprise a natural or nonnatural amino acid with a nucleophilic side chain, such as an amino acid represented by Formula I, Formula II, or Formula III, as previously described herein. In exemplary embodiments, the C-terminal amino acid of Ab is selected from the group consisting of lysine, ornithine, serine, cysteine, and homocysteine. For example, the C-terminal amino acid of Ab can be modified to comprise a lysine residue. In some embodiments, Ab is modified at the C-terminal amino acid to comprise a natural or nonnatural amino acid with an electrophilic side chain such as, for example, Asp and Gin. In some embodiments, an internal amino acid of Ab is substituted with a natural or nonnatural amino acid having a nucleophilic side chain, such as an amino acid represented by Formula I, Formula II, or Formula III, as previously described herein. In exemplary embodiments, the internal amino acid of Ab that is substituted is selected from the group consisting of lysine, ornithine, serine, cysteine, and homocysteine. For example, an internal amino acid of Ab can be substituted with a lysine residue. In some embodiments, an internal amino acid of Ab is substituted with a natural or nonnatural amino acid with an electrophilic side chain, such as, for example, Asp and Glu.

In some embodiments, V comprises a reactive group that is capable of conjugating directly to Ab or to L. In some embodiments, Y comprises a nucleophilic reactive group (e.g. amine, thiol, hydroxyl) that is capable of conjugating to an electrophilic reactive group on Ab or L. In some embodiments, V comprises electrophilic reactive group (e.g. carboxyl group, activated form of a carboxyl group, compound with a leaving group) that is capable of conjugating to a nucleophilic reactive group on Ab or L. In some embodiments, Y is chemically modified to comprise either a nucleophilic reactive group that is capable of conjugating to an electrophilic reactive group on Ab or L. In some embodiments, V is chemically modified to comprise an electrophilic reactive group that is capable of conjugating to a nucleophilic reactive group on Ab or L.

In some embodiments, conjugation can be carried out through organosilanes, e.g. aminosilane treated with glutaraldehyde; carbonyldiimidazole (CDT) activation of silanol groups; or utilization of dendrimers. A variety of dendrimers are known in the art and include poly (amidoamine) (PAMAM) dendrimers, which are synthesized by the divergent method starting from ammonia or ethylenediamine initiator core reagents; a sub-class of PAMAM dendrimers based on a tris-aminoethylene-imine core; radially layered poly(amidoamine-organosilicon) dendrimers (PAMAMOS), which are inverted unimolecular micelles that consist of hydrophilic, nucleophilic polyamidoamine (PAMAM) interiors and hydrophobic organosilicon (OS) exteriors: Poly (Propylene Imine) (PPI) dendrimers, which are generally poly-alkyl amines having primary amines as end groups, while the dendrimer interior consists of numerous of tertiary tris-propylene amines; Poly (Propylene Amine) (POPAM) dendrimers: Diaminobutane (DAB) dendrimers; amphiphilic dendrimers; micellar dendrimers which are unimolecular micelles of water soluble hyper branched polyphenylenes; polytysine dendrimers; and dendrimers based on poly-benzyl ether hyper branched skeleton.

In some embodiments, conjugation can be carried out through olefin metathesis. In some embodiments, Y and Ab, Y and L, or Ab and L both comprise an alkene or alkyne moiety that is capable of undergoing metathesis. In some embodiments a suitable catalyst (e.g. copper, ruthenium) is used to accelerate the metathesis reaction. Suitable methods of performing olefin metathesis reactions are described in the art. See, for example. Schafmeister et al., J. Am. Chem. Soc. 122: 5891-5892 (2000), Walensky et al., Science 305: 1466-1470 (2004), and Blackwell et al., Angew, Chem., Int. Ed. 37: 3281-3284 (1998).

In some embodiments, conjugation can be carried out using click chemistry, A “click reaction” is wide in scope and easy to perform, uses only readily available reagents, and is insensitive to oxygen and water. In some embodiments, the click reaction is a cycloaddition reaction between an alkynyl group and an azido group to form a triazolyl group. In some embodiments, the click reaction uses a copper or ruthenium catalyst. Suitable methods of performing click reactions are described in the art. See, for example, Kolb et al., Drug Discovery Today 8: 1128 (2003): Kolb et al., Angew. Chem. Int. Ed. 40:2004 (2001); Rostovtsev et al., Angew. Chem, Int. Ed. 41:2596 (2002); Torne et al., J Org. Chem. 67:3057 (2002); Munetsch et al., J. Am. Chem. Soc. 126: 12809 (2004); Lewis et al., Angew. Chem. Int. Ed. 41: 1053 (2002); Speers, J. Am. Chem. Soc. 125:4686 (2003); Chan et al. Org. Lett. 6:2853 (2004); Zhang et al., J Am. Chem. Soc. 127: 15998 (2005); and Waser et al., J. Am. Chem. Soc. 127:8294 (2005).

Indirect conjugation via high affinity specific binding partners, e.g. streptavidin/biotin or avidin/biotin or lee tin/carbohydrate is also contemplated.

Chemical Modification of Ab and/or Y

In some embodiments, Ab and/or Y are functionalized to comprise a nucleophilic reactive group or an electrophilic reactive group with an organic derivatizing agent. This derivatizing agent is capable of reacting with selected side chains or the N- or C-terminal residues of targeted amino acids on Ab and functional groups on Y, Reactive groups on Ab and/or Y include, e.g., aldehyde, amino, ester, thiol, a-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, Ab and/or Y can be linked to each other indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, copolymers thereof, and mixed polymers of these amino acids and others, e.g., serines, to confer desirable solubility properties on the resultant loaded carrier.

Cysteinyl residues most commonly are reacted with a-haloaectates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-β-(5-imidozoyi)propionie acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with dig at pH 5.5-7.0 because this agent is relatively specific for the histidyl side chain. Para-bromophenacyl bromide also is useful: the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly. N-acetylimidizole and tetranitromethane are used to form 0-acetyl tyrosyl species and 3-nitro derivatives, respectively.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4,-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Other modifications include hydroxylation of proline and lysine, phosphorylation of hydroxyl groups of seryl or threonyl residues, methylation of the alpha-amino groups of lysine, arginine, and histidine side chains (T. E. Creighton. Proteins: Structure and Molecular Properties, W.H. Freeman & Co., San Francisco, pp. 79-86 (1983)), deamidation of asparagine or glutamine, acetylation of the N-terminal amine, and/or amidation or esterification of the C-terminal carboxylic acid group.

Another type of covalent modification involves chemically or enzymatically coupling glycosides to the peptide. Sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of tyrosine, or tryptophan, or (I) the amide group of glutamine. These methods are described in WO8705330 published 11 Sep. 1987, and in Aplin and Wriston, CRC Crit. Rev. Biochem., pp. 259-306 (1981).

### Structure of L

In some embodiments. L is a bond. In these embodiments, Ab and Y are conjugated together by reacting a nucleophilic reactive moiety on Ab with and electrophilic reactive moiety on Y. In alternative embodiments, Ab and V are conjugated together by reacting an electrophilic reactive moiety on Ab with a nucleophilic moiety on Y. In exemplary embodiments, L is an amide bond that forms upon reaction of an amine on Ab (e.g. an ε-amine of a lysine residue) with a carboxyl group on Y. In alternative embodiments, Ab and or Y are derivatized with a derivatizing agent before conjugation.

In some embodiments, L is a linking group. In some embodiments, L is a bifunctional linker and comprises only two reactive groups before conjugation to Ab and Y. In embodiments where both Ab and Y have electrophilic reactive groups, L comprises two of the same or two different nucleophilic groups (e.g. amine, hydroxyl, thiol) before conjugation to Ab and Y. In embodiments where both Ab and Y have nucleophilic reactive groups, L comprises two of the same or two different electrophic groups (e.g. carboxyl group, activated form of a carboxyl group, compound with a leaving group) before conjugation to Ab and Y. In embodiments where one of Ab or Y has a nucleophilic reactive group and the other of Ab or V has an electrophic reactive group, L comprises one nucleophilic reactive group and one electrophile group before conjugation to Ab and V.

L can be any molecule with at least two reactive groups (before conjugation to Ab and Y) capable of reacting with each of Ab and Y. In some embodiments I, has only two reactive groups and is bifunctional. I., (before conjugation to the peptides) can be represented by Formula VI:

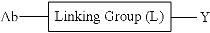

wherein A and B are independently nucleophilic or electrophic reactive groups. In some embodiments A and B are either both nucleophilic groups or both electrophic groups. In some embodiments one of A or B is a nucleophilic group and the other of A or B is an electrophile, group. Nonlimiting combinations of A and B are shown below.

| Both Nucleophilic | | Both Electrophilic | | Nucleophilic/Electrophilic | |
|---|---|---|---|---|---|
| A | B | A | B | A | B |
| Amino | Amino | Carboxyl | Carboxyl | Amino | Carboxyl |
| Amino | Thiol | Carboxyl | Acyl chloride | Amino | Acyl chloride |
| Amino | Hydroxyl | Carboxyl | Anhydride | Amino | Anhydride |
| Thiol | Amino | Carboxyl | Ester | Amino | Ester |
| Thiol | Thiol | Carboxyl | NHS | Amino | NHS |
| Thiol | Hydroxyl | Carboxyl | Halogen | Amino | Halogen |
| Hydroxyl | Amino | Carboxyl | Sulfonate ester | Amino | Sulfonate ester |
| Hydroxyl | Thiol | Carboxyl | Maleimido | Amino | Maleimido |
| Hydroxyl | Hydroxyl | Carboxyl | Haloacetyl | Amino | Haloacetyl |
| | | Carboxyl | Isocyanate | Amino | Isocyanate |
| | | Acyl chloride | Carboxyl | Thiol | Carboxyl |
| | | Acyl chloride | Acyl chloride | Thiol | Acyl chloride |
| | | Acyl chloride | Anhydride | Thiol | Anhydride |
| | | Acyl chloride | Ester | Thiol | Ester |
| | | Acyl chloride | NHS | Thiol | NHS |
| | | Acyl chloride | Halogen | Thiol | Halogen |
| | | Acyl chloride | Sulfonate ester | Thiol | Sulfonate ester |
| | | Acyl chloride | Maleimido | Thiol | Maleimido |
| | | Acyl chloride | Haloacetyl | Thiol | Haloacetyl |
| | | Acyl chloride | Isocyanate | Thiol | Isocyanate |
| | | Anhydride | Carboxyl | Hydroxyl | Carboxyl |
| | | Anhydride | Acyl chloride | Hydroxyl | Acyl chloride |
| | | Anhydride | Anhydride | Hydroxyl | Anhydride |
| | | Anhydride | Ester | Hydroxyl | Ester |
| | | Anhydride | NHS | Hydroxyl | NHS |
| | | Anhydride | Halogen | Hydroxyl | Halogen |
| | | Anhydride | Sulfonate ester | Hydroxyl | Sulfonate ester |
| | | Anhydride | Maleimido | Hydroxyl | Maleimido |
| | | Anhydride | Haloacetyl | Hydroxyl | Haloacetyl |
| | | Anhydride | Isocyanate | Hydroxyl | Isocyanate |
| | | Ester | Carboxyl | | |
| | | Ester | Acyl chloride | | |
| | | Ester | Anhydride | | |
| | | Ester | Ester | | |
| | | Ester | NHS | | |
| | | Ester | Halogen | | |
| | | Ester | Sulfonate ester | | |
| | | Ester | Maleimido | | |
| | | Ester | Haloacetyl | | |
| | | Ester | Isocyanate | | |
| | | NHS | Carboxyl | | |
| | | NHS | Acyl chloride | | |
| | | NHS | Anhydride | | |
| | | NHS | Ester | | |
| | | NHS | NHS | | |
| | | NHS | Halogen | | |
| | | NHS | Sulfonate ester | | |
| | | NHS | Maleimido | | |
| | | NHS | Haloacetyl | | |
| | | NHS | Isocyanate | | |
| | | Halogen | Carboxyl | | |
| | | Halogen | Acyl chloride | | |
| | | Halogen | Anhydride | | |
| | | Halogen | Ester | | |
| | | Halogen | NHS | | |
| | | Halogen | Halogen | | |
| | | Halogen | Sulfonate ester | | |
| | | Halogen | Maleimido | | |
| | | Halogen | Haloacetyl | | |
| | | Halogen | Isocyanate | | |
| | | Sulfonate ester | Carboxyl | | |
| | | Sulfonate ester | Acyl chloride | | |
| | | Sulfonate ester | Anhydride | | |
| | | Sulfonate ester | Ester | | |
| | | Sulfonate ester | NHS | | |
| | | Sulfonate ester | Halogen | | |
| | | Sulfonate ester | Sulfonate ester | | |
| | | Sulfonate ester | Maleimido | | |
| | | Sulfonate ester | Haloacetyl | | |
| | | Sulfonate ester | Isocyanate | | |
| | | Maleimido | Carboxyl | | |
| | | Maleimido | Acyl chloride | | |
| | | Maleimido | Anhydride | | |
| | | Maleimido | Ester | | |
| | | Maleimido | NHS | | |
| | | Maleimido | Halogen | | |
| | | Maleimido | Sulfonate ester | | |
| | | Maleimido | Maleimido | | |
| | | Maleimido | Haloacetyl | | |
| | | Maleimido | Isocyanate | | |
| | | Haloacetyl | Carboxyl | | |
| | | Haloacetyl | Acyl chloride | | |
| | | Haloacetyl | Anhydride | | |
| | | Haloacetyl | Ester | | |
| | | Haloacetyl | NHS | | |
| | | Haloacetyl | Halogen | | |
| | | Haloacetyl | Sulfonate ester | | |
| | | Haloacetyl | Maleimido | | |
| | | Haloacetyl | Haloacetyl | | |
| | | Haloacetyl | Isocyanate | | |
| | | Isocyanate | Carboxyl | | |
| | | Isocyanate | Acyl chloride | | |
| | | Isocyanate | Anhydride | | |
| | | Isocyanate | Ester | | |
| | | Isocyanate | NHS | | |
| | | Isocyanate | Halogen | | |
| | | Isocyanate | Sulfonate ester | | |
| | | Isocyanate | Maleimido | | |
| | | Isocyanate | Haloacetyl | | |
| | | Isocyanate | Isocyanate | | |

In some embodiments, A and B may include alkene and/or alkyne functional groups that are suitable for olefin metathesis reactions. In some embodiments, A and B include moieties that are suitable for click chemistry (e.g. alkene, alkynes, nitriles, azides). Other nonlimiting examples of reactive groups (A and B) include pyridyldithiol, aryl azide, diazirine, carbodiimide, and hydrazide.

In some embodiments, L is hydrophobic. Hydrophobic linkers are known in the art. See, e.g., Bioconjugate Techniques, G. T. Hermanson (Academic Press, San Diego, Calif., 1996), which is incorporated by reference in its entirety. Suitable hydrophobic linking groups known in the art include, for example, 8-hydroxy octanoic acid and 8-mercaptooctanoic acid. Before conjugation to the peptides of the composition, the hydrophobic linking group comprises at least two reactive groups (A and B), as described herein and as shown below:

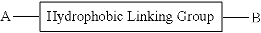

In some embodiments, the hydrophobic linking group comprises either a maleimido or an iodoacetyl group and either a carboxylic acid or an activated carboxylic acid (e.g. NHS ester) as the reactive groups. In these embodiments, the maleimido or iodoacetyl group can be coupled to a thiol moiety on Ab or Y and the carboxylic acid or activated carboxylic acid can be coupled to an amine on Ab or Y with or without the use of a coupling reagent. Any coupling agent known to one skilled in the art can be used to couple the carboxylic acid with the free amine such as, for example, DCC, DIC, HATU, HBTU, TBTU, and other activating agents described herein. In specific embodiments, the hydrophilic linking group comprises an aliphatic chain of 2 to 100 methylene groups wherein A and B are carboxyl groups or derivatives thereof (e.g. succinic acid). In other specific embodiments the L is iodoacetic acid.

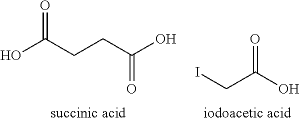 [SMILES: O=C(O)CCC(=O)O ; O=C(O)CI]

In some embodiments, the linking group is hydrophilic such as, for example, polyalkylene glycol. Before conjugation to the peptides of the composition, the hydrophilic linking group comprises at least two reactive groups (A and B), as described herein and as shown below:

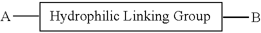

In specific embodiments, the linking group is polyethylene glycol (PEG). The PEG in certain embodiments has a molecular weight of about 100 Daltons to about 10,000 Daltons, e.g. about 500 Daltons to about 5000 Daltons. The PEG in some embodiments has a molecular weight of about 10,000 Daltons to about 40,000 Daltons.

In some embodiments, the hydrophilic linking group comprises either a maleimido or an iodoacetyl group and either a carboxylic acid or an activated carboxylic acid (e.g. NHS ester) as the reactive groups. In these embodiments, the maleimido or iodoacetyl group can be coupled to a thiol moiety on Ab or Y and the carboxylic acid or activated carboxylic acid can be coupled to an amine on Ab or Y with or without the use of a coupling reagent, Any appropriate coupling agent known to one skilled in the art can be used to couple the carboxylic acid with the amine such as, for example, DCC, DIC, HATU, HBTU, TBTU, and other activating agents described herein In some embodiments, the linking group is maleimido-PEG(20 kDa)-COOH, iodoacetyl-PEG(20 kDa)-COOH, maleimido-PEG(20 kDa)-NHS, or iodoacetyl-PEG(20 kDa)-NHS.

In some embodiments, the linking group is comprised of an amino acid, a dipeptide, a tripeptide, or a polypeptide, wherein the amino acid, dipeptide, tripeptide, or polypeptide comprises at least two activating groups, as described herein. In some embodiments, the linking group (L) comprises a moiety selected from the group consisting of: amino, ether, thioether, maleimido, disulfide, amide, ester, thioester, alkene, cycloalkene, alkyne, trizoyl, carbamate, carbonate, cathepsin B-cleavable, and hydrazone.

In some embodiments, L comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers may be selected according to their expected solubility (hydrophilicity) so as to provide a more soluble conjugate. In some embodiments, L provides a functional group that is subject to cleavage by an enzyme or other catalyst or hydrolytic conditions found in the target tissue or organ or cell. In some embodiments, the length of L is long enough to reduce the potential for steric hindrance.

### Stability of L In Vivo

In some embodiments, L is stable in vivo. In some embodiments, L is stable in blood serum for at least 5 minutes, e.g. less than 25%, 20%, 15%, 10% or 5% of the conjugate is cleaved when incubated in serum for a period of 5 minutes. In other embodiments, L is stable in blood serum for at least 10, or 20, or 25, or 30, or 60, or 90, or 120 minutes, or 3, 4, 5, 6, 7, 8, 9, 10, 12, 15, 18 or 24 hours. In these embodiments, L does not comprise a functional group that is capable of undergoing hydrolysis in vivo. In some exemplary embodiments, L is stable in blood serum for at least about 72 hours. Nonlimiting examples of functional groups that are not capable of undergoing significant hydrolysis in vivo include amides, ethers, and thioethers. For example, the following compound is not capable of undergoing significant hydrolysis in vivo:

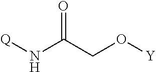

In some embodiments, L is hydrolyzable in vivo. In these embodiments, L comprises a functional group that is capable of undergoing hydrolysis in vivo, Nonlimiting examples of functional groups that are capable of undergoing hydrolysis in vivo include esters, anhydrides, and thioesters. For example the following compound is capable of undergoing hydrolysis in vivo because it comprises an ester group:

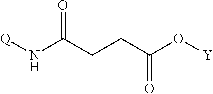

In some exemplary embodiments L is labile and undergoes substantial hydrolysis within 3 hours in blood plasma at 37° C., with complete hydrolysis within 6 hours. In some exemplary embodiments, L is not labile.

In some embodiments, L is metastable in vivo. In these embodiments, L comprises a functional group that is capable of being chemically or enzymatically cleaved in vivo (e.g., an acid-labile, reduction-labile, or enzyme-labile functional group), optionally over a period of time. In these embodiments, L can comprise, for example, a hydrazone moiety, a disulfide moiety, or a cathepsin-cleavable moiety. When L is metastable, and without intending to be bound by any particular theory, the Ab-L-Y conjugate is stable in an extracellular environment, e,g., stable in blood serum for the time periods described above, but labile in the intracellular environment or conditions that mimic the intracellular environment, so that it cleaves upon entry into a cell. In some embodiments when L is metastable, L is stable in blood serum for at least about 24, 25, 26, 27, 28, 29, 30, 31, 32, 33, 34, 35, 36, 42, or 48 hours, for example, at least about 48, 54, 60, 66, or 72 hours, or about 24-48, 48-72, 24-60, 36-48, 36-72, or 48-72 hours.

In some embodiments, L is metastable in vivo. In these embodiments, L comprises a functional group that is capable of being chemically or enzymatically cleaved in vivo (e.g., an acid-labile, reduction-labile, or enzyme-labile functional group), optionally over a period of time. In these embodiments, L can comprise, for example, a hydrazone moiety, a disulfide moiety, or a cathepsin-cleavable moiety. When L is metastable, and without intending to be bound by any particular theory, the Ab-L-Y conjugate is stable in an extracellular environment, e.g., stable in blood serum for the time periods described above, but labile in the intracellular environment or conditions that mimic the intracellular environment, so that it cleaves upon entry into a cell. In some embodiments when L is metastable, L is stable in blood serum for at least about 24, 25, 26, 27, 28, 29, 30, 31, 32, 33, 34, 35, 36, 42, or 48 hours, for example, at least about 48, 54, 60, 66, or 72 hours, or about 24-48, 48-72, 24-60, 36-48, 36-72, or 48-72 hours.

### Ab-L-Y Conjugates

### Conjugation of Ab and Y

Conjugation of Ab to Y through L can be carried out an any position within Ab, including any of positions 1-29, a position within a C-terminal extension, or the C-terminal amino acid, provided that the activity of Ab is retained, if not enhanced. In some embodiments, Y is conjugated to Ab through L at one or more of positions 10, 20, 24, 30, 37, 38, 39, 40, 41, 32, or 43. In specific embodiments, Y is conjugated to Ab through L at position 10 and/or 40 of Ab.

### Activity

### Activity at the Antibody-Binding Receptor and the Nuclear Receptor

In some embodiments, Ab-L-Y exhibits activity at both the Ab-binding receptor and a nuclear receptor. In some embodiments, the activity (e.g., the EC₈₀ or the relative activity or potency) of Ab at the Ab-binding receptor is within about 100-fold, about 75-fold, about 60-fold, about 50-fold, about 40-fold, about 30-fold, about 20-fold, about 10-fold, or about 5 fold different (higher or lower) from the activity (e.g., the EC₈₀ or the relative activity or potency) of Y at a nuclear hormone receptor. In some embodiments, the Ab-binding potency of Ab is within about 25-, about 20-, about 15-, about 10-, or about 5-fold different (higher or lower) from the potency of Y.

In some embodiments, the ratio of the relative activity or the EC₈₀ or the potency of the Ab at the Ab-binding receptor divided by the relative activity or the EC₈₀ or potency of Y at a nuclear hormone receptor is less than, or is about, X, wherein X is selected from 100, 75, 60, 50, 40, 30, 20, 15, 10, or 5. In some embodiments, the ratio of the EC₈₀ or potency or relative activity of Ab at the Ab-binding receptor divided by the EC₈₀ or potency or relative activity of Y at a nuclear hormone receptor is about 1 less than 5 (e.g., about 4, about 3, about 2, about 1). In some embodiments, the ratio of the Ab-binding potency of Ab compared to the nuclear hormone potency of Y is less than, or is about, Z, wherein Z is selected from 100, 75, 60, 50, 40, 30, 20, 15, 10, and 5. In some embodiments, the ratio of the Ab-binding potency of Ab compared to the nuclear potency Y is less than 5 (e.g., about 4, about 3, about 2, about 1). In some embodiments, Ab has an EC₈₀ at the Ab-binding receptor which is 2- to 10-fold (e.g., 2-fold, 3-fold, 4-fold, 5-fold, 6-fold, 7-fold, 8-fold, 9-fold, 10-fold) greater than the EC₈₀ of Y at a nuclear receptor.

In some embodiments, the ratio of the relative activity or potency or the EC₈₀ of Y at a nuclear hormone receptor divided by the relative activity or potency or the EC₈₀ of Ab at the Ab-binding receptor is less than, or is about, V, wherein V is selected from 100, 75, 60, 50, 40, 30, 20, 15, 10, or 5. In some embodiments, the ratio of the EC₈₀ or potency or relative activity of Y at a nuclear receptor divided by the EC₈₀ or potency or relative activity of Ab at the Ab-binding receptor is less than 5 (e.g., about 4, about 3, about 2, about 1). In some embodiments, the ratio of the nuclear potency of Y compared to the Ab-binding potency of Ab is less than, or is about, W, wherein W is selected from 100, 75, 60, 50, 40, 30, 20, 15, 10, and 5. In some embodiments, the ratio of the nuclear potency of Y compared to the Ab-binding potency of Ab is less than 5 (e.g., about 4, about 3, about 2, about 1). In some embodiments, Y has an EC₈₀ at a nuclear receptor which is about 2- to about 10-fold (e.g., 2-fold, 3-fold, 4-fold, 5-fold, 6-fold, 7-fold, 8-fold, 9-fold, 10-fold) greater than the EC₈₀ of Ab at the Ab-binding receptor.

In some embodiments, Y exhibits at least 0.1% (e.g., about 0.5% or more, about 1% or more, about 5% or more, about 10% or more, or more) of the activity of endogenous ligand at a nuclear receptor (nuclear potency) and Ab exhibits at least 0.1% (e.g., about 0.5% or more, about 1% or more, about 5% or more, about 10% or more, or more) of the activity of native antibody at the antibody-binding receptor (antibody potency).

### Prodrugs of Ab-L-Y

In some aspects of the invention, prodrugs of Ab-L-Y are provided wherein the prodrug comprises a dipeptide prodrug element (A-B) covalently linked to an active site of Ab via an amide linkage, as disclosed in International Patent Application No, PCT U.S. Pat. No. 0,968,745 (filed on Dec. 18, 2009), which is incorporated herein by reference in its entirety. Subsequent removal of the dipeptide under physiological conditions and in the absence of enzymatic activity, restores full activity to the Ab-L-Y conjugate.

In some embodiments a prodrug of Ab-L-Y is provided having the general structure of A-B-Ab-L-Y. In these embodiments A is an amino acid or a hydroxy acid and B is an N-alkylated amino acid linked to Ab through formation of an amide bond between a carboxyl of B (in A-B) and an amine of Ab. Furthermore, in some embodiments, A, B, or the amino acid of Ab to which A-B is linked, is a non-coded amino acid, and chemical cleavage of A-B from Ab is at least about 90% complete within about 1 to about 720 hours in PBS under physiological conditions. In another embodiment, chemical cleavage of A-B from Ab is at least about 50% complete within about 1 hour or about 1 week in PBS under physiological conditions.

In some embodiment the dipeptide prodrug element (A-B) comprises a compound having the general structure below:

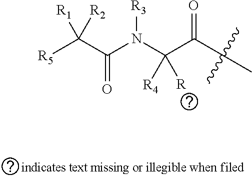

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (Ci-C₄ alkyl)NHC(NH₂⁺)NH₂, (C₀-C₄ alkyl)(C₃-C₆ cycloalkyl), (C₀-C₄ alkyl)(C₂-C₈ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₈ heterocyclic), (C₀-C4 alkyl)(C₆-C₁₀ aryl)R₇, and (C₁-C₄ alkyl)(C₃-C₀ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In some embodiments, the dipeptide prodrug element is linked to the amino terminus of Ab. In other embodiments, the dipeptide prodrug is linked to an internal amino acid of Ab, as described in International Patent Application No. PCT U.S. Pat. No. 0,968,745.

In some embodiments, Y is azide. In other embodiments, Y is cycloalkyne. In specific embodiments, the cyclooctyne has a structure of:

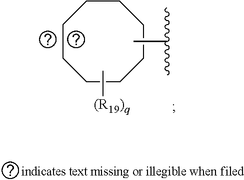

each R₁₉ is independently selected from the group consisting of C₁-C₆ alkyl, C₁-C₆ alkoxy, ester, ether, thioether, aminoalkyl, halogen, alkyl ester, aryl ester, amide, aryl amide, alkyl halide, alkyl amine, alkyl sulfonic acid, alkyl nitro, thioester, sulfonyl ester, halosulfonyl, nitrile, alkyl nitrile, and nitro; and
q is 0, 1, 2, 3, 4, 5, 6, 7, 8, 9, 10 or 11.

In certain embodiments of compounds of Formula (IV) and (VI), V is a hydroxylamine, methyl, aldehyde, protected aldehyde, ketone, protected ketone, thioester, ester, dicarbonyl, hydrazine, amidine, imine, diamine, keto-amine, keto-alkyne, and ene-di one.

In certain embodiments of compounds of Formula (I), (III), (IV), (V), and (VI), each L, L₁, L₂, L₃, and L₄ is independently a cleavable linker or non-cleavable linker. In certain embodiments of compounds of Formula (I), (III), (IV), (V), and (VI), each L, L₁, L₂, L₃, and L₄ is independently a oligo(ethylene glycol) derivatized linker.

In certain embodiments of compounds of Formula (I), (III), (IV), (V), and (VI), each alkylene, alkylene′, alkylene″, and alkylene independently is —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —CH₂CH₂CH₂CH₂CH₂CH₂CH₂CH₂CH₂—, —CH₂CH₂CH₂CH₂CH₂CH₂CH₂CH₂CH₂CH₂—, —CH₂CH₂CH₂CH₂CH₂CH₂CH₂CH₂CH₂CH₂CH₂—, or —CH₂CH₂CH₂CH₂CH₂CH₂CH₂CH₂CH₂CH₂CH₂CH₂—. In certain embodiments of compounds of Formula (XIV), (XV), (XVI), (XVII), and (XVIII), each n, n′, n″, N′″, and n″″ is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In certain embodiments of compounds of Formula (VIII) or (IX), R₁ is a polypeptide. In certain embodiments of compounds of Formula (VIII) or (IX), R₂ is a polypeptide. In certain embodiments of compounds of Formula (VIII) or (IX), the polypeptide is an antibody. In certain embodiments of compounds of Formula (VIII) or (IX), the antibody is herceptin.

Such non-natural amino acid NRL linked derivatives include NRL linked derivatives having the structure of Formula (X), (XI), (XII) or (XIII):

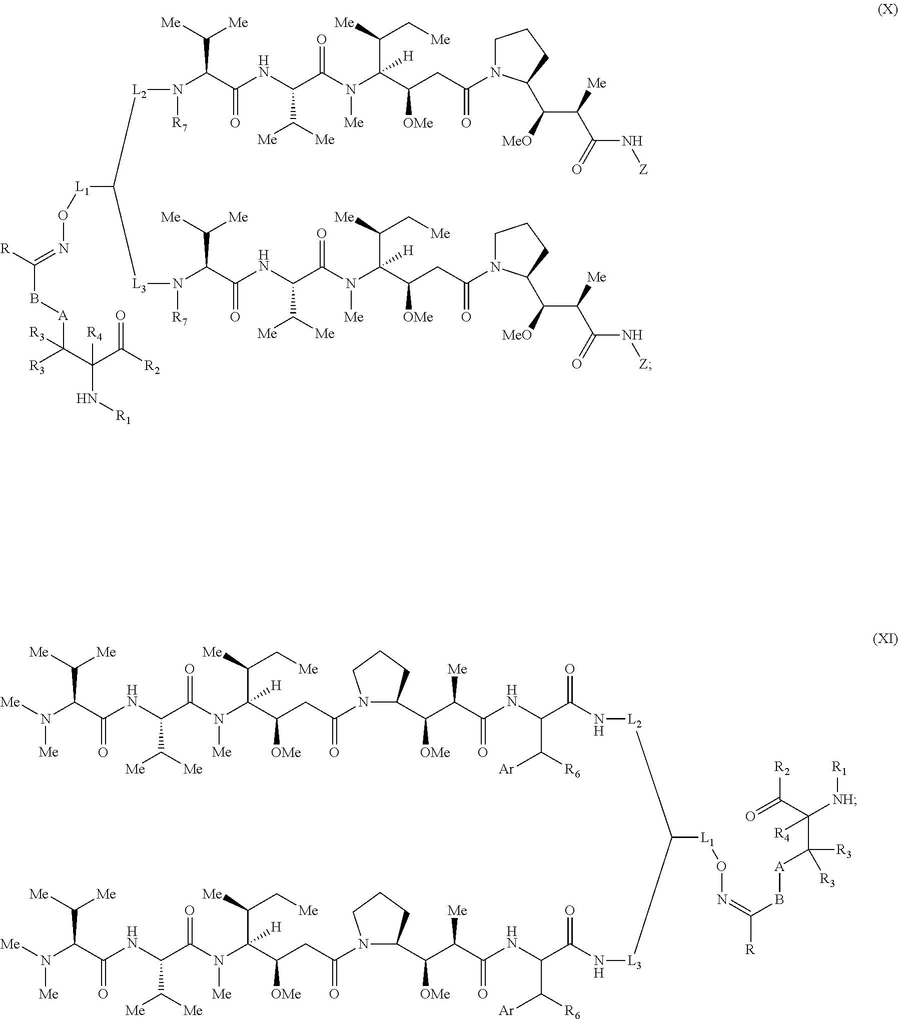

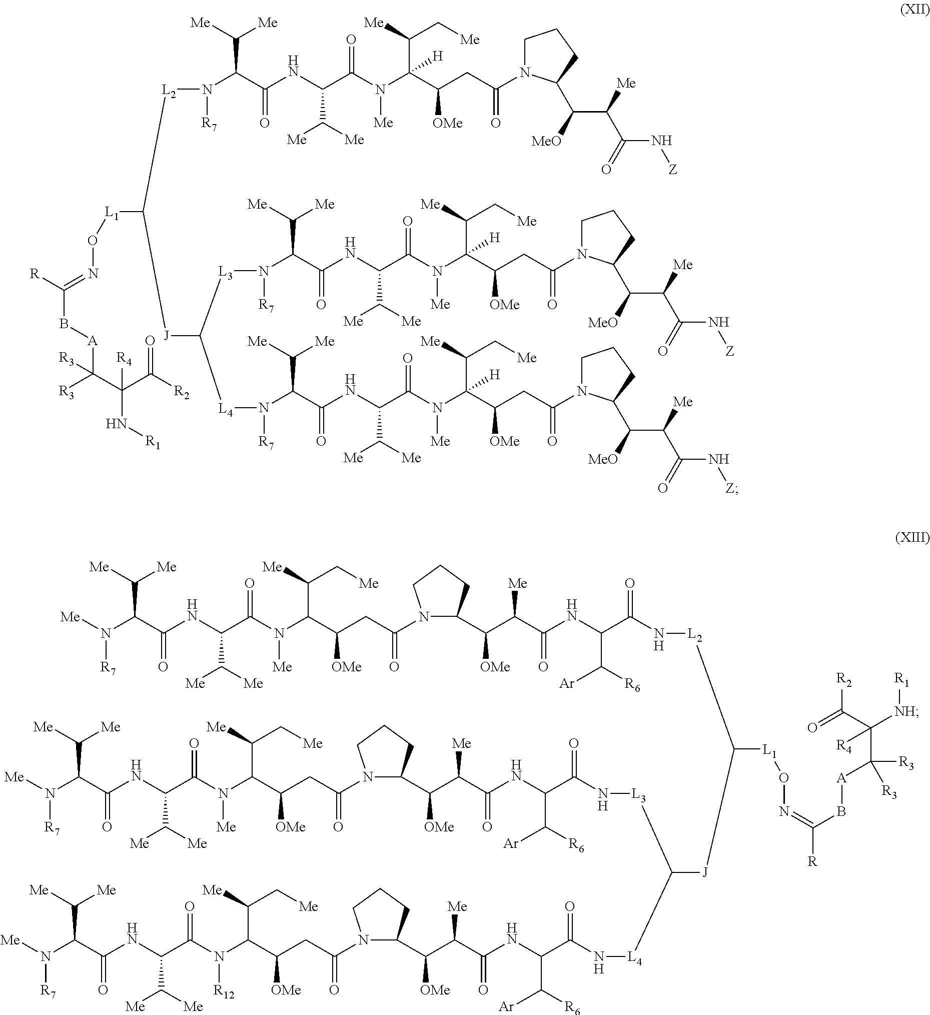

wherein:
A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene;
B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-, —S—, —S-(alkylene or substituted alkylene)-, —S(O)ₖ— where k is 1, 2, or 3, —S(O)ₖ(alkylene or substituted alkylene)-, —C(O)—, —C(O)-(alkylene or substituted alkylene)-, —C(S)—, —C(S)-(alkylene or substituted alkylene)-, —N(R′)—, —NR′-(alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-, —N(R′)C(O)O—, —S(O)ₖN(R′)—, —N(R′)C(O)N(R′)—, —N(R′)C(S)N(R′)—, —N(R′)S(O)ₖN(R′)—, —C(R′)₂—N═N—, and —C(R′)₂—N(R′)—N(R′)—, where each R′ is independently H, alkyl, or substituted alkyl;
R is H, alkyl, substituted alkyl, cycloalkyl, or substituted cycloalkyl;
R₁ is H, an amino protecting group, resin, at least one amino acid, polypeptide, or polynucleotide;
R₂ is OH, an ester protecting group, resin, at least one amino acid, polypeptide, or polynucleotide;
R₃ and R₄ are each independently H, halogen, lower alkyl, or substituted lower alkyl, or R₃ and R₄ or two R₃ groups optionally form a cycloalkyl or a heterocycloalkyl;
Z has the structure of;

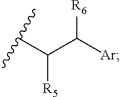

R₅ is H, CO₂H, C₁-C₆alkyl, or thiazole;
R₆ is OH or H;
Ar is phenyl or pyridine;
R₇ is C₁-C₆alkyl or hydrogen;
L₁, L₂, L₃, and L₄ are each linkers independently selected from the group consisting of a bond, -alkylene-, -(alkylene-O)ₙ-alkylene-J-, -alkylene′-J-(alkylene-O)ₙ-alkylene-, -J-(alkylene-O)ₙ-alkylene-, -(alkylene-O)ₙ-alkylene-J-(alkylene-O)_{n′}-alkylene-J′-, -(alkylene-O)ₙ-alkylene-J-alkylene′-, —W—, -alkylene-W—, alkylene′-J-(alkylene-NMe)-alkylene-W—, -J-(alkylene-NMe)-alkylene-W—, -J-alkylene-NMe-alkylene″-NMe-alkylene″-W-, and -alkylene-J-alkylene′-NMe-alkylene″-NMe-alkylene′″—W—;
   W has the structure of:

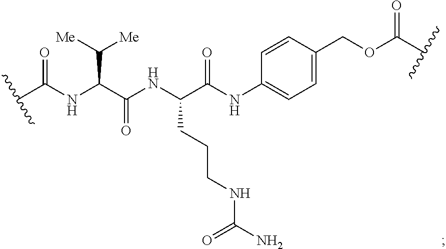 [SMILES: *C(=O)N[C@H](C(=O)N[C@@H](CCCNC(N)=O)C(=O)Nc1ccc(COC(*)=O)cc1)C(C)C]

each J and J′ independently have the structure of:

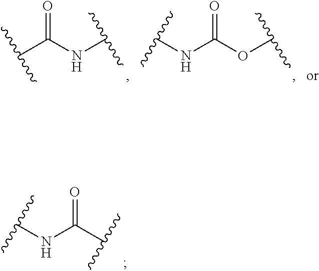 [SMILES: *NC(*)=O ; *NC(=O)O* ; *NC(*)=O]

and
   each n and n′ are independently integers greater than or equal to one.

In certain embodiments of compounds of Formula (X), (XI), (XII) or (XIII), R₅ is thiazole or carboxylic acid. In certain embodiments of compounds of Formula (X), (XI), (XII) or (XIII), R₆ is H. In certain embodiments of compounds of Formula (X), (XI), (XII) or (XIII), Ar is phenyl. In certain embodiments of compounds of Formula (X), (XI), (XII) or (XIII), R₇ is methyl. In certain embodiments of compounds of Formula (X), (XI),

(XII) or (XIII), n and n′ are integers from 0 to 20. In certain embodiments of compounds of Formula (X), (XI), (XII) or (XIII), n and n′ are integers from 0 to 10. In certain embodiments of compounds of Formula (X), (XI), (XII) or (XIII), n and n′ are integers from 0 to 5.

In certain embodiments of compounds of Formula (X), (XI), (XII) or (XIII), R₅ is thiazole. In certain embodiments of compounds of Formula (X), (XI), (XII) or (XIII), R₅ is hydrogen. In certain embodiments of compounds of Formula (X), (XI), (XII) or (XIII), R₅ is methyl, ethyl, propyl, iso-propyl, butyl, iso-butyl, sec-butyl, tert-butyl, pentyl, or hexyl. In certain embodiments of compounds of Formula (X), (XI), (XII) or (XIII), R₅ is —NH-(alkylene-O)ₙ—NH₂, wherein alkylene is —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —CH₂CH₂CH₂CH₂CH₂CH₂CH₂CH₂CH₂—, —CH₂CH₂CH₂CH₂CH₂CH₂CH₂CH₂CH₂CH₂—, —CH₂CH₂CH₂CH₂CH₂CH₂CH₂CH₂CH₂CH₂CH₂—, or —CH₂CH₂CH₂CH₂CH₂CH₂CH₂CH₂CH₂CH₂CH₂CH₂—. In certain embodiments of Formula (X), (XI), (XII) or (XIII), alkylene is methylene, ethylene, propylene, butylenes, pentylene, hexylene, or heptylene.

In certain embodiments of compounds of Formula (X), (XI), (XII) or (XIII), R₅ is —NH-(alkylene-O)ₙ—NH₂, wherein n is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In certain embodiments of compounds of Formula (X), (XI), (XII) or (XIII), R₆ is H. In some embodiments of compounds of Formula (X), (XI), (XII) or (XIII), R₆ is hydroxy.

In certain embodiments of compounds of Formula (X), (XI), (XII) or (XIII), Ar is phenyl.

In certain embodiments of compounds of Formula (X), (XI), (XII) or (XIII), R₇ is methyl, ethyl, propyl, iso-propyl, butyl, sec-butyl iso-butyl, tert-butyl, pentyl, or hexyl. In certain embodiments of compounds of Formula (X), (XI), (XII) or (XIII), R₇ is hydrogen.

In certain embodiments of compounds of Formula (X), (XI), (XII) or (XIII), each L₁, L₂, L₃, and L₄ is independently a cleavable linker or non-cleavable linker. In certain embodiments of compounds of Formula (X), (XI), (XII) or (XIII), each L₁, L₂, L₃, and L₄ is independently a oligo(ethylene glycol) derivatized linker.

In certain embodiments of compounds of Formula (X), (XI), (XII) or (XIII), each alkylene, alkylene, alkylene“, and alkylene”′ independently is —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —CH₂CH₂CH₂CH₂CH₂CH₂CH₂CH₂CH₂—, —CH₂CH₂CH₂CH₂CH₂CH₂CH₂CH₂CH₂CH₂—, —CH₂CH₂CH₂CH₂CH₂CH₂CH₂CH₂CH₂CH₂CH₂—, or CH₂CH₂CH₂CH₂CH₂CH₂CH₂CH₂CH₂CH₂CH₂CH₂—. In certain embodiments of compounds of Formula (X), (XI), (XII) or (XIII), alkylene is methylene, ethylene, propylene, butylenes, pentylene, hexylene, or heptylene.

In certain embodiments of compounds of Formula (X), (XI), (XII) or (XIII), each n and n′ independently is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In certain embodiments of compounds of Formula (X), (XI), (XII) or (XIII), R₁ is a polypeptide. In certain embodiments of compounds of Formula (X), (XI), (XII) or (XIII), R₂ is a polypeptide. In certain embodiments of compounds of Formula (X), (XI), (XII) or (XIII), the polypeptide is an antibody. In certain embodiments of compounds of Formula (X), (XI), (XII) or (XIII), the antibody is herceptin,

In certain embodiments, compounds of Formula (X), (XI), (XII) or (XIII) are stable in aqueous solution for at least 1 month under mildly acidic conditions. In certain embodiments, compounds of Formula (X), (XI), (XII) or (XIII) are stable for at least 2 weeks under mildly acidic conditions. In certain embodiments, compound of Formula (X), (XI), (XII) or (XIII) are stable for at least 5 days under mildly acidic conditions. In certain embodiments, such acidic conditions are pH 2 to 8. Such non-natural amino acids may be in the form of a salt, or may be incorporated into a non-natural amino acid polypeptide, polymer, polysaccharide, or a polynucleotide and optionally post translationally modified,

Oxime-based non-natural amino acids may be synthesized by methods already described in the art, or by methods described herein, including: (a) reaction of a hydroxylamine-containing non-natural amino acid with a carbonyl- or dicarbonyl-containing reagent; (b) reaction of a carbonyl- or dicarbonyl-containing non-natural amino acid with a hydroxylamine-containing reagent; or (c) reaction of an oxime-containing non-natural amino acid with certain carbonyl- or dicarbonyl-containing reagents.

Chemical Structure and Synthesis of Non-Natural Amino Acid Linked Nuclear Receptor Ligand Derivatives: Alkylated Aromatic Amine Linked Nuclearly Receptor Ligand Derivatives

In one aspect are NRL linker derivatives for the chemical derivatization of non-natural amino acids based upon the reactivity of an aromatic amine group. In further or additional embodiments, at least one of the aforementioned non-natural amino acids is incorporated into a NRL linker derivative, that is, such embodiments are non-natural amino acid linked NRL derivatives. In further or additional embodiments, the NRL linker derivatives are functionalized on their sidechains such that their reaction with a derivatizing non-natural amino acid generates an amine linkage. In further or additional embodiments, the NRL linker derivatives are selected from NRL linker derivatives having aromatic amine sidechains. In further or additional embodiments, the NRL linker derivatives comprise a masked sidechain, including a masked aromatic amine group. In further or additional embodiments, the non-natural amino acids are selected from amino acids having aromatic amine sidechains. In further or additional embodiments, the non-natural amino acids comprise a masked sidechain, including a masked aromatic amine group.

In another aspect are carbonyl-substituted NRL linker derivatives such as, by way of example, aldehydes, and ketones, for the production of derivatized non-natural amino acid polypeptides based upon an amine linkage. In a further embodiment are aldehyde-substituted NRL linker derivatives used to derivatize aromatic amine-containing non-natural amino acid polypeptides via the formation of an amine linkage between the derivatizing NRL linker and the aromatic amine-containing non-natural amino acid polypeptide.

In further or additional embodiments, the non-natural amino acids comprise aromatic amine sidechains where the aromatic amine is selected from an aryl amine or a heteroaryl amine. In a further or additional embodiment, the non-natural amino acids resemble a natural amino acid in structure but contain aromatic amine groups. In another or further embodiment the non-natural amino acids resemble phenylalanine or tyrosine (aromatic amino acids). In one embodiment, the non-natural amino acids have properties that are distinct from those of the natural amino acids. In one embodiment, such distinct properties are the chemical reactivity of the sidechain; in a further embodiment this distinct chemical reactivity permits the sidechain of the non-natural amino acid to undergo a reaction while being a unit of a polypeptide even though the sidechains of the naturally-occurring amino acid units in the same polypeptide do not undergo the aforementioned reaction. In a further embodiment, the sidechain of the non-natural amino acid has a chemistry orthogonal to those of the naturally-occurring amino acids. In a further embodiment, the sidechain of the non-natural amino acid comprises a nucleophile-containing moiety; in a further embodiment, the nucleophile-containing moiety on the sidechain of the non-natural amino acid can undergo a reaction to generate an amine-linked derivatized NRL. In a further embodiment, the sidechain of the non-natural amino acid comprises an electrophile-containing moiety; in a further embodiment, the electrophile-containing moiety on the sidechain of the non-natural amino acid can undergo nucleophilic attack to generate an amine-linked derivatized NRL. In any of the aforementioned embodiments in this paragraph, the non-natural amino acid may exist as a separate molecule or may be incorporated into a polypeptide of any length; if the latter, then the polypeptide may further incorporate naturally-occurring or non-natural amino acids.

Modification of non-natural amino acids described herein using reductive alkylation or reductive amination reactions have any or all of the following advantages. First, aromatic amines can be reductively alkylated with carbonyl-containing compounds, including aldehydes, and ketones, in a pH range of about 4 to about 10 (and in certain embodiments in a pH range of about 4 to about 7) to generate substituted amine, including secondary and tertiary amine, linkages. Second, under these reaction conditions the chemistry is selective for non-natural amino acids as the sidechains of naturally occurring amino acids are unreactive. This allows for site-specific derivatization of polypeptides which have incorporated non-natural amino acids containing aromatic amine moieties or protected aldehyde moieties, including, by way of example, recombinant proteins. Such derivatized polypeptides and proteins can thereby be prepared as defined homogeneous products. Third, the mild conditions needed to effect the reaction of an aromatic amine moiety on an amino acid, which has been incorporated into a polypeptide, with an aldehyde-containing reagent generally do not irreversibly destroy the tertiary structure of the polypeptide (excepting, of course, where the purpose of the reaction is to destroy such tertiary structure). Similarly, the mild conditions needed to effect the reaction of an aldehyde moiety on an amino acid, which has been incorporated into a polypeptide and deprotected, with an aromatic amine-containing reagent generally do not irreversibly destroy the tertiary structure of the polypeptide (excepting, of course, where the purpose of the reaction is to destroy such tertiary structure). Fourth, the reaction occurs rapidly at room temperature, which allows the use of many types of polypeptides or reagents that would otherwise be unstable at higher temperatures. Fifth, the reaction occurs readily is aqueous conditions, again allowing use of polypeptides and reagents incompatible (to any extent) with non-aqueous solutions. Six, the reaction occurs readily even when the ratio of polypeptide or amino acid to reagent is stoichiometric, stoichiometric-like, or near-stoichiometric, so that it is unnecessary to add excess reagent or polypeptide to obtain a useful amount of reaction product. Seventh, the resulting amine can be produced regioselectively and/or regiospecifically, depending upon the design of the amine and carbonyl portions of the reactants. Finally, the reductive alkylation of aromatic amines with aldehyde-containing reagents, and the reductive amination of aldehydes with aromatic amine containing reagents, generates amine, including secondary and tertiary amine, linkages which are stable under biological conditions,

Non-natural amino acids with nucleophilic reactive groups, such as, by way of example only, an aromatic amine group (including secondary and tertiary amine groups), a masked aromatic amine group (which can be readily converted into a aromatic amine group), or a protected aromatic amine group (which has reactivity similar to a aromatic amine group upon deprotection) allow for a variety of reactions to link molecules via various reactions, including but not limited to, reductive alkylation reactions with aldehyde containing NRL linked derivatives. Such alkylated non-natural amino acid linked NRL derivatives include amino acids having the structure of Formula (XXV), (XXVI), (XXVII), (XXVIII), (XXIX), or (XXX):

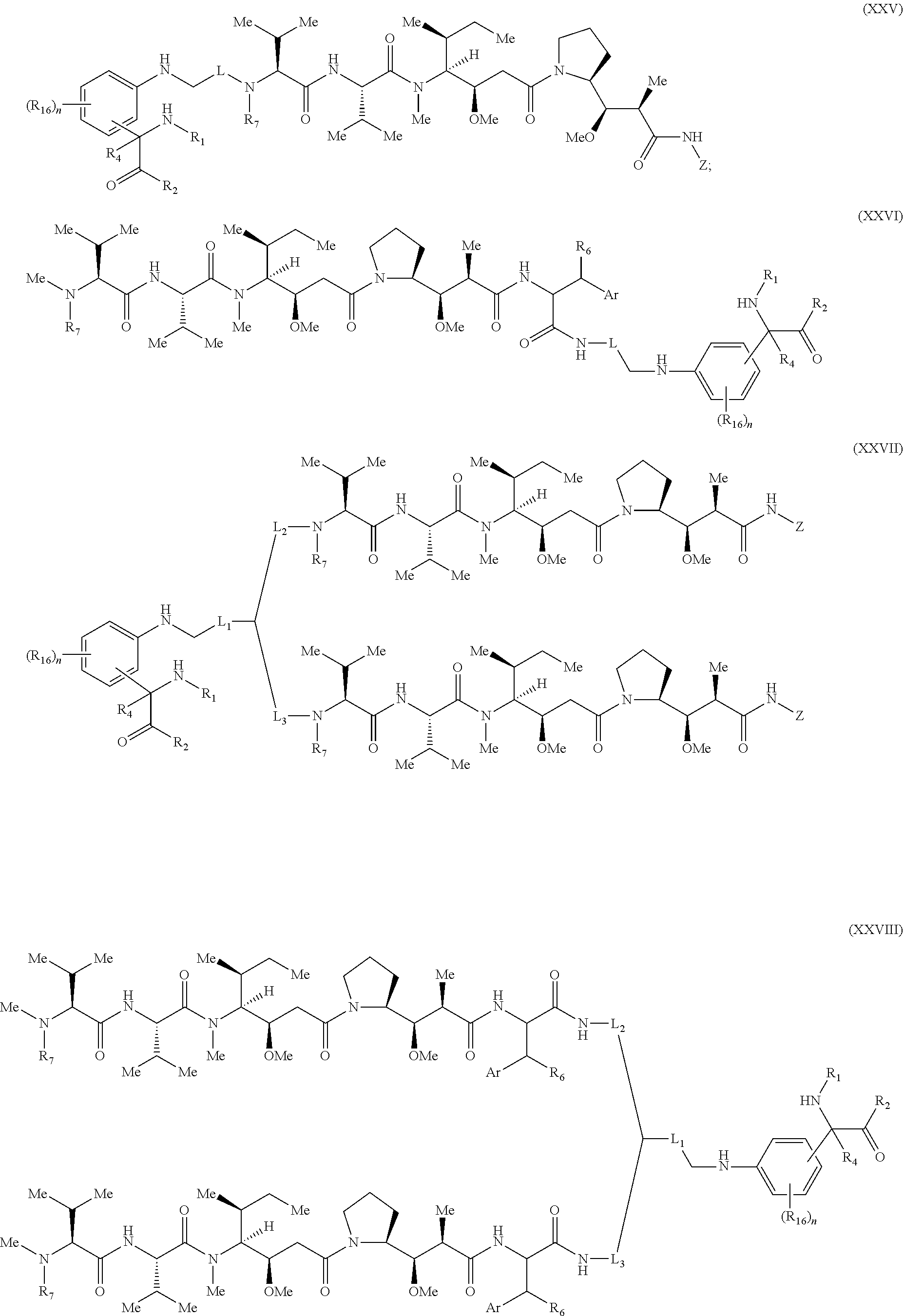

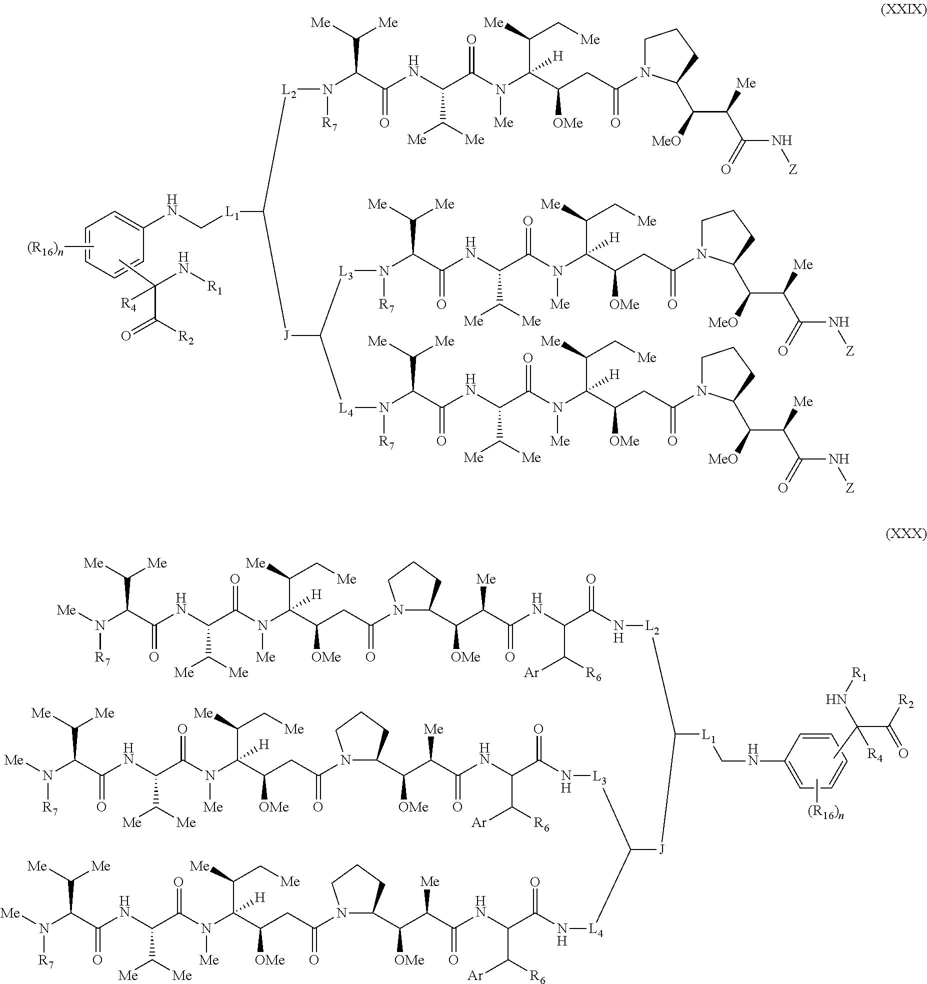

wherein:
Z has the structure of:

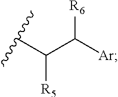

R₅ is H, CO₂H, C₁-C₆alkyl, or thiazole;
R₆ is OH or H;
Ar is phenyl or pyridine;
R₁ is H, an amino protecting group, resin, at least one amino acid, polypeptide, or polynucleotide;
R₂ is OH, an ester protecting group, resin, at least one amino acid, polypeptide, or polynucleotide;
R₄ is H, halogen, lower alkyl, or substituted lower alkyl;
R₇ is C₁-C₆alkyl or hydrogen;
L, L₁, L₂, L₃, and L₄ are each linkers selected from the group consisting of a bond, -alkylene-, -alkylene-C(O)—, -(alkylene-O)ₙ-alkylene-, -(alkylene-O)ₙ-alkylene-C(O)—, -(alkylene-O)ₙ—(CH₂)_{n′}—NHC(O)—(CH₂)_{n′}—C(Me)₂-S—S—(CH₂)_{n″}—NHC(O)-(alkylene-O)_{n′″}-alkylene-, -(alkylene-O)ₙ-alkylene-W—, -alkylene-C(O)—W—, -(alkylene-O)ₙ-alkylene-J-, -alkylene′-J-(alkylene-O)ₙ-alkylene-, -(alkylene-O)ₙ-alkylene-J-alkylene′, -J-(alkylene-O)ₙ-alkylene-, -(alkylene-O)ₙ-alkylene-J-(alkylene-O)ₙ′-alkylene-J′-, —W—, -alkylene-W—, alkylene′-J-(alkylene-NMe)ₙ-alkylene-W—, and J-(alkylene-NMe)ₙ-alkylene-W—, -(alkylene-O)ₙ-alkylene-U-alkylene-C(O)—, -(alkylene-O)ₙ-alkylene-U-alkylene-; -J-alkylene-NMe-alkylene′-NMe-alkylene″-W-, and -alkylene-J-alkylene′-NMe-alkylene″-NM-alkylene′″-W—;
   W has the structure of:

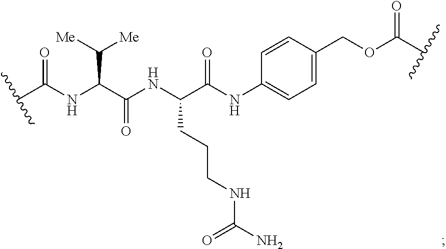 [SMILES: *C(=O)N[C@H](C(=O)N[C@@H](CCCNC(N)=O)C(=O)Nc1ccc(COC(*)=O)cc1)C(C)C]

U has the structure of:

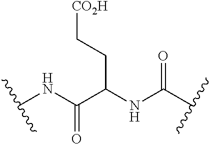 [SMILES: *NC(=O)C(CCC(=O)O)NC(*)=O]

each J and J′ independently have the structure of:

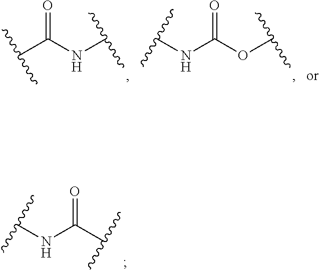 [SMILES: *NC(*)=O ; *NC(=O)O* ; *NC(*)=O]

each n and n′ are independently integers greater than or equal to one; and
each R₁₆ is independently selected from the group consisting of hydrogen, halogen, alkyl, NO₂, CN, and substituted alkyl.
   Such alkylated non-natural amino acid linked NRL derivatives may also be in the form of a salt, or may be incorporated into a non-natural amino acid polypeptide, polymer, polysaccharide, or a polynucleotide and optionally reductively alkylated,

### Pharmaceutical Compositions

### Salts

In some embodiments, the Ab-L-Y conjugates described herein are in the form of a salt, e.g., a pharmaceutically acceptable salt. As used herein the term “pharmaceutically acceptable salt” refers to salts of compounds that retain the biological activity of the parent compound, and which are not biologically or otherwise undesirable. Such salts can be prepared in situ during the final isolation and purification of the conjugate, or separately prepared by reacting a free base function with a suitable acid. Many of the compounds disclosed herein are capable of forming acid and/or base salts by virtue of the presence of amino and/or carboxyl groups or groups similar thereto.

Pharmaceutically acceptable acid addition salts may be prepared from inorganic and organic acids. Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate, and undecanoate. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Examples of acids which can be employed to form pharmaceutically acceptable acid addition salts include, for example, an inorganic acid, e.g., hydrochloric acid, hydrobromic acid, sulphuric acid, and phosphoric acid, and an organic acid, e.g., oxalic acid, maleic acid, succinic acid, and citric acid.

Basic addition salts also can be prepared in situ during the final isolation and purification of the source of salicylic acid, or by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethyl ammonium, methylammonium, dimethylammonium, trimethylammonium, triethylammonium, diethylammonium, and ethylammonium, amongst others. Other representative organic amines useful for the formation of base addition salts include, for example, ethylenediamine, ethanolamine, diethanolamine, piperidine, piperazine, and the like. Salts derived from organic bases include, but are not limited to, salts of primary, secondary and tertiary amines.

Further, basic nitrogen-containing groups can be quaternized with the conjugate of the present disclosure as lower alkyl halides such as methyl, ethyl, propyl, and butyl chlorides, bromides, and iodides; long chain halides such as decyl, lauryl, myristyl, and stearyl chlorides, bromides, and iodides; arylalkyl halides like benzyl and phenethyl bromides and others. Water or oil-soluble or dispersible products are thereby obtained.

### Formulations

In accordance with some embodiments, a pharmaceutical composition is provided wherein the composition comprises a Ab-L-Y conjugate of the present disclosure, or pharmaceutically acceptable salt thereof, and a pharmaceutically acceptable carrier. The pharmaceutical composition can comprise any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, viscosity-increasing agents, water-absorbing agents, water-miscible cosolvents, water softeners, or wetting agents.

In some embodiments, the pharmaceutical composition comprises any one or a combination of the following components: acacia, acesulfame potassium, acetyl tributyl citrate, acetyltriethyl citrate, agar, albumin, alcohol, dehydrated alcohol, denatured alcohol, dilute alcohol, aleuritic acid, alginic acid, aliphatic polyesters, alumina, aluminum hydroxide, aluminum stearate, amylopectin, α-amylose, ascorbic acid, ascorbyl palmitate, aspartame, bacteriostatic water for injection, bentonite, bentonite magma, benzalkonium chloride, benzethonium chloride, benzoic acid, benzyl alcohol, benzyl benzoate, bronopol, butylated hydroxyanisole, butylated hydroxytoluene, butylparaben, butylparaben sodium, calcium alginate, calcium ascorbate, calcium carbonate, calcium cyclamate, dibasic anhydrous calcium phosphate, dibasic dehydrate calcium phosphate, tribasic calcium phosphate, calcium propionate, calcium silicate, calcium sorbate, calcium stearate, calcium sulfate, calcium sulfate hemihydrate, canola oil, carbomer, carbon dioxide, carboxymethyl cellulose calcium, carboxymethyl cellulose sodium, β-carotene, carrageenan, castor oil, hydrogenated castor oil, cationic emulsifying wax, cellulose acetate, cellulose acetate phthalate, ethyl cellulose, microcrystalline cellulose, powdered cellulose, silicified microcrystalline cellulose, sodium carboxymethyl cellulose, cetostearyl alcohol, cetrimide, cetyl alcohol, chlorhexidine, chlorobutanol, chlorocresol, cholesterol, chlorhexidine acetate, chlorhexidine gluconate, chlorhexidine hydrochloride, chlorodifluoroethane (HCFC), chlorodifluoromethane, chlorofluorocarbons(CFC)chlorophenoxyethanol, chloroxylenol, corn syrup solids, anhydrous citric acid, citric acid monohydrate, cocoa butter, coloring agents, corn oil, cottonseed oil, cresol, m-cresol, o-cresol, p-cresol, croscarmellose sodium, crospovidone, cyclamic acid, cyclodextrins, dextrates, dextrin, dextrose, dextrose anhydrous, diazolidinyl urea, dibutyl phthalate, dibutyl sebacate, diethanolamine, diethyl phthalate, difluoroethane (HFC), dimethyl-β-cyclodextrin, cyclodextrin-type compounds such as Captisol®, dimethyl ether, dimethyl phthalate, dipotassium edentate, disodium edentate, disodium hydrogen phosphate, docusate calcium, docusate potassium, docusate sodium, dodecyl gallate, dodecyltrimethylammonium bromide, edentate calcium disodium, edtic acid, eglumine, ethyl alcohol, ethylcellulose, ethyl gallate, ethyl laurate, ethyl maltol, ethyl oleate, ethylparaben, ethylparaben potassium, ethylparaben sodium, ethyl vanillin, fructose, fructose liquid, fructose milled, fructose pyrogen-free, powdered fructose, fumaric acid, gelatin, glucose, liquid glucose, glyceride mixtures of saturated vegetable fatty acids, glycerin, glyceryl behenate, glyceryl monooleate, glyceryl monostearate, self-emulsifying glyceryl monostearate, glyceryl palmitostearate, glycine, glycols, glycofurol, guar gum, heptafluoropropane (HFC), hexadecyltrimethylannnonium bromide, high fructose syrup, human serum albumin, hydrocarbons (HC), dilute hydrochloric acid, hydrogenated vegetable oil, type II, hydroxyethyl cellulose, 2-hydroxyethyl-β-cyclodextrin, hydroxypropyl cellulose, low-substituted hydroxypropyl cellulose, 2-hydroxypropyl-β-cyclodextrin, hydroxypropyl methylcellulose, hydroxypropyl methylcellulose phthalate, imidurea, indigo carmine, ion exchangers, iron oxides, isopropyl alcohol, isopropyl myristate, isopropyl palmitate, isotonic saline, kaolin, lactic acid, lactitol, lactose, lanolin, lanolin alcohols, anhydrous lanolin, lecithin, magnesium aluminum silicate, magnesium carbonate, normal magnesium carbonate, magnesium carbonate anhydrous, magnesium carbonate hydroxide, magnesium hydroxide, magnesium lauryl sulfate, magnesium oxide, magnesium silicate, magnesium stearate, magnesium trisilicate, magnesium trisilicate anhydrous, malic acid, malt, maltitol, maltitol solution, maltodextrin, maltol, maltose, mannitol, medium chain triglycerides, meglumine, menthol, methylcellulose, methyl methacrylate, methyl oleate, methylparaben, methylparaben potassium, methylparaben sodium, microcrystalline cellulose and carboxymethylcellulose sodium, mineral oil, light mineral oil, mineral oil and lanolin alcohols, oil, olive oil, monoethanolamine, montmorillonite, octyl gallate, oleic acid, palmitic acid, paraffin, peanut oil, petrolatum, petrolatum and lanolin alcohols, pharmaceutical glaze, phenol, liquified phenol, phenoxyethanol, phenoxypropanol, phenylethyl alcohol, phenylmercuric acetate, phenylmercuric borate, phenylmercuric nitrate, polacrilin, polacrilin potassium, poloxamer, polydextrose, polyethylene glycol, polyethylene oxide, polyacrylates, polyethylene-polyoxypropylene-block polymers, polymethacrylates, polyoxyethylene alkyl ethers, polyoxyethylene castor oil derivatives, polyoxyethylene sorbitol fatty acid esters, polyoxyethylene stearates, polyvinyl alcohol, polyvinyl pyrrolidone, potassium alginate, potassium benzoate, potassium bicarbonate, potassium bisulfite, potassium chloride, postassium citrate, potassium citrate anhydrous, potassium hydrogen phosphate, potassium metabisulfite, monobasic potassium phosphate, potassium propionate, potassium sorbate, povidone, propanol, propionic acid, propylene carbonate, propylene glycol, propylene glycol alginate, propyl gallate, propylparaben, propylparaben potassium, propylparaben sodium, protamine sulfate, rapeseed oil, Ringer's solution, saccharin, saccharin ammonium, saccharin calcium, saccharin sodium, safflower oil, saponite, serum proteins, sesame oil, colloidal silica, colloidal silicon dioxide, sodium alginate, sodium ascorbate, sodium benzoate, sodium bicarbonate, sodium bisulfite, sodium chloride, anhydrous sodium citrate, sodium citrate dehydrate, sodium chloride, sodium cyclamate, sodium edentate, sodium dodecyl sulfate, sodium lauryl sulfate, sodium metabisulfite, sodium phosphate, dibasic, sodium phosphate, monobasic, sodium phosphate, tribasic, anhydrous sodium propionate, sodium propionate, sodium sorbate, sodium starch glycolate, sodium stearyl fumarate, sodium sulfite, sorbic acid, sorbitan esters (sorbitan fatty esters), sorbitol, sorbitol solution 70%, soybean oil, spermaceti wax, starch, corn starch, potato starch, pregelatinized starch, sterilizable maize starch, stearic acid, purified stearic acid, stearyl alcohol, sucrose, sugars, compressible sugar, confectioner's sugar, sugar spheres, invert sugar, Sugartab, Sunset Yellow FCF, synthetic paraffin, talc, tartaric acid, tartrazine, tetrafluoroethane (HFC), theobroma oil, thimerosal, titanium dioxide, alpha tocopherol, tocopheryl acetate, alpha tocopheryl acid succinate, beta-tocopherol, delta-tocopherol, gamma-tocopherol, tragacanth, triacetin, tributyl citrate, triethanolamine, triethyl citrate, trimethyl- -cyclodextrin, trimethyltetradecylammonium bromide, tris buffer, trisodium edentate, vanillin, type Ihydrogenated vegetable oil, water, soft water, hard water, carbon dioxide-free water, pyrogen-free water, water for injection, sterile water for inhalation, sterile water for injection, sterile water for irrigation, waxes, anionic emulsifying wax, carnauba wax, cationic emulsifying wax, cetyl ester wax, microcrystalline wax, nonionic emulsifying wax, suppository wax, white wax, yellow wax, white petrolatum, wool fat, xanthan gum, xylitol, zein, zinc propionate, zinc salts, zinc stearate, or any excipient in the Handbook of Pharmaceutical Excipients, Third Edition, A. H. Kibbe (Pharmaceutical Press, London, UK, 2000), which is incorporated by reference in its entirety, Remington's Pharmaceutical Sciences, Sixteenth Edition, E. W. Martin (MackPublishing Co., Easton, Pa., 1980), which is incorporated by reference in its entirety, discloses various components used in formulating pharmaceutically acceptable compositions and known techniques for the preparation thereof. Except insofar as any conventional agent is incompatible with the pharmaceutical compositions, its use in pharmaceutical compositions is contemplated. Supplementary active ingredients also can be incorporated into the compositions.

In some embodiments, the foregoing component(s) may be present in the pharmaceutical composition at any concentration, such as, for example, at least A, wherein A is 0.0001% w/v, 0.001% w/v, 0.01% w/v, 0.1% w/v, 1% w/v, 2% w/v, 5% w/v, 10% w/v, 20% w/v, 30% w/v, 40% w/v, 50% w/v, 60% w/v, 70% w/v, 80% w/v, or 90% w/v. In some embodiments, the foregoing component(s) may be present in the pharmaceutical composition at any concentration, such as, for example, at most B, wherein B is 90% w/v, 80% w/v, 70% w/v, 60% w/v, 50% w/v, 40% w/v, 30% w/v, 20% w/v, 10% w/v, 5% w/v, 2% w/v, 1% w/v, 0.1% w/v, 0.001% w/v, or 0.0001%. In other embodiments, the foregoing component(s) may be present in the pharmaceutical composition at any concentration range, such as, for example from about A to about B. In some embodiments, A is 0,0001% and B is 90%.

The pharmaceutical compositions may be formulated to achieve a physiologically compatible pH. In some embodiments, the pH of the pharmaceutical composition may be at least 5, at least 5,5, at least 6, at least 6.5, at least 7, at least 7.5, at least 8, at least 8.5, at least 9, at least 9.5, at least 10, or at least 10.5 up to and including pH 11, depending on the formulation and route of administration. In certain embodiments, the pharmaceutical compositions may comprise buffering agents to achieve a physiological compatible pH. The buffering agents may include any compounds capable of buffering at the desired pH such as, for example, phosphate buffers (e.g., PBS), triethanolamine, Tris, bicine, TAPS, tricine, HEPES, TES, MOPS, PIPES, cacodylate, MES, and others. In certain embodiments, the strength of the buffer is at least 0.5 mM, at least 1 mM, at least 5 mM, at least 10 mM, at least 20 mM, at least 30 mM, at least 40 mM, at least 50 mM, at least 60 mM, at least 70 mM, at least 80 mM, at least 90 mM, at least 100 mM, at least 120 mM, at least 150 mM, or at least 200 mM. In some embodiments, the strength of the buffer is no more than 300 mM (e.g., at most 200 mM, at most 100 mM, at most 90 mM, at most 80 mM, at most 70 mM, at most 60 mM, at most 50 mM, at most 40 mM, at most 30 mM, at most 20 mM, at most 10 mM, at most 5 mM, at most 1 mM).

### Routes of Administration

The following discussion on routes of administration is merely provided to illustrate exemplary embodiments and should not be construed as limiting the scope in any way.

Formulations suitable for oral administration can consist of (a) liquid solutions, such as an effective amount of the conjugate of the present disclosure dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the conjugate of the present disclosure in a flavor, usually sucrose and acacia or tragacanth, as well as pastilles comprising the conjugate of the present disclosure in an inert base, such as gelatin and glycerin, or sucrose and acacia, emulsions, gels, and the like containing, in addition to, such excipients as are known in the art.

The conjugates of the disclosure, alone or in combination with other suitable components, can be delivered via pulmonary administration and can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa. In some embodiments, the conjugate is formulated into a powder blend or into microparticles or nanoparticles. Suitable pulmonary formulations are known in the art. See, e.g., Qian et al., Int J Pharm 366: 218-220 (2009); Adjei and Garren, Pharmaceutical Research, 7(6): 565-569 (1990); Kawashima et al., J Controlled Release 62(1-2): 279-287 (1999); Liu et al., Pharm Res 10(2): 228-232 (1993); International Patent Application Publication Nos, WO 2007133747 and WO 2007141411,

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The conjugate of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl -imidazoline quaternary ammonium salts, and (e) mixtures thereof.

The parenteral formulations will typically contain from about 0.5% to about 25% by weight of Ab-L-Y conjugate of the present disclosure in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Additionally, the conjugate of the present disclosures can be made into suppositories for rectal administration by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

It will be appreciated by one of skill in the art that, in addition to the above-described pharmaceutical compositions, the conjugate of the disclosure can be formulated as inclusion complexes, such as cyclodextrin inclusion complexes, or liposomes,

### Dose

The Ab-L-Y conjugates of the disclosure are believed to be useful in methods of treating an immunological disease or medical. For purposes of the disclosure, the amount or dose of the conjugate of the present disclosure administered should be sufficient to effect, e.g., a therapeutic or prophylactic response, in the subject or animal over a reasonable time frame. For example, the dose of the conjugate of the present disclosure should be sufficient to stimulate cAMP secretion from cells as described herein or sufficient to decrease blood glucose levels, fat levels, food intake levels, or body weight of a mammal, in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular conjugate of the present disclosure and the condition of the animal (e.g., human), as well as the body weight of the animal (e.g., human) to be treated.

Many assays for determining an administered dose are known in the art. For purposes herein, an assay, which comprises comparing the extent to which blood glucose levels are lowered upon administration of a given dose of the conjugate of the present disclosure to a mammal among a set of mammals of which is each given a different dose of the conjugate, could be used to determine a starting dose to be administered to a mammal. The extent to which blood glucose levels are lowered upon administration of a certain dose can be assayed by methods known in the art, including, for instance, the methods described herein in the Examples section.

The dose of the conjugate of the present disclosure also will be determined by the existence, nature and extent of any adverse side effects that might accompany the administration of a particular conjugate of the present disclosure. Typically, the attending physician will decide the dosage of the conjugate of the present disclosure with which to treat each individual patient, taking into consideration a variety of factors, such as age, body weight, general health, diet, sex, conjugate of the present disclosure to be administered, route of administration, and the severity of the condition being treated. By way of example and not intending to limit the invention, the dose of the conjugate of the present disclosure can be about 0.0001 to about 1 g/kg body weight of the subject being treated/day, from about 0.0001 to about 0.001 g/kg body weight/day, or about 0.01 mg to about 1 g/kg body weight/day.

In some embodiments, the pharmaceutical composition comprises any of the conjugates disclosed herein at a purity level suitable for administration to a patient. In some embodiments, the conjugate has a purity level of at least about 90%, about 91%, about 92%, about 93%, about 94%, about 95%, about 96%, about 97%, about 98% or about 99%, and a pharmaceutically acceptable diluent, carrier or excipient. The pharmaceutical composition in some aspects comprise the conjugate of the present disclosure at a concentration of at least A, wherein A is about 0.001 mg/ml, about 0.01 mg/ml, 0 about 1 mg/ml, about 0.5 mg/ml, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml or higher. In some embodiments, the pharmaceutical composition comprises the conjugate at a concentration of at most B, wherein B is about 30 mg/ml, about 25 mg/ml, about 24 mg/ml, about 23, mg/ml, about 22 mg/ml, about 21 mg/ml, about 20 mg/ml, about 19 mg/ml, about 18 mg/ml, about 17 mg/ml, about 16 mg/ml, about 15 mg/ml, about 14 mg/ml, about 13 mg/ml, about 12 mg/ml, about 11 mg/ml, about 10 mg/ml, about 9 mg/ml, about 8 mg/ml, about 7 mg/ml, about 6 mg/ml, about 5 mg/ml, about 4 mg/ml, about 3 mg/ml, about 2 mg/ml, about 1 mg/ml, or about 0.1 mg/ml. In some embodiments, the compositions may contain an conjugate at a concentration range of A to B mg/ml, for example, about 0.001 to about 30.0 mg/ml,

### Targeted Forms

One of ordinary skill in the art will readily appreciate that the Ab-L-Y conjugates of the disclosure can be modified in any number of ways, such that the therapeutic or prophylactic efficacy of the conjugate of the present disclosures is increased through the modification. For instance, the conjugate of the present disclosure can be further conjugated either directly or indirectly through a linker to a targeting moiety. The practice of conjugating compounds, e.g., glucagon conjugates described herein, to targeting moieties is known in the art. See, for instance, Wadhwa et al., J Drug Targeting, 3, 111-127 (1995) and U.S. Pat. No. 5,087,616. One of ordinary skill in the art recognizes that sites on the peptide of the present disclosures (Ab), which are not necessary for the function of the peptide of the present disclosures, are ideal sites for attaching a linker and/or a targeting moiety, provided that the linker and/or targeting moiety, once attached to the peptide of the present disclosures (Ab), does not interfere with the function of the peptide of the present disclosures.

### Controlled Release Formulations

Alternatively, the glucagon conjugates described herein can be modified into a depot form, such that the manner in which the conjugate of the present disclosures is released into the body to which it is administered is controlled with respect to time and location within the body (see, for example, U.S. Pat. No. 4,450,150). Depot forms of conjugate of the present disclosures can be, for example, an implantable composition comprising the conjugate of the present disclosures and a porous or non-porous material, such as a polymer, wherein the conjugate of the present disclosures is encapsulated by or diffused throughout the material and/or degradation of the non-porous material. The depot is then implanted into the desired location within the body and the conjugate of the present disclosures are released from the implant at a predetermined rate.

The pharmaceutical composition in certain aspects is modified to have any type of in vivo release profile. In some aspects, the pharmaceutical composition is an immediate release, controlled release, sustained release, extended release, delayed release, or bi-phasic release formulation. Methods of formulating peptides or conjugates for controlled release are known in the art. See, for example, Qian et al., J Pharm 374: 46-52 (2009) and International Patent Application Publication Nos. WO 2008130158, WO2004033036; WO2000032218; and WO 1999040942.

The instant compositions may further comprise, for example, micelles or liposomes, or some other encapsulated form, or may be administered in an extended release form to provide a prolonged storage and/or delivery effect. The disclosed pharmaceutical formulations may be administered according to any regime including, for example, daily (1 time per day, 2 times per day, 3 times per day, 4 times per day, 5 times per day, 6 times per day), every two days, every three days, every four days, every five days, every six days, weekly, bi-weekly, every three weeks, monthly, or bi-monthly.

### Kits

The Ab-L-Y conjugates of the present disclosure can be provided in accordance with one embodiment as part of a kit. Accordingly, in some embodiments, a kit for administering a Ab-L-Y conjugate to a patient in need thereof is provided wherein the kit comprises a Ab-L-Y conjugate as described herein.

In one embodiment the kit is provided with a device for administering the Ab-L-Y conjugate composition to a patient, e.g. syringe needle, pen device, jet injector or other needle-free injector. The kit may alternatively or in addition include one or more containers, e.g., vials, tubes, bottles, single or multi-chambered pre-filled syringes, cartridges, infusion pumps (external or implantable), jet injectors, pre-filled pen devices and the like, optionally containing the glucagon conjugate in a lyophilized form or in an aqueous solution. The kits in some embodiments comprise instructions for use. In accordance with one embodiment the device of the kit is an aerosol dispensing device, wherein the composition is prepackaged within the aerosol device. In another embodiment the kit comprises a syringe and a needle, and in one embodiment the sterile glucagon composition is prepackaged within the syringe.

In one embodiment the invention provides a compound of Formula (I): Ab-L-Y (I); wherein Ab comprises an anti prostate-specific membrane antigen (αPSMA) antibody or fragment thereof, further comprising a non-naturally encoded amino acid; L comprises a linker, linking group or a bond; Y comprises a nuclear receptor ligand; and wherein L is conjugated to Ab via a covalent linkage between said non-naturally encoded amino acid and L. In some embodiments, the present invention provides a compound of Formula (I): Ab-L-Y; wherein Y is an antagonist. In an additional embodiment, the present invention provides a compound of Formula (I): Ab-L-Y; wherein Y is an anti-androgenic molecule. In some embodiments, the present invention provides a compound of Formula (I): Ab-L-Y; wherein L is a cleavable, non-cleavable or degradable linker. In some embodiments, the present invention provides a compound of Formula (I): Ab-L-Y; wherein L is intracellularly cleavable or degradable. In some embodiments, the present invention provides a compound of Formula (I): Ab-L-Y; wherein the non-naturally encoded amino acid comprises a functional group selected from ketone and azide.

The following examples are given merely to illustrate the present invention and not in any way to limit its scope.

### EXAMPLES

### Example 1

### Synthesis of Compound 1

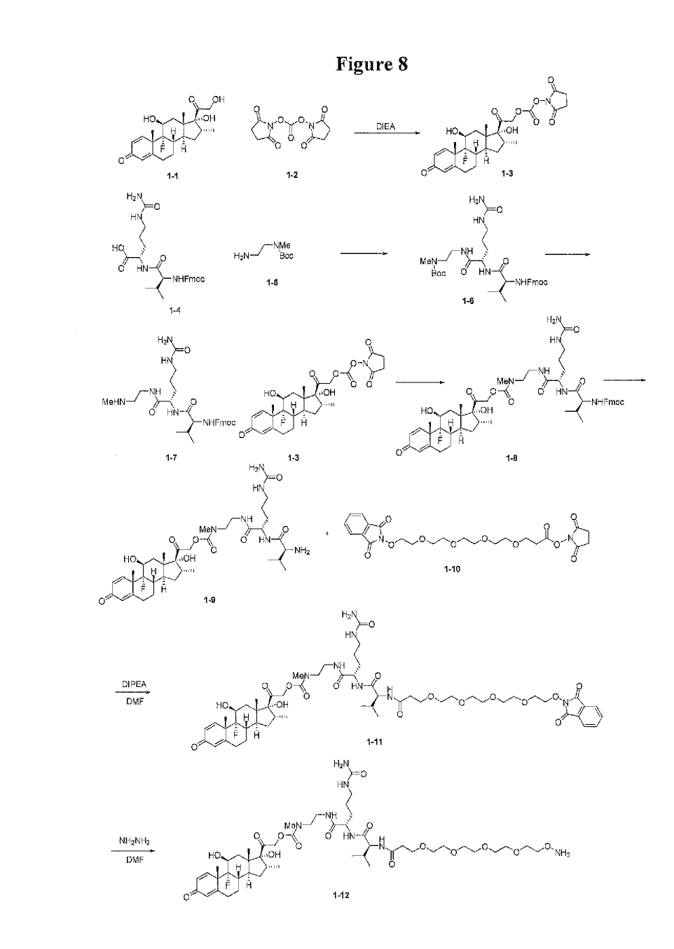

1. Detailed Synthesis of Compound 1 shown in FIG. 8

1a. Synthesis of Compound 1-3

To a mixture of Dexamethasone 1-1 (0.4 g, 1.02 mmol) and N, N′-disuccinimidyl carbonate (0.4 g, 1.33 mmol) in DCM (4 ml) and THF (4 ml) was added DIEA (0.36 ml, 2.04 mmol) at room temperature. The mixture was stirred at room temperature overnight. The mixture was concentrated and the crude product was purified by column chromatography. 0.13 g of 1-3 was obtained as white solid (24%). LCMS m/z=534 [M+H]+

1b. Synthesis of Compound 1-7

To a mixture of 1-4 (0.3 g, 0.6 mmol), 1-5 (0.12 g, 0.66 mmol) and EDC (0.2 g, 1.2 mmol) in DMF (6 ml) was added 1N NaHCO3 (1.8 mmol) solution at 0° C. The mixture was stirred at room temperature overnight. It was extracted with EtOAc (3×30 ml). Washed with 0.5M HCl and brine. The organic layer was dried over anhydrous MgSO4. It was filtered and concentrated under reduced pressure to give the product 1-6 as white solid,

A mixture of 1-6 (0.1 g) and 4N HCl in dioxane (1 ml) was stirred at room temperature for 1 hour. It was concentrated under reduced pressure to give the product 1-7 as white solid. The product was used without further purification. LCMS m/z=553 [M+H]+

1c. Synthesis of Compound 1-9

To a mixture of 1-3 (0.1 g, 0.18 mmol) and 1-7 (99.8 mg, 0.18 mmol) in DMF (3 ml) was added DIEA (0.16 ml, 0.9 mmol) at room temperature. The mixture was stirred at room temperature overnight. The crude product was purified by prep HPLC to give 65 mg of 1-8. It was dissolve into THF (1 ml) and Et2NH was added at room temperature. The mixture was stirred at room temperature for 2 hours and it was concentrated under reduced pressure to give the product 1-9 as white solid. The product was used without further purification. LCMS m/z=749 [M+H]+

1d. Synthesis of Compound 1-12

To a mixture of 1-9 (22 mg, 0.029 mmol) and 1-10 (16.4 mg, 0.032 mmol) in DMF (3 ml) was added DIEA (0.16 ml, 0.9 mmol) at room temperature. The mixture was stirred at room temperature for 4 hours. The crude product was purified by prep HPLC to give 15 mg of 1-11. LCMS m/z=1142 [M]+

1-11 was dissolved into DMF (1 ml) and NH2NH2 (6.3 mg) was added at room temperature. The mixture was stirred at room temperature for 1.5 hours and it was concentrated under reduced pressure. The crude product was purified by prep HPLC. 3 mg of 1-12 was obtained as white solid. LCMS m/z=1012 [M]+

### Example 2

### Synthesis of Compound 2

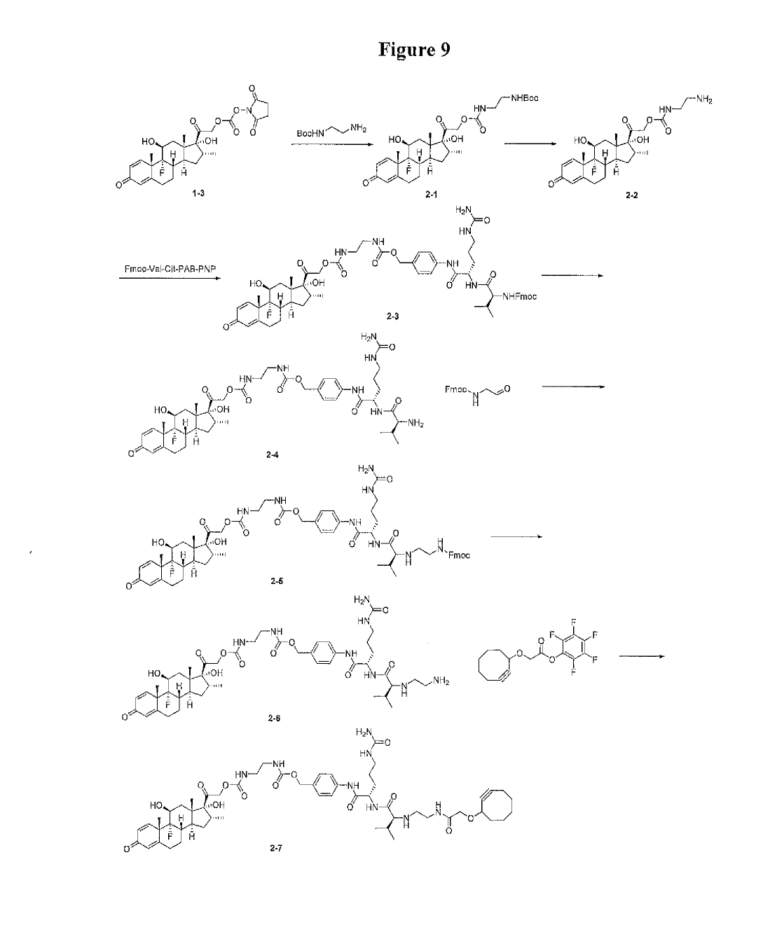

2. Detailed Synthesis of Compound 1 shown in FIG. 9

2a. Synthesis of Compound 2-2

To a mixture of 1-3 (0.1 g, 0.19 mmol) and tert-butyl 2-aminoethylcarbamate (30 mg, 0.19 mmol) in acetonitrile (2 ml) was added DIEA (0.098 ml, 0.56 mmol) at room temperature. The mixture was stirred at room temperature overnight. The white precipitate was filtered and washed with ether to give the product 2-1 as white solid,

A mixture of 2-1 (0.1 g) and 4N HCl in dioxane (1 ml) was stirred at room temperature for 1 hour. It was concentrated under reduced pressure to give the product 2-2 as white solid. The product was used without further purification. LCMS m/z=479 [M+1-1]+

2b. Synthesis of Compound 2-4

To a mixture of 2-2 (0.09 g, 0.188 mmol) and Fmoc-Val-Cit-PAB-PNP (0.159 g, 0.21 mmol) in DMF (1 ml) was added DIEA (0.16 ml, 0.94 mmol) at room temperature. The mixture was stirred at room temperature overnight. The crude product was purified by HPLC to give 0.1 g of 2-3 as white solid.

To a mixture of 2-3 (67 mg, 0.061 mmol) in THF (1 ml) was added Et2NH at room temperature. The mixture was stirred at room temperature for 2 hours and it was concentrated under reduced pressure and washed with ether. The product 2-4 was used without further purification. LCMS m/z=884 [M]+

2c. Synthesis of Compound 2-6

To a mixture of 2-4 (50 mg, 0.057 mmol) and NaOAc (36.7 mg, 0.45 mmol) in MeOH (3 ml) was added (9H-fluoren-9-yl)methyl 2-oxoethylcarbamate (19 mg, 0.068 mmol) at 0° C. The mixture was stirred at 0° C. for 0.5 hour. NaCNBH3 (9.2 mg, 0.15 mmol) was added. The mixture was stirred at 0° C. for another 15 minutes and was allowed to warm to room temperature for 4 hours. The reaction mixture was concentrated and purified by HPLC to 2-5 as white solid.

To a mixture of 2-5 (25 mg, 0.022 mmol) in THF (1 ml) was added Et2NH (31.8 mg, 0.44 mmol) at room temperature. The mixture was stirred at room temperature for 2 hours and it was concentrated under reduced pressure and washed with ether. The product 2-6 was used without further purification. LCMS m/z=927 [M]+

2d. Synthesis of Compound 2-7

To a mixture of 2-6 (14 mg, 0.015 mmol) and perfluorophenyl 2-(cyclooct-2-ynyloxy) acetate (5.2 mg, 0.015 mmol) in DMF (1 ml) was added DIEA (13 □l, 0.075 mmol) at room temperature. The mixture was stirred at room temperature overnight. The crude product was purified by HPLC to give 4 mg of 2-7 as white solid. LCMS m/z=1091 [M]+

### Example 3

### Synthesis of Compound 3

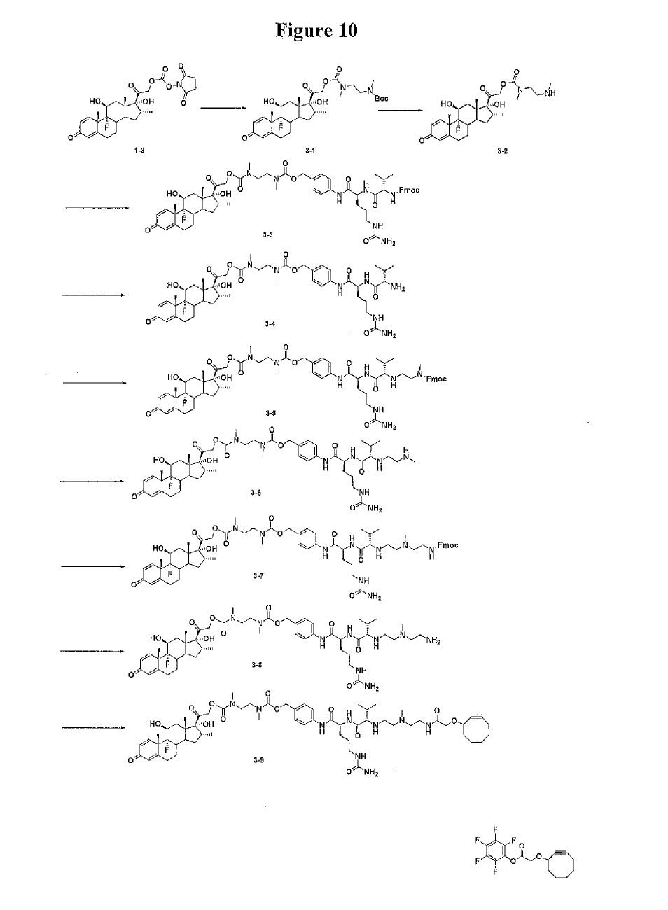

3. Detailed Synthesis of Compound 3 shown in FIG. 10.

3a. Synthesis of Compound 3-1

To the solution of compound 3 (600 mg, 1.125 mmol) in 0.5 mL of DMF was added tert-butyl methyl (2-(methylamino)ethyl)carbamate (127 mg, 0.675 mmol). The resulting solution was stirred at room temperature for 2 hrs. The reaction mixture was diluted with EtOAc and washed with H2O, brine, dried over Na2SO4, and then concentrated to dryness. The residue was purified by flash column chromatography to give 170 mg of compound 3-1. MS (ESI) m/z 607 [M+H].

3b. Synthesis of Compound 3-2

Compound 3-1 (170 mg) was treated with 50% TFA in DCM. The reaction was concentrated to dry after 30 min. The product was directly used in next step without further purification.

3c. Synthesis of Compound 3-3

To the solution of compound 3-3 (0,28 mmol) in 1,5 mL of DMF was added Fmoc-Val-Cit-PAB-OPNP (215 mg, 0,28 mmol), HOBt (21.4 mg, 0.14 mmol) and DIEA (99 □l, 0.56 mmol). The resulting solution was stirred at room temperature for 2 hrs. The reaction mixture was purified by HPLC to give 270 mg of compound 3-3. MS (EST) m/z 912 [M+H].

3d. Synthesis of Compound 3-4

Compound 3-3 (270 mg) was dissolved in 15 mL THF and 2 mL DMF, 5 mL of diethylamine was added to get a clear solution. The reaction was done in 1 hr. The reaction mixture was concentrated and purified by HPLC to get 180 mg of compound 3-4.

3e. Synthesis of Compound 3-5

To the solution of compound 3-4 (180 mg, 0.1974 mmol) in 1.5 mL of MeOH was added NaOAc (164 mg, 2 mmol) at 0° C., followed by (9H-Fluoren-9-yl)methyl methyl 2-oxoethylcarbamate (59 mg, 0.2 mmol). The resulting solution was stirred at 0° C. for 30 min. 11 mg of NaBH3CN was added at 0° C. The reaction mixture was stirred at 0° C. for 30 min and room temperature for 1 hr. The crude product was purified by HPLC to get 150 mg of compound 3-5. MS (ESI) m/z 1192 [M+H]

3f. Synthesis of Compound 3-6

Compound 3-5 (150 mg) was dissolved in 15 mL THF and 2 mL DMF. 5 mL of diethylamine was added to get a clear solution. The reaction was done in 1 hr. The reaction mixture was concentrated and purified by HPLC to get 110 mg of compound 3-6. MS (ESI) m/z 969 [M+H]

3g. Synthesis of Compound 3-7

To the solution of compound 3-6 (110 mg, 0.114 mmol) in 1.5 mL of MeOH was added NaOAc (93.5 mg, 1.14 mmol) at 0° C., followed by (9H-Fluoren-9-yl)methyl 2-oxoethylcarbamate (32 mg, 0.114 mmol). The resulting solution was stirred at 0° C. for 30 min. 7 mg of NaBH3CN was added at 0° C. The reaction mixture was stirred at 0° C. for 30 min and room temperature for 1 hr. The crude product was purified by HPLC to get 40 mg of compound 3-7. MS (ESI) m/z 1235 [M+H]

3h. Synthesis of Compound 3-8

Compound 3-7 (40 mg) was dissolved in 15 mL THF and 2 mL DMF. 5 mL of diethylamine was added to get a clear solution. The reaction was done in 1 hr. The reaction mixture was concentrated and purified by HPLC to get 12 mg of compound 3-8. MS (ESI) m/z 1012 [M+H], 507 [M+2H]

3i. Synthesis of Compound 3-9

To the solution of compound 3-8 (12 mg) in 1 mL DMF was added perfluorophenyl 2-(cyclooct-2-ynyloxy)acetate 4.5 mg. The reaction mixture was stirred at room temperature for 2 hrs and purified by HPLC to get 13 mg of compound 3-9. MS (ESI) m/z 1177 [M+1-1], 589 [M+2H].

### Example 4

### Synthesis of Compound 4

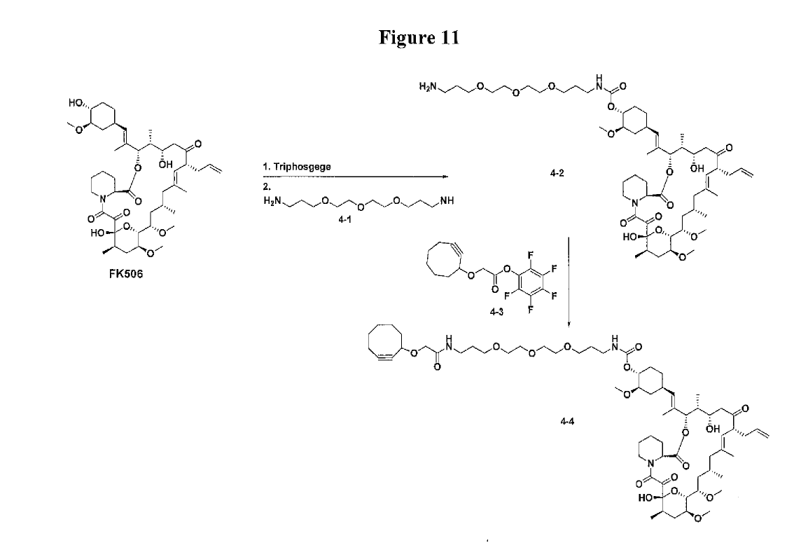

4. Detailed Synthesis of Compound 4 shown in FIG. 11,

4a. Synthesis of Compound 4-2

The reaction mixture of FK506 (140 mg, 0.17 mmol) in dichloromethane (4 mL) was treated with 4-DMAP (82 mg, 0.67 mmol). The solution of triphosgene (20 mg) in dichloromethane (2 mL) was slowly added at -78° C. (dry ice+acetone bath). The reaction mixture was stirred at -78° C. for 1 hour. Compound 4-1 (45 mg, 0.2 mmol) in dichloromethane (1.5 mL) was slowly added at -78° C. After addition, the reaction was stirred at −78° C. for 1 hour and then gradually increases to room temperature. The reaction mixture was treated with 1N HCl to adjust pH to 2. The reaction mixture was purified by prep-HPLC to get 35 mg of compound 4-2. MS (ESI) m/z 1051 [M+H]

4b. Synthesis of Compound 4-4

The reaction mixture of compound 4-2 (11 mg) in DMF (1 mL) was treated with active ester 4-3 (6.96 mg, 0.02 mmol) and DIEA (2.4 uL). The reaction was stirred at 0° C. for 1 hour and then increase to room temperature. The reaction mixture was adjust pH=2 and purified by prep-HPLC to give 9.1 mg of compound 4-4. MS (ESI) m/z 1215 [M+H]

### Example 5

### Synthesis of Compound 5

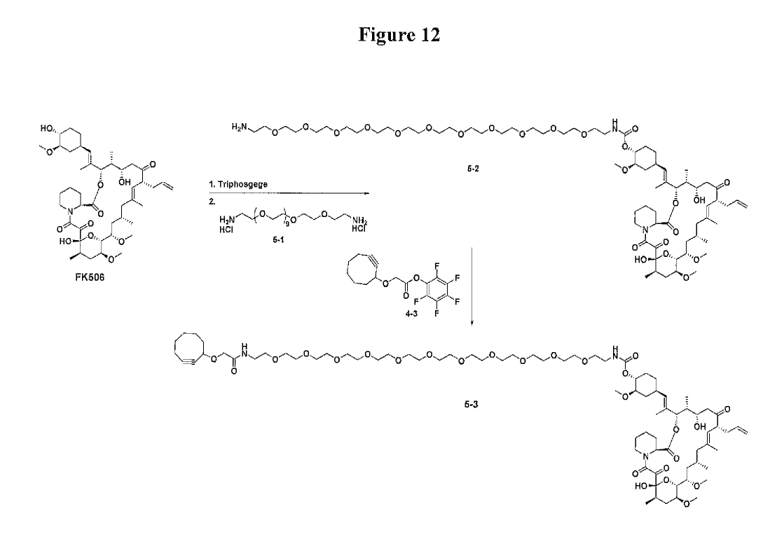

5. Detailed Synthesis of Compound 4 Shown in FIG. 12.

5a. Synthesis of Compound 5-2

The reaction mixture of FK506 (140 mg, 0.17 mmol) in dichloromethane (4 mL) was treated with 4-DMAP (82 mg, 0.67 mmol). The solution of triphosgene (20 mg, 0.051 mmol) in dichloromethane (2 mL) was slowly added at −78° C. (dry ice+acetone bath). The reaction mixture was stirred at −78° C. for 1 hour. Compound 5-1 (45 mg, 0.2 mmol) in dichloromethane (1.5 mL) was slowly added at -78° C. After addition, the reaction was stirred at −78° C. for 1 hour and then gradually increases to room temperature. The reaction mixture was treated with 1N HCl to adjust pH to 2. The reaction mixture was purified by prep-HPLC to give 78.3 mg of compound 5-2. MS (ESI) m/z 1375 [M+H]

5b. Synthesis of Compound 5-3

The reaction mixture of compound 5-2 (34.4 mg, 0.023 mmol) in DMF (1 mL) was treated with active ester (8 mg and 6 mg two portion) and DIEA (11.4 uL). The reaction was stirred at 0° C. for 1 hour and then increase to room temperature. The reaction mixture was adjust pH=2 and purified by prep-HPLC to give 11.1 mg of compound 5-3. MS (EST) m/z 1539 [M+H]

### Example 6

### Synthesis of Compound 6

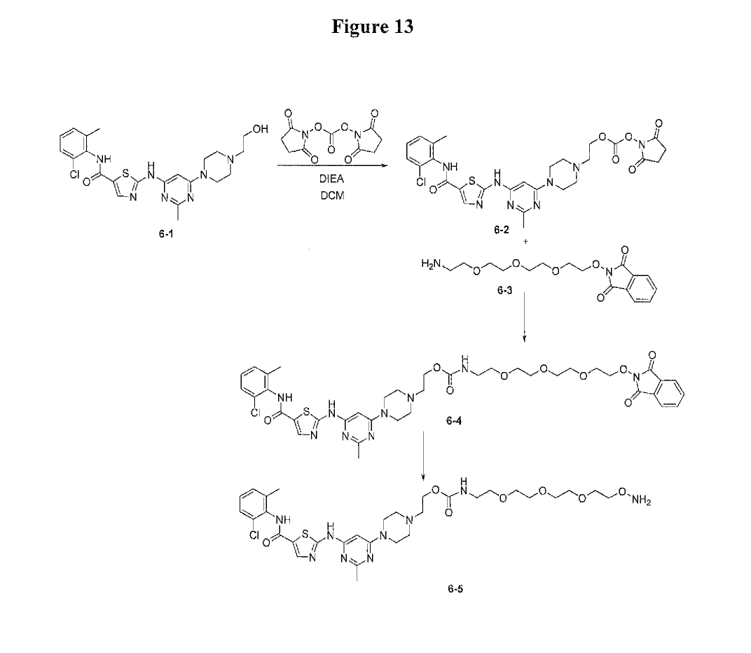

6. Detailed Synthesis of Compound 6 Shown in FIG. 13.

6a. Synthesis of Compound 6-2

To a mixture of Dasatinib 6-1 (0.1 g, 0.20 mmol) and N, N′-disuccinimidyl carbonate (0.102 g, 0.41 mmol) in DCM (8 ml) was added DIEA (0.11 ml, 0.61 mmol) at room temperature. The mixture was stirred at room temperature overnight. The mixture was concentrated and the crude product was purified by column chromatography to give 6-2 as white solid. LCMS m/z=629 [M]+

6b. Synthesis of Compound 6-5

To a mixture of 6-2 (50 mg, 0.079 mmol) and 6-3 (29.6 mg, 0.087 mmol) in DCM (5 ml) was added DIEA (0.041 ml, 0.24 mmol) at room temperature. The mixture was stirred at room temperature overnight. The crude product was purified by HPLC to give product 6-4 as white solid. (56%) LCMS m/z=852 [M]+

6-4 (38 mg, 0.045 mmol) was dissolve into DMF (1 ml) and NH2NH2 (14.4 mg) was added at room temperature. The mixture was stirred at room temperature for 4 hours and it was concentrated under reduced pressure. The crude product was purified by prep HPLC, 8 mg of 6-5 was obtained as white solid, LCMS m/z=722 [M]+

### Example 7

### αPSMA-Anti-Androgenic Conjugate 72 h Prostate Cancer Cell Line Viability Study

Anti-tumor efficacy of αPSMA-anti-androgenic conjugate is tested on prostate cancer cell lines LNCaP and MDA-PCa-2b. The two prostate cancer cell lines and PC-3 cells, used as negative control, are cultured and then treated either with αPSMA-anti-androgenic conjugate, the antibody alone, the anti-androgenic compound alone, and 72 hours following treatment cell viability is measured using the Dojindo cell counting kit-8 (WST-8 based).

### Example 8

### Human Clinical Trial of αPSMA-Anti-Androgenic Conjugate

Human Clinical Trial of the Safety and/or Efficacy of αPSMA-anti-androgenic conjugate for Prostate Cancer Therapy

Objective: To compare the safety and pharmacokinetics of administered composition comprising αPSMA-anti-androgenic conjugate.

Study Design: This study will be a Phase I, single-center, open-label, randomized dose escalation study followed by a Phase II study in prostate cancer patients. Patients should not have had exposure to αPSMA-anti-androgenic conjugate prior to the study entry, Patients must not have received treatment for their cancer within 2 weeks of beginning the trial. Treatments include the use of chemotherapy, hematopoietic growth factors, and biologic therapy such as monoclonal antibodies. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive i.v. αPSMA-anti-androgenic conjugate on days 1, 8, and 15 of each 28-day cycle, Doses of αPSMA-anti-androgenic conjugate may be held or modified for toxicity based on assessments as outlined below. Treatment repeats every 28 days in the absence of unacceptable toxicity. Cohorts of 3-6 patients receive escalating doses of αPSMA-anti-androgenic conjugate until the maximum tolerated dose (MTD) for αPSMA-anti-androgenic conjugate is determined. The MTD is defined as the dose preceding that at which 2 of 3 or 2 of 6 patients experience dose-limiting toxicity, Dose limiting toxicities are determined according to the definitions and standards set by the National Cancer Institute (NCI) Common Terminology for Adverse Events (CTCAE) Version 3.0 (Aug. 9, 2006).

Phase II: Patients receive αPSMA-anti-androgenic conjugate as in phase I at the MTD determined in phase I. Treatment repeats every 4 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling: serial blood is drawn by direct vein puncture before and after administration of αPSMA-anti-androgenic conjugate. Venous blood samples (5 mL) for determination of serum concentrations are obtained at about 10 minutes prior to dosing and at approximately the following times after dosing: days 1, 8, and 15. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 8, and 15. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles, Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH. After completion of study treatment, patients are followed periodically for 4 weeks.

Assays for nuclear receptor activity are known throughout the art. Nuclear receptor activity assays include, but are not limited to, Life Technologies GeneBLAzer®TR alpha DA(Division Arrested) cells and TR alpha-UAS-bla HEK 293T cells contain the ligand-binding domain (LBD) of the human Thyroid hormone receptor alpha(TR alpha) fused to the DNA-binding domain of GAL4 stably integrated in the GeneBLAzer®UAS-bla HEK 293T cell line. GeneBLAzer®UAS-bla HEK 293T cells stably express a beta-lactamase reporter gene under the transcriptional control of an upstream activator sequence (UAS). When an agonist binds to the LBD of the GAL4 (DBD)-TR alpha (LBD) fusion protein, the protein binds to the UAS, resulting in expression of beta-lactamase. Division Arrested (DA) cells are available in two configurations—an Assay Kit (which includes cells and sufficient substrate to analyze 1×384-well plate), and a tube of cells sufficient to analyze 10×384-well plates. DA cells are irreversibly division arrested using a low-dose treatment of Mitomycin-C, and have no apparent toxicity or change in cellular signal transduction.

TR alpha-UAS-bla HEK 293T cells contain the ligand-binding domain (LBD) of the human Thyroid hormone receptor alpha(TR alpha) fused to the DNA-binding domain of GAL4 stably integrated in the GeneBLAzer®UAS-bla HEK 293T cell line, GeneBLAzer®UAS-bla HEK 293T cells stably express a beta-lactamase reporter gene under the transcriptional control of an upstream activator sequence (UAS). When an agonist binds to the LBD of the GAL4 (DBD)-TR alpha (LBD) fusion protein, the protein binds to the UAS, resulting in expression of beta-lactamase. TR alpha-UASbla HEK 293T 293 cells are functionally validated for Z′ and EC50 concentrations of T3 Thryoid hormone; TR beta-UAS-bla HEK 293T cells contain the ligand-binding domain (LBD) of the human Thyroid hormone receptor beta(TR beta) fused to the DNA-binding domain of GAL4 stably integrated in the GeneBLAzer®UASbla HEK 293T cell line. GeneBLAzer®UAS-bla HEK 293T cells stably express a betalactamase reporter gene under the transcriptional control of an upstream activator sequence (UAS). When an agonist binds to the LBD of the GAL4 (DBD)-TR beta (LBD) fusion protein, the protein binds to the UAS, resulting in expression of beta-lactamase. Division Arrested (DA) cells are available in two configurations—an Assay Kit (which includes cells and sufficient substrate to analyze 1×384-well plate), and a tube of cells sufficient to analyze 10×384-well plates; and the Silencer® Select Human Nuclear Hormone Receptor siRNA Library V4, as well as numerous other biochemical nuclear receptor assays and cell-based nuclear receptor reporter assays that are commercially available.

It is understood that the examples and embodiments described herein are for illustrative purposes only and that various modifications or changes in light thereof will be suggested to those of ordinary skill in the art and are to be included within the spirit and purview of this application and scope of the appended claims. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A compound of Formula (I): 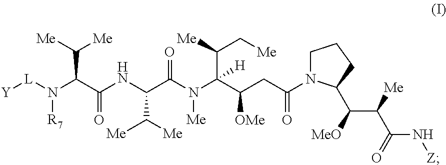
wherein Y comprises an anti prostate-specific membrane antigen (anti-PSMA) antibody or fragment thereof, further comprising a non-naturally encoded amino acid;
L comprises a linker, linking group or a bond;
and wherein L is conjugated to Ab via a covalent linkage between said non-naturally encoded amino acid and L.

2. The compound of claim 1 wherein Y is an antagonist.

3. The compound of claim 2 wherein Y is an anti-androgenic molecule.

4. The compound of claim 1 wherein L is a cleavable, non-cleavable or degradable linker.

5. The compound of claim 1 wherein L is intracellularly cleavable or degradable.

6. The compound of claim 1 wherein the non-naturally encoded amino acid comprises a functional group selected from ketone and azide.


SEQUENCE LISTING (12 sequences)

SEQ ID NO: 1 - protein, 16 aa, Homo sapiens
RSSQSLVHRNGNTYLH

SEQ ID NO: 2 - protein, 7 aa, Homo sapiens
TVSNRFS

SEQ ID NO: 3 - protein, 9 aa, Homo sapiens
SQSSHVPPT

SEQ ID NO: 4 - protein, 5 aa, Homo sapiens
NYGVN

SEQ ID NO: 5 - protein, 17 aa, Homo sapiens
WINPNTGEPTFDDDFKG

SEQ ID NO: 6 - protein, 11 aa, Homo sapiens
SRGKNEAWFAY

SEQ ID NO: 7 - protein, 5 aa, Homo sapiens
NYGMN

SEQ ID NO: 8 - protein, 17 aa, Homo sapiens
WINTYTREPTYADDFKG

SEQ ID NO: 9 - protein, 12 aa, Homo sapiens
DITAVVPTGFDY

SEQ ID NO: 10 - protein, 11 aa, Homo sapiens
RASENIYSNLA

SEQ ID NO: 11 - protein, 7 aa, Homo sapiens
AASNLAD

SEQ ID NO: 12 - protein, 9 aa, Homo sapiens
QHFWTTPWA